(19) United States
(12) Patent Application Publication
(10) Pub. No.: US 2024/0344060 A1
(43) Pub. Date: Oct. 17, 2024

(54) COMPOSITIONS AND METHODS FOR THE MODULATION OF ADAPTIVE IMMUNITY

(71) Applicant: Locanabio, Inc., San Diego, CA (US)

(72) Inventors: David A. NELLES, San Diego, CA (US); Ranjan BATRA, San Diego, CA (US); Eugene YEO, San Diego, CA (US)

(21) Appl. No.: 18/505,709

(22) Filed: Nov. 9, 2023

Related U.S. Application Data
(63) Continuation of application No. 16/434,787, filed on Jun. 7, 2019, now abandoned.
(60) Provisional application No. 62/682,276, filed on Jun. 8, 2018.

(51) Int. Cl. C12N 15/11; A61K 48/00

(52) U.S. Cl. CPC C12N 15/11; A61K 48/005; C12N 2310/20

(57) ABSTRACT

Disclosed are compositions and methods for simultaneously providing a gene therapy and preventing an adaptive immune response to a cell modified by the gene therapy by the immune system of a subject. In some embodiments, compositions of the disclosure modify a level of expression of an RNA molecule associated with a disease or disorder as well as inhibit expression or activity of a component of an adaptive immune response to mask the modified cell from a subject's immune system.

### RELATED APPLICATIONS

This application is a continuation of U.S. application Ser. No. 16/434,787, filed Jun. 7, 2019, which claims priority to, and the benefit of, U.S. Patent Application No. 62/682,276, filed Jun. 8, 2018, the contents of which are herein incorporated by reference in their entirety. The contents of International Application No. PCT/US2019/036021, filed Jun. 7, 2019, U.S. patent application Ser. No. 16/434,689, filed Jun. 7, 2019, and U.S. Patent Application No. 62/682,271, filed Jun. 8, 2018, are herein incorporated by reference in their entirety.

### FIELD OF THE DISCLOSURE

The disclosure is directed to molecular biology, and more, specifically, to compositions and methods for modifying expression and activity of RNA molecules involved in an adaptive immune response.

### INCORPORATION OF SEQUENCE LISTING

The instant application contains a Sequence Listing which has been submitted in XML format via Patent Center and is hereby incorporated by reference in its entirety. Said XML copy, created on Nov. 9, 2023 is named “LOCN-003_C01US_ST26.xml” and is 12,263,677 bytes in size.

### BACKGROUND

There has been a long-felt but unmet need in the art for simultaneously providing a gene therapy and suppressing the adaptive immune response that may arise when the gene therapy is delivered by, for example, a viral vector. The disclosure provides compositions and methods for specifically targeting RNA molecules in a sequence-specific manner that provides a gene therapy in vivo while masking the modified cells from the immune system of a subject, thereby preventing an adaptive immune response to the modified cell.

### SUMMARY

The disclosure provides a composition comprising a nucleic acid sequence comprising a guide RNA (gRNA) sequence that specifically binds a target RNA sequence, wherein the target RNA sequence encodes a protein component of an adaptive immune response, and wherein the gRNA sequence comprises a spacer sequence comprising a portion of a nucleic acid sequence encoding the protein component, and wherein the protein component is selected from the group consisting of Beta-2-microglobulin (β2M), Human Leukocyte Antigen A (HLA-A), Human Leukocyte Antigen B (HLA-B), Human Leukocyte Antigen C (HLA-C), Cluster of Differentiation 28 (CD28), Cluster of Differentiation 80 (CD80), Cluster of Differentiation 86 (CD86), Inducible T-cell Costimulator (ICOS), ICOS Ligand (ICOSLG), OX40L, Interleukin 12 (IL12), and CC Chemokine Receptor 7 (CCR7).

The disclosure also provides a composition comprising (a) a first sequence comprising a guide RNA (gRNA) that specifically binds a target sequence within an RNA molecule, wherein the target sequence comprises a sequence encoding a component of an adaptive immune response and (b) a sequence encoding a fusion protein, the sequence comprising a sequence encoding a first RNA-binding polypeptide and a sequence encoding a second RNA-binding polypeptide, wherein neither the first RNA-binding polypeptide nor the second RNA-binding polypeptide comprises a significant DNA-nuclease activity, wherein the first RNA-binding polypeptide and the second RNA-binding polypeptide are not identical, and wherein the second RNA-binding polypeptide comprises an RNA-nuclease activity.

The disclosure provides a composition comprising: (a) a first sequence comprising a guide RNA (gRNA) that specifically binds a first target sequence within a first RNA molecule, wherein the first target sequence comprises a sequence encoding a component of an adaptive immune response and (b) a second sequence comprising a second guide RNA (gRNA) that specifically binds a second target sequence within a second RNA molecule and (c) a sequence encoding a fusion protein, the sequence comprising a sequence encoding a first RNA-binding polypeptide and a sequence encoding a second RNA-binding polypeptide, wherein neither the first RNA-binding polypeptide nor the second RNA-binding polypeptide comprises a significant DNA-nuclease activity, wherein the first RNA-binding polypeptide and the second RNA-binding polypeptide are not identical, and wherein the second RNA-binding polypeptide comprises an RNA-nuclease activity.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the first target sequence or the second target sequence comprises at least one repeated sequence.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the first sequence comprising a first promoter capable of expressing the gRNA in a eukaryotic cell and/or the second sequence comprising a second promoter capable of expressing the gRNA in a eukaryotic cell. In some embodiments, the first promoter and the second promoter are identical. In some embodiments, the first promoter and the second promoter are not identical.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response, and a second sequence comprising a second guide RNA (gRNA) that specifically binds a second target sequence within a second RNA molecule, the first sequence and second sequence comprising a promoter capable of expressing the first gRNA and the second gRNA in a eukaryotic cell.

In some embodiments of the compositions of the disclosure, including those wherein a gRNA sequence comprises a promoter capable of expressing the gRNA in a eukaryotic cell, the eukaryotic cell is an animal cell. In some embodiments, the animal cell is a mammalian cell. In some embodiments, the animal cell is a human cell.

In some embodiments of the compositions of the disclosure, including those wherein a gRNA sequence comprises a promoter capable of expressing the gRNA in a eukaryotic cell, the promoter is a constitutively active promoter.

In some embodiments of the compositions of the disclosure, including those wherein a gRNA sequence comprises a promoter capable of expressing the gRNA in a eukaryotic cell, the gRNA sequence comprises a sequence isolated or derived from a promoter capable of driving expression of an RNA polymerase. In some embodiments, the promoter sequence is isolated or derived from a U6 promoter.

In some embodiments of the compositions of the disclosure, including those wherein a gRNA sequence comprises a promoter capable of expressing the gRNA in a eukaryotic cell, the promoter comprises a sequence isolated or derived from a promoter capable of driving expression of a transfer RNA (tRNA). In some embodiments, the promoter sequence is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter sequence is isolated or derived from a valine tRNA promoter.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the sequence comprising the first gRNA further comprises a first spacer sequence that specifically binds to the first target RNA sequence. In some embodiments, the first spacer sequence has at least 50%, 55%, 60%, 65%, 70%, 75%, 80%, 87%, 90%, 95%, 97%, 99% or any percentage in between of complementarity to the first target RNA sequence. In some embodiments, the first spacer sequence has 100% complementarity to the target RNA sequence. In some embodiments, the first spacer sequence comprises or consists of 20 nucleotides. In some embodiments, the first spacer sequence comprises or consists of 21 nucleotides. In some embodiments, the first spacer sequence comprises or consists of 20 nucleotides of an amino acid sequence encoding a Beta-2-microglobulin (β2M) protein. In some embodiments, the first spacer sequence comprises or consists of 20 nucleotides of an amino acid sequence of

(SEQ ID NO: 88)

MSRSVALAVL ALLSLSGLEA IQRTPKIQVY SRHPADIEVD
LLKNGERIEK VEHSDLSFSK DWSFYLLYYT EFTPTEKDEY
ACRVNHVTLS QPKIVKWDRD M.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the sequence comprising the first gRNA further comprises a first scaffold sequence that specifically binds to the first RNA binding protein. In some embodiments, the first scaffold sequence comprises a stem-loop structure. In some embodiments, the scaffold sequence comprises or consists of 90 nucleotides. In some embodiments, the scaffold sequence comprises or consists of 93 nucleotides. In some embodiments, the scaffold sequence comprises the sequence

(SEQ ID NO: 12)
GUUUAAGAGCUAUGCUGGAAACAGCAUAGCAAGUUUAAAUAAGGCUAGUC
CGUUAUCAACUUGAAAAAGUGGCACCGAGUCGGUGCUUUUUUU
or
(SEQ ID NO: 13)
GUUUUAGAGCUAGAAAUAGCAAGUUAAAAUAAGGCUAGUCCGUUAUCAAC
UUGAAAAAGUGGCACCGAGUCGGUGCUUUUUUU.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the sequence comprising the second gRNA further comprises a second spacer sequence that specifically binds to the second target RNA sequence. In some embodiments, the second spacer sequence has at least 50%, 55%, 60%, 65%, 70%, 75%, 80%, 87%, 90%, 95%, 97%, 99% or any percentage in between of complementarity to the first target RNA sequence. In some embodiments, the second spacer sequence has 100% complementarity to the target RNA sequence. In some embodiments, the second spacer sequence comprises or consists of 20 nucleotides. In some embodiments, the second spacer sequence comprises or consists of 21 nucleotides. In some embodiments, the second spacer sequence comprises or further comprises a sequence comprising at least 1, 2, 3, 4, 5, 6, or 7 repeats of the sequence CUG (SEQ ID NO: 18), CCUG (SEQ ID NO: 19), CAG (SEQ ID NO: 80), GGGGCC (SEQ ID NO: 81) or any combination thereof.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the sequence comprising the second gRNA further comprises a second scaffold sequence that specifically binds to the first RNA binding protein. In some embodiments, the second scaffold sequence comprises a stem-loop structure. In some embodiments, the scaffold sequence comprises or consists of 85 nucleotides. In some embodiments, the scaffold sequence comprises the sequence

(SEQ ID NO: 12)
GUUUAAGAGCUAUGCUGGAAACAGCAUAGCAAGUUUAAAUAAGGCUAGU
CCGUUAUCAACUUGAAAAAGUGGCACCGAGUCGGUGCUUUUUUU
or
(SEQ ID NO: 13)
GUUUUAGAGCUAGAAAUAGCAAGUUAAAAUAAGGCUAGUCCGUUAUCAA
CUUGAAAAAGUGGCACCGAGUCGGUGCUUUUUUU.

In some embodiments of the compositions of the disclosure, the gRNA does not bind or does not selectively bind to a second sequence within the RNA molecule.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the first gRNA does not bind or does not selectively bind to a second sequence within the first RNA molecule.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second gRNA does not bind or does not selectively bind to a second sequence within the second RNA molecule.

In some embodiments of the compositions of the disclosure, an RNA genome or an RNA transcriptome comprises the RNA molecule.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, an RNA genome or an RNA transcriptome comprises the first RNA molecule or the second RNA molecule.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the first RNA binding protein comprises a CRISPR-Cas protein. In some embodiments, the CRISPR-Cas protein is a Type II CRISPR-Cas protein. In some embodiments, the first RNA binding protein comprises a Cas9 polypeptide or an RNA-binding portion thereof. In some embodiments, the CRISPR-Cas protein is a Type V CRISPR-Cas protein. In some embodiments, the first RNA binding protein comprises a Cpf1 polypeptide or an RNA-binding portion thereof. In some embodiments, the CRISPR-Cas protein is a Type VI CRISPR-Cas protein. In some embodiments, the first RNA binding protein comprises a Cas13 polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and wherein the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the first RNA binding protein comprises a Pumilio and FBF (PUF) protein or an RNA binding portion thereof. In some embodiments, the first RNA binding protein comprises a Pumilio-based assembly (PUMBY) protein or an RNA binding portion thereof.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the first RNA binding protein does not require multimerization for RNA-binding activity. In some embodiments, the first RNA binding protein is not a monomer of a multimer complex. In some embodiments, a multimer protein complex does not comprise the first RNA binding protein.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the first RNA binding protein selectively binds to a target sequence within the RNA molecule. In some embodiments, the first RNA binding protein does not comprise an affinity for a second sequence within the RNA molecule. In some embodiments, the first RNA binding protein does not comprise a high affinity for or selectively bind a second sequence within the RNA molecule. In some embodiments, an RNA genome or an RNA transcriptome comprises the RNA molecule.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the first RNA binding protein comprises between 2 and 1300 amino acids, inclusive of the endpoints.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the sequence encoding the first RNA binding protein further comprises a sequence encoding a nuclear localization signal (NLS). In some embodiments, the sequence encoding a nuclear localization signal (NLS) is positioned 3′ to the sequence encoding the first RNA binding protein. In some embodiments, the first RNA binding protein comprises an NLS at a C-terminus of the protein.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the sequence encoding the first RNA binding protein further comprises a first sequence encoding a first NLS and a second sequence encoding a second NLS. In some embodiments, the sequence encoding the first NLS or the second NLS is positioned 3′ to the sequence encoding the first RNA binding protein. In some embodiments, the first RNA binding protein comprises the first NLS or the second NLS at a C-terminus of the protein.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a nuclease domain. In some embodiments, the second RNA binding protein comprises or consists of an RNAse. In some embodiments, the second RNA binding protein comprises or consists of an RNAse1. In some embodiments, the RNAse1 protein comprises or consists of SEQ ID NO: 20. In some embodiments, the second RNA binding protein comprises or consists of an RNAse4. In some embodiments, the RNAse4 protein comprises or consists of SEQ ID NO: 21. In some embodiments, the second RNA binding protein comprises or consists of an RNAse6. In some embodiments, the RNAse6 protein comprises or consists of SEQ ID NO: 22. In some embodiments, the second RNA binding protein comprises or consists of an RNAse7. In some embodiments, the RNAse7 protein comprises or consists of SEQ ID NO: 23. In some embodiments, the second RNA binding protein comprises or consists of an RNAse8. In some embodiments, the RNAse8 protein comprises or consists of SEQ ID NO: 24. In some embodiments, the second RNA binding protein comprises or consists of an RNAse2. In some embodiments, the RNAse2 comprises or consists of SEQ ID NO: 25. In some embodiments, the second RNA binding protein comprises or consists of an RNAse6PL. In some embodiments, the RNAse6PL protein comprises or consists of SEQ ID NO: 26. In some embodiments, the second RNA binding protein comprises or consists of an RNAseL. In some embodiments, the RNAseL protein comprises or consists of SEQ ID NO: 27. In some embodiments, the second RNA binding protein comprises or consists of an RNAseT2. In some embodiments, the RNAseT2 protein comprises or consists of SEQ ID NO: 28. In some embodiments, the second RNA binding protein comprises or consists of an RNAse11. In some embodiments, the RNAse11 protein comprises or consists of SEQ ID NO: 29. In some embodiments, the second RNA binding protein comprises or consists of an RNAseT2-like. In some embodiments, the RNAseT2-like protein comprises or consists of SEQ ID NO: 30.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a NOB1 polypeptide. In some embodiments, the NOB1 polypeptide comprises or consists of SEQ ID NO: 31.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of an endonuclease. In some embodiments, the second RNA binding protein comprises or consists of an endonuclease V (ENDOV. In some embodiments, the ENDOV comprises or consists of SEQ ID NO: 32. In some embodiments, the second RNA binding protein comprises or consists of an endonuclease G (ENDOG). In some embodiments, the ENDOG comprises or consists of SEQ ID NO: 33. In some embodiments, the second RNA binding protein comprises or consists of an endonuclease D1 (ENDOD1). In some embodiments, the ENDOD1 comprises or consists of SEQ ID NO: 34.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Human flap endonuclease-1 (hFEN1). In some embodiments, the hFEN1 comprises or consists of SEQ ID NO: 35.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a human Schlafen 14 (hSLFN14) polypeptide. In some embodiments, the hSLFN14 comprises or consists of SEQ ID NO: 36.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a human beta-lactamase-like protein 2 (hLACTB2) polypeptide. In some embodiments, the hLACTB2 comprises or consists of SEQ ID NO: 37.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of an apurinic/apyrimidinic (AP) endodeoxyribonuclease (APEX2) polypeptide. In some embodiments, the APEX2 comprises or consists of SEQ ID NO: 38. In some embodiments, the APEX2 comprises or consists of SEQ ID NO: 39.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of an angiogenin (ANG) polypeptide. In some embodiments, the ANG comprises or consists of SEQ ID NO: 40.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a heat responsive protein 12 (HRSP12) polypeptide. In some embodiments, the HRSP12 comprises or consists of SEQ ID NO: 41.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Zinc Finger CCCH-Type Containing 12A (ZC3H12A). In some embodiments, the ZC3H12A comprises or consists of SEQ ID NO: 42. In some embodiments, the ZC3H12A comprises or consists of SEQ ID NO: 43.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Reactive Intermediate Imine Deaminase A (RIDA) polypeptide. In some embodiments, the RIDA polypeptide comprises or consists of SEQ ID NO: 44.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Phospholipase D Family Member 6 (PDL6) polypeptide. In some embodiments, the PDL6 polypeptide comprises or consists of SEQ ID NO: 126.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Endonuclease III-like protein 1 (NTHL) polypeptide. In some embodiments, the NTHL polypeptide comprises or consists of SEQ ID NO: 123.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Mitochondrial ribonuclease P catalytic subunit (KIAA0391) polypeptide. In some embodiments, the KIAA0391 polypeptide comprises or consists of SEQ ID NO: 127.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of an apurinic or apyrimidinic site lyase (APEX1) polypeptide. In some embodiments, the APEX1 polypeptide comprises or consists of SEQ ID NO: 125.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of an argonaute 2 (AGO2) polypeptide. In some embodiments, encoding the AGO2 polypeptide comprises or consists of SEQ ID NO: 128.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a mitochondrial nuclease EXOG (EXOG) polypeptide. In some embodiments, the EXOG polypeptide comprises or consists of SEQ ID NO: 129.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Zinc Finger CCCH-Type Containing 12D (ZC3H12D) polypeptide. In some embodiments, the ZC3H12D polypeptide comprises or consists of SEQ ID NO: 130.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of an endoplasmic reticulum to nucleus signaling 2 (ERN2) polypeptide. In some embodiments, the ERN2 polypeptide comprises or consists of SEQ ID NO: 131.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a pelota mRNA surveillance and ribosome rescue factor (PELO) polypeptide. In some embodiments, the PELO polypeptide comprises or consists of SEQ ID NO: 132.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a YBEY metallopeptidase (YBEY) polypeptide. In some embodiments, the YBEY polypeptide comprises or consists of SEQ ID NO: 133.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule the second RNA binding protein comprises or consists of a cleavage and polyadenylation specific factor 4 like (CPSF4L) polypeptide. In some embodiments, the CPSF4L polypeptide comprises or consists of SEQ ID NO: 134.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of an hCG_2002731 polypeptide. In some embodiments, the hCG_2002731 polypeptide comprises or consists of SEQ ID NO: 135. In some embodiments, the sequence encoding the hCG_2002731 polypeptide comprises or consists of SEQ ID NO: 136.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of an Excision Repair Cross-Complementation Group 1 (ERCC1) polypeptide. In some embodiments, the ERCC1 polypeptide comprises or consists of SEQ ID NO: 137.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a ras-related C3 botulinum toxin substrate 1 isoform (RAC1) polypeptide. In some embodiments, the RAC1 polypeptide comprises or consists of SEQ ID NO: 138.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Ribonuclease A A1 (RAA1) polypeptide. In some embodiments, the RAA1 polypeptide comprises or consists of SEQ ID NO: 139.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Ras Related Protein (RAB1) polypeptide. In some embodiments, the RAB1 polypeptide comprises or consists of SEQ ID NO: 140.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a DNA Replication Helicase/Nuclease 2 (DNA2) polypeptide. In some embodiments, the DNA2 polypeptide comprises or consists of SEQ ID NO: 141.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a FLJ35220 polypeptide. In some embodiments, the FLJ35220 polypeptide comprises or consists of SEQ ID NO: 142.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a FLJ13173 polypeptide. In some embodiments, the FLJ13173 polypeptide comprises or consists of SEQ ID NO: 143.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule the second RNA binding protein comprises or consists of a DNA repair endonuclease XPF (ERCC4) polypeptide. In some embodiments, the ERCC4 polypeptide comprises or consists of SEQ ID NO: 124.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a mutated Rnase1 (Rnase1(K41R)) polypeptide. In some embodiments, the Rnase1(K41R) polypeptide comprises or consists of SEQ ID NO: 116.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a mutated Rnase1 (Rnase1(K41R, D121E)) polypeptide. In some embodiments, the Rnase1 (Rnase1(K41R, D121E)) polypeptide comprises or consists of SEQ ID NO: 117).

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a mutated Rnase1 (Rnase1(K41R, D121E, H119N)) polypeptide. In some embodiments, the Rnase1 (Rnase1(K41R, D121E, H119N)) polypeptide comprises or consists of SEQ ID NO: 118.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a mutated Rnase1 (Rnase1(H119N)) polypeptide. In some embodiments, the Rnase1 (Rnase1(H119N)) polypeptide comprises or consists of SEQ ID NO: 119.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D, H119N)) polypeptide comprises or consists of SEQ ID NO: 120.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D, H119N, K41R, D121E)) polypeptide comprises or consists of SEQ ID NO: 121.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D)) polypeptide comprises or consists of SEQ ID NO: 122.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Teneurin Transmembrane Protein 1 (TENM1) polypeptide. In some embodiments, the TENM1 polypeptide comprises or consists of SEQ ID NO: 144.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Teneurin Transmembrane Protein 1 (TENM2) polypeptide. In some embodiments, the TENM2 polypeptide comprises or consists of SEQ ID NO: 145.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a Ribonuclease Kappa (RNAseK) polypeptide. In some embodiments, the RNAseK protein comprises or consists of SEQ ID NO: 204.

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a transcription activator-like effector nuclease (TALEN) polypeptide or a nuclease domain thereof. In some embodiments, the TALEN polypeptide comprises or consists of:

| (SEQ ID NO: 205) | |
|---|---|
| 1 | MRIGKSSGWL NESVSLEYEH VSPPTRPRDT RRRPRAAGDG GLAHLHRRLA VGYAEDTPRT |
| 61 | EARSPAPRRP LPVAPASAPP APSLVPEPPM PVSLPAVSSP RFSAGSSAAI TDPFPSLPPT |
| 121 | PVLYAMAREL EALSDATWQP AVPLPAEPPT DARRGNTVFD EASASSPVIA SACPQAFASP |
| 181 | PRAPRSARAR RARTGGDAWP APTFLSRPSS SRIGRDVFGK LVALGYSREQ IRKLKQESLS |
| 241 | EIAKYHTTLT GQGFTHADIC RISRRRQSLR VVARNYPELA AALPELTRAH IVDIARQRSG |
| 301 | DLALQALLPV ATALTAAPLR LSASQIATVA QYGERPAIQA LYRLRRKLTR APLHLTPQQV |
| 361 | VAIASNTGGK RALEAVCVQL PVLRAAPYRL STEQVVAIAS NKGGKQALEA VKAHLLDLLG |
| 421 | APYVLDTEQV VAIASHNGGK QALEAVKADL LDLRGAPYAL STEQVVAIAS HNGGKQALEA |
| 481 | VKADLLELRG APYALSTEQV VAIASHNGGK QALEAVKAHL LDLRGVPYAL STEQVVAIAS |
| 541 | HNGGKQALEA VKAQLLDLRG APYALSTAQV VAIASNGGGK QALEGIGEQL LKLRTAPYGL |
| 601 | STEQVVAIAS HDGGKQALEA VGAQLVALRA APYALSTEQV VAIASNKGGK QALEAVKAQL |
| 661 | LELRGAPYAL STAQVVAIAS HDGGNQALEA VGTQLVALRA APYALSTEQV VAIASHDGGK |
| 721 | QALEAVGAQL VALRAAPYAL NTEQVVAIAS SHGGKQALEA VRALFPDLRA APYALSTAQL |
| 781 | VAIASNPGGK QALEAVRALF RELRAAPYAL STEQVVAIAS NHGGKQALEA VRALFRGLRA |
| 841 | APYGLSTAQV VAIASSNGGK QALEAVWALL PVLRATPYDL NTAQIVAIAS HDGGKPALEA |
| 901 | VWAKLPVLRG APYALSTAQV VAIACISGQQ ALEAIEAHMP TLRQASHSLS PERVAAIACI |
| 961 | GGRSAVEAVR QGLPVKAIRR IRREKAPVAG PPPASLGPTP QELVAVLHFF RAHQQPRQAF |
| 1021 | VDALAAFQAT RPALLRLLSS VGVTEIEALG GTIPDATERW QRLLGRLGFR PATGAAAPSP |
| 1081 | DSLQGFAQSL ERTLGSPGMA GQSACSPHRK RPAETAIAPR SIRRSPNNAG QPSEPWPDQL |
| 1141 | AWLQRRKRTA RSHIRADSAA SVPANLHLGT RAQFTPDRLR AEPGPIMQAH TSPASVSFGS |
| 1201 | HVAFEPGLPD PGTPTSADLA SFEAEPFGVG PLDFHLDWLL QILET. |

In some embodiments, the TALEN polypeptide comprises or consists of:

| (SEQ ID NO: 206) | |
|---|---|
| 1 | mdpirsrtps parellpgpq pdrvqptadr ggappaggpl dglparrtms rtrlpsppap |
| 61 | spafsagsfs dllrqfdpsl ldtslldsmp avgtphtaaa paecdevqsg lraaddpppt |
| 121 | vrvavtaarp prakpaprrr aaqpsdaspa aqvdlrtlgy sqqqqekikp kvgstvaqhh |
| 181 | ealvghgfth ahivalsrhp aalgtvavky qdmiaalpea thedivgvgk qwsgaralea |
| 241 | lltvagelrg pplqldtgql vkiakrggvt aveavhasrn altgaplnlt paqvvaiasn |
| 301 | nggkqaletv qrllpvlcqa hgltpaqvva iashdggkqa letmqrllpv lcqahglppd |
| 361 | qvvaiasnig gkqaletvqr llpvlcqahg ltpdqvvaia shgggkqale tvqrllpvlc |
| 421 | qahgltpdqv vaiashdggk qaletvqrll pvlcqahglt pdqvvaiasn gggkqaletv |
| 481 | qrllpvlcqa hgltpdqvva iasnggkqal etvqrllpvl cqahgltpdq vvaiashdgg |
| 541 | kqaletvqrl lpvlcqthgl tpaqvvaias hdggkqalet vqqllpvlcq ahgltpdqvv |
| 601 | aiasniggkq alatvqrllp vlcqahgltp dqvvaiasng ggkqaletvq rllpvlcqah |
| 661 | gltpdqvvai asngggkqal etvqrllpvl cqahgltqvq vvaiasnigg kqaletvqrl |
| 721 | lpvlcqahgl tpaqvvaias hdggkqalet vqrllpvlcq ahgltpdqvv aiasngggkq |
| 781 | aletvqrllp vlcqahgltq eqvvaiasnn ggkqaletvq rllpvlcqah gltpdqvvai |
| 841 | asngggkqal etvqrllpvl cqahgltpaq vvaiasnigg kqaletvqrl lpvlcqdhgl |
| 901 | tlaqvvaias niggkqalet vqrllpvlcq ahgltqdqvv aiasniggkq aletvqrllp |
| 961 | vlcqdhgltp dqvvaiasni ggkqaletvq rllpvlcqdh gltldqvvai asnggkqale |
| 1021 | tvqrllpvlc qdhgltpdqv vaiasnsggk qaletvqrll pvlcqdhglt pnqvvaiasn |
| 1081 | ggkqalesiv aqlsrpdpal aaltndhlva laclggrpam davkkglpha pelirrvnrr |
| 1141 | igertshrva dyaqvvrvle ffqchshpay afdeamtqfg msrnglvqlf rrvgvtelea |
| 1201 | rggtlppasq rwdrilqasg mkrakpspts aqtpdqaslh afadslerdl dapspmhegd |
| 1261 | qtgassrkrs rsdravtgps aqhsfevrvp eqrdalhlpl swrvkrprtr iggglpdpgt |
| 1321 | piaadlaass tvmweqdaap fagaaddfpa fneeelawlm ellpqsgsvg gti. |

In some embodiments of the compositions of the disclosure, including those wherein the composition comprises a first sequence comprising a first guide RNA (gRNA) that specifically binds a first target sequence within a first RNA molecule, wherein the first target sequence comprises a sequence encoding a component of an adaptive immune response and a second sequence comprising a second guide RNA (gRNA) that specifically binds a second target sequence within a second RNA molecule, the second RNA binding protein comprises or consists of a zinc finger nuclease polypeptide or a nuclease domain thereof. In some embodiments, the second RNA binding protein comprises or consists of a ZNF638 polypeptide or a nuclease domain thereof. In some embodiments, the ZNF638 polypeptide polypeptide comprises or consists of:

| (SEQ ID NO: 207) | |
|---|---|
| 1 | MSRPRFNPRG DFPLQRPRAP NPSGMRPPGP FMRPGSMGLP RFYPAGRARG IPHRFAGHES |
| 61 | YQNMGPQRMN VQVTQHRTDP RLTKEKLDFH EAQQKKGKPH GSRWDDEPHI SASVAVKQSS |
| 121 | VTQVTEQSPK VQSRYTKESA SSILASFGLS NEDLEELSRY PDEQLTPENM PLILRDIRMR |
| 181 | KMGRRLPNLP SQSRNKETLG SEAVSSNVID YGHASKYGYT EDPLEVRIYD PEIPTDEVEN |
| 241 | EFQSQQNISA SVPNPNVICN SMFPVEDVFR QMDFPGESSN NRSFFSVESG TKMSGLHISG |
| 301 | GQSVLEPIKS VNQSINQTVS QTMSQSLIPP SMNQQPFSSE LISSVSQQER IPHEPVINSS |
| 361 | NVHVGSRGSK KNYQSQADIP IRSPFGIVKA SWLPKFSHAD AQKMKRLPTP SMMNDYYAAS |
| 421 | PRIFPHLCSL CNVECSHLKD WIQHQNTSTH IESCRQLRQQ YPDWNPEILP SRRNEGNRKE |
| 481 | NETPRRRSHS PSPRRSRRSS SSHRFRRSRS PMHYMYRPRS RSPRICHRFI SRYRSRSRSR |
| 541 | SPYRIRNPFR GSPKCFRSVS PERMSRRSVR SSDRKKALED VVQRSGHGTE FNKQKHLEAA |
| 601 | DKGHSPAQKP KTSSGTKPSV KPTSATKSDS NLGGHSIRCK SKNLEDDTLS ECKQVSDKAV |
| 661 | SLQRKLRKEQ SLHYGSVLLI TELPEDGCTE EDVRKLFQPF GKVNDVLIVP YRKEAYLEME |
| 721 | FKEAITAIMK YIETTPLTIK GKSVKICVPG KKKAQNKEVK KKTLESKKVS ASTLKRDADA |
| 781 | SKAVEIVTST SAAKTGQAKA SVAKVNKSTG KSASSVKSVV TVAVKGNKAS IKTAKSGGKK |
| 841 | SLEAKKTGNV KNKDSNKPVT IPENSEIKTS IEVKATENCA KEAISDAALE ATENEPLNKE |
| 901 | TEEMCVMLVS NLPNKGYSVE EVYDLAKPFG GLKDILILSS HKKAYIEINR KAAESMVKFY |
| 961 | TCFPVLMDGN QLSISMAPEN MNIKDEEAIF ITLVKENDPE ANIDTIYDRF VHLDNLPEDG |
| 1021 | LQCVLCVGLQ FGKVDHHVFI SNRNKAILQL DSPESAQSMY SFLKQNPQNI GDHMLTCSLS |
| 1081 | PKIDLPEVQI EHDPELEKES PGLKNSPIDE SEVQTATDSP SVKPNELEEE STPSIQTETL |
| 1141 | VQQEEPCEEE AEKATCDSDF AVETLELETQ GEEVKEEIPL VASASVSIEQ FTENAEECAL |
| 1201 | NQQMFNSDLE KKGAEIINPK TALLPSDSVF AEERNLKGIL EESPSEAEDF ISGITQTMVE |
| 1261 | AVAEVEKNET VSEILPSTCI VTLVPGIPTG DEKTVDKKNI SEKKGNMDEK EEKEFNTKET |
| 1321 | RMDLQIGTEK AEKNEGRMDA EKVEKMAAMK EKPAENTLFK AYPNKGVGQA NKPDETSKTS |
| 1381 | ILAVSDVSSS KPSIKAVIVS SPKAKATVSK TENQKSFPKS VPRDQINAEK KLSAKEFGLL |
| 1441 | KPTSARSGLA ESSSKFKPTQ SSLTRGGSGR ISALQGKLSK LDYRDITKQS QETEARPSIM |
| 1501 | KRDDSNNKTL AEQNTKNPKS TTGRSSKSKE EPLFPFNLDE FVTVDEVIEE VNPSQAKQNP |
| 1561 | LKGKRKETLK NVPFSELNLK KKKGKTSTPR GVEGELSFVT LDEIGEEEDA AAHLAQALVT |
| 1621 | VDEVIDEEEL NMEEMVKNSN SLFTLDELID QDDCISHSEP KDVTVLSVAE EQDLLKQERL |
| 1681 | VTVDEIGEVE ELPLNESADI TFATLNTKGN EGDTVRDSIG FISSQVPEDP STLVTVDEIQ |
| 1741 | DDSSDLHLVT LDEVTEEDED SLADFNNLKE ELNFVTVDEV GEEEDGDNDL KVELAQSKND |
| 1801 | HPTDKKGNRK KRAVDTKKTK LESLSQVGPV NENVMEEDLK TMIERHLTAK TPTKRVRIGK |
| 1861 | TLPSEKAVVT EPAKGEEAFQ MSEVDEESGL KDSEPERKRK KTEDSSSGKS VASDVPEELD |
| 1921 | FLVPKAGFFC PICSLFYSGE KAMTNHCKST RHKQNTEKFM AKQRKEKEQN EAEERSSR. |

In some embodiments of the compositions of the disclosure, the composition further comprises (a) a sequence comprising a gRNA that specifically binds within an RNA molecule and (b) a sequence encoding a nuclease. In some embodiments, the sequence encoding a nuclease comprises a sequence isolated or derived from a CRISPR/Cas protein. In some embodiments, the CRISPR/Cas protein is isolated or derived from any one of a type I, a type IA, a type IB, a type IC, a type ID, a type IE, a type IF, a type IU, a type III, a type IIIA, a type IIIB, a type IIIC, a type HID, a type IV, a type IVA, a type IVB, a type II, a type IIA, a type IIB, a type IIC, a type V, or a type VI CRISPR/Cas protein. In some embodiments, the sequence encoding a nuclease comprises a sequence isolated or derived from a TALEN or a nuclease domain thereof. In some embodiments, the sequence encoding a nuclease comprises a sequence isolated or derived from a zinc finger nuclease or a nuclease domain thereof. In some embodiments, the target sequence comprises a sequence encoding a component of an adaptive immune response.

The disclosure provides a vector comprising a composition of the disclosure. In some embodiments, the vector is a viral vector. In some embodiments, the vector comprises a sequence isolated or derived from a lentivirus, an adenovirus, an adeno-associated virus (AAV) vector, or a retrovirus. In some embodiments, the vector is replication incompetent.

The disclosure provides a vector comprising a composition of the disclosure. In some embodiments, the vector is a viral vector. In some embodiments, the vector comprises a sequence isolated or derived from an adeno-associated vector (AAV). In some embodiments, the adeno-associated virus (AAV) is an isolated AAV. In some embodiments, the adeno-associated virus (AAV) is a self-complementary adeno-associated virus (scAAV). In some embodiments, the adeno-associated virus (AAV) is a recombinant adeno-associated virus (rAAV). In some embodiments, the adeno-associated virus (AAV) comprises a sequence isolated or derived from an AAV of serotype AAV1, AAV2, AAV3, AAV4, AAV5, AAV6, AAV7, AAV8, AAV9, AAV10, AAV11, or AAV12. In some embodiments, the adeno-associated virus (AAV) comprises a sequence isolated or derived from an AAV of serotype AAV9. In some embodiments, the adeno-associated virus (AAV) comprise a sequence isolated or derived from Anc80.

The disclosure provides a vector comprising a composition of the disclosure. In some embodiments, the vector is a viral vector. In some embodiments, the vector is a retrovirus.

The disclosure provides a vector comprising a composition of the disclosure. In some embodiments, the vector is a viral vector. In some embodiments, the vector is a lentivirus.

The disclosure provides a vector comprising a composition of the disclosure. In some embodiments, the vector is a non-viral vector. In some embodiments, the non-viral vector comprises a nanoparticle, a micelle, a liposome or lipoplex, a polymersome, a polyplex or a dendrimer.

The disclosure provides a composition comprising a vector of the disclosure.

The disclosure provides a cell comprising a vector of the disclosure.

The disclosure provides a cell comprising a cell of the disclosure.

In some embodiments of cells of the disclosure, the cell is a mammalian cell. In some embodiments, the cell is a human cell.

In some embodiments of cells of the disclosure, the cell is an immune cell. In some embodiments, the immune cell is a T lymphocyte (T-cell). In some embodiments, the T-cell is an effector T-cell, a helper T-cell, a memory T-cell, a regulatory T-cell, a natural Killer T-cell, a mucosal-associated invariant T-cell, or a gamma delta T cell.

In some embodiments of cells of the disclosure, the cell is an immune cell. In some embodiments, the immune cell is an antigen-presenting cell. In some embodiments, the antigen-presenting cell is a dendritic cell, a macrophage, or a B cell. In some embodiments, the antigen-presenting cell is a somatic cell.

In some embodiments of cells of the disclosure, the cell is an immune cell. In some embodiments, the cell is a healthy cell. In some embodiments, the cell is not a healthy cell. In some embodiments, the cell is isolated or derived from a subject having a disease or disorder.

The disclosure provides a composition comprising a cell of the disclosure.

The disclosure provides a composition comprising a plurality of cells of the disclosure.

The disclosure provides a method of masking a cell from an adaptive immune response comprising contacting a composition of the disclosure to the cell to produce a modified cell, wherein the composition modifies a level of expression of an RNA molecule of the modified cell and wherein the RNA molecule encodes a component of an adaptive immune response. In some embodiments, the cell is in vivo, in vitro, ex vivo or in situ. In some embodiments, the cell is in vitro or ex vivo. In some embodiments, a plurality of cells comprises the cell. In some embodiments, each cell of the plurality of cells contacts the composition, thereby producing a plurality of modified cells. In some embodiments, the method further comprises administering the modified cell to a subject. In some embodiments, the method further comprises administering the plurality of modified cells to a subject. In some embodiments, the cell is autologous. In some embodiments, the cell is allogeneic. In some embodiments, the plurality of modified cells is autologous. In some embodiments, the plurality of modified cells is allogeneic. In some embodiments, the component of an adaptive immune response comprises or consists of a component of a type I major histocompatibility complex (MHC I), a type II major histocompatibility complex (MHC II), a T-cell receptor (TCR), a costimulatory molecule or a combination thereof. In some embodiments, the MHC I component comprises an α1 chain, an α2 chain, an α3 chain, or a β2M protein. In some embodiments, the component of an adaptive immune response comprises or consists of an MHC I β2M protein. In some embodiments, the MHC II component comprises an α1 chain, an α2 chain, a β1 chain, or a β2 chain. In some embodiments, the TCR component comprises an α-chain and a β-chain. In some embodiments, the costimulatory molecule comprises a Cluster of Differentiation 28 (CD28), a Cluster of Differentiation 80 (CD80), a Cluster of Differentiation 86 (CD86), an Inducible T-cell COStimulator (ICOS), or an ICOS Ligand (ICOSLG) protein. In some embodiments, a protein component of an adaptive immune response is, without limitation, Beta-2-microglobulin (β2M), Human Leukocyte Antigen A (HLA-A), Human Leukocyte Antigen B (HLA-B), Human Leukocyte Antigen C (HLA-C), Cluster of Differentiation 28 (CD28), Cluster of Differentiation 80 (CD80), Cluster of Differentiation 86 (CD86), Inducible T-cell Costimulator (ICOS), ICOS Ligand (ICOSLG), OX40L, Interleukin 12 (IL12), or CC Chemokine Receptor 7 (CCR7).

The disclosure provides a method of preventing or reducing an adaptive immune response in a subject comprising administering a therapeutically effective amount of a composition of the disclosure to the subject, wherein the composition contacts at least one cell in the subject producing a modified cell, wherein the composition modifies a level of expression of an RNA molecule of the modified cell and wherein the RNA molecule encodes a component of an adaptive immune response.

The disclosure provides a method of treating a disease or disorder in a subject comprising administering a therapeutically effective amount of a composition of the disclosure to the subject, wherein the composition contacts at least one cell in the subject producing a modified cell, wherein the composition modifies a level of expression of an RNA molecule of the modified cell and wherein the composition prevents or reduces an adaptive immune response to the modified cell.

In some embodiments of the methods of the disclosure, the component of an adaptive immune response comprises or consists of a component of a type I major histocompatibility complex (MHC I), a type II major histocompatibility complex (MHC II), a T-cell receptor (TCR), a costimulatory molecule or a combination thereof. In some embodiments, the MHC I component comprises an α1 chain, an α2 chain, an α3 chain, or a β2M protein. In some embodiments, the component of an adaptive immune response comprises or consists of an MHC I β2M protein. In some embodiments, the MHC II component comprises an α1 chain, an α2 chain, a β1 chain, or a β2 chain. In some embodiments, the TCR component comprises an α-chain and a β-chain. In some embodiments, the costimulatory molecule comprises a Cluster of Differentiation 28 (CD28), a Cluster of Differentiation 80 (CD80), a Cluster of Differentiation 86 (CD86), an Inducible T-cell COStimulator (ICOS), or an ICOS Ligand (ICOSLG) protein.

In some embodiments of the methods of treating a disease or disorder of the disclosure, the disease or disorder is a genetic disease or disorder. In some embodiments, the disease or disorder is a single gene genetic disease or disorder. In some embodiments, the disease or disorder results from microsatellite instability. In some embodiments, the microsatellite instability occurs in a DNA sequence at least 1, 2, 3, 4, 5 or 6 repeated motifs. In some embodiments, an RNA molecule comprises a transcript of the DNA sequence and wherein the composition binds to a target sequence of the RNA molecule comprising at least 1, 2, 3, 4, 5, or 6 repeated motifs.

In some embodiments of the methods of the disclosure, the composition is administered systemically. In some embodiments, the composition is administered intravenously. In some embodiments, the composition is administered by an injection or an infusion.

In some embodiments of the methods of the disclosure, the composition is administered locally. In some embodiments, the composition is administered by an intraosseous, intraocular, intracerebral, or intraspinal route. In some embodiments, the composition is administered by an injection or an infusion.

In some embodiments of the methods of the disclosure, a therapeutically effective amount of the composition is a single dose.

In some embodiments of the methods of the disclosure, the composition is non-genome integrating.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram depicting an exemplary RNA Endonuclease-C. jejuni Cas9 fusion protein.

FIG. 1B is a graph depicting changes in expression levels of Zika NS5 in the presence of both E43 and E67 CjeCas9-endonuclease fusions with sgRNAs containing the various NS5-targeting spacer sequences as indicated in Table 8. Zika NS5 expression is displayed as fold change relative to the endonuclease loaded with an sgRNA containing a control (Lambda) spacer sequence.

FIG. 2A is a fluorescence microscopy image of cells transfected with CjeCas9-endonuclease fusions loaded with an sgRNA containing a Zika NS5-targeting spacer sequence.

FIG. 2B is a graph depicting changes of expression of Zika NS5 in the presence of CjeCas9-endonuclease fusions loaded with the appropriate Zika NS5-targeting sgRNA as compared to CjeCas9-endonuclease fusions loaded with a non-Zika NS5 targeting sgRNA.

FIG. 3 is a list of exemplary endonucleases for use in the compositions of the disclosure.

FIG. 4 is a schematic diagram depicting a construct encoding an exemplary RNA Endonuclease-C. jejuni Cas9 fusion protein and two gRNA molecules for modulating immune response in the context of a gene therapy. The present invention describes a means to address human disease using a CRISPR-based gene therapy or other non-self protein encoded in AAV while simultaneously altering host gene expression to prevent adaptive immune response to the non-self protein. In one embodiment, the AAV particle (left) carries a pair of guide RNAs and a CRISPR-associated (Cas) protein. The guides target a gene associated with adaptive immune response and a gene (or gene product) to promote therapeutic benefit, respectively. Upon delivery to target tissue, the immune response-targeted guide reduces expression of genes associated with antigen presentation (beta-2-microglobulin, B2M) or co-stimulation of T cells (ICOSLG, CD80, CD86, OX40L, IL12, CCR7). Antigen presentation inhibition prevents formation of T helper (Th) cells specific to the therapeutic transgenes such as Cas proteins while co-stimulation inhibition prevents the activation of Th cells that are specific to the transgene.

### DETAILED DESCRIPTION

The disclosure provides compositions and methods for the simultaneous treatment of disease by targeting RNA molecules of a modified cell while masking the modified cell from an adaptive immune response. By inhibiting or reducing expression of a component of an adaptive immune response in the modified cell, the modified cell is invisible to a host immune system. For example, compositions of the disclosure may simultaneously target an RNA molecule associated with a genetic disease or disorder and an RNA molecule that encodes the β2M subunit of the MHC I. By selectively targeting an RNA molecule that encodes the β2M subunit of the MHC I, the composition prevents the modified cell from displaying one or more antigen peptides derived from an RNA targeting construct, vector, or combination thereof on the surface of the modified cell. Consequently, a subject's immune system does not identify the modified cell as containing foreign sequences and does not attempt to mount an immune response directed at the modified cell. This method increases the therapeutic efficacy of the treatment of the genetic disease or disorder while avoiding a common side effect of gene therapy.

### RNA-Targeting Fusion Protein Compositions

The disclosure provides a composition comprising (a) a sequence comprising a guide RNA (gRNA) that specifically binds a target sequence within an RNA molecule and (b) a sequence encoding a fusion protein, the sequence comprising a sequence encoding a first RNA-binding polypeptide and a sequence encoding a second RNA-binding polypeptide, wherein neither the first RNA-binding polypeptide nor the second RNA-binding polypeptide comprises a significant DNA-nuclease activity, wherein the first RNA-binding polypeptide and the second RNA-binding polypeptide are not identical, and wherein the second RNA-binding polypeptide comprises an RNA-nuclease activity wherein the first RNA-binding polypeptide and the second RNA-binding polypeptide are not identical, and wherein the second RNA-binding polypeptide comprises an RNA-nuclease activity.

In some embodiments of the compositions of the disclosure, the target sequence comprises at least one repeated sequence.

In some embodiments of the compositions of the disclosure, the gRNA sequence comprises a promoter capable of expressing the gRNA in a eukaryotic cell.

In some embodiments of the compositions of the disclosure, the eukaryotic cell is an animal cell. In some embodiments, the animal cell is a mammalian cell. In some embodiments, the animal cell is a human cell.

In some embodiments of the compositions of the disclosure, the promoter is a constitutively active promoter. In some embodiments, the promoter sequence is isolated or derived from a promoter capable of driving expression of an RNA polymerase. In some embodiments, the promoter sequence is isolated or derived from a U6 promoter. In some embodiments, the promoter sequence is isolated or derived from a promoter capable of driving expression of a transfer RNA (tRNA). In some embodiments, the promoter sequence is isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter. In some embodiments, the promoter sequence is isolated or derived from a valine tRNA promoter.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides. In some embodiments, the spacer sequence comprises or consists of the sequence

(SEQ ID NO: 1)

UGGAGCGAGCAUCCCCCAAA,

(SEQ ID NO: 2)

GUUUGGGGGAUGCUCGCUCCA,

(SEQ ID NO: 3)

CCCUCACUGCUGGGGAGUCC,

(SEQ ID NO: 4)

GGACUCCCCAGCAGUGAGGG,

(SEQ ID NO: 5)

GCAACUGGAUCAAUUUGCUG,

(SEQ ID NO: 6)

GCAGCAAAUUGAUCCAGUUGC,

(SEQ ID NO: 7)

GCAUUCUUAUCUGGUCAGUGC,

(SEQ ID NO: 8)

GCACUGACCAGAUAAGAAUG,

(SEQ ID NO: 9)

GAGCAGCAGCAGCAGCAGCAG,

(SEQ ID NO: 10)

GCAGGCAGGCAGGCAGGCAGG,

(SEQ ID NO: 11)

GCCCCGGCCCCGGCCCCGGC,
or

(SEQ ID NO: 84)

GCTGCTGCTGCTGCTGCTGC,

(SEQ ID NO: 74)

GGGGCCGGGGCCGGGGCCGG,

(SEQ ID NO: 75)

GGGCCGGGGCCGGGGCCGGG,

(SEQ ID NO: 76)

GGCCGGGGCCGGGGCCGGGG,

(SEQ ID NO: 77)

GCCGGGGCCGGGGCCGGGGC,

(SEQ ID NO: 78)

CCGGGGCCGGGGCCGGGGCC,
or

(SEQ ID NO: 79)

CGGGGCCGGGGCCGGGGCCG.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides. In some embodiments, the spacer sequence comprises or consists of the sequence

(SEQ ID NO: 14)

GUGAUAAGUGGAAUGCCAUG,

(SEQ ID NO: 15)

CUGGUGAACUUCCGAUAGUG,
or

(SEQ ID NO: 16)

GAGATATAGCCTGGTGGTTC.

In some embodiments of the compositions of the disclosure, the sequence comprising the gRNA further comprises a spacer sequence that specifically binds to the target RNA sequence. In some embodiments, the spacer sequence has at least 50%, 55%, 60%, 65%, 70%, 75%, 80%, 87%, 90%, 95%, 97%, 99% or any percentage in between of complementarity to the target RNA sequence. In some embodiments, the spacer sequence has 100% complementarity to the target RNA sequence. In some embodiments, the spacer sequence comprises or consists of 20 nucleotides. In some embodiments, the spacer sequence comprises or consists of 21 nucleotides. In some embodiments, the spacer sequence comprises or consists of a sequence comprising at least 1, 2, 3, 4, 5, 6, or 7 repeats of the sequence CUG (SEQ ID NO: 18), CCUG (SEQ ID NO: 19), CAG (SEQ ID NO: 80), GGGGCC (SEQ ID NO: 81) or any combination thereof.

In some embodiments of the compositions of the disclosure, the sequence comprising the gRNA further comprises a scaffold sequence that specifically binds to the first RNA binding protein. In some embodiments, the scaffold sequence comprises a stem-loop structure. In some embodiments, the scaffold sequence comprises or consists of 90 nucleotides. In some embodiments, the scaffold sequence comprises or consists of 93 nucleotides. In some embodiments, the scaffold sequence comprises or consists of the sequence GUUUAAGAGCUAUGCUGGAAACAGCAUAGCAAGUUUAAAUAAGGCUAGUCCGUU AUCAACUUGAAAAAGUGGCACCGAGUCGGUGCUUUUUUU (SEQ ID NO: 83). In some embodiments, the scaffold sequence comprises or consists of the sequence GGACAGCAUAGCAAGUUAAAAUAAGGCUAGUCCGUUAUCAACUUGAAAAAGUGG CACCGAGUCGGUGCUUUUU (SEQ ID NO: 17). In some embodiments, the scaffold sequence comprises or consists of the sequence

(SEQ ID NO: 82)
GUUUAAGAGCUAUGCUGGAAACAGCAUAGCAAGUUUAAAUAAGGCUAGUC
CGUUAUCAACUUGAAAAAGUGGCACCGAGUCGGUGCUUUUUUU
or
(SEQ ID NO: 13)
GUUUUAGAGCUAGAAAUAGCAAGUUAAAAUAAGGCUAGUCCGUUAUCAAC
UUGAAAAAGUGGCACCGAGUCGGUGCUUUUUUU.

In some embodiments of the compositions of the disclosure, the gRNA does not bind or does not selectively bind to a second sequence within the RNA molecule.

In some embodiments of the compositions of the disclosure, an RNA genome or an RNA transcriptome comprises the RNA molecule.

In some embodiments of the compositions of the disclosure, the first RNA binding protein comprises a CRISPR-Cas protein. In some embodiments, the CRISPR-Cas protein is a Type II CRISPR-Cas protein. In some embodiments, the first RNA binding protein comprises a Cas9 polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, the first RNA binding protein comprises a CRISPR-Cas protein. In some embodiments, the CRISPR-Cas protein is a Type V CRISPR-Cas protein. In some embodiments, the first RNA binding protein comprises a Cpf1 polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, the first RNA binding protein comprises a CRISPR-Cas protein. In some embodiments, the CRISPR-Cas protein is a Type VI CRISPR-Cas protein. In some embodiments, the first RNA binding protein comprises a Cas13 polypeptide or an RNA-binding portion thereof. In some embodiments, the first RNA binding protein comprises a Cas13d polypeptide or an RNA-binding portion thereof. In some embodiments, the CRISPR-Cas protein comprises a native RNA nuclease activity. In some embodiments, the native RNA nuclease activity is reduced or inhibited. In some embodiments, the native RNA nuclease activity is increased or induced. In some embodiments, the CRISPR-Cas protein comprises a native DNA nuclease activity and the native DNA nuclease activity is inhibited. In some embodiments, the CRISPR-Cas protein comprises a mutation. In some embodiments, a nuclease domain of the CRISPR-Cas protein comprises the mutation. In some embodiments, the mutation occurs in a nucleic acid encoding the CRISPR-Cas protein. In some embodiments, the mutation occurs in an amino acid encoding the CRISPR-Cas protein. In some embodiments, the mutation comprises a substitution, an insertion, a deletion, a frameshift, an inversion, or a transposition. In some embodiments, the mutation comprises a deletion of a nuclease domain, a binding site within the nuclease domain, an active site within the nuclease domain, or at least one essential amino acid residue within the nuclease domain.

In some embodiments of the compositions of the disclosure, the first RNA binding protein comprises a Pumilio and FBF (PUF) protein. In some embodiments, the first RNA binding protein comprises a Pumilio-based assembly (PUMBY) protein. In some embodiments, a PUF1 protein of the disclosure comprises or consists of the amino acid sequence of

| (SEQ ID NO: 208) | |
|---|---|
| MDKSKQMNIN NLSNIPEVID PGITIPIYEE EYENNGESNS QLQQQPQKLG SYRSRAGKFS | 60 |
| NTLSNLLPSI SAKLHHSKKN SHGKNGAEFS SSNNSSQSTV ASKTPRASPS RSKMMESSID | 120 |
| GVTMDRPGSL TPPQDMEKLV HFPDSSNNFL IPAPRGSSDS FNLPHQISRT RNNTMSSQIT | 180 |
| SISSIAPKPR TSSGIWSSNA SANDPMQQHL LQQLQPTTSN NTTNSNTLND YSTKTAYFDN | 240 |
| MVSTSGSQMA DNKMNTNNLA IPNSVWSNTR QRSQSNASSI YTDAPLYEQP ARASISSHYT | 300 |
| IPTQESPLIA DEIDPQSINW VTMDPTVPSI NQISNLLPTN TISISNVFPL QHQQPQLNNA | 360 |
| INLTSTSLAT LCSKYGEVIS ARTLRNLNMA LVEFSSVESA VKALDSLQGK EVSMIGAPSK | 420 |
| ISFAKILPMH QQPPQFLLNS QGLPLGLENN NLQPQPLLQE QLFNGAVTFQ QQGNVSIPVF | 480 |
| NQQSQQSQHQ NHSSGSAGFS NVLHGYNNNN SMHGNNNNSA NEKEQCPFPL PPPNVNEKED | 540 |
| LLREIIELFE ANSDEYQINS LIKKSLNHKG TSDTQNFGPL PEPLSGREFD PPKLRELRKS | 600 |
| IDSNAFSDLE IEQLAIAMLD ELPELSSDYL GNTIVQKLFE HSSDIIKDIM LRKTSKYLTS | 660 |
| MGVHKNGTWA CQKMITMAHT PRQIMQVTQG VKDYCTPLIN DQFGNYVIQC VLKFGFPWNQ | 720 |
| FIFESIIANF WVIVQNRYGA RAVRACLEAH DIVTPEQSIV LSAMIVTYAE YLSTNSNGAL | 780 |
| LVTWFLDTSV LPNRHSILAP RLTKRIVELC GHRLASLTIL KVLNYRGDDN ARKIILDSLF | 840 |
| GNVNAHDSSP PKELTKLLCE TNYGPTFVHK VLAMPLLEDD LRAHIIKQVR KVLTDSTQIQ | 900 |
| PSRRLLEEVG LASPSSTHNK TKQQQQQHHN SSISHMFATP DTSGQHMRGL SVSSVKSGGS | 960 |
| KHTTMNTTTT NGSSASTLSP GQPLNANSNS SMGYFSYPGV FPVSGFSGNA SNGYAMNNDD | 1020 |
| LSSQFDMLNF NNGTRLSLPQ LSLTNHNNTT MELVNNVGSS QPHTNNNNNN NNTNYNDDNT | 1080 |
| VFETLTLHSA N. | 1091 |

In some embodiments, a PUF3 protein of the disclosure comprises or consists of the amino acid sequence of

| (SEQ ID NO: 209) | |
|---|---|
| 1 | MEMNMDMDMD MELASIVSSL SALSHSNNNG GQAAAAGIVN GGAAGSQQIG GFRRSSFTTA |
| 61 | NEVDSEILLL HGSSESSPIF KKTALSVGTA PPFSTNSKKF FGNGGNYYQY RSTDTASLSS |
| 121 | ASYNNYHTHH TAANLGKNNK VNHLLGQYSA SIAGPVYYNG NDNNNSGGEG FFEKFGKSLI |
| 181 | DGTRELESQD RPDAVNTQSQ FISKSVSNAS LDTQNTFEQN VESDKNFNKL NRNTTNSGSL |
| 241 | YHSSSNSGSS ASLESENAHY PKRNIWNVAN TPVFRPSNNP AAVGATNVAL PNQQDGPANN |
| 301 | NFPPYMNGFP PNQFHQGPHY QNFPNYLIGS PSNFISQMIS VQIPANEDTE DSNGKKKKKA |
| 361 | NRPSSVSSPS SPPNNSPFPF AYPNPMMFMP PPPLSAPQQQ QQQQQQQQQE DQQQQQQQEN |
| 421 | PYIYYPTPNP IPVKMPKDEK TFKKRNNKNH PANNSNNANK QANPYLENSI PTKNTSKKNA |
| 481 | SSKSNESTAN NHKSHSHSHP HSQSLQQQQQ TYHRSPLLEQ LRNSSSDKNS NSNMSLKDIF |
| 541 | GHSLEFCKDQ HGSRFIQREL ATSPASEKEV IFNEIRDDAI ELSNDVFGNY VIQKFFEFGS |
| 601 | KIQKNTLVDQ FKGNMKQLSL QMYACRVIQK ALEYIDSNQR IELVLELSDS VLQMIKDQNG |
| 661 | NHVIQKAIET IPIEKLPFIL SSLTGHIYHL STHSYGCRVI QRLLEFGSSE DQESILNELK |
| 721 | DFIPYLIQDQ YGNYVIQYVL QQDQFTNKEM VDIKQEIIET VANNVVEYSK HKFASNVVEK |
| 781 | SILYGSKNQK DLIISKILPR DKNHALNLED DSPMILMIKD QFANYVIQKL VNVSEGEGKK |
| 841 | LIVIAIRAYL DKLNKSNSLG NRHLASVEKL AALVENAEV. |

In some embodiments, a PUF4 protein of the disclosure comprises or consists of the amino acid sequence of

| (SEQ ID NO: 210) | |
|---|---|
| 1 | MSTKGLKEEI DDVPSVDPVV SETVNSALFQ LQLDDPEENA TSNAFANKVS QDSQFANGPP |
| 61 | SQMFPHPQMM GGMGFMPYSQ MMQVPHNPCP FFPPPDFNDP TAPLSSSPLN AGGPPMLFKN |
| 121 | DSLPFQMLSS GAAVATQGGQ NLNPLINDNS MKVLPIASAD PLWTHSNVPG SASVAIEETT |
| 181 | ATLQESLPSK GRESNNKASS FRRQTFHALS PTDLINAANN VTLSKDFQSD MQNFSKAKKP |
| 241 | SVGANNTAKT RTQSISFDNT PSSTSFIPPT NSVSEKLSDF KIETSKEDLI NKTAPAKKES |
| 301 | PTTYGAAYPY GGPLLQPNPI MPGHPHNISS PIYGIRSPFP NSYEMGAQFQ PFSPILNPTS |
| 361 | HSLNANSPIP LTQSPIHLAP VLNPSSNSVA FSDMKNDGGK PTTDNDKAGP NVRMDLINPN |
| 421 | LGPSMQPFHI LPPQQNTPPP PWLYSTPPPF NAMVPPHLLA QNHMPLMNSA NNKHHGRNNN |
| 481 | SMSSHNDNDN IGNSNYNNKD TGRSNVGKMK NMKNSYHGYY NNNNNNNNNN NNNNNSNATN |
| 541 | SNSAEKQRKI EESSRFADAV LDQYIGSIHS LCKDQHGCRF LQKQLDILGS KAADAIFEET |
| 601 | KDYTVELMTD SFGNYLIQKL LEEVTTEQRI VLTKISSPHF VEISLNPHGT RALQKLIECI |
| 661 | KTDEEAQIVV DSLRPYTVQL SKDLNGNHVI QKCLQRLKPE NFQFIFDAIS DSCIDIATHR |
| 721 | HGCCVLQRCL DHGTTEQCDN LCDKLLALVD KLTLDPFGNY VVQYIITKEA EKNKYDYTHK |
| 781 | IVHLLKPRAI ELSIHKFGSN VIEKILKTAI VSEPMILEIL NNGGETGIQS LLNDSYGNYV |
| 841 | LQTALDISHK QNDYLYKRLS EIVAPLLVGP IRNTPHGKRI IGMLHLDS. |

In some embodiments, a PUF5 protein of the disclosure comprises or consists of the amino acid sequence of

| (SEQ ID NO: 211) | |
|---|---|
| 1 | MSDSTGRINS KASDSSSISD HQTADLSIFN GSFDGGAFSS SNIPLFNFMG TGNQRFQYSP |
| 61 | HPFAKSSDPC RLAALTPSTP KGPLNLTPAD FGLADFSVGN ESFADFTANN TSFVGNVQSN |
| 121 | VRSTRLLPAW AVDNSGNIRD DLTLQDVVSN GSLIDFAMDR TGVKFLERHF PEDHDNEMHF |
| 181 | VLFDKLTEQG AVFTSLCRSA AGNFIIQKFV EHATLDEQER LVRKMCDNGL IEMCLDKFAC |
| 241 | RVVQMSIQKF DVSIAMKLVE KISSLDFLPL CTDQCAIHVL QKVVKLLPIS AWSFFVKFLC |
| 301 | RDDNLMTVCQ DKYGCRLVQQ TIDKLSDNPK LHCFNTRLQL LHGLMTSVAR NCFRLSSNEF |
| 361 | ANYVVQYVIK SSGVMEMYRD TIIEKCLLRN ILSMSQDKYA SHVVEGAFLF APPLLLSEMM |
| 421 | DEIFDGYVKD QETNRDALDI LLFHQYGNYV VQQMISICIS ALLGKEERKM VASEMRLYAK |
| 481 | WFDRIKNRVN RHSGRLERFS SGKKIIESLQ KLNVPMTMTN EPMPYWAMPT PLMDISAHFM |
| 541 | NKLNFQKNSV FDE. |

In some embodiments, a PUF6 protein of the disclosure comprises or consists of the amino acid sequence of

| (SEQ ID NO: 212) | |
|---|---|
| 1 | MTPNRRSTDS YNMLGASFDF DPDFSLLSNK THKNKNPKPP VKLLPYRHGS NTTSSDLDNY |
| 61 | IFNSGSGSSD DETPPPAAPI FISLEEVLLN GLLIDFAIDP SGVKFLEANY PLDSEDQIRK |
| 121 | AVFEKLTEST TLFVGLCHSR NGNFIVQKLV ELATPAEQRE LLRQMIDGGL LVMCKDKFAC |
| 181 | RVVQLALQKF DHSNVFQLIQ ELSTFDLAAM CTDQISIHVI QRVVKQLPVD MWTFFVHFLS |
| 241 | SGDSLMAVCQ DKYGCRLVQQ VIDRLAENTK LPCFKFRIQL LHSLMTCIVR NCYRLSSNEF |
| 301 | ANYVIQYVIK SSGIMEMYRD TIIDKCLLRN LLSMSQDKYA SHVIEGAFLF APPALLHEMM |
| 361 | EEIFSGYVKD VELNRDALDI LLFHQYGNYV VQQMISICTA ALIGKEERQL PPAILLLYSG |
| 421 | WYEKMKQRVL QHASRLERFS SGKKIIDSVM RHGVPTAAAI NAQAAPSLME LTAQFDAMFP |
| 481 | SFLAR. |

In some embodiments, a PUF7 protein of the disclosure comprises or consists of the amino acid sequence of

| (SEQ ID NO: 213) | |
|---|---|
| 1 | MTPNRRSTDS YNMLGASFDF DPDFSLLSNK THKNKNPKPP VKLLPYRHGS NTTSSDSDSY |
| 61 | IFNSGSGSSD AETPAPVAPI FISLEDVLLN GQLIDFAIDP SGVKFLEANY PLDSEDQIRK |
| 121 | AVFEKFTEST TLFVGLCHSR NGNFIVQKLV ELATPAEQRE LLRQMIDGGL LAMCKDKFAC |
| 181 | RVVQLALQKF DHSNVFQLIQ ELSTFDLAAM CTDQISIHVI QRVVKQLPVD MWTFFVHFLS |
| 241 | SGDSLMAVCQ DKYGCRLVQQ VIDRLAENPK LPCFKFRIQL LHSLMTCIVR NCYRLSSNEF |
| 301 | ANYVIQYVIK SSGIMEMYRD TIIDKCLLRN LLSMSQDKYA SHVIEGAFLF APPALLHEMM |
| 361 | EEIFSGYVKD VESNRDALDI LLFHQYGNYV VQQMISICTA ALIGKEEREL PPAILLLYSG |
| 421 | WYEKMKQRVL QHASRLERFS SGKKIIDSVM RHGVPTAAAV NAQAAPSLME LTAQFDAMFP |
| 481 | SFLAR. |

In some embodiments, a PUF8 protein of the disclosure comprises or consists of the amino acid sequence of

| (SEQ ID NO: 214) | |
|---|---|
| 1 | MSRPISIGNT CTFDPSASPI ESLGRSIGAQ KIVDSVCGSP IRSYGRHIST NPKNERLPDT |
| 61 | PEFQFATYMH QGGKVIGQNT LHMFGTPPSC YCAQENIPIS SNVGHVLSTI NNNYMNHQYN |
| 121 | GSNMFSNQMT QMLQAQAYND LQMHQAHSQS IRVPVQPSAT GIFSNPYREP TTTDDLLTRY |
| 181 | RANPAMMKNL KLSDIRGALL KFAKDQVGSR FIQQELASSK DRFEKDSIFD EVVSNADELV |
| 241 | DDIFGNYVVQ KFFEYGEERH WARLVDAIID RVPEYAFQMY ACRVLQKALE KINEPLQIKI |
| 301 | LSQIRHVIHR CMKDQNGNHV VQKAIEKVSP QYVQFIVDTL LESSNTIYEM SVDPYGCRVV |
| 361 | QRCLEHCSPS QTKPVIGQIH KRFDEIANNQ YGNYVVQHVI EHGSEEDRMV IVTRVSNNLF |
| 421 | EFATHKYSSN VIEKCLEQGA VYHKSMIVGA ACHHQEGSVP IVVQMMKDQY ANYVVQKMFD |
| 481 | QVTSEQRREL ILTVRPHIPV LRQFPHGKHI LAKLEKYFQK PAVMSYPYQD MQGSH. |

In some embodiments, a PUF9 protein of the disclosure comprises or consists of the amino acid sequence of

| (SEQ ID NO: 215) | |
|---|---|
| 1 | MADPNWAYAP PTNYYADHSI AKPIMISGGH PSQDQGHSPK SESFGQSVTT AFNGMVDNLV |
| 61 | GSPSSSVQQR NYFTTTPFPI SRSPNDRNDD KIMGNGSYGV PIPIPQDGVP QGTPDFQMTP |
| 121 | FLQQGGHLIG GSPNGPVQVS GNWYSGGAGI FSTMQQADPS NGMPGMAAEF VNNENGMPGP |
| 181 | NGMHQQAMIS GSPPFPYQNM MNLTTSFGAM GLGPQQIQQR DPQMFQQPIL HEPIQGMAQN |
| 241 | GFGQQVFFTQ MQNQQHPQGQ AQQQLQQLAQ QHQQQQNSQQ FFGQGPNGMG NGGVMNDWSQ |
| 301 | RSFGMPQQQA QQNGLPPNFS QNPPRRRGPE DPNGQTPKTL QDIKNNVIEF AKDQHGSRFI |
| 361 | QQKLERASLR DKAAIFTPVL ENAEELMTDV FGNYVIQKFF EFGNNEQRNQ LVGTIRGNVM |
| 421 | KLALQMYGCR VIQKALEYVE EKYQHEILGE MEGQVLKCVK DQNGNHVIQK VIERVEPERL |
| 481 | QFIIDAFTKN NSDNVYTLSV HPYGCRVIQR VLEYCNEEQK QPVLDALQIH LKQLVLDQYG |
| 541 | NYVIQHVIEH GSPSDKEQIV QDVISDDLLK FAQHKFASNV IEKCLTFGGH AERNLIIDKV |
| 601 | CGDPNDPSPP LLQMMKDPFA NYVVQKMLDV ADPQHRKKIT LTIKPHIATL RKYNFGKHIL |
| 661 | LKLEKYFAKQ APANSSNSSS NDQIYEHSPF DIPLGADFSN HPF. |

In some embodiments of the compositions of the disclosure, the first RNA binding protein does not require multimerization for RNA-binding activity. In some embodiments, the first RNA binding protein is not a monomer of a multimer complex. In some embodiments, a multimer protein complex does not comprise the first RNA binding protein.

In some embodiments of the compositions of the disclosure, the first RNA binding protein selectively binds to a target sequence within the RNA molecule. In some embodiments, the first RNA binding protein does not comprise an affinity for a second sequence within the RNA molecule. In some embodiments, the first RNA binding protein does not comprise a high affinity for or selectively bind a second sequence within the RNA molecule.

In some embodiments of the compositions of the disclosure, an RNA genome or an RNA transcriptome comprises the RNA molecule.

In some embodiments of the compositions of the disclosure, the first RNA binding protein comprises between 2 and 1300 amino acids, inclusive of the endpoints.

In some embodiments of the compositions of the disclosure, the sequence encoding the first RNA binding protein further comprises a nuclear localization signal (NLS). In some embodiments, the sequence encoding a nuclear localization signal (NLS) is positioned 3′ to the sequence encoding the first RNA binding protein. In some embodiments, the first RNA binding protein comprises an NLS at a C-terminus of the protein.

In some embodiments of the compositions of the disclosure, the sequence encoding the first RNA binding protein further comprises a first sequence encoding a first NLS and a second sequence encoding a second NLS. In some embodiments, the sequence encoding the first NLS or the second NLS is positioned 3′ to the sequence encoding the first RNA binding protein. In some embodiments, the first RNA binding protein comprises the first NLS or the second NLS at a C-terminus of the protein.

In some embodiments of the compositions of the disclosure, the second RNA binding protein comprises or consists of a nuclease domain. In some embodiments, the second RNA binding protein binds RNA in a manner in which it associates with RNA. In some embodiments, the second RNA binding protein associates with RNA in a manner in which it cleaves RNA.

In some embodiments of the compositions of the disclosure, the sequence encoding the second RNA binding protein comprises or consists of an RNAse. In some embodiments, the second RNA binding protein comprises or consists of an RNAse1 polypeptide. In some embodiments, the RNAse1 polypeptide comprises or consists of:
KESRAKKFQRQHMDSDSSPSSSSTYCNQMMRRRNMTQGLCKPVNTFVHEPLVDVQNV CFQEKVTCKNGQGNCYKSNSSMHITDCRLTNGSRYPNCAYRTSPKERHIIVACEGSPYV PVHFDASVEDST (SEQ ID NO: 20). In some embodiments, the second RNA binding protein comprises or consists of an RNAse4 polypeptide. In some embodiments, the RNAse4 polypeptide comprises or consists of:
QDGMYQRFLRQHVHPEETGGSDRYCDLMMQRRKMTLYHCKRFNTFIHEDIWNIRSICS TTNIQCKNGKMNCHEGVVKVTDCRDTGSSRAPNCRYRAIASTRRVVIACEGNPQVPVH FDG (SEQ ID NO: 21). In some embodiments, the second RNA binding protein comprises or consists of an RNAse6 polypeptide. In some embodiments, the RNAse6 polypeptide comprises or consists of:
WPKRLTKAHWFEIQHIQPSPLQCNRAMSGINNYTQHCKHQNTFLHDSFQNVAAVCDLL SIVCKNRRHNCHQSSKPVNMTDCRLTSGKYPQCRYSAAAQYKFFIVACDPPQKSDPPYK LVPVHLDSIL (SEQ ID NO: 22). In some embodiments, the second RNA binding protein comprises or consists of an RNAse7 polypeptide. In some embodiments, the RNAse7 polypeptide comprises or consists of:
APARAGFCPLLLLLLLGLWVAEIPVSAKPKGMTSSQWFKIQHMQPSPQACNSAMKNINK HTKRCKDLNTFLHEPFSSVAATCQTPKIACKNGDKNCHQSHGPVSLTMCKLTSGKYPNC RYKEKRQNKSYVVACKPPQKKDSQQFHLVPVHLDRVL (SEQ ID NO: 23). In some embodiments, the second RNA binding protein comprises or consists of an RNAse8 polypeptide. In some embodiments, the RNAse8 polypeptide comprises or consists of:
TSSQWFKTQHVQPSPQACNSAMSIINKYTERCKDLNTFLHEPFSSVAITCQTPNIACKNSC KNCHQSHGPMSLTMGELTSGKYPNCRYKEKHLNTPYIVACDPPQQGDPGYPLVPVHLD KVV (SEQ ID NO: 24). In some embodiments, the second RNA binding protein comprises or consists of an RNAse2 polypeptide. In some embodiments, the RNAse2 polypeptide comprises or consists of:
KPPQFTWAQWFETQHINMTSQQCTNAMQVINNYQRRCKNQNTFLLTTFANVVNVCGN PNMTCPSNKTRKNCHHSGSQVPLIHCNLTTPSPQNISNCRYAQTPANMFYIVACDNRDQ RRDPPQYPVVPVHLDRII (SEQ ID NO: 25). In some embodiments, the second RNA binding protein comprises or consists of an RNAse6PL polypeptide. In some embodiments, the RNAse6PL polypeptide comprises or consists of:
DKRLRDNHEWKKLIMVQHWPETVCEKIQNDCRDPPDYWTIHGLWPDKSEGCNRSWPF NLEEIKKNWMEITDSSLPSPSMGPAPPRWMRSTPRRSTLAEAWNSTGSWTSTGGCALPP AALPSGDLCCRPSLTAGSRGVGVDLTALHQLLHVHYSATGIIPEECSEPTKPFQIILHHDH TEWVQSIGMPIWGTISSSESAIGKNEESQPACAVLSHDS (SEQ ID NO: 26). In some embodiments, the second RNA binding protein comprises or consists of an RNAseL polypeptide. In some embodiments, the RNAseL polypeptide comprises or consists of:
AAVEDNHLLIKAVQNEDVDLVQQLLEGGANVNFQEEEGGWTPLHNAVQMSREDIVEL LLRHGADPVLRKKNGATPFILAAIAGSVKdLLKLFLSKGADVNECDFYGFTAFMEAAVY GKVKALKFLYKRGANVNLRRKTKEDQERLRKGGATALMDAAEKGHVEVLKILLDEM GADVNACDNMGRNALIHALLSSDDSDVEAITHLLLDHGADVNVRGERGKTPLILAVEK KHLGLVQRLLEQEHIEINDTDSDGKTALLLAVELKLKKIAELLCKRGASTDCGDLVMTA RRNYDHSLVKVLLSHGAKEDFHPPAEDWKPQSSHWGAALKDLHRIYRPMIGKLKFFID EKYKIADTSEGGIYLGFYEKQEVAVKTFCEGSPRAQREVSCLQSSRENSHLVTFYGSESH RGHLFVCVTLCEQTLEACLDVHRGEDVENEEDEFARNVLSSIFKAVQELHLSCGYTHQD LQPQNILIDSKKAAHLADFDKSIKWAGDPQEVKRDLEDLGRLVLYVVKKGSISFEDLKA QSNEEVVQLSPDEETKDLIHRLFHPGEHVRDCLSDLLGHPFFWTWESRYRTLRNVGNES DIKTRKSESEILRLLQPGPSEHSKSFDKWTTKINECVMKKMNKFYEKRGNFYQNTVGDL LKFIRNLGEHIDEEKHKKMKLKIGDPSLYFQKTFPDLVIYVYTKLQNTEYRKHFPQTHSP NKPQCDGAGGASGLASPGC (SEQ ID NO: 27). In some embodiments, the second RNA binding protein comprises or consists of an RNAseT2 polypeptide. In some embodiments, the RNAseT2 polypeptide comprises or consists of:
VQHWPETVCEKIQNDCRDPPDYWTIHGLWPDKSEGCNRSWPFNLEEIKDLLPEMRAYW PDVIHSFPNRSRFWKHEWEKHGTCAAQVDALNSQKKYFGRSLELYRELDLNSVLLKLGI KPSINYYQVADFKDALARVYGVIPKIQCLPPSQDEEVQTIGQIELCLTKQDQQLQNCTEP GEQPSPKQEVWLANGAAESRGLRVCEDGPVFYPPPKKTKH (SEQ ID NO: 28). In some embodiments, the second RNA binding protein comprises or consists of an RNAse11 polypeptide. In some embodiments the RNAse11 polypeptide comprises or consists of:
EASESTMKIIKEEFTDEEMQYDMAKSGQEKQTIEILMNPILLVKNTSLSMSKDDMSSTLL TFRSLHYNDPKGNSSGNDKECCNDMTVWRKVSEANGSCKWSNNFIRSSTEVMRRVHR APSCKFVQNPGISCCESLELENTVCQFTTGKQFPRCQYHSVTSLEKILTVLTGHSLMSWL VCGSKL (SEQ ID NO: 29). In some embodiments, the second RNA binding protein comprises or consists of an RNAseT2-like polypeptide. In some embodiments, the RNAseT2-like polypeptide comprises or consists of:

(SEQ ID NO: 30)
XLGGADKRLRDNHEWKKLIMVQHWPETVCEKIQNDCRDPPDYWTIHGLWP
DKSEGCNRSWPFNLEEIKDLLPEMRAYWPDVIHSFPNRSRFWKHEWEKHG
TCAAQVDALNSQKKYFGRSLELYRELDLNSVLLKLGIKPSINYYQTTEED
LNLDVEPTTEDTAEEVTIHVLLHSALFGEIGPRRW.

In some embodiments of the compositions of the disclosure, the second RNA binding protein comprises or consists of a mutated RNAse. In some embodiments, the second RNA binding protein comprises or consists of a mutated Rnase1 (Rnase1(K41R)) polypeptide. In some embodiments, the Rnase1(K41R) polypeptide comprises or consists of:
KESRAKKFQRQHMDSDSSPSSSSTYCNQMMRRRNMTQGRCRPVNTFVHEPLVDVQNV CFQEKVTCKNGQGNCYKSNSSMHITDCRLTNGSRYPNCAYRTSPKERHIIVACEGSPYV PVHFDASVEDST (SEQ ID NO: 116). In some embodiments, the second RNA binding protein comprises or consists of a mutated Rnase1 (Rnase1(K41R, D121E)) polypeptide. In some embodiments, the Rnase1 (Rnase1(K41R, D121E)) comprises or consists of:
KESRAKKFQRQHMDSDSSPSSSSTYCNQMMRRRNMTQGRCRPVNTFVHEPLVDVQNV CFQEKVTCKNGQGNCYKSNSSMHITDCRLTNGSRYPNCAYRTSPKERHIIVACEGSPYV PVHFEASVEDST (SEQ ID NO: 117). In some embodiments, the second RNA binding protein comprises or consists of a mutated Rnase1 (Rnase1(K41R, D121E, Hi19N)) polypeptide. In some embodiments, the Rnase1 (Rnase1(K41R, D121E, H119N)) polypeptide comprises or consists of:
KESRAKKFQRQHMDSDSSPSSSSTYCNQMMRRRNMTQGRCRPVNTFVHEPLVDVQNV CFQEKVTCKNGQGNCYKSNSSMHITDCRLTNGSRYPNCAYRTSPKERHIIVACEGSPYV PVNFEASVEDST (SEQ ID NO: 118). In some embodiments, the second RNA binding protein comprises or consists of a mutated Rnase1. In some embodiments, the second RNA binding protein comprises or consists of a mutated Rnase1 (Rnase1(H119N)) polypeptide. In some embodiments, the Rnase1 (Rnase1(H119N)) polypeptide comprises or consists of:
KESRAKKFQRQHMDSDSSPSSSSTYCNQMMRRRNMTQGRCKPVNTFVHEPLVDVQNV CFQEKVTCKNGQGNCYKSNSSMHITDCRLTNGSRYPNCAYRTSPKERHIIVACEGSPYV PVNFDASVEDST (SEQ ID NO: 119). In some embodiments, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D, H119N)) polypeptide comprises or consists of:
KESRAKKFQRQHMDSDSSPSSSSTYCNQMMRRRNMTQGDCKPVNTFVHEPLVDVQNV CFQEKVTCKDGQGNCYKSNSSMHITDCRLTADSDYPNCAYRTSPKERHIIVACEGSPYV PVNFDASVEDST (SEQ ID NO: 120). In some embodiments, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D, H119N, K41R, D121E)) polypeptide comprises or consists of:
KESRAKKFQRQHMDSDSSPSSSSTYCNQMMRRRNMTQGDCRPVNTFVHEPLVDVQNV CFQEKVTCKDGQGNCYKSNSSMHITDCRLTADSDYPNCAYRTSPKERHIIVACEGSPYV PVNFEASVEDST (SEQ ID NO: 121). In some embodiments, the second RNA binding protein comprises or consists of a mutated Rnase1 (Rnase1(R39D, N67D, N88A, G89D, R91D, H119N)) polypeptide. In some embodiments, the Rnase1 (Rnase1(R39D, N67D, N88A, G89D, R91D)) polypeptide comprises or consists of:

(SEQ ID NO: 122)
KESRAKKFQRQHMDSDSSPSSSSTYCNQMMRRRNMTQGDCKPVNTFVHEP
LVDVQNVCFQEKVTCKDGQGNCYKSNSSMHITDCRLTADSDYPNCAYRTS
PKERHIIVACEGSPYVPVHFDASVEDST.

In some embodiments, the second RNA binding protein comprises or consists of a mutated Rnase1 (Rnase1 (R39D, N67D, N88A, G89D, R91D, H119N, K41R, D121E)) polypeptide comprises or consists of:

(SEQ ID NO: 225)
KESRAKKFQRQHMDSDSSPSSSSTYCNQMMRRRNMTQGDCRPVNTFVHEP
LVDVQNVCFQEKVTCKDGQGNCYKSNSSMHITDCRLTADSDYPNCAYRTS
PKERHIIVACEGSPYVPVNFEASVEDST.

In some embodiments of the compositions of the disclosure, the second RNA binding protein comprises or consists of a NOB1 polypeptide. In some embodiments, the NOB1 polypeptide comprises or consists of:

(SEQ ID NO: 31)
APVEHVVADAGAFLRHAALQDIGKNIYTIREVVTEIRDKATRRRLAVLPY
ELRFKEPLPEYVRLVTEFSKKTGDYPSLSATDIQVLALTYQLEAEFVGVS
HLKQEPQKVKVSSSIQHPETPLHISGFHLPYKPKPPQETEKGHSACEPEN
LEFSSFMFWRNPLPNIDHELQELLIDRGEDVPSEEEEEEENGFEDRKDDS
DDDGGGWITPSNIKQIQQELEQCDVPEDVRVGCLTTDFAMQNVLLQMGLH
VLAVNGMLIREARSYILRCHGCFKTTSDMSRVFCSHCGNKTLKKVSVTV.

In some embodiments of the compositions of the disclosure, the second RNA binding protein comprises or consists of an endonuclease. In some embodiments, the second RNA binding protein comprises or consists of an endonuclease V (ENDOV). In some embodiments, the ENDOV polypeptide comprises or consists of:
AFSGLQRVGGVDVSFVKGDSVRACASLVVLSFPELEVVYEESRMVSLTAPYVSGFLAFR EVPFLLELVQQLREKEPGLMPQVLLVDGNGVLHHRGFGVACHLGVLTDLPCVGVAKKL LQVDGLENNALHKEKIRLLQTRGDSFPLLGDSGTVLGMALRSHDRSTRPLYISVGHRMS LEAAVRLTCCCCRFRIPEPVRQADICSREHIRKS (SEQ ID NO: 32). In some embodiments, the second RNA binding protein comprises or consists of an endonuclease G (ENDOG) polypeptide. In some embodiments, the ENDOG polypeptide comprises or consists of:
AELPPVPGGPRGPGELAKYGLPGLAQLKSRESYVLCYDPRTRGALWVVEQLRPERLRG DGDRRECDFREDDSVHAYHRATNADYRGSGFDRGHLAAAANHRWSQKAMDDTFYLS NVAPQVPHLNQNAWNNLEKYSRSLTRSYQNVYVCTGPLFLPRTEADGKSYVKYQVIGK NHVAVPTHFFKVLILEAAGGQIELRTYVMPNAPVDEAIPLERFLVPIESIERASGLLFVPNI LARAGSLKAITAGSK (SEQ ID NO: 33). In some embodiments, the second RNA binding protein comprises or consists of an endonuclease D1 (ENDOD1) polypeptide. In some embodiments, the ENDOD1 polypeptide comprises or consists of:
RLVGEEEAGFGECDKFFYAGTPPAGLAADSHVKICQRAEGAERFATLYSTRDRIPVYSA FRAPRPAPGGAEQRWLVEPQIDDPNSNLEEAINEAEAITSVNSLGSKQALNTDYLDSDYQ RGQLYPFSLSSDVQVATFTLTNSAPMTQSFQERWYVNLHSLMDRALTPQCGSGEDLYIL TGTVPSDYRVKDKVAVPEFVWLAACCAVPGGGWAMGFVKHTRDSDIIEDVMVKDLQ KLLPFNPQLFQNNCGETEQDTEKMKKILEVVNQIQDEERMVQSQKSSSPLSSTRSKRSTL LPPEASEGSSSFLGKLMGFIATPFIKLFQLIYYLVVAILKNIVYFLWCVTKQVINGIESCLY RLGSATISYFMAIGEELVSIPWKVLKVVAKVIRALLRILCCLLKAICRVLSIPVRVLVDVA TFPVYTMGAIPIVCKDIALGLGGTVSLLFDTAFGTLGGLFQVVFSVCKRIGYKVTFDNSG EL (SEQ ID NO: 34). In some embodiments, the second RNA binding protein comprises or consists of a Human flap endonuclease-1 (hFEN1) polypeptide. In some embodiments, the hFEN1 polypeptide comprises or consists of:
MGIQGLAKLIADVAPSAIRENDIKSYFGRKVAIDASMSIYQFLIAVRQGGDVLQNEEGET TSHLMGMFYRTIRMMENGIKPVYVFDGKPPQLKSGELAKRSERRAEAEKQLQQAQAAG AEQEVEKFTKRLVKVTKQHNDECKHLLSLMGIPYLDAPSEAEASCAALVKAGKVYAAA TEDMDCLTFGSPVLMRHLTASEAKKLPIQEFHLSRILQELGLNQEQFVDLCILLGSDYCE SIRGIGPKRAVDLIQKHKSIEEIVRRLDPNKYPVPENWLHKEAHQLFLEPEVLDPESVELK WSEPNEEELIKFMCGEKQFSEERIRSGVKRLSKSRQGSTQGRLDDFFKVTGSLSSAKRKE PEPKGSTKKKAKTGAAGKFKRGK (SEQ ID NO: 35). In some embodiments, the second RNA binding protein comprises or consists of a DNA repair endonuclease XPF (ERCC4) polypeptide. In some embodiments, the ERCC4 polypeptide comprises or consists of:

(SEQ ID NO: 124)
MESGQPARRIAMAPLLEYERQLVLELLDTDGLVVCARGLGADRLLYHFLQ
LHCHPACLVLVLNTQPAEEEYFINQLKIEGVEHLPRRVTNEITSNSRYEV
YTQGGVIFATSRILVVDFLTDRIPSDLITGILVYRAHRIIESCQEAFILR
LFRQKNKRGFIKAFTDNAVAFDTGFCHVERVMRNLFVRKLYLWPRFHVAV
NSFLEQHKPEVVEIHVSMTPTMLAIQTAILDILNACLKELKCHNPSLEVE
DLSLENAIGKPFDKTIRHYLDPLWHQLGAKTKSLVQDLKILRTLLQYLSQ
YDCVTFLNLLESLRATEKAFGQNSGWLFLDSSTSMFINARARVYHLPDAK
MSKKEKISEKMEIKEGEGILWG.

In some embodiments of the compositions of the disclosure, the second RNA binding protein comprises or consists of an Endonuclease III-like protein 1 (NTHL) polypeptide. In some embodiments, the NTHL polypeptide comprises or consists of:

(SEQ ID NO: 123)
CSPQESGMTALSARMLTRSRSLGPGAGPRGCREEPGPLRRREAAAEARKS
HSPVKRPRKAQRLRVAYEGSDSEKGEGAEPLKVPVWEPQDWQQQLVNIRA
MRNKKDAPVDHLGTEHCYDSSAPPKVRRYQVLLSLMLSSQTKDQVTAGAM
QRLRARGLTVDSILQTDDATLGKLIYPVGFWRSKVKYIKQTSAILQQHYG
GDIPASVAELVALPGVGPKMAHLAMAVAWGTVSGIAVDTHVHRIANRLRW
TKKATKSPEETRAALEEWLPRELWHEINGLLVGFGQQTCLPVHPRCHACL
NQALCPAAQGL.

In some embodiments of the compositions of the disclosure, the second RNA binding protein comprises or consists of a human Schlafen 14 (hSLFN14) polypeptide. In some embodiments, the hSLFN14 polypeptide comprises or consists of:

(SEQ ID NO: 36)
ESTHVEFKRFTTKKVIPRIKEMLPHYVSAFANTQGGYVLIGVDDKSKEVV
GCKWEKVNPDLLKKEIENCIEKLPTFHFCCEKPKVNFTTKILNVYQKDVL
DGYVCVIQVEPFCCVVFAEAPDSWIMKDNSVTRLTAEQWVVMMLDTQSAP
PSLVTDYNSCLISSASSARKSPGYPIKVHKFKEALQ.

In some embodiments of the compositions of the disclosure, the second RNA binding protein comprises or consists of a human beta-lactamase-like protein 2 (hLACTB2) polypeptide. In some embodiments, the hLACTB2 polypeptide comprises or consists of:

(SEQ ID NO: 37)
TLQGTNTYLVGTGPRRILIDTGEPAIPEYISCLKQALTEFNTAIQEIVVT
HWHRDHSGGIGDICKSINNDTTYCIKKLPRNPQREEIIGNGEQQYVYLKD
GDVIKTEGATLRVLYTPGHTDDHMALLLEEENAIFSGDCILGEGTTVFED
LYDYMNSLKELLKIKADIIYPGHGPVIHNAEAKIQQYISHRNIREQQILT
LFRENFEKSFTVMELVKIIYKNTPENLHEMAKHNLLLHLKKLEKEGKIFS
NTDPDKKWKAHL.

In some embodiments of the compositions of the disclosure, the second RNA binding protein comprises or consists of an apurinic/apyrimidinic (AP) endodeoxyribonuclease (APEX) polypeptide. In some embodiments, the second RNA binding protein comprises or consists of an apurinic/apyrimidinic (AP) endodeoxyribonuclease (APEX2) polypeptide. In some embodiments, the APEX2 polypeptide comprises or consists of:
MLRVVSWNINGIRRPLQGVANQEPSNCAAVAVGRILDELDADIVCLQETKVTRDALTEP LAIVEGYNSYFSFSRNRSGYSGVATFCKDNATPVAAEEGLSGLFATQNGDVGCYGNMD EFTQEELRALDSEGRALLTQHKIRTWEGKEKTLTLINVYCPHADPGRPERLVFKMRFYR LLQIRAEALLAAGSHVIILGDLNTAHRPIDHWDAVNLECFEEDPGRKWMDSLLSNLGCQ SASHVGPFIDSYRCFQPKQEGAFTCWSAVTGARHLNYGSRLDYVLGDRTLVIDTFQASF LLPEVMGSDHCPVGAVLSVSSVPAKQCPPLCTRFLPEFAGTQLKILRFLVPLEQSPVLEQ STLQHNNQTRVQTCQNKAQVRSTRPQPSQVGSSRGQKNLKSYFQPSPSCPQASPDIELPS LPLMSALMTPKTPEEKAVAKVVKGQAKTSEAKDEKELRTSFWKSVLAGPLRTPLCGGH REPCVMRTVKKPGPNLGRRFYMCARPRGPPTDPSSRCNFFLWSRPS (SEQ ID NO: 38). In some embodiments, the APEX2 polypeptide comprises or consists of:
MLRVVSWNINGIRRPLQGVANQEPSNCAAVAVGRILDELDADIVCLQETKVTRDALTEP LAIVEGYNSYFSFSRNRSGYSGVATFCKDNATPVAAEEGLSGLFATQNGDVGCYGNMD EFTQEELRALDSEGRALLTQHKIRTWEGKEKTLTLINVYCPHADPGRPERLVFKMRFYR LLQIRAEALLAAGSHVIILGDLNTAHRPIDHWDAVNLECFEEDPGRKWMDSLLSNLGCQ SASHVGPFIDSYRCFQPKQEGAFTCWSAVTGARHLNYGSRLDYVLGDRTLVIDTFQASF LLPEVMGSDHCPVGAVLSVSSVPAKQCPPLCTRFLPEFAGTQLKILRFLVPLEQSP (SEQ ID NO: 39). In some embodiments, the second RNA binding protein comprises or consists of an apurinic or apyrimidinic site lyase (APEX1) polypeptide. In some embodiments, the APEX1 polypeptide comprises or consists of:

(SEQ ID NO: 125)
PKRGKKGAVAEDGDELRTEPEAKKSKTAAKKNDKEAAGEGPALYEDPPDQ
KTSPSGKPATLKICSWNVDGLRAWIKKKGLDWVKEEAPDILCLQETKCSE
NKLPAELQELPGLSHQYWSAPSDKEGYSGVGLLSRQCPLKVSYGIGDEEH
DQEGRVIVAEFDSFVLVTAYVPNAGRGLVRLEYRQRWDEAFRKFLKGLAS
RKPLVLCGDLNVAHEEIDLRNPKGNKKNAGFTPQERQGFGELLQAVPLAD
SFRHLYPNTPYAYTFWTYMMNARSKNVGWRLDYFLLS.

In some embodiments of the compositions of the disclosure, the second RNA binding protein comprises or consists of an angiogenin (ANG) polypeptide. In some embodiments, the ANG polypeptide comprises or consists of:

(SEQ ID NO: 40)
QDNSRYTHFLTQHYDAKPQGRDDRYCESIMRRRGLTSPCKDINTFIHGNK
RSIKAICENKNGNPHRENLRISKSSFQVTTCKLHGGSPWPPCQYRATAGF
RNVVVACENGLPVHLDQSIFRRP.

In some embodiments of the compositions of the disclosure, the second RNA binding protein comprises or consists of a heat responsive protein 12 (HRSP12) polypeptide. In some embodiments, the HRSP12 polypeptide comprises or consists of:

(SEQ ID NO: 41)
SSLIRRVISTAKAPGAIGPYSQAVLVDRTIYISGQIGMDPSSGQLVSGGV
AEEAKQALKNMGEILKAAGCDFTNVVKTTVLLADINDFNTVNEIYKQYFK
SNFPARAAYQVAALPKGSRIEIEAVAIQGPLTTASL.

In some embodiments of the compositions of the disclosure, the second RNA binding protein comprises or consists of a Zinc Finger CCCH-Type Containing 12A (ZC3H12A) polypeptide. In some embodiments, the ZC3H12A polypeptide comprises or consists of:
GGGTPKAPNLEPPLPEEEKEGSDLRPVVIDGSNVAMSHGNKEVFSCRGILLAVNWFLER GHTDITVFVPSWRKEQPRPDVPITDQHILRELEKKKILVFTPSRRVGGKRVVCYDDRFIV KLAYESDGIVVSNDTYRDLQGERQEWKRFIEERLLMYSFVNDKFMPPDDPLGRHGPSLD NFLRKKPLTLE (SEQ ID NO: 42). In some embodiments, the ZC3H12A polypeptide comprises or consists of:

(SEQ ID NO: 43)
SGPCGEKPVLEASPTMSLWEFEDSHSRQGTPRPGQELAAEEASALELQMK
VDFFRKLGYSSTEIHSVLQKLGVQADTNTVLGELVKHGTATERERQTSPD
PCPQLPLVPRGGGTPKAPNLEPPLPEEEKEGSDLRPVVIDGSNVAMSHGN
KEVFSCRGILLAVNWFLERGHTDITVFVPSWRKEQPRPDVPITDQHILRE
LEKKKILVFTPSRRVGGKRVVCYDDRFIVKLAYESDGIVVSNDTYRDLQG
ERQEWKRFIEERLLMYSFVNDKFMPPDDPLGRHGPSLDNFLRKKPLTLEH
RKQPCPYGRKCTYGIKCRFFHPERPSCPQRSVADELRANALLSPPRAPSK
DKNGRRPSPSSQSSSLLTESEQCSLDGKKLGAQASPGSRQEGLTQTYAPS
GRSLAPSGGSGSSFGPTDWLPQTLDSLPYVSQDCLDSGIGSLESQMSELW
GVRGGGPGEPGPPRAPYTGYSPYGSELPATAAFSAFGRAMGAGHFSVPAD
YPPAPPAFPPREYWSEPYPLPPPTSVLQEPPVQSPGAGRSPWGRAGSLAK
EQASVYTKLCGVFPPHLVEAVMGRFPQLLDPQQLAAEILSYKSQHPSE.

In some embodiments of the compositions of the disclosure, the second RNA binding protein comprises or consists of a Reactive Intermediate Imine Deaminase A (RIDA) polypeptide. In some embodiments, the RIDA polypeptide comprises or consists of:

(SEQ ID NO: 44)
SSLIRRVISTAKAPGAIGPYSQAVLVDRTIYISGQIGMDPSSGQLVSGGV
AEEAKQALKNMGEILKAAGCDFTNVVKTTVLLADINDFNTVNEIYKQYFK
SNFPARAAYQVAALPKGSRIEIEAVAIQGPLTTASL.

In some embodiments of the compositions of the disclosure, the second RNA binding protein comprises or consists of a Phospholipase D Family Member 6 (PDL6) polypeptide. In some embodiments, the PDL6 polypeptide comprises or consists of:

(SEQ ID NO: 126)
EALFFPSQVTCTEALLRAPGAELAELPEGCPCGLPHGESALSRLLRALLA
ARASLDLCLFAFSSPQLGRAVQLLHQRGVRVRVVTDCDYMALNGSQIGLL
RKAGIQVRHDQDPGYMHHKFAIVDKRVLITGSLNWTTQAIQNNRENVLIT
EDDEYVRLFLEEFERIWEQFNPTKYTFFPPKKSHGSCAPPVSRAGGRLLS
WHRTCGTSSESQT.

In some embodiments of the compositions of the disclosure, the second RNA binding protein comprises or consists of a mitochondrial ribonuclease P catalytic subunit (KIAA0391) polypeptide. In some embodiments, the KIAA0391 polypeptide comprises or consists of:

(SEQ ID NO: 127)
KARYKTLEPRGYSLLIRGLIHSDRWREALLLLEDIKKVITPSKKNYNDCI
QGALLHQDVNTAWNLYQELLGHDIVPMLETLKAFFDFGKDIKDDNYSNKL
LDILSYLRNNQLYPGESFAHSIKTWFESVPGKQWKGQFTTVRKSGQCSGC
GKTIESIQLSPEEYECLKGKIMRDVIDGGDQYRKTTPQELKRFENFIKSR
PPFDVVIDGLNVAKMFPKVRESQLLLNVVSQLAKRNLRLLVLGRKHMLRR
SSQWSRDEMEEVQKQASCFFADDISEDDPFLLYATLHSGNHCRFITRDLM
RDHKACLPDAKTQRLFFKWQQGHQLAIVNRFPGSKLTFQRILSYDTVVQT
TGDSWHIPYDEDLVERCSCEVPTKWLCLHQKT.

In some embodiments of the compositions of the disclosure, the second RNA binding protein comprises or consists of an argonaute 2 (AGO2) polypeptide.

In some embodiments of the compositions of the disclosure, the AGO2 polypeptide comprises or consists of:

(SEQ ID NO: 128)
SVEPMFRHLKNTYAGLQLVVVILPGKTPVYAEVKRVGDTVLGMATQCVQM
KNVQRTTPQTLSNLCLKINVKLGGVNNILLPQGRPPVFQQPVIFLGADVT
HPPAGDGKKPSIAAVVGSMDAHPNRYCATVRVQQHRQEIIQDLAAMVREL
LIQFYKSTRFKPTRIIFYRDGVSEGQFQQVLHHELLAIREACIKLEKDYQ
PGITFIVVQKRHHTRLFCTDKNERVGKSGNIPAGTTVDTKITHPTEFDFY
LCSHAGIQGTSRPSHYHVLWDDNRFSSDELQILTYQLCHTYVRCTRSVSI
PAPAYYAHLVAFRARYHLVDKEHDSAEGSHTSGQSNGRDHQALAKAVQVH
QDTLRTMYFA.

In some embodiments of the compositions of the disclosure, the second RNA binding protein comprises or consists of a mitochondrial nuclease EXOG (EXOG) polypeptide. In some embodiments, the EXOG polypeptide comprises or consists of:

(SEQ ID NO: 129)
QGAEGALTGKQPDGSAEKAVLEQFGFPLTGTEARCYTNHALSYDQAKRVP
RWVLEHISKSKIMGDADRKHCKFKPDPMPPTFSAFNEDYVGSGWSRGHMA
PAGNNKFSSKAMAETFYLSNIVPQDFDNNSGYWNRIEMYCRELTERFEDV
WVVSGPLTLPQTRGDGKKIVSYQVIGEDNVAVPSHLYKVILARRSSVSTE
PLALGAFVVPNEAIGFQPQLTEFQVSLQDLEKLSGLVFFPHLDRTSDIRN
ICSVDTCKLLDFQEFTLYLSTRKIEGARSVLRLEKIMENLKNAEIEPDDY
FMSRYEKKLEELKAKEQSGTQIRKPS.

In some embodiments of the compositions of the disclosure, the second RNA binding protein comprises or consists of a Zinc Finger CCCH-Type Containing 12D (ZC3H12D) polypeptide. In some embodiments, the ZC3H12D polypeptide comprises or consists of:

(SEQ ID NO: 130)
EHPSKMEFFQKLGYDREDVLRVLGKLGEGALVNDVLQELIRTGSRPGALE
HPAAPRLVPRGSCGVPDSAQRGPGTALEEDFRTLASSLRPIVIDGSNVAM
SHGNKETFSCRGIKLAVDWFRDRGHTYIKVFVPSWRKDPPRADTPIREQH
VLAELERQAVLVYTPSRKVHGKRLVCYDDRYIVKVAYEQDGVIVSNDNYR
DLQSENPEWKWFIEQRLLMFSFVNDRFMPPDDPLGRHGPSLSNFLSRKPK
PPEPSWQHCPYGKKCTYGIKCKFYHPERPHHAQLAVADELRAKTGARPGA
GAEEQRPPRAPGGSAGARAAPREPFAHSLPPARGSPDLAALRGSFSRLAF
SDDLGPLGPPLPVPACSLTPRLGGPDWVSAGGRVPGPLSLPSPESQFSPG
DLPPPPGLQLQPRGEHRPRDLHGDLLSPRRPPDDPWARPPRSDRFPGRSV
WAEPAWGDGATGGLSVYATEDDEGDARARARIALYSVFPRDQVDRVMAAF
PELSDLARLILLVQRCQSAGAPLGKP.

In some embodiments of the compositions of the disclosure, the second RNA binding protein comprises or consists of an endoplasmic reticulum to nucleus signaling 2 (ERN2) polypeptide. In some embodiments, the ERN2 polypeptide comprises or consists of:

(SEQ ID NO: 131)
RQQQPQVVEKQQETPLAPADFAHISQDAQSLHSGASRRSQKRLQSPSKQA
QPLDDPEAEQLTVVGKISFNPKDVLGRGAGGTFVFRGQFEGRAVAVKRLL
RECFGLVRREVQLLQESDRHPNVLRYFCTERGPQFHYIALELCRASLQEY
VENPDLDRGGLEPEVVLQQLMSGLAHLHSLHIVHRDLKPGNILITGPDSQ
GLGRVVLSDFGLCKKLPAGRCSFSLHSGIPGTEGWMAPELLQLLPPDSPT
SAVDIFSAGCVFYYVLSGGSHPFGDSLYRQANILTGAPCLAHLEEEVHDK
VVARDLVGAMLSPLPQPRPSAPQVLAHPFFWSRAKQLQFFQDVSDWLEKE
SEQEPLVRALEAGGCAVVRDNWHEHISMPLQTDLRKFRSYKGTSVRDLLR
AVRNKKHHYRELPVEVRQALGQVPDGFVQYFTNRFPRLLLHTHRAMRSCA
SESLFLPYYPPDSEARRPCPGATGR.

In some embodiments of the compositions of the disclosure, the second RNA binding protein comprises or consists of a pelota mRNA surveillance and ribosome rescue factor (PELO) polypeptide. In some embodiments, the PELO polypeptide comprises or consists of:

(SEQ ID NO: 132)
KLVRKNIEKDNAGQVTLVPEEPEDMWHTYNLVQVGDSLRASTIRKVQTES
STGSVGSNRVRTTLTLCVEAIDFDSQACQLRVKGTNIQENEYVKMGAYHT
IELEPNRQFTLAKKQWDSVVLERIEQACDPAWSADVAAVVMQEGLAHICL
VTPSMTLTRAKVEVNIPRKRKGNCSQHDRALERFYEQVVQAIQRHIHFDV
VKCILVASPGFVREQFCDYLFQQAVKTDNKLLLENRSKFLQVHASSGHKY
SLKEALCDPTVASRLSDTKAAGEVKALDDFYKMLQHEPDRAFYGLKQVEK
ANEAMAIDTLLISDELFRHQDVATRSRYVRLVDSVKENAGTVRIFSSLHV
SGEQLSQLTGVAAILRFPVPELSDQEGDSSSEED.

In some embodiments of the compositions of the disclosure, the second RNA binding protein comprises or consists of a YBEY metallopeptidase (YBEY) polypeptide. In some embodiments, the YBEY polypeptide comprises or consists of:

(SEQ ID NO: 133)
SLVIRNLQRVIPIRRAPLRSKIEIVRRILGVQKFDLGIICVDNKNIQHIN
RIYRDRNVPTDVLSFPFHEHLKAGEFPQPDFPDDYNLGDIFLGVEYIFHQ
CKENEDYNDVLTVTATHGLCHLLGFTHGTEAEWQQMFQKEKAVLDELGRR
TGTRLQPLTRGLFGGS.

In some embodiments of the compositions of the disclosure, the second RNA binding protein comprises or consists of a cleavage and polyadenylation specific factor 4 like (CPSF4L) polypeptide. In some embodiments, the CPSF4L comprises or consists of:

(SEQ ID NO: 134)
QEVIAGLERFTFAFEKDVEMQKGTGLLPFQGMDKSASAVCNFFTKGLCEK
GKLCPFRHDRGEKMVVCKHWLRGLCKKGDHCKFLHQYDLTRMPECYFYSK
FGDCSNKECSFLHVKPAFKSQDCPWYDQGFCKDGPLCKYRHVPRIMCLNY
LVGFCPEGPKCQFAQKIREFKLLPGSKI.

In some embodiments of the compositions of the disclosure, the second RNA binding protein comprises or consists of an hCG_2002731 polypeptide. In some embodiments, the hCG_2002731 polypeptide comprises or consists of:
KLVRKNIEKDNAGQVTLVPEEPEDMWHTYNLVQVGDSLRASTIRKVQTESSTGSVGSN RVRTTLTLCVEAIDFDSQACQLRVKGTNIQENEYVKMGAYHTIELEPNRQFTLAKKQW DSVVLERIEQACDPAWSADVAAVVMQEGLAHICLVTPSMTLTRAKVEVNIPRKRKGNC SQHDRALERFYEQVVQAIQRHIHFDVVKCILVASPGFVREQFCDYMFQQAVKTDNKLLL ENRSKFLQVHASSGHKYSLKEALCDPTVASRLSDTKAAGEVKALDDFYKMLQHEPDRA FYGLKQVEKANEAMAIDTLLISDELFRHQDVATRSRYVRLVDSVKENAGTVRIFSSLHV SGEQLSQLTGVAAILRFPVPELSDQEGDSSSEED (SEQ ID NO: 135). In some embodiments, the hCG_2002731 polypeptide comprises or consists of:

(SEQ ID NO: 136)
DPAWSADVAAVVMQEGLAHICLVTPSMTLTRAKVEVNIPRKRKGNCSQHD
RALERFYEQVVQAIQRHIHFDVVKCILVASPGFVREQFCDYMFQQAVKTD
NKLLLENRSKFLQVHASSGHKYSLKEALCDPTVASRLSDTKAAGEVKALD
DFYKMLQHEPDRAFYGLKQVEKANEAMAIDTLLISDELFRHQDVATRSRY
VRLVDSVKENAGTVRIFSSLHVSGEQLSQLTGVAAILRFPVPELSDQEGD
SSSEED.

In some embodiments of the compositions of the disclosure, the second RNA binding protein comprises or consists of an Excision Repair Cross-Complementation Group 1 (ERCC1) polypeptide. In some embodiments, the ERCC1 polypeptide comprises or consists of:

(SEQ ID NO: 137)
MDPGKDKEGVPQPSGPPARKKFVIPLDEDEVPPGVRGNPVLKFVRNVPWE
FGDVIPDYVLGQSTCALFLSLRYHNLHPDYIHGRLQSLGKNFALRVLLVQ
VDVKDPQQALKELAKMCILADCTLILAWSPEEAGRYLETYKAYEQKPADL
LMEKLEQDFVSRVTECLTTVKSVNKTDSQTLLTTFGSLEQLIAASREDLA
LCPGLGPQK.

In some embodiments of the compositions of the disclosure, the second RNA binding protein comprises or consists of a ras-related C3 botulinum toxin substrate 1 isoform (RAC1) polypeptide. In some embodiments, the RAC1 polypeptide comprises or consists of:

(SEQ ID NO: 138)
KESRAKKFQRQHMDSDSSPSSSSTYCNQMMRRRNMTQGRCKPVNTFVHEP
LVDVQNVCFQEKVTCKNGQGNCYKSNSSMHITDCRLTNGSRYPNCAYRTS
PKERHIIVACEGSPYVPVHFDASVEDST.

In some embodiments of the compositions of the disclosure, the second RNA binding protein comprises or consists of a Ribonuclease A A1 (RAA1) polypeptide. In some embodiments, the RAA1 polypeptide comprises or consists of:

(SEQ ID NO: 139)
QDNSRYTHFLTQHYDAKPQGRDDRYCESIMRRRGLTSPCKDINTFIHGNK
RSIKAICENKNGNPHRENLRISKSSFQVTTCKLHGGSPWPPCQYRATAGF
RNVVVACENGLPVHLDQSIFRRP.

In some embodiments of the compositions of the disclosure, the second RNA binding protein comprises or consists of a Ras Related Protein (RAB1) polypeptide. In some embodiments, the RAB1 polypeptide comprises or consists of:

(SEQ ID NO: 140)
GLGLVQPSYGQDGMYQRFLRQHVHPEETGGSDRYCNLMMQRRKMTLYHCK
RFNTFIHEDIWNIRSICSTTNIQCKNGKMNCHEGVVKVTDCRDTGSSRAP
NCRYRAIASTRRVVIACEGNPQVPVHFDG.

In some embodiments of the compositions of the disclosure, the second RNA binding protein comprises or consists of a DNA Replication Helicase/Nuclease 2 (DNA2) polypeptide. In some embodiments, the DNA2 polypeptide comprises or consists of:

(SEQ ID NO: 141)
XSAVDNILLKLAKFKIGFLRLGQIQKVHPAIQQFTEQEICRSKSIKSLAL
LEELYNSQLIVATTCMGINHPIFSRKIFDFCIVDEASQISQPICLGPLFF
SRRFVLVGDHQQLPPLVLNREARALGMSESLFKRLEQNKSAVVQLTVQYR
MNSKIMSLSNKLTYEGKLECGSDKVANAVINLRHFKDVKLELEFYADYSD
NPWLMGVFEPNNPVCFLNTDKVPAPEQVEKGGVSNVTEAKLIVFLTSIFV
KAGCSPSDIGIIAPYRQQLKIINDLLARSIGMVEVNTVDKYQGRDKSIVL
VSFVRSNKDGTVGELLKDWRRLNVAITRAKHKLILLGCVPSLNCYPPLEK
LLNHLNSEKLISFFFCIWSHLIALL.

In some embodiments of the compositions of the disclosure, the second RNA binding protein comprises or consists of a FLJ35220 polypeptide. In some embodiments, the FLJ35220 polypeptide comprises or consists of:

(SEQ ID NO: 142)
MALRSHDRSTRPLYISVGHRMSLEAAVRLTCCCCRFRIPEPVRQADICSR
EHIRKSLGLPGPPTPRSPKAQRPVACPKGDSGESSALC.

In some embodiments of the compositions of the disclosure, the second RNA binding protein comprises or consists of a FLJ13173 polypeptide. In some embodiments, the FLJ13173 polypeptide comprises or consists of:

(SEQ ID NO: 143)
CYTNHALSYDQAKRVPRWVLEHISKSKIMGDADRKHCKFKPDPNIPPTFS
AFNEDYVGSGWSRGHMAPAGNNKFSSKAMAETFYLSNIVPQDFDNNSGYW
NRIEMYCRELTERFEDVWVVSGPLTLPQTRGDGKKIVSYQVIGEDNVAVP
SHLYKVILARRSSVSTEPLALGAFVVPNEAIGFQPQLTEFQVSLQDLEKL
SGLVFFPHLDRT.

In some embodiments of the compositions of the disclosure, the second RNA binding protein comprises or consists of Teneurin Transmembrane Protein (TENM) polypeptide. In some embodiments, the second RNA binding protein comprises or consists of Teneurin Transmembrane Protein 1 (TENM1) polypeptide. In some embodiments, the TENM1 polypeptide comprises or consists of:
VTVSQMTSVLNGKTRRFADIQLQHGALCFNIRYGTTVEEEKNHVLEIARQRAVAQAWT KEQRRLQEGEEGIRAWTEGEKQQLLSTGRVQGYDGYFVLSVEQYLELSDSANNIHFMR QSEIGRR (SEQ ID NO: 144). In some embodiments, the second RNA binding protein comprises or consists of Teneurin Transmembrane Protein 2 (TENM2) polypeptide. In some embodiments, the TENM2 polypeptide comprises or consists of:

(SEQ ID NO: 145)
TVSQPTLLVNGKTRRFTNIEFQYSTLLLSIRYGLTPDTLDEEKARVLDQA
RQRALGTAWAKEQQKARDGREGSRLWTEGEKQQLLSTGRVQGYEGYYVLP
VEQYPELADSSSNIQFLRQNEMGKR.

In some embodiments of the compositions of the disclosure, the second RNA binding protein comprises or consists of Ribonuclease Kappa (RNAseK) polypeptide. In some embodiments, the RNAseK polypeptide comprises or consists of:

(SEQ ID NO: 204)
MGWLRPGPRPLCPPARASWAFSHRFPSPLAPRRSPTPFFMASLLCCGPKL
AACGIVLSAWGVIMLIMLGIFFNVHSAVLIEDVPFTEKDFENGPQNIYNL
YEQVSYNCFIAAGLYLLLGGFSFCQVRLNKRKEYMVR.

In some embodiments of the compositions of the disclosure, the second RNA binding protein comprises or consists of a transcription activator-like effector nuclease (TALEN) polypeptide or a nuclease domain thereof.

In some embodiments of the compositions of the disclosure, the second RNA binding protein comprises or consists a zinc finger nuclease polypeptide or a nuclease domain thereof. In some embodiments, the second RNA binding protein comprises or consists of a ZNF638 polypeptide or a nuclease domain thereof.

In some embodiments of the compositions of the disclosure, the second RNA binding protein comprises or consists of a PIN domain derived from the human SMG6 protein, also commonly known as telomerase-binding protein EST1A isoform 3, NCBI Reference Sequence: NP_001243756.1. In some embodiments, the PIN from hSMG6 is used herein in the form of a Cas fusion protein and as an internal control.

### Guide RNA

The terms guide RNA (gRNA) and single guide RNA (sgRNA) are used interchangeably throughout the disclosure.

Guide RNAs (gRNAs) of the disclosure may comprise of a spacer sequence and a scaffolding sequence. In some embodiments, a guide RNA is a single guide RNA (sgRNA) comprising a contiguous spacer sequence and scaffolding sequence. In some embodiments, the spacer sequence and the scaffolding sequence are contiguous. In some embodiments, a scaffold sequence comprises a “direct repeat” (DR) sequence. DR sequences refer to the repetitive sequences in the CRISPR locus (naturally-occurring in a bacterial genome or plasmid) that are interspersed with the spacer sequences. It is well known that one would be able to infer the DR sequence of a corresponding Cas protein if the sequence of the associated CRISPR locus is known. In some embodiments, the spacer sequence and the scaffolding sequence are not contiguous. In some embodiments, a sequence encoding a guide RNA of the disclosure comprises or consists of a spacer sequence and a scaffolding sequence, that are separated by a linker sequence. In some embodiments, the linker sequence may comprise or consist of 1, 2, 3, 4, 5, 6, 7, 8, 9, 10, 15, 20, 25, 30, 35, 40, 45, 50 or any number of nucleotides in between. In some embodiments, the linker sequence may comprise at least 1, 2, 3, 4, 5, 6, 7, 8, 9, 10, 15, 20, 25, 30, 35, 40, 45, 50 or any number of nucleotides in between.

Guide RNAs (gRNAs) of the disclosure may comprise non-naturally occurring nucleotides. In some embodiments, a guide RNA of the disclosure or a sequence encoding the guide RNA comprises or consists of modified or synthetic RNA nucleotides. Exemplary modified RNA nucleotides include, but are not limited to, pseudouridine (Ψ), dihydrouridine (D), inosine (I), and 7-methylguanosine (m7G), hypoxanthine, xanthine, xanthosine, 7-methylguanine, 5, 6-Dihydrouracil, 5-methylcytosine, 5-methylcytidine, 5-hydropxymethylcytosine, isoguanine, and isocytosine.

Guide RNAs (gRNAs) of the disclosure may bind modified RNA within a target sequence. Within a target sequence, guide RNAs (gRNAs) of the disclosure may bind modified RNA. Exemplary epigenetically or post-transcriptionally modified RNA include, but are not limited to, 2′-O-Methylation (2′-OMe) (2′-O-methylation occurs on the oxygen of the free 2′-OH of the ribose moiety), N6-methyladenosine (m6A), and 5-methylcytosine (m5C).

In some embodiments of the compositions of the disclosure, a guide RNA of the disclosure comprises at least one sequence encoding a non-coding C/D box small nucleolar RNA (snoRNA) sequence. In some embodiments, the snoRNA sequence comprises at least one sequence that is complementary to the target RNA, wherein the target sequence of the RNA molecule comprises at least one 2′-OMe. In some embodiments, the snoRNA sequence comprises at least one sequence that is complementary to the target RNA, wherein the at least one sequence that is complementary to the target RNA comprises a box C motif (RUGAUGA) and a box D motif (CUGA).

Spacer sequences of the disclosure bind to the target sequence of an RNA molecule. Spacer sequences of the disclosure may comprise a CRISPR RNA (crRNA). Spacer sequences of the disclosure comprise or consist of a sequence having sufficient complementarity to a target sequence of an RNA molecule to bind selectively to the target sequence. Upon binding to a target sequence of an RNA molecule, the spacer sequence may guide one or more of a scaffolding sequence and a fusion protein to the RNA molecule. In some embodiments, a sequence having sufficient complementarity to a target sequence of an RNA molecule to bind selectively to the target sequence has at least 50%, 55%, 60%, 65%, 70%, 75%, 80%, 85%, 90%, 95%, 96, 97%, 98%, 99%, or any percentage identity in between to the target sequence. In some embodiments, a sequence having sufficient complementarity to a target sequence of an RNA molecule to bind selectively to the target sequence has 100% identity the target sequence.

Scaffolding sequences of the disclosure bind the first RNA-binding polypeptide of the disclosure. Scaffolding sequences of the disclosure may comprise a trans acting RNA (tracrRNA). Scaffolding sequences of the disclosure comprise or consist of a sequence having sufficient complementarity to a target sequence of an RNA molecule to bind selectively to the target sequence. Upon binding to a target sequence of an RNA molecule, the scaffolding sequence may guide a fusion protein to the RNA molecule. In some embodiments, a sequence having sufficient complementarity to a target sequence of an RNA molecule to bind selectively to the target sequence has at least 50%, 55%, 60%, 65%, 70%, 75%, 80%, 85%, 90%, 95%, 96, 97%, 98%, 99%, or any percentage identity in between to the target sequence. In some embodiments, a sequence having sufficient complementarity to a target sequence of an RNA molecule to bind selectively to the target sequence has 100% identity the target sequence. Alternatively, or in addition, in some embodiments, scaffolding sequences of the disclosure comprise or consist of a sequence that binds to a first RNA binding protein or a second RNA binding protein of a fusion protein of the disclosure. In some embodiments, scaffolding sequences of the disclosure comprise a secondary structure or a tertiary structure. Exemplary secondary structures include, but are not limited to, a helix, a stem loop, a bulge, a tetraloop and a pseudoknot. Exemplary tertiary structures include, but are not limited to, an A-form of a helix, a B-form of a helix, and a Z-form of a helix. Exemplary tertiary structures include, but are not limited to, a twisted or helicized stem loop. Exemplary tertiary structures include, but are not limited to, a twisted or helicized pseudoknot. In some embodiments, scaffolding sequences of the disclosure comprise at least one secondary structure or at least one tertiary structure. In some embodiments, scaffolding sequences of the disclosure comprise one or more secondary structure(s) or one or more tertiary structure(s).

In some embodiments of the compositions of the disclosure, a guide RNA or a portion thereof selectively binds to a tetraloop motif in an RNA molecule of the disclosure. In some embodiments, a target sequence of an RNA molecule comprises a tetraloop motif. In some embodiments, the tetraloop motif is a “GRNA” motif comprising or consisting of one or more of the sequences of GAAA, GUGA, GCAA or GAGA.

In some embodiments of the compositions of the disclosure, a guide RNA or a portion thereof that binds to a target sequence of an RNA molecule hybridizes to the target sequence of the RNA molecule. In some embodiments, a guide RNA or a portion thereof that binds to a first RNA binding protein or to a second RNA binding protein covalently binds to the first RNA binding protein or to the second RNA binding protein. In some embodiments, a guide RNA or a portion thereof that binds to a first RNA binding protein or to a second RNA binding protein non-covalently binds to the first RNA binding protein or to the second RNA binding protein.

In some embodiments of the compositions of the disclosure, a guide RNA or a portion thereof comprises or consists of between 10 and 100 nucleotides, inclusive of the endpoints. In some embodiments, a spacer sequence of the disclosure comprises or consists of between 10 and 30 nucleotides, inclusive of the endpoints. In some embodiments, a scaffold sequence of the disclosure comprises or consists of 15, 16, 17, 18, 19, 20, 21, 22, 23, 24, 25, 26, 27, 28, 29 or 30 nucleotides. In some embodiments, the spacer sequence of the disclosure comprises or consists of 20 nucleotides. In some embodiments, the spacer sequence of the disclosure comprises or consists of 21 nucleotides. In some embodiments, a scaffold sequence of the disclosure comprises or consists of between 10 and 100 nucleotides, inclusive of the endpoints. In some embodiments, a scaffold sequence of the disclosure comprises or consists of 30, 35, 40, 45, 50, 55, 60, 65, 70, 76, 80, 87, 90, 95, 100 or any number of nucleotides in between. In some embodiments, the scaffold sequence of the disclosure comprises or consists of between 85 and 95 nucleotides, inclusive of the endpoints. In some embodiments, the scaffold sequence of the disclosure comprises or consists of 85 nucleotides. In some embodiments, the scaffold sequence of the disclosure comprises or consists of 90 nucleotides. In some embodiments, the scaffold sequence of the disclosure comprises or consists of 93 nucleotides.

In some embodiments of the compositions of the disclosure, a guide RNA or a portion thereof not comprise a nuclear localization sequence (NLS).

In some embodiments of the compositions of the disclosure, a guide RNA or a portion thereof not comprise a sequence complementary to a protospacer adjacent motif (PAM).

Therapeutic or pharmaceutical compositions of the disclosure do not comprise a PAMmer oligonucleotide. In other embodiments, optionally, non-therapeutic or non-pharmaceutical compositions may comprise a PAMmer oligonucleotide. The term “PAMmer” refers to an oligonucleotide comprising a PAM sequence that is capable of interacting with a guide nucleotide sequence-programmable RNA binding protein. Non-limiting examples of PAMmers are described in O'Connell et al. Nature 516, pages 263-266 (2014), incorporated herein by reference. A PAM sequence refers to a protospacer adjacent motif comprising about 2 to about 10 nucleotides. PAM sequences are specific to the guide nucleotide sequence-programmable RNA binding protein with which they interact and are known in the art. For example, Streptococcus pyogenes PAM has the sequence 5′-NGG-3′, where “N” is any nucleobase followed by two guanine (“G”) nucleobases. Cas9 of Francisella novicida recognizes the canonical PAM sequence 5′-NGG-3′, but has been engineered to recognize the PAM 5′-YG-3′ (where “Y” is a pyrimidine), thus adding to the range of possible Cas9 targets. The Cpf1 nuclease of Francisella novicida recognizes the PAM 5′-TTTN-3′ or 5′-YTN-3′.

In some embodiments of the compositions of the disclosure, a guide RNA or a portion thereof comprises a sequence complementary to a protospacer flanking sequence (PFS). In some embodiments, including those wherein a guide RNA or a portion thereof comprises a sequence complementary to a PFS, the first RNA binding protein may comprise a sequence isolated or derived from a Cas13 protein. In some embodiments, including those wherein a guide RNA or a portion thereof comprises a sequence complementary to a PFS, the first RNA binding protein may comprise a sequence encoding a Cas13 protein or an RNA-binding portion thereof. In some embodiments, the guide RNA or a portion thereof does not comprise a sequence complementary to a PFS.

In some embodiments of the compositions of the disclosure, a guide RNA sequence of the disclosure comprises a promoter to drive expression of the guide RNA. In some embodiments, a vector comprising a guide RNA sequence of the disclosure comprises a promoter to drive expression of the guide RNA. In some embodiments, the promoter is a constitutive promoter. In some embodiments, a promoter is a tissue-specific and/or cell-type specific promoter. In some embodiments, a promoter is an inducible promoter. In some embodiments, a promoter is a hybrid or a recombinant promoter. In some embodiments, a promoter is a promoter capable of driving expression in a mammalian cell. In some embodiments, a promoter is a promoter capable of expression in a human cell. In some embodiments, a promoter is a promoter capable of expressing the guide RNA sequence and restricting the expression to the nucleus of the cell. In some embodiments, a promoter is a human RNA polymerase promoter or a promoter sequence isolated or derived from a a human RNA polymerase promoter. In some embodiments, a promoter is a U6 promoter or a sequence isolated or derived from a sequence encoding a U6 promoter. In some embodiments, a promoter is a human tRNA promoter or a promoter sequence isolated or derived from a sequence a human tRNA promoter. In some embodiments, a promoter is a human valine tRNA promoter or a promoter sequence isolated or derived from a human valine tRNA promoter.

In some embodiments of the compositions of the disclosure, a promoter further comprises a regulatory element. In some embodiments, a vector comprising a promoter which further comprises a regulatory element. In some embodiments, a regulatory element enhances expression of the guide RNA. Exemplary regulatory elements include, but are not limited to, an enhancer element, an intron, an exon, or a combination thereof.

In some embodiments of the compositions of the disclosure, a vector of the disclosure comprises one or more of a guide RNA sequence, a promoter to drive expression of the guide RNA and a regulatory element to enhance expression of the guide RNA. In some embodiments of the compositions of the disclosure, the vector further comprises a nucleic acid sequence encoding a fusion protein of the disclosure.

### Fusion Proteins

Fusion proteins of the disclosure comprise a first RNA binding protein and a second RNA binding protein. In some embodiments, along a sequence encoding the fusion protein, the sequence encoding the first RNA binding protein is positioned 5′ of the sequence encoding the second RNA binding protein. In some embodiments, along a sequence encoding the fusion protein, the sequence encoding the first RNA binding protein is positioned 3′ of the sequence encoding the second RNA binding protein.

In some embodiments of the compositions of the disclosure, the sequence encoding the first RNA binding protein comprises a sequence isolated or derived from a protein capable of binding an RNA molecule. In some embodiments, the sequence encoding the first RNA binding protein comprises a sequence isolated or derived from a protein capable of selectively binding an RNA molecule and not binding a DNA molecule, a mammalian DNA molecule or any DNA molecule. In some embodiments, the sequence encoding the first RNA binding protein comprises a sequence isolated or derived from a protein capable of binding an RNA molecule and inducing a break in the RNA molecule. In some embodiments, the sequence encoding the first RNA binding protein comprises a sequence isolated or derived from a protein capable of binding an RNA molecule, inducing a break in the RNA molecule, and not binding a DNA molecule, a mammalian DNA molecule or any DNA molecule. In some embodiments, the sequence encoding the first RNA binding protein comprises a sequence isolated or derived from a protein capable of binding an RNA molecule, inducing a break in the RNA molecule, and neither binding nor inducing a break in a DNA molecule, a mammalian DNA molecule or any DNA molecule.

In some embodiments of the compositions of the disclosure, the sequence encoding the first RNA binding protein comprises a sequence isolated or derived from a protein with no DNA nuclease activity.

In some embodiments of the compositions of the disclosure, the sequence encoding the first RNA binding protein comprises a sequence isolated or derived from a protein having DNA nuclease activity, wherein the DNA nuclease activity does not induce a break in a DNA molecule, a mammalian DNA molecule or any DNA molecule when a composition of the disclosure is contacted to an RNA molecule or introduced into a cell or into a subject of the disclosure.

In some embodiments of the compositions of the disclosure, the sequence encoding the first RNA binding protein comprises a sequence isolated or derived from a protein having DNA nuclease activity, wherein the DNA nuclease activity is inactivated and wherein the DNA nuclease activity does not induce a break in a DNA molecule, a mammalian DNA molecule or any DNA molecule when a composition of the disclosure is contacted to an RNA molecule or introduced into a cell or into a subject of the disclosure. In some embodiments, the sequence encoding the first RNA binding protein comprises a mutation that inactivates or decreases the DNA nuclease activity to a level at which the DNA nuclease activity does not induce a break in a DNA molecule, a mammalian DNA molecule or any DNA molecule when a composition of the disclosure is contacted to an RNA molecule or introduced into a cell or into a subject of the disclosure. In some embodiments, the sequence encoding the first RNA binding protein comprises a mutation that inactivates or decreases the DNA nuclease activity and the mutation comprises one or more of a substitution, inversion, transposition, insertion, deletion, or any combination thereof to a nucleic acid sequence or amino acid sequence encoding the first RNA binding protein or a nuclease domain thereof.

In some embodiments of the compositions of the disclosure, the sequence encoding the first RNA binding protein of an RNA-guided fusion protein disclosed herein comprises a sequence isolated or derived from a CRISPR Cas protein. In some embodiments, the CRISPR Cas protein comprises a Type II CRISPR Cas protein. In some embodiments, the Type II CRISPR Cas protein comprises a Cas9 protein. Exemplary Cas9 proteins of the disclosure may be isolated or derived from any species, including, but not limited to, a bacteria or an archaea. Exemplary Cas9 proteins of the disclosure may be isolated or derived from any species, including, but not limited to, Streptococcus pyogenes, Haloferax mediteranii, Mycobacterium tuberculosis, Francisella tularensis subsp. novicida, Pasteurella multocida, Neisseria meningitidis, Campylobacter jejune, Streptococcus thermophilus, Campylobacter lari CF89-12, Mycoplasma gallisepticum str. F, Nitratifractor salsuginis str. DSM 16511, Parvibaculum lavamentivorans, Roseburia intestinalis, Neisseria cinerea, a Gluconacetobacter diazotrophicus, an Azospirillum B510, a Sphaerochaeta globus str. Buddy, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Treponema denticola, Legionella pneumophila str. Paris, Sutterella wadsworthensis, Corynebacter diphtherias, Streptococcus aureus, and Francisella novicida.

Exemplary wild type S. pyogenes Cas9 proteins of the disclosure may comprise or consist of the amino acid sequence:

| (SEQ ID NO: 147) | |
|---|---|
| 1 | MDKKYSIGLD IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNREKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGQGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVDH |
| 841 | IVPQSFLKDD SIDNKVLTRS DKNRGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD. |

Nuclease inactivated S. pyogenes Cas9 proteins may comprise a substitution of an Alanine (A) for a Aspartic Acid (D) at position 10 and an alanine (A) for a Histidine (H) at position 840. Exemplary nuclease inactivated S. pyogenes Cas9 proteins of the disclosure may comprise or consist of the amino acid sequence (D10A and H840A bolded and underlined):

| (SEQ ID NO: 148) | |
|---|---|
| 1 | MDKKYSIGLA IGTNSVGWAV ITDEYKVPSK KFKVLGNTDR HSIKKNLIGA LLFDSGETAE |
| 61 | ATRLKRTARR RYTRRKNRIC YLQEIFSNEM AKVDDSFFHR LEESFLVEED KKHERHPIFG |
| 121 | NIVDEVAYHE KYPTIYHLRK KLVDSTDKAD LRLIYLALAH MIKFRGHFLI EGDLNPDNSD |
| 181 | VDKLFIQLVQ TYNQLFEENP INASGVDAKA ILSARLSKSR RLENLIAQLP GEKKNGLFGN |
| 241 | LIALSLGLTP NFKSNFDLAE DAKLQLSKDT YDDDLDNLLA QIGDQYADLF LAAKNLSDAI |
| 301 | LLSDILRVNT EITKAPLSAS MIKRYDEHHQ DLTLLKALVR QQLPEKYKEI FFDQSKNGYA |
| 361 | GYIDGGASQE EFYKFIKPIL EKMDGTEELL VKLNREDLLR KQRTFDNGSI PHQIHLGELH |
| 421 | AILRRQEDFY PFLKDNREKI EKILTFRIPY YVGPLARGNS RFAWMTRKSE ETITPWNFEE |
| 481 | VVDKGASAQS FIERMTNFDK NLPNEKVLPK HSLLYEYFTV YNELTKVKYV TEGMRKPAFL |
| 541 | SGEQKKAIVD LLFKTNRKVT VKQLKEDYFK KIECFDSVEI SGVEDRFNAS LGTYHDLLKI |
| 601 | IKDKDFLDNE ENEDILEDIV LTLTLFEDRE MIEERLKTYA HLFDDKVMKQ LKRRRYTGWG |
| 661 | RLSRKLINGI RDKQSGKTIL DFLKSDGFAN RNFMQLIHDD SLTFKEDIQK AQVSGQGDSL |
| 721 | HEHIANLAGS PAIKKGILQT VKVVDELVKV MGRHKPENIV IEMARENQTT QKGQKNSRER |
| 781 | MKRIEEGIKE LGSQILKEHP VENTQLQNEK LYLYYLQNGR DMYVDQELDI NRLSDYDVDA |
| 841 | IVPQSFLKDD SIDNKVLTRS DKNRGKSDNV PSEEVVKKMK NYWRQLLNAK LITQRKFDNL |
| 901 | TKAERGGLSE LDKAGFIKRQ LVETRQITKH VAQILDSRMN TKYDENDKLI REVKVITLKS |
| 961 | KLVSDFRKDF QFYKVREINN YHHAHDAYLN AVVGTALIKK YPKLESEFVY GDYKVYDVRK |
| 1021 | MIAKSEQEIG KATAKYFFYS NIMNFFKTEI TLANGEIRKR PLIETNGETG EIVWDKGRDF |
| 1081 | ATVRKVLSMP QVNIVKKTEV QTGGFSKESI LPKRNSDKLI ARKKDWDPKK YGGFDSPTVA |
| 1141 | YSVLVVAKVE KGKSKKLKSV KELLGITIME RSSFEKNPID FLEAKGYKEV KKDLIIKLPK |
| 1201 | YSLFELENGR KRMLASAGEL QKGNELALPS KYVNFLYLAS HYEKLKGSPE DNEQKQLFVE |
| 1261 | QHKHYLDEII EQISEFSKRV ILADANLDKV LSAYNKHRDK PIREQAENII HLFTLTNLGA |
| 1321 | PAAFKYFDTT IDRKRYTSTK EVLDATLIHQ SITGLYETRI DLSQLGGD. |

Nuclease inactivated S. pyogenes Cas9 proteins may comprise deletion of a RuvC nuclease domain or a portion thereof, an HNH domain, a DNAse active site, a ββα-metal fold or a portion thereof comprising a DNAse active site or any combination thereof.

Other exemplary Cas9 proteins or portions thereof may comprise or consist of the following amino acid sequences.

In some embodiments the Cas9 protein can be S. pyogenes Cas9 and may comprise or consist of the amino acid sequence:

(SEQ ID NO: 149)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVIEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVVVDKGRDFATVRKVLSMPQVNIVKKTE
VQTGGFSKESILPKRNSDKLIARKKDWDPKKYGGFDSPTVAYSVLVVAKV
EKGKSKKLKSVKELLGITIMERSSFEKNPIDFLEAKGYKEVKKDLIIKLP
KYSLFELENGRKRMLASAGELQKGNELALPSKYVNFLYLASHYEKLKGSP
EDNEQKQLFVEQHKHYLDEIIEQISEFSKRVILADANLDKVLSAYNKHRD
KPIREQAENIIHLFTLTNLGAPAAFKYFDTTIDRKRYTSTKEVLDATLIH
QSITGLYETRIDLSQLGGD

In some embodiments the Cas9 protein can be S. aureus Cas9 and may comprise or consist of the amino acid sequence:

(SEQ ID NO: 150)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

In some embodiments the Cas9 protein can be S. thermophiles CRISPR1 Cas9 and may comprise or consist of the amino acid sequence:

(SEQ ID NO: 151)
MSDLVLGLDIGIGSVGVGILNKVTGEIIHKNSRIFPAAQAENNLVRRTNR
QGRRLARRKKHRRVRLNRLFEESGLITDFTKISINLNPYQLRVKGLTDEL
SNEELFIALKNMVKHRGISYLDDASDDGNSSVGDYAQIVKENSKQLETKT
PGQIQLERYQTYGQLRGDFTVEKDGKKHRLINVFPTSAYRSEALRILQTQ
QEFNPQITDEFINRYLEILTGKRKYYHGPGNEKSRTDYGRYRTSGETLDN
IFGILIGKCTFYPDEFRAAKASYTAQEFNLLNDLNNLTVPTETKKLSKEQ
KNQIINYVKNEKAMGPAKLFKYIAKLLSCDVADIKGYRIDKSGKAEIHTF
EAYRKMKTLETLDIEQMDRETLDKLAYVLTLNTEREGIQEALEHEFADGS
FSQKQVDELVQFRKANSSIFGKGWHNFSVKLMMELIPELYETSEEQMTIL
TRLGKQKTTSSSNKTKYIDEKLLTEEIYNPVVAKSVRQAIKIVNAAIKEY
GDFDNIVIEMARETNEDDEKKAIQKIQKANKDEKDAAMLKAANQYNGKAE
LPHSVFHGHKQLATKIRLWHQQGERCLYTGKTISIHDLINNSNQFEVDHI
LPLSITFDDSLANKVLVYATANQEKGQRTPYQALDSMDDAWSFRELKAFV
RESKTLSNKKKEYLLTEEDISKFDVRKKFIERNLVDTRYASRVVLNALQE
HFRAHKIDTKVSVVRGQFTSQLRRHWGIEKTRDTYHHHAVDALIIAASSQ
LNLWKKQKNTLVSYSEDQLLDIETGELISDDEYKESVFKAPYQHFVDTLK
SKEFEDSILFSYQVDSKFNRKISDATIYATRQAKVGKDKADETYVLGKIK
DIYTQDGYDAFMKIYKKDKSKFLMYRHDPQTFEKVIEPILENYPNKQIND
KGKEVPCNPFLKYKEEHGYIRKYSKKGNGPEIKSLKYYDSKLGNHIDITP
KDSNNKVVLQSVSPWRADVYFNKTTGKYEILGLKYADLQFDKGTGTYKIS
QEKYNDIKKKEGVDSDSEFKFTLYKNDLLLVKDTETKEQQLFRFLSRTMP
KQKHYVELKPYDKQKFEGGEALIKVLGNVANSGQCKKGLGKSNISIYKVR
TDVLGNQHIIKNEGDKPKLDF.

In some embodiments the Cas9 protein can be N. meningitidis Cas9 and may comprise or consist of the amino acid sequence:

(SEQ ID NO: 152)
MAAFKPNPINYILGLDIGIASVGWAMVEIDEDENPICLIDLGVRVFERAE
VPKTGDSLAMARRLARSVRRLTRRRAHRLLRARRLLKREGVLQAADFDEN
GLIKSLPNTPWQLRAAALDRKLTPLEWSAVLLHLIKHRGYLSQRKNEGET
ADKELGALLKGVADNAHALQTGDFRTPAELALNKFEKESGHIRNQRGDYS
HTFSRKDLQAELILLFEKQKEFGNPHVSGGLKEGIETLLMTQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSPELQDEIGTAFSLFKTDEDITGRLK
DRIQPEILEALLKHISFDKFVQISLKALRRIVPLMEQGKRYDEACAEIYG
DHYGKKNTEEKIYLPPIPADEIRNPVVLRALSQARKVINGVVRRYGSPAR
IHIETAREVGKSFKDRKEIEKRQEENRKDREKAAAKFREYFPNFVGEPKS
KDILKLRLYEQQHGKCLYSGKEINLGRLNEKGYVEIDHALPFSRTWDDSF
NNKVLVLGSENQNKGNQTPYEYFNGKDNSREWQEFKARVETSRFPRSKKQ
RILLQKFDEDGFKERNLNDTRYVNRFLCQFVADRMRLTGKGKKRVFASNG
QITNLLRGFWGLRKVRAENDRHHALDAVVVACSTVAMQQKITRFVRYKEM
NAFDGKTIDKETGEVLHQKTHFPQPWEFFAQEVMIRVFGKPDGKPEFEEA
DTPEKLRTLLAEKLSSRPEAVHEYVTPLFVSRAPNRKMSGQGHMETVKSA
KRLDEGVSVLRVPLTQLKLKDLEKMVNREREPKLYEALKARLEAHKDDPA
KAFAEPFYKYDKAGNRTQQVKAVRVEQVQKTGVVVVRNHNGIADNATMVR
VDVFEKGDKYYLVPIYSWQVAKGILPDRAVVQGKDEEDWQLIDDSFNFKF
SLHPNDLVEVITKKARMFGYFASCHRGTGNINIRIHDLDHKIGKNGILEG
IGVKTALSFQKYQIDELGKEIRPCRLKKRPPVR.

In some embodiments the Cas9 protein can be Parvibaculum. lavamentivorans Cas9 and may comprise or consist of the amino acid sequence:

(SEQ ID NO: 153)
MERIFGFDIGTTSIGFSVIDYSSTQSAGNIQRLGVRIFPEARDPDGTPLN
QQRRQKRMMRRQLRRRRIRRKALNETLHEAGFLPAYGSADWPVVMADEPY
ELRRRGLEEGLSAYEFGRAIYHLAQHRHFKGRELEESDTPDPDVDDEKEA
ANERAATLKALKNEQTTLGAWLARRPPSDRKRGIHAHRNVVAEEFERLWE
VQSKFHPALKSEEMRARISDTIFAQRPVFWRKNTLGECRFMPGEPLCPKG
SWLSQQRRMLEKLNNLAIAGGNARPLDAEERDAILSKLQQQASMSWPGVR
SALKALYKQRGEPGAEKSLKFNLELGGESKLLGNALEAKLADMFGPDWPA
HPRKQEIRHAVHERLWAADYGETPDKKRVIILSEKDRKAHREAAANSFVA
DFGITGEQAAQLQALKLPTGWEPYSIPALNLFLAELEKGERFGALVNGPD
WEGWRRTNFPHRNQPTGEILDKLPSPASKEERERISQLRNPTVVRTQNEL
RKVVNNLIGLYGKPDRIRIEVGRDVGKSKREREEIQSGIRRNEKQRKKAT
EDLIKNGIANPSRDDVEKWILWKEGQERCPYTGDQIGFNALFREGRYEVE
HIWPRSRSFDNSPRNKTLCRKDVNIEKGNRMPFEAFGHDEDRWSAIQIRL
QGMVSAKGGTGMSPGKVKRFLAKTMPEDFAARQLNDTRYAAKQILAQLKR
LWPDMGPEAPVKVEAVTGQVTAQLRKLWTLNNILADDGEKTRADHRHHAI
DALTVACTHPGMTNKLSRYWQLRDDPRAEKPALTPPWDTIRADAEKAVSE
IVVSHRVRKKVSGPLHKETTYGDTGTDIKTKSGTYRQFVTRKKIESLSKG
ELDEIRDPRIKEIVAAHVAGRGGDPKKAFPPYPCVSPGGPEIRKVRLTSK
QQLNLMAQTGNGYADLGSNHHIAIYRLPDGKADFEIVSLFDASRRLAQRN
PIVQRTRADGASFVMSLAAGEAIMIPEGSKKGIWIVQGVVVASGQVVLER
DTDADHSTTTRPMPNPILKDDAKKVSIDPIGRVRPSND.

In some embodiments the Cas9 protein can be Corynebacter diphtheria Cas9 and may comprise or consist of the amino acid sequence:

(SEQ ID NO: 154)
MKYHVGIDVGTFSVGLAAIEVDDAGMPIKTLSLVSHIHDSGLDPDEIKSA
VTRLASSGIARRTRRLYRRKRRRLQQLDKFIQRQGWPVIELEDYSDPLYP
WKVRAELAASYIADEKERGEKLSVALRHIARHRGWRNPYAKVSSLYLPDG
PSDAFKAIREEIKRASGQPVPETATVGQMVTLCELGTLKLRGEGGVLSAR
LQQSDYAREIQEICRMQEIGQELYRKIIDVVFAAESPKGSASSRVGKDPL
QPGKNRALKASDAFQRYRIAALIGNLRVRVDGEKRILSVEEKNLVFDHLV
NLTPKKEPEWVTIAEILGIDRGQLIGTATMTDDGERAGARPPTHDTNRSI
VNSRIAPLVDWWKTASALEQHAMVKALSNAEVDDFDSPEGAKVQAFFADL
DDDVHAKLDSLHLPVGRAAYSEDTLVRLTRRMLSDGVDLYTARLQEFGIE
PSWTPPTPRIGEPVGNPAVDRVLKTVSRWLESATKTWGAPERVIIEHVRE
GFVTEKRAREMDGDMRRRAARNAKLFQEMQEKLNVQGKPSRADLWRYQSV
QRQNCQCAYCGSPITFSNSEMDHIVPRAGQGSTNTRENLVAVCHRCNQSK
GNTPFAIWAKNTSIEGVSVKEAVERTRHWVTDTGMRSTDFKKFTKAVVER
FQRATMDEEIDARSMESVAWMANELRSRVAQHFASHGTTVRVYRGSLTAE
ARRASGISGKLKFFDGVGKSRLDRRHHAIDAAVIAFTSDYVAETLAVRSN
LKQSQAHRQEAPQWREFTGKDAEHRAAWRVWCQKMEKLSALLTEDLRDDR
VVVMSNVRLRLGNGSAHKETIGKLSKVKLSSQLSVSDIDKASSEALWCAL
TREPGFDPKEGLPANPERHIRVNGTHVYAGDNIGLFPVSAGSIALRGGYA
ELGSSFHHARVYKITSGKKPAFAMLRVYTIDLLPYRNQDLFSVELKPQTM
SMRQAEKKLRDALATGNAEYLGWLVVDDELVVDTSKIATDQVKAVEAELG
TIRRWRVDGFFSPSKLRLRPLQMSKEGIKKESAPELSKIIDRPGWLPAVN
KLFSDGNVTVVRRDSLGRVRLESTAHLPVTWKVQ.

In some embodiments the Cas9 protein can be Streptococcus pasteurianus Cas9 and may comprise or consist of the amino acid sequence:

(SEQ ID NO: 155)
MTNGKILGLDIGIASVGVGIIEAKTGKVVHANSRLFSAANAENNAERRGF
RGSRRLNRRKKHRVKRVRDLFEKYGIVTDFRNLNLNPYELRVKGLTEQLK
NEELFAALRTISKRRGISYLDDAEDDSTGSTDYAKSIDENRRLLKNKTPG
QIQLERLEKYGQLRGNFTVYDENGEAHRLINVFSTSDYEKEARKILETQA
DYNKKITAEFIDDYVEILTQKRKYYHGPGNEKSRTDYGRFRTDGTTLENI
FGILIGKCNFYPDEYRASKASYTAQEYNFLNDLNNLKVSTETGKLSTEQK
ESLVEFAKNTATLGPAKLLKEIAKILDCKVDEIKGYREDDKGKPDLHTFE
PYRKLKFNLESINIDDLSREVIDKLADILTLNIIREGIEDAIKRNLPNQF
TEEQISEIIKVRKSQSTAFNKGWHSFSAKLMNELIPELYATSDEQMTILT
RLEKFKVNKKSSKNTKTIDEKEVTDEIYNPVVAKSVRQTIKIINAAVKKY
GDFDKIVIEMPRDKNADDEKKFIDKRNKENKKEKDDALKRAAYLYNSSDK
LPDEVFHGNKQLETKIRLWYQQGERCLYSGKPISIQELVHNSNNFEIDHI
LPLSLSFDDSLANKVLVYAWTNQEKGQKTPYQVIDSMDAAWSFREMKDYV
LKQKGLGKKKRDYLLTTENIDKIEVKKKFIERNLVDTRYASRVVLNSLQS
ALRELGKDTKVSVVRGQFTSQLRRKWKIDKSRETYHHHAVDALIIAASSQ
LKLWEKQDNPMFVDYGKNQVVDKQTGEILSVSDDEYKELVFQPPYQGFVN
TISSKGFEDEILFSYQVDSKYNRKVSDATIYSTRKAKIGKDKKEETYVLG
KIKDIYSQNGFDTFIKKYNKDKTQFLMYQKDSLTWENVIEVILRDYPTTK
KSEDGKNDVKCNPFEEYRRENGLICKYSKKGKGTPIKSLKYYDKKLGNCI
DITPEESRNKVILQSINPWRADVYFNPETLKYELMGLKYSDLSFEKGTGN
YHISQEKYDAIKEKEGIGKKSEFKFTLYRNDLILIKDIASGEQEIYRFLS
RTMPNVNHYVELKPYDKEKFDNVQELVEALGEADKVGRCIKGLNKPNISI
YKVRTDVLGNKYFVKKKGDKPKLDFKNNKK.

In some embodiments the Cas9 protein can be Neisseria cinerea Cas9 and may comprise or consist of the amino acid sequence:

(SEQ ID NO: 156)
MAAFKPNPMNYILGLDIGIASVGWAIVEIDEEENPIRLIDLGVRVFERAE
VPKTGDSLAAARRLARSVRRLTRRRAHRLLRARRLLKREGVLQAADFDEN
GLIKSLPNTPWQLRAAALDRKLTPLEWSAVLLHLIKHRGYLSQRKNEGET
ADKELGALLKGVADNTHALQTGDFRTPAELALNKFEKESGHIRNQRGDYS
HTFNRKDLQAELNLLFEKQKEFGNPHVSDGLKEGIETLLMTQRPALSGDA
VQKMLGHCTFEPTEPKAAKNTYTAERFVWLTKLNNLRILEQGSERPLTDT
ERATLMDEPYRKSKLTYAQARKLLDLDDTAFFKGLRYGKDNAEASTLMEM
KAYHAISRALEKEGLKDKKSPLNLSPELQDEIGTAFSLFKTDEDITGRLK
DRVQPEILEALLKHISFDKFVQISLKALRRIVPLMEQGNRYDEACTEIYG
DHYGKKNIEEKIYLPPIPADEIRNPVVLRALSQARKVINGVVRRYGSPAR
IHIETAREVGKSFKDRKEIEKRQEENRKDREKSAAKFREYFPNFVGEPKS
KDILKLRLYEQQHGKCLYSGKEINLGRLNEKGYVEIDHALPFSRTWDDSF
NNKVLALGSENQNKGNQTPYEYFNGKDNSREWQEFKARVETSRFPRSKKQ
RILLQKFDEDGFKERNLNDTRYINRFLCQFVADHMLLTGKGKRRVFASNG
QITNLLRGFWGLRKVRAENDRHHALDAVVVACSTIAMQQKITRFVRYKEM
NAFDGKTIDKETGEVLHQKAHFPQPWEFFAQEVMIRVFGKPDGKPEFEEA
DTPEKLRTLLAEKLSSRPEAVHKYVTPLFISRAPNRKMSGQGHMETVKSA
KRLDEGISVLRVPLTQLKLKDLEKMVNREREPKLYEALKARLEAHKDDPA
KAFAEPFYKYDKAGNRTQQVKAVRVEQVQKTGVWVHNHNGIADNATIVRV
DVFEKGGKYYLVPIYSWQVAKGILPDRAVVQGKDEEDWTVMDDSFEFKFV
LYANDLIKLTAKKNEFLGYFVSLNRATGAIDIRTHDTDSTKGKNGIFQSV
GVKTALSFQKYQIDELGKEIRPCRLKKRPPVR.

In some embodiments the Cas9 protein can be Campylobacter lari Cas9 and may comprise or consist of the amino acid sequence:

(SEQ ID NO: 157)
MRILGFDIGINSIGWAFVENDELKDCGVRIFTKAENPKNKESLALPRRNA
RSSRRRLKRRKARLIAIKRILAKELKLNYKDYVAADGELPKAYEGSLASV
YELRYKALTQNLETKDLARVILHIAKHRGYMNKNEKKSNDAKKGKILSAL
KNNALKLENYQSVGEYFYKEFFQKYKKNTKNFIKIRNTKDNYNNCVLSSD
LEKELKLILEKQKEFGYNYSEDFINEILKVAFFQRPLKDFSHLVGACTFF
EEEKRACKNSYSAWEFVALTKIINEIKSLEKISGEIVPTQTINEVLNLIL
DKGSITYKKFRSCINLHESISFKSLKYDKENAENAKLIDFRKLVEFKKAL
GVHSLSRQELDQISTHITLIKDNVKLKTVLEKYNLSNEQINNLLEIEFND
YINLSFKALGMILPLMREGKRYDEACEIANLKPKTVDEKKDFLPAFCDSI
FAHELSNPVVNRAISEYRKVLNALLKKYGKVHKIHLELARDVGLSKKARE
KIEKEQKENQAVNAWALKECENIGLKASAKNILKLKLWKEQKEICIYSGN
KISIEHLKDEKALEVDHIYPYSRSFDDSFINKVLVFTKENQEKLNKTPFE
AFGKNIEKWSKIQTLAQNLPYKKKNKILDENFKDKQQEDFISRNLNDTRY
IATLIAKYTKEYLNFLLLSENENANLKSGEKGSKIHVQTISGMLTSVLRH
TWGFDKKDRNNHLHHALDAIIVAYSTNSIIKAFSDFRKNQELLKARFYAK
ELTSDNYKHQVKFFEPFKSFREKILSKIDEIFVSKPPRKRARRALHKDTF
HSENKIIDKCSYNSKEGLQIALSCGRVRKIGTKYVENDTIVRVDIFKKQN
KFYAIPIYAMDFALGILPNKIVITGKDKNNNPKQWQTIDESYEFCFSLYK
NDLILLQKKNMQEPEFAYYNDFSISTSSICVEKHDNKFENLTSNQKLLFS
NAKEGSVKVESLGIQNLKVFEKYIITPLGDKIKADFQPRENISLKTSKKY
GLR.

In some embodiments the Cas9 protein can be T. denticola Cas9 and may comprise or consist of the amino acid sequence:

(SEQ ID NO: 158)
MKKEIKDYFLGLDVGTGSVGWAVTDTDYKLLKANRKDLWGMRCFETAETA
EVRRLHRGARRRIERRKKRIKLLQELFSQEIAKTDEGFFQRMKESPFYAE
DKTILQENTLFNDKDFADKTYHKAYPTINHLIKAWIENKVKPDPRLLYLA
CHNIIKKRGHFLFEGDFDSENQFDTSIQALFEYLREDMEVDIDADSQKVK
EILKDSSLKNSEKQSRLNKILGLKPSDKQKKAITNLISGNKINFADLYDN
PDLKDAEKNSISFSKDDFDALSDDLASILGDSFELLLKAKAVYNCSVLSK
VIGDEQYLSFAKVKIYEKHKTDLTKLKNVIKKHFPKDYKKVFGYNKNEKN
NNNYSGYVGVCKTKSKKLIINNSVNQEDFYKFLKTILSAKSEIKEVNDIL
TEIETGTFLPKQISKSNAEIPYQLRKMELEKILSNAEKHFSFLKQKDEKG
LSHSEKIIMLLTFKIPYYIGPINDNHKKFFPDRCWVVKKEKSPSGKTTPW
NFFDHIDKEKTAEAFITSRTNFCTYLVGESVLPKSSLLYSEYTVLNEINN
LQIIIDGKNICDIKLKQKIYEDLFKKYKKITQKQISTFIKHEGICNKTDE
VIILGIDKECTSSLKSYIELKNIFGKQVDEISTKNMLEEIIRWATIYDEG
EGKTILKTKIKAEYGKYCSDEQIKKILNLKFSGWGRLSRKFLETVTSEMP
GFSEPVNIITAMRETQNNLMELLSSEFTFTENIKKINSGFEDAEKQFSYD
GLVKPLFLSPSVKKMLWQTLKLVKEISHITQAPPKKIFIEMAKGAELEPA
RTKTRLKILQDLYNNCKNDADAFSSEIKDLSGKIENEDNLRLRSDKLYLY
YTQLGKCMYCGKPIEIGHVFDTSNYDIDHIYPQSKIKDDSISNRVLVCSS
CNKNKEDKYPLKSEIQSKQRGFWNFLQRNNFISLEKLNRLTRATPISDDE
TAKFIARQLVETRQATKVAAKVLEKMFPETKIVYSKAETVSMFRNKFDIV
KCREINDFHHAHDAYLNIVVGNVYNTKFTNNPWNFIKEKRDNPKIADTYN
YYKVFDYDVKRNNITAWEKGKTIITVKDMLKRNTPIYTRQAACKKGELFN
QTIMKKGLGQHPLKKEGPFSNISKYGGYNKVSAAYYTLIEYEEKGNKIRS
LETIPLYLVKDIQKDQDVLKSYLTDLLGKKEFKILVPKIKINSLLKINGF
PCHITGKTNDSFLLRPAVQFCCSNNEVLYFKKIIRFSEIRSQREKIGKTI
SPYEDLSFRSYIKENLWKKTKNDEIGEKEFYDLLQKKNLEIYDMLLTKHK
DTIYKKRPNSATIDILVKGKEKFKSLIIENQFEVILEILKLFSATRNVSD
LQHIGGSKYSGVAKIGNKISSLDNCILIYQSITGIFEKRIDLLKV.

In some embodiments the Cas9 protein can be S. mutans Cas9 and may comprise or consist of the amino acid sequence:

(SEQ ID NO: 159)
MKKPYSIGLDIGTNSVGWAVVTDDYKVPAKKMKVLGNTDKSHIEKNLLGA
LLFDSGNTAEDRRLKRTARRRYTRRRNRILYLQEIFSEEMGKVDDSFFHR
LEDSFLVIEDKRGERHPIFGNLEEEVKYHENFPTIYHLRQYLADNPEKVD
LRLVYLALAHIIKFRGHFLIEGKFDTRNNDVQRLFQEFLAVYDNTFENSS
LQEQNVQVEEILTDKISKSAKKDRVLKLFPNEKSNGRFAEFLKLIVGNQA
DFKKHFELEEKAPLQFSKDTYEEELEVLLAQIGDNYAELFLSAKKLYDSI
LLSGILTVTDVGTKAPLSASMIQRYNEHQMDLAQLKQFIRQKLSDKYNEV
FSDVSKDGYAGYIDGKTNQEAFYKYLKGLLNKIEGSGYFLDKIEREDFLR
KQRTFDNGSIPHQIHLQEMRAIIRRQAEFYPFLADNQDRIEKLLTFRIPY
YVGPLARGKSDFAWLSRKSADKITPWNFDEIVDKESSAEAFINRMTNYDL
YLPNQKVLPKHSLLYEKFTVYNELTKVKYKTEQGKTAFFDANMKQEIFDG
VFKVYRKVTKDKLMDFLEKEFDEFRIVDLTGLDKENKVFNASYGTYHDLC
KILDKDFLDNSKNEKILEDIVLTLTLFEDREMIRKRLENYSDLLTKEQVK
KLERRHYTGWGRLSAELIHGIRNKESRKTILDYLIDDGNSNRNFMQLIND
DALSFKEEIAKAQVIGETDNLNQVVSDIAGSPAIKKGILQSLKIVDELVK
IMGHQPENIVVEMARENQFTNQGRRNSQQRLKGLTDSIKEFGSQILKEHP
VENSQLQNDRLFLYYLQNGRDMYTGEELDIDYLSQYDIDHIIPQAFIKDN
SIDNRVLTSSKENRGKSDDVPSKDVVRKMKSYWSKLLSAKLITQRKFDNL
TKAERGGLTDDDKAGFIKRQLVETRQITKHVARILDERFNTETDENNKKI
RQVKIVTLKSNLVSNFRKEFELYKVREINDYHHAHDAYLNAVIGKALLGV
YPQLEPEFVYGDYPHFHGHKENKATAKKFFYSNIMNFFKKDDVRTDKNGE
IIWKKDEHISNIKKVLSYPQVNIVKKVEEQTGGFSKESILPKGNSDKLIP
RKTKKFYWDTKKYGGFDSPIVAYSILVIADIEKGKSKKLKTVKALVGVTI
MEKMTFERDPVAFLERKGYRNVQEENIIKLPKYSLFKLENGRKRLLASAR
ELQKGNEIVLPNHLGTLLYHAKNIHKVDEPKHLDYVDKHKDEFKELLDVV
SNFSKKYTLAEGNLEKIKELYAQNNGEDLKELASSFINLLTFTAIGAPAT
FKFFDKNIDRKRYTSTTEILNATLIHQSITGLYETRIDLNKLGGD

In some embodiments the Cas9 protein can be S. thermophilus CRISPR 3 Cas9 and may comprise or consist of the amino acid sequence:

(SEQ ID NO: 160)
MTKPYSIGLDIGTNSVGWAVTTDNYKVPSKKMKVLGNTSKKYIKKNLLGV
LLFDSGITAEGRRLKRTARRRYTRRRNRILYLQEIFSTEMATLDDAFFQR
LDDSFLVPDDKRDSKYPIFGNLVEEKAYHDEFPTIYHLRKYLADSTKKAD
LRLVYLALAHMIKYRGHFLIEGEFNSKNNDIQKNFQDFLDTYNAIFESDL
SLENSKQLEEIVKDKISKLEKKDRILKLFPGEKNSGIFSEFLKLIVGNQA
DFRKCFNLDEKASLHFSKESYDEDLETLLGYIGDDYSDVFLKAKKLYDAI
LLSGFLTVTDNETEAPLSSAMIKRYNEHKEDLALLKEYIRNISLKTYNEV
FKDDTKNGYAGYIDGKTNQEDFYVYLKKLLAEFEGADYFLEKIDREDFLR
KQRTFDNGSIPYQIHLQEMRAILDKQAKFYPFLAKNKERIEKILTFRIPY
YVGPLARGNSDFAWSIRKRNEKITPWNFEDVIDKESSAEAFINRMTSFDL
YLPEEKVLPKHSLLYETFNVYNELTKVRFIAESMRDYQFLDSKQKKDIVR
LYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKL
SRRHYTGWGKLSAKLINGIRDEKSGNTILDYLIDDGISNRNFMQLIHDDA
LSFKKKIQKAQIIGDEDKGNIKEVVKSLPGSPAIKKGILQSIKIVDELVK
VMGGRKPESIVVEMARENQYTNQGKSNSQQRLKRLEKSLKELGSKILKEN
IPAKLSKIDNNALQNDRLYLYYLQNGKDMYTGDDLDIDRLSNYDIDHIIP
QAFLKDNSIDNKVLVSSASNRGKSDDVPSLEVVKKRKTFWYQLLKSKLIS
QRKFDNLTKAERGGLSPEDKAGFIQRQLVETRQITKHVARLLDEKFNNKK
DENNRAVRTVKIITLKSTLVSQFRKDFELYKVREINDFHHAHDAYLNAVV
ASALLKKYPKLEPEFVYGDYPKYNSFRERKSATEKVYFYSNIMNIFKKSI
SLADGRVIERPLIEVNEETGESVWNKESDLATVRRVLSYPQVNVVKKVEE
QNHGLDRGKPKGLFNANLSSKPKPNSNENLVGAKEYLDPKKYGGYAGISN
SFTVLVKGTIEKGAKKKITNVLEFQGISILDRINYRKDKLNFLLEKGYKD
IELIIELPKYSLFELSDGSRRMLASILSTNNKRGEIHKGNQIFLSQKFVK
LLYHAKRISNTINENHRKYVENHKKEFEELFYYILEFNENYVGAKKNGKL
LNSAFQSWQNHSIDELCSSFIGPTGSERKGLFELTSRGSAADFEFLGVKI
PRYRDYTPSSLLKDATLIHQSVTGLYETRIDLAKLGEG

In some embodiments the Cas9 protein can be C. jejuni Cas9 and may comprise or consist of the amino acid sequence:

(SEQ ID NO: 161)
MARILAFDIGISSIGWAFSENDELKDCGVRIFTKVENPKTGESLALPRRL
ARSARKRLARRKARLNHLKHLIANEFKLNYEDYQSFDESLAKAYKGSLIS
PYELRFRALNELLSKQDFARVILHIAKRRGYDDIKNSDDKEKGAILKAIK
QNEEKLANYQSVGEYLYKEYFQKFKENSKEFTNVRNKKESYERCIAQSFL
KDELKLIFKKQREFGFSFSKKFEEEVLSVAFYKRALKDFSHLVGNCSFFT
DEKRAPKNSPLAFWVALTRIINLLNNLKNTEGILYTKDDLNALLNEVLKN
GTLTYKQTKKLLGLSDDYEFKGEKGTYFIEFKKYKEFIKALGEHNLSQDD
LNEIAKDITLIKDEIKLKKALAKYDLNQNQIDSLSKLEFKDHLNISFKAL
KLVTPLMLEGKKYDEACNELNLKVAINEDKKDFLPAFNETYYKDEVTNPV
VLRAIKEYRKVLNALLKKYGKVHKINIELAREVGKNHSQRAKIEKEQNEN
YKAKKDAELECEKLGLKINSKNILKLRLFKEQKEFCAYSGEKIKISDLQD
EKMLEIDHIYPYSRSFDDSYMNKVLVFTKQNQEKLNQTPFEAFGNDSAKW
QKIEVLAKNLPTKKQKRILDKNYKDKEQKNFKDRNLNDTRYIARLVLNYT
KDYLDFLPLSDDENTKLNDTQKGSKVHVEAKSGMLTSALRHTWGFSAKDR
NNHLHHAIDAVIIAYANNSIVKAFSDFKKEQESNSAELYAKKISELDYKN
KRKFFEPFSGFRQKVLDKIDEIFVSKPERKKPSGALHEETFRKEEEFYQS
YGGKEGVLKALELGKIRKVNGKIVKNGDMFRVDIFKHKKTNKFYAVPIYT
MDFALKVLPNKAVARSKKGEIKDWILMDENYEFCFSLYKDSLILIQTKDM
QEPEFVYYNAFTSSTVSLIVSKHDNKFETLSKNQKILFKNANEKEVIAKS
IGIQNLKVFEKYIVSALGEVTKAEFRQREDFKK

In some embodiments the Cas9 protein can be P. multocida Cas9 and may comprise or consist of the amino acid sequence:

(SEQ ID NO: 162)
MQTTNLSYILGLDLGIASVGWAVVEINENEDPIGLIDVGVRIFERAEVPK
TGESLALSRRLARSTRRLIRRRAHRLLLAKRFLKREGILSTIDLEKGLPN
QAWELRVAGLERRLSAIEWGAVLLHLIKHRGYLSKRKNESQTNNKELGAL
LSGVAQNHQLLQSDDYRTPAELALKKFAKEEGHIRNQRGAYTHTFNRLDL
LAELNLLFAQQHQFGNPHCKEHIQQYMTELLMWQKPALSGEAILKMLGKC
THEKNEFKAAKHTYSAERFVWLTKLNNLRILEDGAERALNEEERQLLINH
PYEKSKLTYAQVRKLLGLSEQAIFKHLRYSKENAESATFMELKAWHAIRK
ALENQGLKDTWQDLAKKPDLLDEIGTAFSLYKTDEDIQQYLTNKVPNSVI
NALLVSLNFDKFIELSLKSLRKILPLMEQGKRYDQACREIYGHHYGEANQ
KTSQLLPAIPAQEIRNPVVLRTLSQARKVINAIIRQYGSPARVHIETGRE
LGKSFKERREIQKQQEDNRTKRESAVQKFKELFSDFSSEPKSKDILKFRL
YEQQHGKCLYSGKEINIHRLNEKGYVEIDHALPFSRTWDDSFNNKVLVLA
SENQNKGNQTPYEWLQGKINSERWKNFVALVLGSQCSAAKKQRLLTQVID
DNKFIDRNLNDTRYIARFLSNYIQENLLLVGKNKKNVFTPNGQITALLRS
RWGLIKARENNNRHHALDAIVVACATPSMQQKITRFIRFKEVHPYKIENR
YEMVDQESGEIISPHFPEPWAYFRQEVNIRVFDNHPDTVLKEMLPDRPQA
NHQFVQPLFVSRAPTRKMSGQGHMETIKSAKRLAEGISVLRIPLTQLKPN
LLENMVNKEREPALYAGLKARLAEFNQDPAKAFATPFYKQGGQQVKAIRV
EQVQKSGVLVRENNGVADNASIVRTDVFIKNNKFFLVPIYTWQVAKGILP
NKAIVAHKNEDEWEEMDEGAKFKFSLFPNDLVELKTKKEYFFGYYIGLDR
ATGNISLKEHDGEISKGKDGVYRVGVKLALSFEKYQVDELGKNRQICRPQ
QRQPVR

In some embodiments the Cas9 protein can be F. novicida Cas9 and may comprise or consist of the amino acid sequence:

(SEQ ID NO: 163)
MNFKILPIAIDLGVKNTGVFSAFYQKGTSLERLDNKNGKVYELSKDSYTL
LMNNRTARRHQRRGIDRKQLVKRLFKLIWTEQLNLEWDKDTQQAISFLFN
RRGFSFITDGYSPEYLNIVPEQVKAILMDIFDDYNGEDDLDSYLKLATEQ
ESKISEIYNKLMQKILEFKLMKLCTDIKDDKVSTKTLKEITSYEFELLAD
YLANYSESLKTQKFSYTDKQGNLKELSYYHHDKYNIQEFLKRHATINDRI
LDTLLTDDLDIWNFNFEKFDFDKNEEKLQNQEDKDHIQAHLHHFVFAVNK
IKSEMASGGRHRSQYFQEITNVLDENNHQEGYLKNFCENLHNKKYSNLSV
KNLVNLIGNLSNLELKPLRKYFNDKIHAKADHWDEQKFIETYCHWILGEW
RVGVKDQDKKDGAKYSYKDLCNELKQKVTKAGLVDFLLELDPCRTIPPYL
DNNNRKPPKCQSLILNPKFLDNQYPNWQQYLQELKKLQSIQNYLDSFETD
LKVLKSSKDQPYFVEYKSSNQQIASGQRDYKDLDARILQFIFDRVKASDE
LLLNEIYFQAKKLKQKASSELEKLESSKKLDEVIANSQLSQILKSQHTNG
IFEQGTFLHLVCKYYKQRQRARDSRLYIMPEYRYDKKLHKYNNTGRFDDD
NQLLTYCNHKPRQKRYQLLNDLAGVLQVSPNFLKDKIGSDDDLFISKWLV
EHIRGFKKACEDSLKIQKDNRGLLNHKINIARNTKGKCEKEIFNLICKIE
GSEDKKGNYKHGLAYELGVLLFGEPNEASKPEFDRKIKKFNSIYSFAQIQ
QIAFAERKGNANTCAVCSADNAHRMQQIKIIEPVEDNKDKIILSAKAQRL
PAIPTRIVDGAVKKMATILAKNIVDDNWQNIKQVLSAKHQLHIPIIIESN
AFEFEPALADVKGKSLKDRRKKALERISPENIFKDKNNRIKEFAKGISAY
SGANLTDGDFDGAKEELDHIIPRSHKKYGTLNDEANLICVTRGDNKNKGN
RIFCLRDLADNYKLKQFETTDDLEIEKKIADTIWDANKKDFKFGNYRSFI
NLTPQEQKAFRHALFLADENPIKQAVIRAINNRNRTFVNGTQRYFAEVLA
NNIYLRAKKENLNTDKISFDYFGIPTIGNGRGIAEIRQLYEKVDSDIQAY
AKGDKPQASYSHLIDAMLAFCIAADEHRNDGSIGLEIDKNYSLYPLDKNT
GEVFTKDIFSQIKITDNEFSDKKLVRKKAIEGFNTHRQMTRDGIYAENYL
PILIHKELNEVRKGYTWKNSEEIKIFKGKKYDIQQLNNLVYCLKFVDKPI
SIDIQISTLEELRNILTTNNIAATAEYYYINLKTQKLHEYYIENYNTALG
YKKYSKEMEFLRSLAYRSERVKIKSIDDVKQVLDKDSNFIIGKITLPFKK
EWQRLYREWQNTTIKDDYEFLKSFFNVKSITKLHKKVRKDFSLPISTNEG
KFLVKRKTWDNNFIYQILNDSDSRADGTKPFIPAFDISKNEIVEAIIDSF
TSKNIFWLPKNIELQKVDNKNIFAIDTSKWFEVETPSDLRDIGIATIQYK
IDNNSRPKVRVKLDYVIDDDSKINYFMNHSLLKSRYPDKVLEILKQSTII
EFESSGFNKTIKEMLGMKLAGIYNETSNN

In some embodiments the Cas9 protein can be Lactobacillus buchneri Cas9 and may comprise or consist of the amino acid sequence:

(SEQ ID NO: 164)
MKVNNYHIGLDIGTSSIGWVAIGKDGKPLRVKGKTAIGARLFQEGNPAAD
RRMFRTTRRRLSRRKWRLKLLEEIFDPYITPVDSTFFARLKQSNLSPKDS
RKEFKGSMLFPDLTDMQYHKNYPTIYHLRHALMTQDKKFDIRMVYLAIHH
IVKYRGNFLNSTPVDSFKASKVDFVDQFKKLNELYAAINPEESFKINLAN
SEDIGHQFLDPSIRKFDKKKQIPKIVPVMMNDKVTDRLNGKIASEIIHAI
LGYKAKLDVVLQCTPVDSKPWALKFDDEDIDAKLEKILPEMDENQQSIVA
ILQNLYSQVTLNQIVPNGMSLSESMIEKYNDHHDHLKLYKKLIDQLADPK
KKAVLKKAYSQYVGDDGKVIEQAEFWSSVKKNLDDSELSKQIMDLIDAEK
FMPKQRTSQNGVIPHQLHQRELDEIIEHQSKYYPWLVEINPNKHDLHLAK
YKIEQLVAFRVPYYVGPMITPKDQAESAETVFSWMERKGTETGQITPWNF
DEKVDRKASANRFIKRMTTKDTYLIGEDVLPDESLLYEKFKVLNELNMVR
VNGKLLKVADKQAIFQDLFENYKHVSVKKLQNYIKAKTGLPSDPEISGLS
DPEHFNNSLGTYNDFKKLFGSKVDEPDLQDDFEKIVEWSTVFEDKKILRE
KLNEITWLSDQQKDVLESSRYQGWGRLSKKLLTGIVNDQGERIIDKLWNT
NKNFMQIQSDDDFAKRIHEANADQMQAVDVEDVLADAYTSPQNKKAIRQV
VKVVDDIQKAMGGVAPKYISIEFTRSEDRNPRRTISRQRQLENTLKDTAK
SLAKSINPELLSELDNAAKSKKGLTDRLYLYFTQLGKDIYTGEPINIDEL
NKYDIDHILPQAFIKDNSLDNRVLVLTAVNNGKSDNVPLRMFGAKMGHFW
KQLAEAGLISKRKLKNLQTDPDTISKYAMHGFIRRQLVETSQVIKLVANI
LGDKYRNDDTKIIEITARMNHQMRDEFGFIKNREINDYHHAFDAYLTAFL
GRYLYHRYIKLRPYFVYGDFKKFREDKVTMRNFNFLHDLTDDTQEKIADA
ETGEVIWDRENSIQQLKDVYHYKFMLISHEVYTLRGAMFNQTVYPASDAG
KRKLIPVKADRPVNVYGGYSGSADAYMAIVRIHNKKGDKYRVVGVPMRAL
DRLDAAKNVSDADFDRALKDVLAPQLTKTKKSRKTGEITQVIEDFEIVLG
KVMYRQLMIDGDKKFMLGSSTYQYNAKQLVLSDQSVKTLASKGRLDPLQE
SMDYNNVYlEILDKVNQYFSLYDMNKFRHKLNLGFSKFISFPNHNVLDGN
TKVSSGKREILQEILNGLHANPTFGNLKDVGITTPFGQLQQPNGILLSDE
TKIRYQSPTGLFERTVSLKDL

In some embodiments the Cas9 protein can be Listeria innocua Cas9 and may comprise or consist of the amino acid sequence:

(SEQ ID NO: 165)
MKKPYTIGLDIGTNSVGWAVLTDQYDLVKRKMKIAGDSEKKQIKKNFWGV
RLFDEGQTAADRRMARTARRRIERRRNRISYLQGIFAEEMSKTDANFFCR
LSDSFYVDNEKRNSRHPFFATIEEEVEYHKNYPTIYHLREELVNSSEKAD
LRLVYLALAHIIKYRGNFLIEGALDTQNTSVDGIYKQFIQTYNQVFASGI
EDGSLKKLEDNKDVAKILVEKVTRKEKLERILKLYPGEKSAGMFAQFISL
IVGSKGNFQKPFDLIEKSDIECAKDSYEEDLESLLALIGDEYAELFVAAK
NAYSAVVLSSIITVAETETNAKLSASMIERFDTHEEDLGELKAFIKLHLP
KHYEEIFSNTEKHGYAGYIDGKTKQADFYKYMKMTLENIEGADYFIAKIE
KENFLRKQRTFDNGAIPHQLHLEELEAILHQQAKYYPFLKENYDKIKSLV
TFRIPYFVGPLANGQSEFAWLTRKADGEIRPWNIEEKVDFGKSAVDFIEK
MTNKDTYLPKENVLPKHSLCYQKYLVYNELTKVRYINDQGKTSYFSGQEK
EQIFNDLFKQKRKVKKKDLELFLRNMSHVESPTIEGLEDSFNSSYSTYHD
LLKVGIKQEILDNPVNTEMLENIVKILTVFEDKRMIKEQLQQFSDVLDGV
VLKKLERRHYTGWGRLSAKLLMGIRDKQSHLTILDYLMNDDGLNRNLMQL
INDSNLSFKSIIEKEQVTTADKDIQSIVADLAGSPAIKKGILQSLKIVDE
LVSVMGYPPQTIVVEMARENQTTGKGKNNSRPRYKSLEKAIKEFGSQILK
EHPTDNQELRNNRLYLYYLQNGKDMYTGQDLDIHNLSNYDIDHIVPQSFI
TDNSIDNLVLTSSAGNREKGDDVPPLEIVRKRKVFWEKLYQGNLMSKRKF
DYLTKAERGGLTEADKARFIHRQLVETRQITKNVANILHQRFNYEKDDHG
NTMKQVRIVTLKSALVSQFRKQFQLYKVRDVNDYHHAHDAYLNGVVANTL
LKVYPQLEPEFVYGDYHQFDWFKANKATAKKQFYTNIMLFFAQKDRIIDE
NGEILWDKKYLDTVKKVMSYRQMNIVKKTEIQKGEFSKATIKPKGNSSKL
IPRKTNWDPMKYGGLDSPNMAYAVVIEYAKGKNKLVFEKKIIRVTIMERK
AFEKDEKAFLEEQGYRQPKVLAKLPKYTLYECEEGRRRMLASANEAQKGN
QQVLPNHLVTLLHHAANCEVSDGKSLDYIESNREMFAELLAHVSEFAKRY
TLAEANLNKINQLFEQNKEGDIKAIAQSFVDLMAFNAMGAPASFKFFETT
IERKRYNNLKELLNSTIIYQSITGLYESRKRLDD

In some embodiments the Cas9 protein can be L. pneumophilia Cas9 and may comprise or consist of the amino acid sequence:

(SEQ ID NO: 166)
MESSQILSPIGIDLGGKFTGVCLSHLEAFAELPNHANTKYSVILIDHNNF
QLSQAQRRATRHRVRNKKRNQFVKRVALQLFQHILSRDLNAKEETALCHY
LNNRGYTYVDTDLDEYIKDETTINLLKELLPSESEHNFIDWFLQKMQSSE
FRKILVSKVEEKKDDKELKNAVKNIKNFITGFEKNSVEGHRHRKVYFENI
KSDITKDNQLDSIKKKIPSVCLSNLLGHLSNLQWKNLHRYLAKNPKQFDE
QTFGNEFLRMLKNFRHLKGSQESLAVRNLIQQLEQSQDYISILEKTPPEI
TIPPYEARTNTGMEKDQSLLLNPEKLNNLYPNWRNLIPGIIDAHPFLEKD
LEHTKLRDRKRIISPSKQDEKRDSYILQRYLDLNKKIDKFKIKKQLSFLG
QGKQLPANLIETQKEMETHFNSSLVSVLIQIASAYNKEREDAAQGIWFDN
AFSLCELSNINPPRKQKILPLLVGAILSEDFINNKDKWAKFKIFWNTHKI
GRTSLKSKCKEIEEARKNSGNAFKIDYEEALNHPEHSNNKALIKIIQTIP
DIIQAIQSHLGHNDSQALIYHNPFSLSQLYTILETKRDGFHKNCVAVTCE
NYWRSQKTEIDPEISYASRLPADSVRPFDGVLARMMQRLAYEIAMAKWEQ
IKHIPDNSSLLIPIYLEQNRFEFEESFKKIKGSSSDKTLEQAIEKQNIQW
EEKFQRIINASMNICPYKGASIGGQGEIDHIYPRSLSKKHFGVIFNSEVN
LIYCSSQGNREKKEEHYLLEHLSPLYLKHQFGTDNVSDIKNFISQNVANI
KKYISFHLLTPEQQKAARHALFLDYDDEAFKTITKFLMSQQKARVNGTQK
FLGKQIMEFLSTLADSKQLQLEFSIKQITAEEVHDHRELLSKQEPKLVKS
RQQSFPSHAIDATLTMSIGLKEFPQFSQELDNSWFINHLMPDEVHLNPVR
SKEKYNKPNISSTPLFKDSLYAERFIPVWVKGETFAIGFSEKDLFEIKPS
NKEKLFTLLKTYSTKNPGESLQELQAKSKAKWLYFPINKTLALEFLHHYF
HKEIVTPDDTTVCHFINSLRYYTKKESITVKILKEPMPVLSVKFESSKKN
VLGSFKHTIALPATKDWERLFNHPNFLALKANPAPNPKEFNEFIRKYFLS
DNNPNSDIPNNGHNIKPQKHKAVRKVFSLPVIPGNAGTMMRIRRKDNKGQ
PLYQLQTIDDTPSMGIQINEDRLVKQEVLMDAYKTRNLSTIDGINNSEGQ
AYATFDNWLTLPVSTFKPEIIKLEMKPHSKTRRYIRITQSLADFIKTIDE
ALMIKPSDSIDDPLNMPNEIVCKNKLFGNELKPRDGKMKIVSTGKIVTYE
FESDSTPQWIQTLYVTQLKKQP

In some embodiments the Cas9 protein can be N. lactamica Cas9 and may comprise or consist of the amino acid sequence:

(SEQ ID NO: 167)
MAAFKPNPMNYILGLDIGIASVGWAMVEVDEEENPIRLIDLGVRVFERAE
VPKTGDSLAMARRLARSVRRLTRRRAHRLLRARRLLKREGVLQDADFDEN
GLVKSLPNTPWQLRAAALDRKLTCLEWSAVLLHLVKHRGYLSQRKNEGET
ADKELGALLKGVADNAHALQTGDFRTPAELALNKFEKESGHIRNQRGDYS
HTFSRKDLQAELNLLFEKQKEFGNPHVSDGLKEDIETLLMAQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSTELQDEIGTAFSLFKTDKDITGRLK
DRVQPEILEALLKHISFDKFVQISLKALRRIVPLMEQGKRYDEACAEIYG
DHYCKKNAEEKIYLPPIPADEIRNPVVLRALSQARKVINCVVRRYGSPAR
IHIETAREVGKSFKDRKEIEKRQEENRKDREKAAAKFREYFPNFVGEPKS
KDILKLRLYEQQHGKCLYSGKEINLVRLNEKGYVEIDHALPFSRTWDDSF
NNKVLVLGSENQNKGNQTPYEYFNGKDNSREWQEFKARVETSRFPRSKKQ
RILLQKFDEEGFKERNLNDTRYVNRFLCQFVADHILLTGKGKRRVFASNG
QITNLLRGFWGLRKVRIENDRHHALDAVVVACSTVAMQQKITRFVRYKEM
NAFDGKTIDKETGEVLHQKAHFPQPWEFFAQEVMIRVFGKPDGKPEFEEA
DTPEKLRTLLAEKLSSRPEAVHEYVTPLFVSRAPNRKMSGQGHMETVKSA
KRLDEGISVLRVPLTQLKLKGLEKMVNREREPKLYDALKAQLETHKDDPA
KAFAEPFYKYDKAGSRTQQVKAVRIEQVQKTGVWVRNHNGIADNATMVRV
DVFEKGGKYYLVPIYSWQVAKGILPDRAVVAFKDEEDWTVMDDSFEFRFV
LYANDLIKLTAKKNEFLGYFVSLNRATGAIDIRTHDTDSTKGKNGIFQSV
GVKTALSFQKNQIDELGKEIRPCRLKKRPPVR

In some embodiments the Cas9 protein can be N. meningitides Cas9 and may comprise or consist of the amino acid sequence:

(SEQ ID NO: 168)
MAAFKPNPINYILGLDIGIASVGWAMVEIDEDENPICLIDLGVRVFERAE
VPKTGDSLAMARRLARSVRRLTRRRAHRLLRARRLLKREGVLQAADFDEN
GLIKSLPNTPWQLRAAALDRKLTPLEWSAVLLHLIKHRGYLSQRKNEGET
ADKELGALLKGVADNAHALQTGDFRTPAELALNKFEKESGHIRNQRGDYS
HTFSRKDLQAELILLFEKQKEFGNPHVSGGLKEGIETLLMTQRPALSGDA
VQKMLGHCTFEPAEPKAAKNTYTAERFIWLTKLNNLRILEQGSERPLTDT
ERATLMDEPYRKSKLTYAQARKLLGLEDTAFFKGLRYGKDNAEASTLMEM
KAYHAISRALEKEGLKDKKSPLNLSPELQDEIGTAFSLFKTDEDITGRLK
DRIQPEILEALLKHISFDKFVQISLKALRRIVPLMEQGKRYDEACAEIYG
DHYGKKNTEEKIYLPPIPADEIRNPVVLRALSQARKVINGVVRRYGSPAR
IHIETAREVGKSFKDRKEIEKRQEENRKDREKAAAKFREYFPNFVGEPKS
KDILKLRLYEQQHGKCLYSGKEINLGRLNEKGYVEIDHALPFSRTWDDSF
NNKVLVLGSENQNKGNQTPYEYFNGKDNSREWQEFKARVETSRFPRSKKQ
RILLQKFDEDGFKERNLNDTRYVNRFLCQFVADRMRLTGKGKKRVFASNG
QITNLLRGFWGLRKVRAENDRHHALDAVVVACSTVAMQQKITRFVRYKEM
NAFDGKTIDKETGEVLHQKTHFPQPWEFFAQEVMIRVFGKPDGKPEFEEA
DTPEKLRTLLAEKLSSRPEAVHEYVTPLFVSRAPNRKMSGQGHMETVKSA
KRLDEGVSVLRVPLTQLKLKDLEKMVNREREPKLYEALKARLEAHKDDPA
KAFAEPFYKYDKAGNRTQQVKAVRVEQVQKTGVWVRNHNGIADNATMVRV
DVFEKGDKYYLVPIYSWQVAKGILPDRAVVQGKDEEDWQLIDDSFNFKFS
LHPNDLVEVITKKARMFGYFASCHRGTGNINIRIHDLDHKIGKNGILEGI
GVKTALSFQKYQIDELGKEIRPCRLKKRPPVR

In some embodiments the Cas9 protein can be B. longum Cas9 and may comprise or consist of the amino acid sequence:

(SEQ ID NO: 169)
MLSRQLLGASHLARPVSYSYNVQDNDVHCSYGERCFMRGKRYRIGIDVGL
NSVGLAAVEVSDENSPVRLLNAQSVIHDGGVDPQKNKEAITRKNMSGVAR
RTRRMRRRKRERLHKLDMLLGKFGYPVIEPESLDKPFEEWHVRAELATRY
IEDDELRRESISIALRHMARHRGWRNPYRQVDSLISDNPYSKQYGELKEK
AKAYNDDATAAEEESTPAQLVVAMLDAGYAEAPRLRWRTGSKKPDAEGYL
PVRLMQEDNANELKQIFRVQRVPADEWKPLFRSVFYAVSPKGSAEQRVGQ
DPLAPEQARALKASLAFQEYRIANVITNLRIKDASAELRKLTVDEKQSIY
DQLVSPSSEDITWSDLCDFLGFKRSQLKGVGSLTEDGEERISSRPPRLTS
VQRIYESDNKIRKPLVAWWKSASDNEHEAMIRLLSNTVDIDKVREDVAYA
SAIEFIDGLDDDALTKLDSVDLPSGRAAYSVETLQKLTRQMLTTDDDLHE
ARKTLFNVTDSWRPPADPIGEPLGNPSVDRVLKNVNRYLMNCQQRWGNPV
SVNIEHVRSSFSSVAFARKDKREYEKNNEKRSIFRSSLSEQLRADEQMEK
VRESDLRRLEAIQRQNGQCLYCGRTITFRTCEMDHIVPRKGVGSTNTRTN
FAAVCAECNRMKSNTPFAIWARSEDAQTRGVSLAEAKKRVTMFTFNPKSY
APREVKAFKQAVIARLQQTEDDAAIDNRSIESVAWMADELHRRIDWYFNA
KQYVNSASIDDAEAETMKTTVSVFQGRVTASARRAAGIEGKIHFIGQQSK
TRLDRRHHAVDASVIAMMNTAAAQTLMERESLRESQRLIGLMPGERSWKE
YPYEGTSRYESFHLWLDNMDVLLELLNDALDNDRIAVMQSQRYVLGNSIA
HDATIHPLEKVPLGSAMSADLIRRASTPALWCALTRLPDYDEKEGLPEDS
HREIRVHDTRYSADDEMGFFASQAAQIAVQEGSADIGSAIHHARVYRCWK
TNAKGVRKYFYGMIRVFQTDLLRACHDDLFTVPLPPQSISMRYGEPRVVQ
ALQSGNAQYLGSLVVGDEIEMDFSSLDVDGQIGEYLQFFSQFSGGNLAWK
HWVVDGFFNQTQLRIRPRYLAAEGLAKAFSDDVVPDGVQKIVTKQGWLPP
VNTASKTAVRIVRRNAFGEPRLSSAHHMPCSWQWRHE

In some embodiments the Cas9 protein can be A. muciniphila Cas9 and may comprise or consist of the amino acid sequence:

(SEQ ID NO: 170)
MSRSLTFSFDIGYASIGWAVIASASHDDADPSVCGCGTVLFPKDDCQAFK
RREYRRLRRNIRSRRVRIERIGRLLVQAQIITPEMKETSGHPAPFYLASE
ALKGHRTLAPIELWHVLRWYAHNRGYDNNASWSNSLSEDGGNGEDTERVK
HAQDLMDKHGTATMAETICRELKLEEGKADAPMEVSTPAYKNLNTAFPRL
IVEKEVRRILELSAPLIPGLTAEIIELIAQHHPLTTEQRGVLLQHGIKLA
RRYRGSLLFGQLIPRFDNRIISRCPVTWAQVYEAELKKGNSEQSARERAE
KLSKVPTANCPEFYEYRMARILCNIRADGEPLSAEIRRELMNQARQEGKL
TKASLEKAISSRLGKETETNVSNYFTLHPDSEEALYLNPAVEVLQRSGIG
QILSPSVYRIAANRLRRGKSVTPNYLLNLLKSRGESGEALEKKIEKESKK
KEADYADTPLKPKYATGRAPYARTVLKKVVEEILDGEDPTRPARGEAHPD
GELKAHDGCLYCLLDTDSSVNQHQKERRLDTMTNNHLVRHRMLILDRLLK
DLIQDFADGQKDRISRVCVEVGKELTTFSAMDSKKIQRELTLRQKSHTDA
VNRLKRKLPGKALSANLIRKCRIAMDMNWTCPFTGATYGDHELENLELEH
IVPHSFRQSNALSSLVLTWPGVNRMKGQRTGYDFVEQEQENPVPDKPNLH
ICSLNNYRELVEKLDDKKGHEDDRRRKKKRKALLMVRGLSHKHQSQNHEA
MKEIGMTEGMMTQSSHLMKLACKSIKTSLPDAHIDMIPGAVTAEVRKAWD
VFGVFKELCPEAADPDSGKILKENLRSLTHLHHALDACVLGLIPYIIPAH
HNGLLRRVLAMRRIPEKLIPQVRPVANQRHYVLNDDGRMMLRDLSASLKE
NIREQLMEQRVIQHVPADMGGALLKETMQRVLSVDGSGEDAMVSLSKKKD
GKKEKNQVKASKLVGVFPEGPSKLKALKAAIEIDGNYGVALDPKPVVIRH
IKVFKRIMALKEQNGGKPVRILKKGMLIHLTSSKDPKHAGVWRIESIQDS
KGGVKLDLQRAHCAVPKNKTHECNWREVDLISLLKKYQMKRYPTSYTGT
PR

In some embodiments the Cas9 protein can be O. laneus Cas9 and may comprise or consist of the amino acid sequence:

(SEQ ID NO: 171)
METTLGIDLGTNSIGLALVDQEEHQILYSGVRIFPEGINKDTIGLGEKEE
SRNATRRAKRQMRRQYFRKKLRKAKLLELLIAYDMCPLKPEDVRRWKNWD
KQQKSTVRQFPDTPAFREWLKQNPYELRKQAVTEDVTRPELGRILYQMIQ
RRGFLSSRKGKEEGKIFTGKDRMVGIDETRKNLQKQTLGAYLYDIAPKNG
EKYRFRTERVRARYTLRDMYIREFEIIWQRQAGHLGLAHEQATRKKNIFL
EGSATNVRNSKLITHLQAKYGRGHVLIEDTRITVTFQLPLKEVLGGKIEI
EEEQLKFKSNESVLFWQRPLRSQKSLLSKCVFEGRNFYDPVHQKWIIAGP
TPAPLSHPEFEEFRAYQFINNIIYGKNEHLTAIQREAVFELMCTESKDFN
FEKIPKHLKLFEKFNFDDTTKVPACTTISQLRKLFPHPVWEEKREEIWHC
FYFYDDNTLLFEKLQKDYALQTNDLEKIKKIRLSESYGNVSLKAIRRINP
YLKKGYAYSTAVLLGGIRNSFGKRFEYFKEYEPEIEKAVCRILKEKNAEG
EVIRKIKDYLVHNRFGFAKNDRAFQKLYHHSQAITTQAQKERLPETGNLR
NPIVQQGLNELRRTVNKLLATCREKYGPSFKFDHIHVEMGRELRSSKTER
EKQSRQIRENEKKNEAAKVKLAEYGLKAYRDNIQKYLLYKEIEEKGGTVC
CPYTGKTLNISHTLGSDNSVQIEHIIPYSISLDDSLANKTLCDATFNREK
GELTPYDFYQKDPSPEKWGASSWEEIEDRAFRLLPYAKAQRFIRRKPQES
NEFISRQLNDTRYISKKAVEYLSAICSDVKAFPGQLTAELRHLWGLNNIL
QSAPDITFPLPVSAENHREYYVITNEQNEVIRLFPKQGETPRIEKGELLL
TGEVERKVFRCKGMQEFQTDVSDGKYWRRIKLSSSVTWSPLFAPKPISAD
GQIVLKGRIEKGVFVCNQLKQKLKTGLPDGSYWISLPVISQTFKEGESVN
NSKLTSQQVQLFGRVREGIFRCHNYQCPASGADGNFWCTLDTDTAQPAFT
PIKNAPPGVGGGQIILTGDVDDKGIFHADDDLHYELPASLPKGKYYGIFT
VESCDPTLIPIELSAPKTSKGENLIEGNIWVDEHTGEVRFDPKKNREDQR
HHAIDAIVIALSSQSLFQRLSTYNARRENKKRGLDSTEHFPSPWPGFAQD
VRQSVVPLLVSYKQNPKTLCKISKTLYKDGKKIHSCGNAVRGQLHKETVY
GQRTAPGATEKSYHIRKDIRELKTSKHIGKVVDITIRQMLLKHLQENYHI
DITQEFNIPSNAFFKEGVYRIFLPNKHGEPVPIKKIRMKEELGNAERLKD
NINQYVNPRNNHHVMIYQDADGNLKEEIVSFWSVIERQNQGQPIYQLPRE
GRNIVSILQINDTFLIGLKEEEPEVYRNDLSTLSKHLYRVQKLSGMYYTF
RHHLASTLNNEREEFRIQSLEAWKRANPVKVQIDEIGRITFLNGPLC.

In some embodiments of the compositions of the disclosure, the sequence encoding the first RNA binding protein comprises a sequence isolated or derived from a CRISPR Cas protein. In some embodiments, the CRISPR Cas protein comprises a Type V CRISPR Cas protein. In some embodiments, the Type V CRISPR Cas protein comprises a Cpf1 protein. Exemplary Cpf1 proteins of the disclosure may be isolated or derived from any species, including, but not limited to, a bacteria or an archaea. Exemplary Cpf1 proteins of the disclosure may be isolated or derived from any species, including, but not limited to, Francisella tularensis subsp. novicida, Acidaminococcus sp. BV3L6 and Lachnospiraceae bacterium sp. ND2006. Exemplary Cpf1 proteins of the disclosure may be nuclease inactivated.

Exemplary wild type Francisella tularensis subsp. Novicida Cpf1 (FnCpf1) proteins of the disclosure may comprise or consist of the amino acid sequence:

| (SEQ ID NO: 172) | |
|---|---|
| 1 | MSIYQEFVNK YSLSKTLRFE LIPQGKTLEN IKARGLILDD EKRAKDYKKA KQIIDKYHQF |
| 61 | FIEEILSSVC ISEDLLQNYS DVYFKLKKSD DDNLQKDFKS AKDTIKKQIS EYIKDSEKFK |
| 121 | NLFNQNLIDA KKGQESDLIL WLKQSKDNGI ELFKANSDIT DIDEALEIIK SFKGWTTYFK |
| 181 | GFHENRKNVY SSNDIPTSII YRIVDDNLPK FLENKAKYES LKDKAPEAIN YEQIKKDLAE |
| 241 | ELTFDIDYKT SEVNQRVFSL DEVFEIANFN NYLNQSGITK FNTIIGGKFV NGENTKRKGI |
| 301 | NEYINLYSQQ INDKTLKKYK MSVLFKQILS DTESKSFVID KLEDDSDVVT TMQSFYEQIA |
| 361 | AFKTVEEKSI KETLSLLFDD LKAQKLDLSK IYFKNDKSLT DLSQQVFDDY SVIGTAVLEY |
| 421 | ITQQIAPKNL DNPSKKEQEL IAKKTEKAKY LSLETIKLAL EEFNKHRDID KQCRFEEILA |
| 481 | NFAAIPMIFD EIAQNKDNLA QISIKYQNQG KKDLLQASAE DDVKAIKDLL DQTNNLLHKL |
| 541 | KIFHISQSED KANILDKDEH FYLVFEECYF ELANIVPLYN KIRNYITQKP YSDEKFKLNF |
| 601 | ENSTLANGWD KNKEPDNTAI LFIKDDKYYL GVMNKKNNKI FDDKAIKENK GEGYKKIVYK |
| 661 | LLPGANKMLP KVFFSAKSIK FYNPSEDILR IRNHSTHTKN GSPQKGYEKF EFNIEDCRKF |
| 721 | IDFYKQSISK HPEWKDFGFR FSDTQRYNSI DEFYREVENQ GYKLTFENIS ESYIDSVVNQ |
| 781 | GKLYLFQIYN KDFSAYSKGR PNLHTLYWKA LFDERNLQDV VYKLNGEAEL FYRKQSIPKK |
| 841 | ITHPAKEAIA NKNKDNPKKE SVFEYDLIKD KRFTEDKFFF HCPITINFKS SGANKFNDEI |
| 901 | NLLLKEKAND VHILSIDRGE RHLAYYTLVD GKGNIIKQDT FNIIGNDRMK TNYHDKLAAI |
| 961 | EKDRDSARKD WKKINNIKEM KEGYLSQVVH EIAKLVIEYN AIVVFEDLNF GFKRGRFKVE |
| 1021 | KQVYQKLEKM LIEKLNYLVF KDNEFDKTGG VLRAYQLTAP FETFKKMGKQ TGIIYYVPAG |
| 1081 | FTSKICPVTG FVNQLYPKYE SVSKSQEFFS KFDKICYNLD KGYFEFSFDY KNFGDKAAKG |
| 1141 | KWTIASFGSR LINFRNSDKN HNWDTREVYP TKELEKLLKD YSIEYGHGEC IKAAICGESD |
| 1201 | KKFFAKLTSV LNTILQMRNS KTGTELDYLI SPVADVNGNF FDSRQAPKNM PQDADANGAY |
| 1261 | HIGLKGLMLL GRIKNNQEGK KLNLVIKNEE YFEFVQNRNN. |

Exemplary wild type Lachnospiraceae bacterium sp. ND2006 Cpf1 (LbCpf1) proteins of the disclosure may comprise or consist of the amino acid sequence:

| (SEQ ID NO: 173) | |
|---|---|
| 1 | AASKLEKFTN CYSLSKTLRF KAIPVGKTQE NIDNKRLLVE DEKRAEDYKG VKKLLDRYYL |
| 61 | SFINDVLHSI KLKNLNNYIS LFRKKTRTEK ENKELENLEI NLRKEIAKAF KGAAGYKSLF |
| 121 | KKDIIETILP EAADDKDEIA LVNSFNGFTT AFTGFFDNRE NMFSEEAKST SIAFRCINEN |
| 181 | LTRYISNMDI FEKVDAIFDK HEVQEIKEKI LNSDYDVEDF FEGEFFNFVL TQEGIDVYNA |
| 241 | IIGGFVTESG EKIKGLNEYI NLYNAKTKQA LPKFKPLYKQ VLSDRESLSF YGEGYTSDEE |
| 301 | VLEVFRNTLN KNSEIFSSIK KLEKLFKNFD EYSSAGIFVK NGPAISTISK DIFGEWNLIR |
| 361 | DKWNAEYDDI HLKKKAVVTE KYEDDRRKSF KKIGSFSLEQ LQEYADADLS VVEKLKEIII |
| 421 | QKVDEIYKVY GSSEKLFDAD FVLEKSLKKN DAVVAIMKDL LDSVKSFENY IKAFFGEGKE |
| 481 | TNRDESFYGD FVLAYDILLK VDHIYDAIRN YVTQKPYSKD KFKLYFQNPQ FMGGWDKDKE |
| 541 | TDYRATILRY GSKYYLAIMD KKYAKCLQKI DKDDVNGNYE KINYKLLPGP NKMLPKVFFS |
| 601 | KKWMAYYNPS EDIQKIYKNG TFKKGDMFNL NDCHKLIDFF KDSISRYPKW SNAYDFNFSE |
| 661 | TEKYKDIAGF YREVEEQGYK VSFESASKKE VDKLVEEGKL YMFQIYNKDF SDKSHGTPNL |
| 721 | HTMYFKLLFD ENNHGQIRLS GGAELFMRRA SLKKEELVVH PANSPIANKN PDNPKKTTTL |
| 781 | SYDVYKDKRF SEDQYELHIP IAINKCPKNI FKINTEVRVL LKHDDNPYVI GIDRGERNLL |
| 841 | YIVVVDGKGN IVEQYSLNEI INNFNGIRIK TDYHSLLDKK EKERFEARQN WTSIENIKEL |
| 901 | KAGYISQVVH KICELVEKYD AVIALEDLNS GFKNSRVKVE KQVYQKFEKM LIDKLNYMVD |
| 961 | KKSNPCATGG ALKGYQITNK FESFKSMSTQ NGFIFYIPAW LTSKIDPSTG FVNLLKTKYT |
| 1021 | SIADSKKFIS SFDRIMYVPE EDLFEFALDY KNFSRTDADY IKKWKLYSYG NRIRIFAAAK |
| 1081 | KNNVFAWEEV CLTSAYKELF NKYGINYQQG DIRALLCEQS DKAFYSSFMA LMSLMLQMRN |
| 1141 | SITGRTDVDF LISPVKNSDG IFYDSRNYEA QENAILPKNA DANGAYNIAR KVLWAIGQFK |
| 1201 | KAEDEKLDKV KIAISNKEWL EYAQTSVK. |

Exemplary wild type Acidaminococcus sp. BV3L6 Cpf1 (AsCpf1) proteins of the disclosure may comprise or consist of the amino acid sequence:

| (SEQ ID NO: 174) | |
|---|---|
| 1 | MTQFEGFTNL YQVSKTLRFE LIPQGKTLKH IQEQGFIEED KARNDHYKEL KPIIDRIYKT |
| 61 | YADQCLQLVQ LDWENLSAAI DSYRKEKTEE TRNALIEEQA TYRNAIHDYF IGRTDNLTDA |
| 121 | INKRHAEIYK GLFKAELFNG KVLKQLGTVT TTEHENALLR SFDKFTTYFS GFYENRKNVF |
| 181 | SAEDISTAIP HRIVQDNFPK FKENCHIFTR LITAVPSLRE HFENVKKAIG IFVSTSIEEV |
| 241 | FSFPFYNQLL TQTQIDLYNQ LLGGISREAG TEKIKGLNEV LNLAIQKNDE TAHIIASLPH |
| 301 | RFIPLFKQIL SDRNTLSFIL EEFKSDEEVI QSFCKYKTLL RNENVLETAE ALFNELNSID |
| 361 | LTHIFISHKK LETISSALCD HWDTLRNALY ERRISELTGK ITKSAKEKVQ RSLKHEDINL |
| 421 | QEIISAAGKE LSEAFKQKTS EILSHAHAAL DQPLPTTLKK QEEKEILKSQ LDSLLGLYHL |
| 481 | LDWFAVDESN EVDPEFSARL TGIKLEMEPS LSFYNKARNY ATKKPYSVEK FKLNFQMPTL |
| 541 | ASGWDVNKEK NNGAILFVKN GLYYLGIMPK QKGRYKALSF EPTEKTSEGF DKMYYDYFPD |
| 601 | AAKMIPKCST QLKAVTAHFQ THTTPILLSN NFIEPLEITK EIYDLNNPEK EPKKFQTAYA |
| 661 | KKTGDQKGYR EALCKWIDFT RDFLSKYTKT TSIDLSSLRP SSQYKDLGEY YAELNPLLYH |
| 721 | ISFQRIAEKE IMDAVETGKL YLFQIYNKDF AKGHHGKPNL HTLYWTGLFS PENLAKTSIK |
| 781 | LNGQAELFYR PKSRMKRMAH RLGEKMLNKK LKDQKTPIPD TLYQELYDYV NHRLSHDLSD |
| 841 | EARALLPNVI TKEVSHEIIK DRRFTSDKFF FHVPITLNYQ AANSPSKFNQ RVNAYLKEHP |
| 901 | ETPIIGIDRG ERNLIYITVI DSTGKILEQR SLNTIQQFDY QKKLDNREKE RVAARQAWSV |
| 961 | VGTIKDLKQG YLSQVIHEIV DLMIHYQAVV VLENLNFGFK SKRTGIAEKA VYQQFEKMLI |
| 1021 | DKLNCLVLKD YPAEKVGGVL NPYQLTDQFT SFAKMGTQSG FLFYVPAPYT SKIDPLTGFV |
| 1081 | DPFVWKTIKN HESRKHFLEG FDFLHYDVKT GDFILHFKMN RNLSFQRGLP GFMPAWDIVF |
| 1141 | EKNETQFDAK GTPFIAGKRI VPVIENHRFT GRYRDLYPAN ELIALLEEKG IVFRDGSNIL |
| 1201 | PKLLENDDSH AIDTMVALIR SVLQMRNSNA ATGEDYINSP VRDLNGVCFD SRFQNPEWPM |
| 1261 | DADANGAYHI ALKGQLLLNH LKESKDLKLQ NGISNQDWLA YIQELRN. |

In some embodiments of the compositions of the disclosure, the sequence encoding the first RNA binding protein comprises a sequence isolated or derived from a CRISPR Cas protein. In some embodiments, the CRISPR Cas protein comprises a Type VI CRISPR Cas protein or portion thereof. In some embodiments, the Type VI CRISPR Cas protein comprises a Cas13 protein or portion thereof. Exemplary Cas13 proteins of the disclosure may be isolated or derived from any species, including, but not limited to, a bacteria or an archaea. Exemplary Cas13 proteins of the disclosure may be isolated or derived from any species, including, but not limited to, Leptotrichia wadei, Listeria seeligeri serovar 1/2b (strain ATCC 35967/DSM 20751/CIP 100100/SLCC 3954), Lachnospiraceae bacterium, Clostridium aminophilum DSM 10710, Carnobacterium gallinarum DSM 4847, Paludibacter propionicigenes WB4, Listeria weihenstephanensis FSL R9-0317, Listeria weihenstephanensis FSL R9-0317, bacterium FSL M6-0635 (Listeria newyorkensis), Leptotrichia wadei F0279, Rhodobacter capsulatus SB 1003, Rhodobacter capsulatus R121, Rhodobacter capsulatus DE442 and Corynebacterium ulcerans. Exemplary Cas13 proteins of the disclosure may be DNA nuclease inactivated. Exemplary Cas13 proteins of the disclosure include, but are not limited to, Cas13a, Cas13b, Cas13c, Cas13d and orthologs thereof. Exemplary Cas13b proteins of the disclosure include, but are not limited to, subtypes 1 and 2 referred to herein as Csx27 and Csx28, respectively.

Exemplary Cas13a proteins include, but are not limited to:

| Cas13a | Cas13a | | | |
|---|---|---|---|---|
| number | abbreviation | Organism name | Accession number | Direct Repeat sequence |
| Cas13a1 | LshCas13a | Leptotrichia | WP_018451595.1 | CCACCCCAATATCGAAGGGGACTAA |
| | | shahii | | AAC (SEQ ID NO: 175) |
| Cas13a2 | LwaCas13a | Leptotrichia | WP_021746774.1 | GATTTAGACTACCCCAAAAACGAAG |
| | | wadei | | GGGACTAAAAC (SEQ ID NO: 176) |
| Cas13a3 | LseCas13a | Listeria seeligeri | WP_012985477.1 | GTAAGAGACTACCTCTATATGAAAG |
| | | | | AGGACTAAAAC (SEQ ID NO: 177) |
| Cas13a4 | LbmCas13a | Lachnospiraceae | WP_044921188.1 | GTATTGAGAAAAGCCAGATATAGTT |
| | | bacterium | | GGCAATAGAC (SEQ ID NO: 178) |
| | | MA2020 | | |
| Cas13a5 | LbnCas13a | Lachnospiraceae | WP_022785443.1 | GTTGATGAGAAGAGCCCAAGATAG |
| | | bacterium | | AGGGCAATAAC (SEQ ID NO: 179) |
| | | NK4A179 | | |
| Cas13a6 | CamCas13a | [Clostridium] | WP_031473346.1 | GTCTATTGCCCTCTATATCGGGCTGT |
| | | aminophilum | | TCTCCAAAC (SEQ ID NO: 180) |
| | | DSM 10710 | | |
| Cas13a7 | CgaCas13a | Carnobacterium | WP_034560163.1 | ATTAAAGACTACCTCTAAATGTAAG |
| | | gallinarum DSM | | AGGACTATAAC (SEQ ID NO: 181) |
| | | 4847 | | |
| Cas13a8 | Cga2Cas13a | Carnobacterium | WP_034563842.1 | AATATAAACTACCTCTAAATGTAAG |
| | | gallinarum DSM | | AGGACTATAAC (SEQ ID NO: 182) |
| | | 4847 | | |
| Cas13a9 | Pprcas13a | Paludibacter | WP_013443710.1 | CTTGTGGATTATCCCAAAATTGAAG |
| | | propionicigenes | | GGAACTACAAC (SEQ ID NO: 183) |
| | | WB4 | | |
| Cas13a10 | LweCas13a | Listeria | WP_036059185.1 | GATTTAGAGTACCTCAAAATAGAAG |
| | | weihenstephanensis | | AGGTCTAAAAC (SEQ ID NO: 184) |
| | | FSL R9-0317 | | |
| Cas13a11 | LbfCas13a | Listeriaceae | WP_036091002.1 | GATTTAGAGTACCTCAAAACAAAAG |
| | | bacterium FSL | | AGGACTAAAAC (SEQ ID NO: 185) |
| | | M6-0635 | | |
| | | (Listeria | | |
| | | newyorkensis) | | |
| Cas13a12 | Lwa2cas13a | Leptotrichia | WP_021746774.1 | GATATAGATAACCCCAAAAACGAA |
| | | wadei F0279 | | GGGATCTAAAAC (SEQ ID NO: 186) |
| Cas13a13 | RcsCas13a | Rhodobacter | WP_013067728.1 | GCCTCACATCACCGCCAAGACGACG |
| | | capsulatus SB | | GCGGACTGAAC (SEQ ID NO: 187) |
| | | 1003 | | |
| Cas13a14 | RcrCas13a | Rhodobacter | WP_023911507.1 | GCCTCACATCACCGCCAAGACGACG |
| | | capsulatus R121 | | GCGGACTGAAC (SEQ ID NO: 188) |
| Cas13a15 | RcdCas13a | Rhodobacter | WP_023911507.1 | GCCTCACATCACCGCCAAGACGACG |
| | | capsulatus | | GCGGACTGAAC (SEQ ID NO: 189) |
| | | DE442 | | |

Exemplary wild type Cas13a proteins of the disclosure may comprise or consist of the amino acid sequence:

| (SEQ ID NO: 190) | |
|---|---|
| 1 | MGNLFGHKRW YEVRDKKDFK IKRKVKVKRN YDGNKYILNI NENNNKEKID NNKFIRKYIN |
| 61 | YKKNDNILKE FTRKFHAGNI LFKLKGKEGI IRIENNDDFL ETEEVVLYIE AYGKSEKLKA |
| 121 | LGITKKKIID EAIRQGITKD DKKIEIKRQE NEEEIEIDIR DEYTNKTLND CSIILRIIEN |
| 181 | DELETKKSIY EIFKNINMSL YKIIEKIIEN ETEKVFENRY YEEHLREKLL KDDKIDVILT |
| 241 | NFMEIREKIK SNLEILGFVK FYLNVGGDKK KSKNKKMLVE KILNINVDLT VEDIADFVIK |
| 301 | ELEFWNITKR IEKVKKVNNE FLEKRRNRTY IKSYVLLDKH EKFKIERENK KDKIVKFFVE |
| 361 | NIKNNSIKEK IEKILAEFKI DELIKKLEKE LKKGNCDTEI FGIFKKHYKV NFDSKKFSKK |
| 421 | SDEEKELYKI IYRYLKGRIE KILVNEQKVR LKKMEKIEIE KILNESILSE KILKRVKQYT |
| 481 | LEHIMYLGKL RHNDIDMTTV NTDDFSRLHA KEELDLELIT FFASTNMELN KIFSRENINN |
| 541 | DENIDFFGGD REKNYVLDKK ILNSKIKIIR DLDFIDNKNN ITNNFIRKFT KIGTNERNRI |
| 601 | LHAISKERDL QGTQDDYNKV INIIQNLKIS DEEVSKALNL DVVFKDKKNI ITKINDIKIS |
| 661 | EENNNDIKYL PSFSKVLPEI LNLYRNNPKN EPFDTIETEK IVLNALIYVN KELYKKLILE |
| 721 | DDLEENESKN IFLQELKKTL GNIDEIDENI IENYYKNAQI SASKGNNKAI KKYQKKVIEC |
| 781 | YIGYLRKNYE ELFDFSDFKM NIQEIKKQIK DINDNKTYER ITVKTSDKTI VINDDFEYII |
| 841 | SIFALLNSNA VINKIRNRFF ATSVWLNTSE YQNIIDILDE IMQLNTLRNE CITENWNLNL |
| 901 | EEFIQKMKEI EKDFDDFKIQ TKKEIFNNYY EDIKNNILTE FKDDINGCDV LEKKLEKIVI |
| 961 | FDDETKFEID KKSNILQDEQ RKLSNINKKD LKKKVDQYIK DKDQEIKSKI LCRIIFNSDF |
| 1021 | LKKYKKEIDN LIEDMESENE NKFQEIYYPK ERKNELYIYK KNLFLNIGNP NFDKIYGLIS |
| 1081 | NDIKMADAKF LFNIDGKNIR KNKISEIDAI LKNLNDKLNG YSKEYKEKYI KKLKENDDFF |
| 1141 | AKNIQNKNYK SFEKDYNRVS EYKKIRDLVE FNYLNKIESY LIDINWKLAI QMARFERDMH |
| 1201 | YIVNGLRELG IIKLSGYNTG ISRAYPKRNG SDGFYTTTAY YKFFDEESYK KFEKICYGFG |
| 1261 | IDLSENSEIN KPENESIRNY ISHFYIVRNP FADYSIAEQI DRVSNLLSYS TRYNNSTYAS |
| 1321 | VFEVFKKDVN LDYDELKKKF KLIGNNDILE RLMKPKKVSV LELESYNSDY IKNLIIELLT |
| 1381 | KIENTNDTL |

Exemplary Cas13b proteins include, but are not limited to:

| Species | Cas13b Accession | Cas13b Size (aa) |
|---|---|---|
| Paludibacter propionicigenes WB4 | WP_013446107.1 | 1155 |
| Prevotella sp. P5-60 | WP_044074780.1 | 1091 |
| Prevotella sp. P4-76 | WP_044072147.1 | 1091 |
| Prevotella sp. P5-125 | WP_044065294.1 | 1091 |
| Prevotella sp. P5-119 | WP_042518169.1 | 1091 |
| Capnocytophaga canimorsus Cc5 | WP_013997271.1 | 1200 |
| Phaeodactylibacter xiamenensis | WP_044218239.1 | 1132 |
| Porphyromonas gingivalis W83 | WP_005873511.1 | 1136 |
| Porphyromonas gingivalis F0570 | WP_021665475.1 | 1136 |
| Porphyromonas gingivalis ATCC 33277 | WP_012458151.1 | 1136 |
| Porphyromonas gingivalis F0185 | ERJ81987.1 | 1136 |
| Porphyromonas gingivalis F0185 | WP_021677657.1 | 1136 |
| Porphyromonas gingivalis SJD2 | WP_023846767.1 | 1136 |
| Porphyromonas gingivalis F0568 | ERJ65637.1 | 1136 |
| Porphyromonas gingivalis W4087 | ERJ87335.1 | 1136 |
| Porphyromonas gingivalis W4087 | WP_021680012.1 | 1136 |
| Porphyromonas gingivalis F0568 | WP_021663197.1 | 1136 |
| Porphyromonas gingivalis | WP_061156637.1 | 1136 |
| Porphyromonas gulae | WP_039445055.1 | 1136 |
| Bacteroides pyogenes F0041 | ERI81700.1 | 1116 |
| Bacteroides pyogenes JCM 10003 | WP_034542281.1 | 1116 |
| Alistipes sp. ZOR0009 | WP_047447901.1 | 954 |
| Flavobacterium branchiophilum FL-15 | WP_014084666.1 | 1151 |
| Prevotella sp. MA2016 | WP_036929175.1 | 1323 |
| Myroides odoratimimus CCUG 10230 | EHO06562.1 | 1160 |
| Myroides odoratimimus CCUG 3837 | EKB06014.1 | 1158 |
| Myroides odoratimimus CCUG 3837 | WP_006265509.1 | 1158 |
| Myroides odoratimimus CCUG 12901 | WP_006261414.1 | 1158 |
| Myroides odoratimimus CCUG 12901 | EHO08761.1 | 1158 |
| Myroides odoratimimus (NZ_CP013690.1) | WP_058700060.1 | 1160 |
| Bergeyella zoohelcum ATCC 43767 | EKB54193.1 | 1225 |
| Capnocytophaga cynodegmi | WP_041989581.1 | 1219 |
| Bergeyella zoohelcum ATCC 43767 | WP_002664492.1 | 1225 |
| Flavobacterium sp. 316 | WP_045968377.1 | 1156 |
| Psychroflexus torquis ATCC 700755 | WP_015024765.1 | 1146 |
| Flavobacterium columnare ATCC 49512 | WP_014165541.1 | 1180 |
| Flavobacterium columnare | WP_060381855.1 | 1214 |
| Flavobacterium columnare | WP_063744070.1 | 1214 |
| Flavobacterium columnare | WP_065213424.1 | 1215 |
| Chryseobacterium sp. YR477 | WP_047431796.1 | 1146 |
| Riemerella anatipestifer ATCC 11845 = DSM | WP_004919755.1 | 1096 |
| 15868 | | |
| Riemerella anatipestifer RA-CH-2 | WP_015345620.1 | 949 |
| Riemerella anatipestifer | WP_049354263.1 | 949 |
| Riemerella anatipestifer | WP_061710138.1 | 951 |
| Riemerella anatipestifer | WP_064970887.1 | 1096 |
| Prevotella saccharolytica F0055 | EKY00089.1 | 1151 |
| Prevotella saccharolytica JCM 17484 | WP_051522484.1 | 1152 |
| Prevotella buccae ATCC 33574 | EFU31981.1 | 1128 |
| Prevotella buccae ATCC 33574 | WP_004343973.1 | 1128 |
| Prevotella buccae D17 | WP_004343581.1 | 1128 |
| Prevotella sp. MSX73 | WP_007412163.1 | 1128 |
| Prevotella pallens ATCC 700821 | EGQ18444.1 | 1126 |
| Prevotella pallens ATCC 700821 | WP_006044833.1 | 1126 |
| Prevotella intermedia ATCC 25611 = DSM 20706 | WP_036860899.1 | 1127 |
| Prevotella intermedia | WP_061868553.1 | 1121 |
| Prevotella intermedia 17 | AFJ07523.1 | 1135 |
| Prevotella intermedia | WP_050955369.1 | 1133 |
| Prevotella intermedia | BAU18623.1 | 1134 |
| Prevotella intermedia ZT | KJJ86756.1 | 1126 |
| Prevotella aurantiaca JCM 15754 | WP_025000926.1 | 1125 |
| Prevotella pleuritidis F0068 | WP_021584635.1 | 1140 |
| Prevotella pleuritidis JCM 14110 | WP_036931485.1 | 1117 |
| Prevotella falsenii DSM 22864 = JCM 15124 | WP_036884929.1 | 1134 |
| Porphyromonas gulae | WP_039418912.1 | 1176 |
| Porphyromonas sp. COT-052 OH4946 | WP_039428968.1 | 1176 |
| Porphyromonas gulae | WP_039442171.1 | 1175 |
| Porphyromonas gulae | WP_039431778.1 | 1176 |
| Porphyromonas gulae | WP_046201018.1 | 1176 |
| Porphyromonas gulae | WP_039434803.1 | 1176 |
| Porphyromonas gulae | WP_039419792.1 | 1120 |
| Porphyromonas gulae | WP_039426176.1 | 1120 |
| Porphyromonas gulae | WP_039437199.1 | 1120 |
| Porphyromonas gingivalis TDC60 | WP_013816155.1 | 1120 |
| Porphyromonas gingivalis ATCC 33277 | WP_012458414.1 | 1120 |
| Porphyromonas gingivalis A7A1-28 | WP_058019250.1 | 1176 |
| Porphyromonas gingivalis JCVI SC001 | EOA10535.1 | 1176 |
| Porphyromonas gingivalis W50 | WP_005874195.1 | 1176 |
| Porphyromonas gingivalis | WP_052912312.1 | 1176 |
| Porphyromonas gingivalis AJW4 | WP_053444417.1 | 1120 |
| Porphyromonas gingivalis | WP_039417390.1 | 1120 |
| Porphyromonas gingivalis | WP_061156470.1 | 1120 |

Exemplary wild type Bergeyella zoohelcum ATCC 43767 Cas13b (BzCas13b) proteins of the disclosure may comprise or consist of the amino acid sequence:

| (SEQ ID NO: 191) | |
|---|---|
| 1 | menktslgnn iyynpfkpqd ksyfagyfna amentdsvfr elgkrlkgke ytsenffdai |
| 61 | fkenislvey eryvkllsdy fpmarlldkk evpikerken fkknfkgiik avrdlrnfyt |
| 121 | hkehgeveit deifgvldem lkstvltvkk kkvktdktke ilkksiekql dilcqkkley |
| 181 | lrdtarkiee krrnqrerge kelvapfkys dkrddliaai yndafdvyid kkkdslkess |
| 241 | kakyntksdp qqeegdlkip iskngvvfll slfltkqeih afkskiagfk atvideatvs |
| 301 | eatvshgkns icfmatheif shlaykklkr kvrtaeinyg eaenaeqlsv yaketlmmqm |
| 361 | ldelskvpdv vyqnlsedvq ktfiedwney lkenngdvgt meeeqvihpv irkryedkfn |
| 421 | yfairfldef aqfptlrfqv hlgnylhdsr pkenlisdrr ikekitvfgr lselehkkal |
| 481 | fikntetned rehyweifpn pnydfpkeni svndkdfpia gsildrekqp vagkigikvk |
| 541 | llnqqyvsev dkavkahqlk qrkaskpsiq niieeivpin esnpkeaivf ggqptaylsm |
| 601 | ndihsilyef fdkwekkkek lekkgekelr keigkelekk ivgkiqaqiq qiidkdtnak |
| 661 | ilkpyqdgns taidkeklik dlkqeqnilq klkdeqtvre keyndfiayq dknreinkvr |
| 721 | drnhkqylkd nlkrkypeap arkevlyyre kgkvavwlan dikrfmptdf knewkgeqhs |
| 781 | llqkslayye qckeelknll pekvfqhlpf klggyfqqky lyqfytcyld krleyisglv |
| 841 | qqaenfksen kvfkkvenec fkflkkqnyt hkeldarvqs ilgypifler gfmdekptii |
| 901 | kgktfkgnea lfadwfryyk eyqnfqtfyd tenyplvele kkqadrkrkt kiyqqkkndv |
| 961 | ftllmakhif ksvfkqdsid qfsledlyqs reerlgnqer arqtgerntn yiwnktvdlk |
| 1021 | lcdgkitven vklknvgdfi kyeydqrvqa flkyeeniew qaflikeske eenypyvver |
| 1081 | eieqyekvrr eellkevhli eeyilekvkd keilkkgdnq nfkyyilngl lkqlknedve |
| 1141 | sykvfnlnte pedvninqlk qeatdleqka fvltyirnkf ahnqlpkkef wdycqekygk |
| 1201 | iekektyaey faevfkkeke alik. |

In some embodiments of the compositions of the disclosure, the sequence encoding the first RNA binding protein, or RNA-guided target RNA binding protein, comprises a sequence isolated or derived from a CasRX/Cas13d protein. CasRX/Cas13d is an effector of the type VI-D CRISPR-Cas systems. In some embodiments, the CasRX/Cas13d protein is an RNA-guided RNA endonuclease enzyme that can cut or bind RNA. In some embodiments, the CasRX/Cas13d protein can include one or more higher eukaryotes and prokaryotes nucleotide-binding (HEPN) domains. In some embodiments, the CasRX/Cas13d protein can include either a wild-type or mutated HEPN domain. In some embodiments, the CasRX/Cas13d protein includes a mutated HEPN domain that cannot cut RNA but can process guide RNA. In some embodiments, the CasRX/Cas13d protein does not require a protospacer flanking sequence.

Exemplary CasRX/Cas13d proteins may comprise or consist of the sequence:

### CasRX/Cas13d Gut_metagenome_contig6049000251:

| (SEQ ID NO: 54) | |
|---|---|
| LYLTSFGKGN AAVIEQKIEP ENGYRVTGMQ ITPSITVNKA TDESVRFRVK RKIAQKDEFI | 60 |
| ADNPMHEGRH RIEPSAGSDM LGLKTKLEKY YFGKEFDDNL HIQIIYNILD IEKILAVYST | 120 |
| NITA. | 124 |

Exemplary CasRX/Cas13d proteins may comprise or consist of the sequence:

### CasRX/Cas13d Gut_metagenome_contig546000275:

| (SEQ ID NO: 57) | |
|---|---|
| MDSYRPKLYK LIDFCIFKHY HEYTEISEKN VDTLRAAVSE EQKESFYADE AKRLWGIFDK | 60 |
| QFLGFCKKIN VWVNGSHEKE ILGYIDKDAY RKKSDVSYFS KFLYAMSFFL DGKEINDLLT | 120 |
| TLINKFDNIA SFISTAKELD AEIDRILEKK LDPVTGKPLK GKNSFRNFIA NNVIENKRFI | 180 |
| YVIKFCNPKN VLKLVKNTKV TEFVLKRMPE SQIDRYYSSC IDTEKNPSVD KKISDLAEMI | 240 |
| KKIAFDDFRN VRQKTRTREE SLEKERFKAV IGLYLTVVYL LIKNLVNVNS RYVMAFHCLE | 300 |
| RDAKLYGINI GKNYIELTED LCRENENSRS AYLARNKRLR DCVKQNIDNA KNMKSKEK. | 358 |

Exemplary CasRX/Cas13d proteins may comprise or consist of the sequence:

### CasRX/Cas13d Gut_metagenome_contig4114000374:

| (SEQ ID NO: 61) | |
|---|---|
| DTKINPQTWL YQLENTPDLD NEYRDTLDHF FDERFNEINE HFVTQNATNL CIMKEVFPDE | 60 |
| DFKSIADLYY DFIVVKSYKN IGFSIKKLRE KMLELPEAKR VTSTEMDSVR SKLYKLIDFC | 120 |
| IFKHYHEKPE TVEMIVSMLR AYTSEDMKE. | 149 |

Exemplary CasRX/Cas13d proteins may comprise or consist of the sequence:

### CasRX/Cas13d Gut_metagenome_contig721000619:

| (SEQ ID NO: 67) | |
|---|---|
| KEGSTMAKNE KKKSTAKALG LKSSFVVNND IYMTSFGKGN KAVLEKKITE NTIENKSDTT | 60 |
| YFDVINRDPK GFTLEGRRIA DMTAFSNDPK YHVNVVNGKF LEDQLGARSE LEKKVFGRTF | 120 |
| DDNVHIQLIH NILDIEKIMA QYVSDIVYLL HNTIKRDMND DIMGYISIRN SFDDFCHPER | 180 |
| IPDRKAKDNL QKQHDIFFDE ILKCGRLAYF GNAFFEDGSD NKEIAKLKRY KEIYHIIALM | 240 |
| GSLRQSYFHG ENSDKNFQGP TWAYTLESNL TGKYKEFKDT LDKTFDERYE MISKDFGSTN | 300 |
| MVNLQILEEL LKMLYGNVSP. | 320 |

Exemplary CasRX/Cas13d proteins may comprise or consist of the sequence:

### CasRX/Cas13d Gut_metagenome_contig2002000411:

| (SEQ ID NO: 69) | |
|---|---|
| EKQNKAKYQA IISLYLMVMY QIVKNMIYVN SRYVIAFHCL ERDSNQLLGR FNSRDASMYN | 60 |
| KLTQKFITDK YLNDGAQGCS KKVGNYLSHN ITCCSDELRK EYRNQVDHFA VVRMIGKYAA | 120 |
| DIGKFSTWFE LYHYVMQRII FDKRNPLSET ERTYKQLIAK HHTYCKDLVK ALNTPFGYNL | 180 |
| ARYKNLSIGE LFDRNNYNAK TKET. | 204 |

Exemplary CasRX/Cas13d proteins may comprise or consist of the sequence:

### CasRX/Cas13d Gut_metagenome_contig13552000311:

| (SEQ ID NO: 71) | |
|---|---|
| LIDFLIYDLY YNRKPARIEE IVDKLRESVN DEEKESIYSA ETKYVYEALG KVLVRSLKKY | 60 |
| LNGATIRDLK NRYDAKTANR IWDISEHSKS GHVNCFCKLI YMMTLMLDGK EINDLLTTLV | 120 |
| NKFDNIASFI DVMDELGLEH SFTDNYKMFA DSKAICLDLQ FINSFARMSK IDDEKSKRQL | 180 |
| FRDALVVLDI GDKNEDWIEK YLTSDIFKRD ENGNKIDGEK RDFRNFIANN VIKSARFKYL | 240 |
| VKYSSADGMI KLKKNEKLIS FVLEQLPETQ IDRYYESCGL DCAVADRKVR IEKLTGLIRD | 300 |
| MRFDNFRGVN YSNDACKKDK QAKAKYQAII SLYLMVLYQI VKNMIYVNSR YVIAFHCLER | 360 |
| DLLFFNIELD NSYQYSNCNE LTEKFIKDKY MKEGALGFNM KAGRYLTKNI GNCSNELRKI | 420 |
| YRNQVDHFAV VRKIGNYAAD IASVGSWFE. | 449 |

Exemplary CasRX/Cas13d proteins may comprise or consist of the sequence:

### CasRX/Cas13d Gut_metagenome_contig10037000527:

| (SEQ ID NO: 72) | |
|---|---|
| YMDQNFANSD AWAIHVYRNK IQHLDAVRHA DMYIGDIREF HSWFELYHYI IQRRIIDQYA | 60 |
| YESTPGSSRD GSAIIDEERL NPATRRYFRL ITTYKT. | 96 |

Exemplary CasRX/Cas13d proteins may comprise or consist of the sequence:

### CasRX/Cas13d Gut_metagenome_contig238000329:

| (SEQ ID NO: 73) | |
|---|---|
| RYDKDRSKIY TMMDFVIYRY YIDNNNDSID FINKLRSSID EKSKEKLYNE EANRLWNKLK | 60 |
| EYMLYIKEFN GKLASRTPDR DGNISEFVES LPKIHRLLPR GQKISNFSKL MYLLTMFLDG | 120 |
| KEINDLLTTL INKFENIQGF LDIMPEINVN AKFEPEYVFF NKSHEIAGEL KLIKGFAQMG | 180 |
| EPAATLKLEM TADAIKILGT EKEDAELIKL AESLFKDENG KLLGNKQHGM RNFIGNNVIK | 240 |
| SKRFHYLIRY GDPAHLHKIA TNKNVVRFVL GRIADMQKKQ GQKGKNQIDR YYEVCVGNKD | 300 |
| IKKTIEEKID ALTDIIVNMN YDQFEKKKAV IENQNRGKTF EEKNKYKRDN AEREKFKKII | 360 |
| SLYLTVIYHI LKNIVNVNSR YILGFHCLER DKQLYIEKYN KDKLDGFVAL TKFCLGDEER | 420 |
| YEDLKAKAQA SIQALETANP KLYAKYMNYS DEEKKEEFKK QLNRERVKNA RNAYLKNIKN | 480 |
| YIMIRLQLRD QTDSSGYLCG EFRDKVAHLE VARHAHEYI. | 519 |

Exemplary CasRX/Cas13d proteins may comprise or consist of the sequence:

### CasRX/Cas13d Gut_metagenome_contig2643000492:

| (SEQ ID NO: 84) | |
|---|---|
| NGEIVSLAEK EAFSAKIADK NIGCKIENKQ FRHPKGYDVI ADNPIYKGSP RQDMLGLKET | 60 |
| LEKRYFSPSD SIDNVRVQVA HNILDIEKIL AEYITNAVYS FDNIAGFGKD IIGDDFSPVY | 120 |
| TYDKFEKSDR YEYFKNLLNN SRLGYYGQAF FECDDSKENK KKKDAIKCYN IIALLSGLRH | 180 |
| W. | 181 |

Exemplary CasRX/Cas13d proteins may comprise or consist of the sequence:

### CasRX/Cas13d Gut_metagenome_contig874000057:

| (SEQ ID NO: 85) | |
|---|---|
| MSKNKESYAK GMGLKSALVS GSKVYMTSFE GGNDAKLEKV VENSEIVSLA EKESFSAEIF | 60 |
| KKNIGCKIEN KKFKHPKRYD VIADNPLYKG SVRQDMLGLK ETLEKRYFNS ADGTDNVCIQ | 120 |
| VIHNILDIEK ILAEYITNAV YSFDNIAGFG EDIIGMGGFK PIYTYKQFKE PDKYNKKFDD | 180 |
| ILNNSRLGYY GKAFFEKNDL KHNPNKKKRD KNPYILKYDN ECYYIIALLS GLRHWNIHSH | 240 |
| AKDDLVSYRW LYNLDSILNR EYISTLNYLY DDIADELTES FSKNSSANVN YIAETLNIDP | 300 |
| SEFAQQYFRF SIMKEQKNMG FNVSKLREIM LDRKELSDIR DNHRVFDSIR SKLYTMMDFV | 360 |
| IYRYYIEEAA KTEAENRNLP ENEKKISEKD FFVINLRGSF DENQKEKLYI EEAKRLWEKL | 420 |
| KDIMLKIKEF RGEKVKEYKK. | 440 |

Exemplary CasRX/Cas13d proteins may comprise or consist of the sequence:

### CasRX/Cas13d Gut_metagenome_contig4781000489:

| (SEQ ID NO: 86) | |
|---|---|
| LDKQLDYEYI RTLNYMFNDI ADELTRTFSK NSAANVNYIA ETLNIDPNKF AEQYFRFSIM | 60 |
| KEQKNLGFNL TKLRESMLDR RELSDIRDNH NVFDSIRPKL YTMMDFVIYK HYIDEAKKTE | 120 |
| AENKSLPDDR KNLSEKD. | 137 |

Exemplary CasRX/Cas13d proteins may comprise or consist of the sequence:

### CasRX/Cas13d Gut_metagenome_contig12144000352:

| (SEQ ID NO: 87) | |
|---|---|
| RMGEPVANTK RVMMIDAVKI LGTDLSDDEL KEMADSFFKD SDGNLLKKGK HGMRNFITNN | 60 |
| VIKNKRFHYL IRYGDPAHLH EIAKNEA. | 87 |

Exemplary CasRX/Cas13d proteins may comprise or consist of the sequence:

### CasRX/Cas13d Gut_metagenome_contig5590000448:

| (SEQ ID NO: 88) | |
|---|---|
| VHNNEEKDLI KYTWLYNLDK YLDAEYITTL NYMYNDIGDE LTDSFSKNSA ANINYIAETL | 60 |
| GIDPKTFAEQ YFRFSIMKEQ KNLGFNLTKL REVMLDRKDM SEIRENHNDF DSIRAKVYTM | 120 |
| MDFVIYRYYI EEAAKVNAAN KSLPDNEKSL SEKDIFVISL RGSFNEDQKD RLYYDEAQRL | 180 |
| WSKVGKLMLK IKKFRGKDTR KYKNMGTPRI RRLIPEGRDI STFSKLMYAL TMFLDGKEIN | 240 |
| DLLTTLINKF DNIQSFLKVM PLIGVNAKFA EEYSFFNNSE KIADELRLIK SFARMGEPVA | 300 |
| DARRAMYIDA IRILGTDLSD DELKALADSF SLDENGNKLG KGKHGMRNFI INNVITNKRF | 360 |
| HYLIRYGNPV HLHEIAKNEA VVKFVLGRIA DIQKKQGQNG KNQIDRYYET CIGK. | 414 |

Exemplary CasRX/Cas13d proteins may comprise or consist of the sequence:

### CasRX/Cas13d Gut_metagenome_contig525000349:

| (SEQ ID NO: 89) | |
|---|---|
| MSKKENRKSY VKGLGLKSTL VSDSKVYLTT FADGSNAKLE KCVENNKIIC ISNDKEAFAA | 60 |
| SIANKNVGYK IKNDEKFRHP KGYDIISNNP LLHNNSVQQD MLGLKNVLEK RYFGKSSGGD | 120 |
| NNLCIQIIHN IIDIEKILSE YIPNVVYAFN NIAGFKDEHN NIIDIIGTQT YNSSYTYADF | 180 |
| SKDKSDKKYI EFQKLLKNKR LGYWGKAFFT GQGNNAKVRQ ENQCFHIIAL LISLRNWATH | 240 |
| SNELDKHTKR TWLYKLDDTN ILNAEYVKTL NYLYDTIADE LTKSFSKNGA VNVNYLAKKY | 300 |
| NIKDDLPGFS EQYFRFSIMK EQKNLGFNIS KLRENMLDFK DMSVI. | 345 |

Exemplary CasRX/Cas13d proteins may comprise or consist of the sequence:

### CasRX/Cas13d Gut_metagenome_contig7229000302:

| (SEQ ID NO: 90) | |
|---|---|
| KKISSLTKFC LGESDEKKLK ALAKKSLEEL KTTNSKLYEN YIKYSDERKA EEAKRQINRE | 60 |
| RAKTAMNAHL RNTKWNDIMY GQLKDLADSK SRICSEFRNK AAHLEVARYA HMYINDISEV | 120 |
| KSYFRLYHYI MQRRIIDVIE NNPKAKYEGK VKVYFEDVKK NKKYNKNLLK LMCVPFGYCI | 180 |
| PRFKNLSIEQ MFDMNETDNS DKKKEK. | 206 |

Exemplary CasRX/Cas13d proteins may comprise or consist of the sequence:

### CasRX/Cas13d Gut_metagenome_contig3227000343:

| (SEQ ID NO: 91) | |
|---|---|
| IGDISEVNSY FQLYHYIMQR ILIDKIGSKT TGKAKEYFDS VIVNKKYDDR LLKLLCSPLG | 60 |
| YCLTRYKDLS IEALFDMNEA AKYDKLNKER KNKKK. | 95 |

Exemplary CasRX/Cas13d proteins may comprise or consist of the sequence:

### CasRX/Cas13d Gut_metagenome_contig7030000469:

| (SEQ ID NO: 92) | |
|---|---|
| SIRSKLYTMM DFVIYRYYIE ESAKAAAENK PSESDSFVIR LRGSFNENQK EELYIEEAER | 60 |
| LWKKFGEIML KIKEFRGEKV KEYKKEVPRI ERILPHGKDI SAFSKLMYML SMFLD. | 115 |

Exemplary CasRX/Cas13d proteins may comprise or consist of the sequence:

CasRX/Cas13d gut_metagenome_P17E0k2120140920,_c87000043:

| (SEQ ID NO: 93) | |
|---|---|
| MYFSKMIYML TYFLDGKEIN DLLTTLISKF DNIKEFLKIM KSSAVDVECE LTAGYKLFND | 60 |
| SQRITNELFI VKNIASMRKP AASAKLTMFR DALTILGIDD KITDDRISEI LKLKEKGKGI | 120 |
| HGLRNFITNN VIESSRFVYL IKYANAQKIR EVAKNEKVVM FVLGGIPDTQ IERYYKSCVE | 180 |
| FPDMNSSLEA KRSELARMIK NISFDDFKNV KQQAKGRENV AKERAKAVIG LYLT. | 234 |

Exemplary CasRX/Cas13d proteins may comprise or consist of the sequence:

CasRX/Cas13d Metagenomic Hit (No Protein Accession): Contig emb|OBVH01003037.1, Human Gut Metagenome Sequence (Also Found in WGS Contigs emb|OBXZ01000094.1| and emb|OBJF01000033.1|):

| (SEQ ID NO: 94) | |
|---|---|
| MAKKKRITAK ERKQNHRELL MKKADSNAEK EKAKKPVVEN KPDTAISKDN TPKPNKEIKK | 60 |
| SKAKLAGVKW VIKANDDVAY ISSFGKGNNS VLEKRIMGDV SSNVNKDSHM YVNPKYTKKN | 120 |
| YEIKNGFSSG SSLVTYPNKP DKNSGMDALC LKPYFEKDFF GHIFTDNMHI QAIYNIFDIE | 180 |
| KILAKHITNI IYTVNSFDRN YNQSGNDTIG FGLNYRVPYS EYGGGKDSNG EPKNQSKWEK | 240 |
| RDNFIKFYNE SKPHLGYYEN IFYDHGEPIS EEKFYNYLNI LNFIRNNTFH YKDDDIELYS | 300 |
| ENYSEEFVFI NCLNKFVKNK FKNVNKNFIS NEKNNLYIIL NAYGKDTENV EVVKKYSKEL | 360 |
| YKLSVLKTNK NLGVNVKKLR ESAIEYGYCP LPYDKEKEVA KLSSVKHKLY KTYDFVITHY | 420 |
| LNSNDKLLLE IVETLRLSKN DDEKENVYKK YAEKLFKADD VINPIKAISK LFARKGNKLF | 480 |
| KEKIIIKKEY IEDVSIDKNI YDFTKVIFFM TCFLDGKEIN DLLTNIISKL QVIEDHNNVI | 540 |
| KFISNNKDAV YKDYSDKYAI FRNAGKIATE LEAIKSIARM ENKIENAPQE PLLKDALLSL | 600 |
| GVSDDTKVLE NTYNKYFDSK EKTDKQSQKV STFLMNNVIN NNRFKYVIKY INPADINGLA | 660 |
| KNRYLVKFVL SKIPEEQIDS YYKLFSNEEE PGCEEKIKLL TKKISKLNFQ TLFENNKIPN | 720 |
| VEKEKKKAII TLYFTIVYIL VKNLVNINGL YTLALYFVER DGYFYKDICG KKDKKKSYND | 780 |
| VDYLLLPEIF SGSKYREETK NLKLPKEKDR DIMKKYLPND KDREKYNKFF TAYRNNIVHL | 840 |
| NIIAKLSELT KNIDKDINSY FDIYHYCTQR VMFNYCKEKN DVVLAKMKDL AHIKSDCNEF | 900 |
| SSKHTYPFSS AVLRFMNLPF AYNVPRFKNL SYKKFFDKQ. | 939 |

Exemplary CasRX/Cas13d proteins may comprise or consist of the sequence:

CasRX/Cas13d Metagenomic Hit (No Protein Accession): Contig tpg|DJXD01000002.1| (Uncultivated Ruminococcus Assembly, UBA7013, from Sheep Gut Metagenome):

| (SEQ ID NO: 95) | |
|---|---|
| MKKQKSKKTV SKTSGLKEAL SVQGTVIMTS FGKGNMANLS YKIPSSQKPQ NLNSSAGLKN | 60 |
| VEVSGKKIKF QGRHPKIATT DNPLFKPQPG MDLLCLKDKL EMHYFGKTFD DNIHIQLIYQ | 120 |
| ILDIEKILAV HVNNIVFTLD NVLHPQKEEL TEDFIGAGGW RINLDYQTLR GQTNKYDRFK | 180 |
| NYIKRKELLY FGEAFYHENE RRYEEDIFAI LTLLSALRQF CFHSDLSSDE SDHVNSFWLY | 240 |
| QLEDQLSDEF KETLSILWEE VTERIDSEFL KTNTVNLHIL CHVFPKESKE TIVRAYYEFL | 300 |
| IKKSFKNMGF SIKKLREIML EQSDLKSFKE DKYNSVRAKL YKLFDFIITY YYDHHAFEKE | 360 |
| ALVSSLRSSL TEENKEEIYI KTARTLASAL GADFKKAAAD VNAKNIRDYQ KKANDYRISF | 420 |
| EDIKIGNTGI GYFSELIYML TLLLDGKEIN DLLTTLINKF DNIISFIDIL KKLNLEFKFK | 480 |
| PEYADFFNMT NCRYTLEELR VINSIARMQK PSADARKIMY RDALRILGMD NRPDEEIDRE | 540 |
| LERTMPVGAD GKFIKGKQGF RNFIASNVIE SSRFHYLVRY NNPHKTRTLV KNPNVVKFVL | 600 |
| EGIPETQIKR YFDVCKGQEI PPTSDKSAQI DVLARIISSV DYKIFEDVPQ SAKINKDDPS | 660 |
| RNFSDALKKQ RYQAIVSLYL TVMYLITKNL VYVNSRYVIA FHCLERDAFL HGVTLPKMNK | 720 |
| KIVYSQLTTH LLTDKNYTTY GHLKNQKGHR KWYVLVKNNL QNSDITAVSS FRNIVAHISV | 780 |
| VRNSNEYISG IGELHSYFEL YHYLVQSMIA KNNWYDTSHQ PKTAEYLNNL KKHHTYCKDF | 840 |
| VKAYCIPFGY VVPRYKNLTI NELFDRNNPN PEPKEEV. | 877 |

An exemplary direct repeat sequence of CasRX/Cas13d Metagenomic hit (no protein accession): contig tpg|DJXD01000002.1| (uncultivated Ruminococcus assembly, UBA7013, from sheep gut metagenome) (SEQ ID NO: 95) comprises or consists of the nucleic acid sequence:

### CasRX/Cas13d DR:

(SEQ ID NO: 96)

| | |
|---|---|
| caactacaac cccgtaaaaa tacggggttc tgaaac. | 36 |

Exemplary CasRX/Cas13d proteins may comprise or consist of the sequence:

CasRX/Cas13d Metagenomic hit (no protein accession): contig OGZC01000639.1 (human gut metagenome assembly):

| (SEQ ID NO: 97) | |
|---|---|
| MKKKNIRATR EALKAQKIKK SQENEALKKQ KLAEEAAQKR REELEKKNLA QWEETSAEGR | 60 |
| RSRVKAVGVK SVFVVGDDLY LATFGNGNET VLEKKITPDG KITTFPEEET FTAKLKFAQT | 120 |
| EPTVATSIGI SNGRIVLPEI SVDNPLHTTM QKNTIKRSAG EDILQLKDVL ENRYFDRSFN | 180 |
| DDLHIRLIYN ILDIEKILAE YTTNAVFAID NVSGCSDDFL SNFSTRNQWD EFQNPEQHRE | 240 |
| HFGNKDNVIC SVKKQQDLFF NFFKNNRIGY FGKAFFHAES ERKIVKKTEK EVYHILTLIG | 300 |
| SLRQWITHST EGGISRLWLY QLEDALSREY QETMNNCYNS TIYGLQKDFE KTNAPNLNFL | 360 |
| AEILGKNASE LAEPYFRFII TKEYKNLGFS IKTLREMLLD QPDLQEIREN HNVYDSIRSK | 420 |
| LYKMIDFVLV YAYSNERKSK ADALASNLRS AITEDAKKRI YQNEADQLWT SYQELFKRIR | 480 |
| GFKGAQVKEY SSKNMPIPIQ KQIQNILKPA EQVTYFTKLM YLLTMFLDGK EINDLLTTLI | 540 |
| NKFDNISSLL KTMEQLELQT TFKEDYTFFQ QSSRLCKEIT QLKSFARMGN PISNLKEVMM | 600 |
| VDAIQILGTE KSEQELQSMA CFFFRDKNGK KLNTGEHGMR NFIGNNVISN TRFQYLIRYG | 660 |
| NPQKLHTLSQ NETVVRFVLS RIAKNQRVQG MNGKNQIDRY YETCGGTNSW SVSEEEKINF | 720 |
| LCKILTNMSY DQFQDVKQSG AEITAEEKRK KERYKAIISL YLTVLYQLIK NLVNINARYI | 780 |
| IAFHCLERDA ILYSSKFNTS INLKKRYTAL TEMILGYETD EKARRKDTRT VYEKAEAAKN | 840 |
| RHLKNVKWNC KTRENLENAD KNAIVAFRNI VAHLWIIRDA DRFITGMGAM KRYFDCYHYL | 900 |
| LQRELGYILE KSNQGSEYTK KSLEKVQQYH SYCKDFLHML CLPFAYCIPR YKNLSIAELF | 960 |
| DRHEPEAEPK EEASSVNNSQ FITT. | 984 |

Exemplary CasRX/Cas13d proteins may comprise or consist of the sequence:

CasRX/Cas13d Metagenomic Hit (No Protein Accession): Contig emb|OHBM01000764.1 (Human Gut Metagenome Assembly):

| (SEQ ID NO: 98) | |
|---|---|
| XXXXXXXXXX XXXXXXXXXX XXXXXXXXXX XXXXXXXXXX XXXXXXXXXX XXXXXXXXXX | 60 |
| XXXXXXXXXX XXXXXXXXXX XXXXXXXXXX XXXXXXXXXX XXXXXXXXXX XXXXXXXXXX | 120 |
| XXXXXXXXXX XXXXXXXXXX XXXXXXXXXX XXXXXXXXXX XXXXXXXXXX XXXXXXXXXX | 180 |
| XXXXXXXXXX XXXXXXXXXX XXXXXXXXXX XXXXXXXXXX XXHPLQKRYR YLTSTNLKSF | 240 |
| ETYKNNLVNK KKFDLDRVKK IPQLAYFGSA FYNTPEDTSA KITKTKIKSN EEIYYTFMLL | 300 |
| STARNFSAHY LDRNRAKSSD AEDFDGTSVI MYNLDNEELY KKLYNKKVHM ALTGMKKVLD | 360 |
| ANFNKKVEHL NNSFIKNSAK DFVILCEVLG IKSRDEKTKF VKDYYDFVVR KNYKHLGFSV | 420 |
| KELRELLFAN HDSNKYIKEF DKISNKKFDS VRSRLNRLAD YIIYDYYNKN NAKVSDLVKY | 480 |
| LRAAADDEQK KKIYLNESIN LVKSGILERI KKILPKLNGK IIGNMQPDST ITASMLHNTG | 540 |
| KDWHPISENA HYFTKWIYTL TLFMDGKEIN DLVTTLINKF DNIASFIEVL KSQSVCTHFS | 600 |
| EERKMFIDSA EICSELSAMN SFARMEAPGA SSKRAMFVEA ARILGDNRSK EELEEYFDTL | 660 |
| FDKSASKKEK GFRNFIRNNV VDSNRFKYLT RYTDTSSVKA FSNNKALVKF AIKDIPQEQI | 720 |
| LRYYNSCFGA SERYYNDGMS DKLVEAIGKI NLMQFNGVIQ QADRNMLPEE KKKANAQKEK | 780 |
| YKSIIRLYLT VCYLFFKNLV YVNSRYYSAF YNLEKDRSLF EINGELKPTG KFDEGHYTGL | 840 |
| VKLFIDNGWI NPRASAYLTV NLANSDETAI RTFRNTAEHL EALRNADKYL NDLKQFDSYF | 900 |
| EIYHYITQRN IKEKCEMLKE QTVKYNNDLL KYHGYSKDFV KALCVPFGYN LPRFKNLSID | 960 |
| ALFDKNDKRE KLKKGFED. | 978 |

Exemplary CasRX/Cas13d proteins may comprise or consist of the sequence:

CasRX/Cas13d Metagenomic Hit (No Protein Accession): Contig emb|OHCP01000044.1 (Human Gut Metagenome Assembly):

| (SEQ ID NO: 99) | |
|---|---|
| MAKKITAKQK REEKERLNKQ KWAKNDSVII VPETKEEIKT GEIQDNNRKR SRQKSQAKAM | 60 |
| GLKAVLSFDN KIAIASFVSS KNAKSSHIER ITDKEGTTIS VNSKMFESSV NKRDINIEKR | 120 |
| ITIEEPQQDG TIKKEEKGVK STTCNPYFKV GGKDYIGIKE IAEEHFFGRA FPNENLRVQI | 180 |
| AYNIFDVQKI LGTFVNNIIY SFYNLSRDEV QSDNDVIGML YSISDYDRQK ETETFLQAKS | 240 |
| LLKQTEAYYA YFDDVFKKNK KPDKNKEGDN SKQYQENLRH NFNILRVLSF LRQICMHAEV | 300 |
| HVSDDEGCTR TQNYTDSLEA LFNISKAFGK KMPELKTLID NIYSKGINAI NDEFVKNGKN | 360 |
| NLYILSKVYP NEKREVLLRE YYNFVVCKEG SNIGISTRKL KETMIAQNMP SLKEENTYRN | 420 |
| KLYTVMNFIL VRELKNCATI REQMIKELRA NMDEEEGRDR IYSKYAKEIY LYVKDKLKLM | 480 |
| LNVFKEEAEG IIIPGKEDPV KFSHGKLDKK EIESFCLTTK NTEDITKVIY FLCKFLDGKE | 540 |
| INELCCAMMN KLDGISDLIE TAKQCGEDVE FVDQFKCLSK CATMSNQIRI VKNISRMKKE | 600 |
| MTIDNDTIFL DALELLGRKI EKYQKDKNGD YVKDEKGKKV YTKDYNNFQD MFFEGKNHRV | 660 |
| RNFVSNNVIK SKWFSYVVRY NKPAECQALM RNSKLVKFAL DELPDSQIEK YYISVFGEKS | 720 |
| SSSNEEMRRE LLKKLCDFSV RGFLDEIVLL SEDEMKQKDK FSEKEKKKSL IRLYLTIVYL | 780 |
| ITKSMVKINT RFSIACATYE RDYILLCQSE KAERAWEKGA TAFALTRKFL NHDKPTFEQY | 840 |
| YTREREISAM PQEKRKELRK ENDQLLKKTH YSKHAYCYIV DNVNNLTGAV ANDNGRGLPC | 900 |
| LSEKNDNANL FLEMRNKIVH LNVVHDMVKY INEIKNITSY YAFFCYVLQR MIIGNNSNEQ | 960 |
| NKFKAKYSKT LQEFGTYSKD LMWVLNLPFA YNLPRYKNLS NEQLFYDEEE RMEKIVGRKN | 1020 |
| DSR. | 1023 |

Exemplary CasRX/Cas13d proteins may comprise or consist of the sequence:

CasRX/Cas13d Metagenomic Hit (No Protein Accession): Contig emb|OGDF01008514.1| (Human Gut Metagenome Assembly):

| (SEQ ID NO: 100) | |
|---|---|
| MTETKPKRED IAKTPAAKSR SKAAGLKSTF AVNGSVLLTS FGRGNDAVPE KLITEKAVSE | 60 |
| INTVKPRFSV EKPATSYSSS FGIKSHISAT ADNPLAGRAP VGEDAIHAKE VLEQRVFGKT | 120 |
| FSDDNIHIQL IYNILDIRKI LSTYANNVVF TINSMRRLDE YDREQDYLGY LYTGNSYERL | 180 |
| LDIADKYAVD GEDWRNTAAG ISNDFEKKQF QTINGFWDLL DMIEPYMCYF SEAFFCETTV | 240 |
| KDPDSGRIVP CLEQRSDGDI YNILRILSIV RQTCMHDNAS MRTVMFTLGQ NSVRDRKNGF | 300 |
| DELAELLDYL YDEKIDIVNR DFLRNQKNNI ELLSRIYGSS ADSPERDRLV QNFYDFRVLS | 360 |
| QDKNLGFSIK KLREKLLDSP ALSVVRSKKY DTMRSKIYSL IDFMIYRKFS ENHVAVDDFV | 420 |
| EELRSLLTED EKESAYSRWA ETLINDGFAQ EILVKLLPQT DPAVIGKIKG KKLLNDSIAG | 480 |
| IKLKKDASFF TKIINVLCMF QDGKEINELV SSLVNKFANI QSFVDVMRSQ GIDSGFTADY | 540 |
| AMFAESGRIS RELHILKGIA RMQHSIAGLG DVKIYGSDDK FHGVSRRVYT DAAYILGFGE | 600 |
| RSEDNDGYVD DYVSSKLLGG ADKNLRNFIT NNVIKNRRFL YTVRYMNPKR AKKLVQNDAL | 660 |
| VVLALSGIPE TQIDRYYKSC IEKRSFNPDL NEKIAALSEM ITTLKIDDFE DVKQNPEKNA | 720 |
| NYEAKKNQRI SKERYKACIG LYLTVLYLIC KNLVKINARY SIAIGCLERD TQLHGVDFKG | 780 |
| AAYMTRDVFI AKGWINPKKP TVKSIKEQYA FLTPYIFTTY RNMIAHLAAV TNAYKYIPQM | 840 |
| DRFKSWFHLY HTVIQHSLIQ QYEYDRDYGR KGAPVVSERV LQLLEQCREH SNYSRDLLHI | 900 |
| LNLPFGYNLP RYLNLSSEKY FDANAI. | 926 |

Exemplary CasRX/Cas13d proteins may comprise or consist of the sequence:

CasRX/Cas13d Metagenomic Hit (No Protein Accession): Contig emb|OGPN01002610.1 (Human Gut Metagenome Assembly):

| (SEQ ID NO: 101) | |
|---|---|
| MAKKITAKQK REEKERLNKQ KWAKQDTPVV PKSKTEEKPV AASDDKLLKT TQVKKVQTKS | 60 |
| KAKAMGLKTV LSFDDKIAIA SFVNDKKTKL PHIERITDKS GTTIHENARM FDSSVDEQNV | 120 |
| NIEKRMTIEE KQNDGTFKKD EKDVKATICN PYFKTCGKDY IGIKDVAEKY FFGKTFPNEN | 180 |
| LRVQIAYNVF DIQKILGTYV NNIIYSFYNL RRDGKSDVDI IGSLYAFADF DNQLKDKPAF | 240 |
| REAKDLLKNT EAYFSYFGDV FKKSKKGKKD ENNEDYEKNL RHNFNVLRVL SFLRQICTHA | 300 |
| YVKCTGGAKN NGDSTKVEAE SLDALFNITE YFAKTAPELS KTINEIYKEG IDRINNDFVT | 360 |
| NGKNNLYILS KVYPDMQRNE LVKKYYQFVV CKEGNNVGIN TRKLKESIIS QHPWITTPQD | 420 |
| NNKANDYESC RHKLYTIMCF ILVAELDAHE SIRDNMVAEL RANMDGDDGR DAIYEKYAKD | 480 |
| IYHIVKDKLL AMQKVFDEEL VPVKVEGKND PQQFTHGKLG KKEIESFCLS DKNTSDIAKV | 540 |
| VYFLCNFLDG KEINELCCAM MNKFDGIGDL IDTAKQCGEE VKFIEEFACL SNCRKITNDI | 600 |
| RVAKSISKMK NKVNIDNDII YLDAIELLGR KIEKYQKDEN GKILLGTDGK RLYTQEYKYF | 660 |
| NDMFFNAGNH KVRNFIANNV MQSKWFFYVV RYNKPAECQI IMRNKTLVKF TLDDLPDMQI | 720 |
| QRYYSSVFGD NNMPAVDEMR KRLLDKINQF SVRGFLDELD EIVLMSDEES KRNKSSEKEQ | 780 |
| KKSLIRLYLT IAYLITKSMV KINTRFSIAC AMYERDYALL CQSEMKGGPW DGGAQALAVT | 840 |
| RKFLNHDREV FDRYCAREAE IARLPSEERK PLRKANDKLL KQTHYTNHSY TYIVNNLNSF | 900 |
| TDIDYCAKDV GLPAPNDKND NASILGEMRN DIAHLNIVHD MVKYIEELKD ISSYYAFYCY | 960 |
| VLQRRLVGKD PNCQNKFKAK YAKELNDYGT YNKNLMWMLN LPFAYNLPRY KNLSSEFLFY | 1020 |
| DMEYNKKDDE. | 1030 |

Exemplary CasRX/Cas13d proteins may comprise or consist of the sequence:

CasRX/Cas13d Metagenomic Hit (No Protein Accession): From Contig emb|OBLI01020244 and emb|OBLI01038679 (from Pig Gut Metagenome):

| (SEQ ID NO: 102) | |
|---|---|
| MAKKITAKQR REERERQNKQ KWAKKQADAT AVFECEADIK PADSKDEDCT NIYIKREKKK | 60 |
| TQAKAMGLKT VLGFDNKIAI ASFMSSKDSK SSHIERITDP NGKTIREDVR MFDSNVDECS | 120 |
| INLEKRMTVE ERQKDGTIKK DEKDVKSTIC NPYSNECGKD YIGIKSVAEE LFFGRTFPND | 180 |
| NLRVQIAYNI FDIQKILGTY INNIIYSFYN LSRDESQSDN DVIGTLYMLK DFDGQKETDT | 240 |
| FRQARALLER TEAYYSYFDN VFKKIDKNKK KSDDCKRERN EILRYNFNVL RVLSFLRQIC | 300 |
| AHAQVKISNE HDREKGGGLV DSLDALFNIS RFFDAVAPEL NEVINSVYSK GIDDINDNFV | 360 |
| KNGKNNFYIL SKIYPEVARE DLLREYYYFV VSKEGNNIGI STKKLKEAII VQDMSYIKSE | 420 |
| DYDTYRNKLY TVLCFILVKE LNERTTIREQ MVADLRANMN GDIGREDIYS KYAKIIYAQV | 480 |
| KPRFDTMKSA FEEEAKDVIV PDKKKPVKFS HGKLDKNEIE RFCITSANTD SVAKIIYFLC | 540 |
| KFLDGKEINE LCCAMMNKLD GINDLIETAE QCGAKVEFVD KFSVLSNCET ISDQIRIVKS | 600 |
| ISKMKKEIAI DNDTIFLDAL ELLGRKIDKY KKDATGKYLK DENGKYLYSK EYDDFQYMFF | 660 |
| KDSHRVRNFI SNSVIKSKWF SYIVRYNQPS ECRAIMKNKT LVKFALDELP DLQIQRYFVA | 720 |
| LYGDEDLPSY GEMRKILLKK LHDFSIKGFL DEIVLLSDLD MESQDKYCEK EQKKSLFRLY | 780 |
| LTIAYLITKS MVKINTRFSI ACATYERDYA LLCASNKQER AWSSGATALA LTRRFLNQDK | 840 |
| LIFEKHYARE GEISKLPKEE RKAMRKVNDQ LLKRTHFSKH SYCYIVDNVN RLTGGECRTD | 900 |
| KRVLPVLNEK NDNAGILLDF RKTIAHLNVV HKMVDYVDEI KGITSYYAFF CYVLQRMLVG | 960 |
| NNLNEKNAIK EKYSATVKSF GTYSKDFMWL INLPFAYNLP RYKNLSNEQL FYDEEERNET | 1020 |
| EEQIDRL. | 1027 |

Exemplary CasRX/Cas13d proteins may comprise or consist of the sequence:

### CasRX/Cas13d Metagenomic Hit (No Protein Accession): Contig OIZX01000427.1:

| (SEQ ID NO: 103) | |
|---|---|
| MAKKKKTARQ LREEMQQQRK QAIQKQQEQR QEKAAAARET AAPEQPAAAP VPKRQRKSLA | 60 |
| KAAGLKSNFI LDPQRRTTVM TAFGQGSTAI LEKQIVDRAI SDLQPVQQFQ VEPASAAKYR | 120 |
| LKNSRVRFPN VTADDPLYRR KDGGFVPGMD ALRRKNVLEQ RFFGKSFADN IHIQMIYSIL | 180 |
| DIHKILAAAS GHIVHLLNIV NGSKDRDFIG MLAAHVLYNE LNEEAKRSIA DFCKSPRLIY | 240 |
| YSAAFYETLD NGKSERRSNE DIFNILALMT CLRNFSSHHS IAIKVKDYSA AGLYNLRRLG | 300 |
| PDMKKMLDTF YTEAFIQLNQ SFQDHNTTNL TCLFDILNIS DSARQKQLAE EFYRYVVFKE | 360 |
| QKNLGFSVRK LREEMLLLPD AAVIADKRYD TCRSKLYNLM DFLILRVYRT GRADRCDKLP | 420 |
| EALRAALTDE EKAVVYHKEA LSLWNEMRTL ILDGLLPQMT PENLSRLSGQ KRKGELSLDD | 480 |
| AMLKECLYEP GPVPEDAAPE EANAEYFCRM IYLATLFMDG KEINTLLTTL ISKFENIAAF | 540 |
| LQTMEQLNIE AELGPEYAMF TRSRAVAEQL RVINSFALMK KPQVNAKQQL YRAAVTLLGT | 600 |
| EDPDGVTDEM LCIDPVTGKM LPPNQRHHGD TGLRNFIANN VVESRRFQYL IRYSDPAQLH | 660 |
| QLASNKKLVR FVLSSIPDTQ INRYYETCGQ TRLAGRAAKV EFLTDMIAAI RFDQFRDVNQ | 720 |
| KERGANTQKE RYKAMLGLYQ TVLYLAVKNL VNINARYVMA FHCVERDMFL YDGELTDPKG | 780 |
| ESVSAFLAVN GKKGVQPQYL LLTQLFIRRD YLKRSACEQI QHNMENISDR LLREYRNAVA | 840 |
| HLNVIAHLAD YSADMREITS YYGLYHYLMQ RHLFKRHAWQ IRQPERPTEE EQKLIEQEQK | 900 |
| QLAWEKALFD KTLQYHSYNK DLVKALNAPF GYNLARYKNL SIEPLFSKEA APAAEIKATH | 960 |
| A. | 961 |

Exemplary CasRX/Cas13d proteins may comprise or consist of the sequence:

### CasRX/Cas13d Metagenomic Hit (No Protein Accession): Contig OCTW011587266.1:

| (SEQ ID NO: 104) | |
|---|---|
| MKQNDRENNN KIKKSAAKAV GVKSLARLSD GSTVVSSFGK GAAAELESLI TGGEIRKLSD | 60 |
| KAILEITDDT QNKNAYNVKS SRIPNLTART DKLSDKSGMD DLGFKRELEL EVFGQCFDDS | 120 |
| IHIQIAHAVF DIQKSLAAVI PNVLYTLNNL DRSYSTDNTS DKKDIIGNTL NYQHSYESFN | 180 |
| VEKRGEFTEY YNAAKDRFSY FPDILCVLEK VNGKDRYQPK SEKDAFNVLS SVNMLRNSLF | 240 |
| HFAPKSNDGK ARIAVFKNQF DSDFSHITST VNKIYSAKIA GVNENFLNNE GNNLYIILKA | 300 |
| TNWDIKKIVP QLYRFSVLKS DKNMGFNMRK LREFAVESKN IDLSRLNDKF LTNNRKKLYK | 360 |
| VIDFIIYYHL NKVLKDSFVD DFVAALRASQ SEEEKEKLYA QYSERLFADE GLKSAIKKAV | 420 |
| DMISDTKSNI FKMKTPLDKA LIENIKVNSD ASDFCKLIYV FTRFLDGKEI NILLNSLIKK | 480 |
| FQDIHSFNTT VKKLSENNLI INADYVDDYS LFEQSGTVAR ELMLIKSISK MDFGLDNINL | 540 |
| SFMYDDALRT LGVSDENLPE VKREYFGKTK NLSAYIRNNV LENRRFKYVI KYIHPSDVQK | 600 |
| IACNKAIAGF VLNRMPDTQI KRYYDSLINK GATDIQAQAK ALLDCITGIS FDAIKDDKHL | 660 |
| HKSKEKSPQR SADRERKKAM LTLYYTIVYI FVKQMLHINS LYTIGFFYLE RDQRFIYSRA | 720 |
| KKENKNPSKN SYLNDFRSVT AYFIPSEIMK RIEKNENKGF LEDFEALWNS CGKTSRLRKE | 780 |
| DVLLYARYIS PDHALKNYKM ILNSYRNKIA HINVIMSAGK YTGGIKRMDS YFSVFQHLVQ | 840 |
| CDILSNPNNK GKCFESESLK PLLLDMKFDG TDEKLYSKRL TRALNIPFGY NVPRYKNLTF | 900 |
| EKIYLKSSIN E. | 911 |

Exemplary CasRX/Cas13d proteins may comprise or consist of the sequence:

CasRX/Cas13d Metagenomic Hit (No Protein Accession): Contig emb|OGNF01009141.1:

| (SEQ ID NO: 105) | |
|---|---|
| MADIDKKKSS AKAAGLKSTF VLENNKLLMT SFGNGNKAVI EKIIDEKVDS INEPEVFSVT | 60 |
| PCDKKFELQP AKRGLAADSL VDNPLKSKKT AGDDAIHSRK FLERQFFDGN TFNDNIHIQL | 120 |
| IYNILDIEKI LSVHVNDIVY SVNNILSRGE GMEYNDYIGT LNLKSFETYK NNLVNKKKFD | 180 |
| LDRVKKIPQL AYFGSAFYNT PEDTSAKITK TKIKSNEEIY YTFMLLSTAR NFSAHYLDRN | 240 |
| RAKSSDAEDF DGTSVIMYNL DNEELYKKLY NKKVHMALTG MKKVLDANFN KKVEHLNNSF | 300 |
| IKNSAKDFVI LCEVLGIKSR DEKTKFVKDY YDFVVRKNYK HLGFSVKELR ELLFANHDSN | 360 |
| KYIKEFDKIS NKKFDSVRSR LNRLADYIIY DYYNKNNAKV SDLVKYLRAA ADDEQKKKIY | 420 |
| LNESINLVKS GILERIKKIL PKLNGKIIGN MQPDSTITAS MLHNTGKDWH PISENAHYFT | 480 |
| KWIYTLTLFM DGKEINDLVT TLINKFDNIA SFIEVLKSQS VCTHFSEERK MFIDSAEICS | 540 |
| ELSAMNSFAR MEAPGASSKR AMFVEAARIL GDNRSKEELE EYFDTLFDKS ASKKEKGFRN | 600 |
| FIRNNVVDSN RFKYLTRYTD TSSVKAFSNN KALVKFAIKD IPQEQILRYY NSCFGASERY | 660 |
| YNDGMSDKLV EAIGKINLMQ FNGVIQQADR NMLPEEKKKA NAQKEKYKSI IRLYLTVCYL | 720 |
| FFKNLVYVNS RYYSAFYNLE KDRSLFEING ELKPTGKFDE GHYTGLVKLF IDNGWINPRA | 780 |
| SAYLTVNLAN SDETAIRTFR NTAEHLEALR NADKYLNDLK QFDSYFEIYH YITQRNIKEK | 840 |
| CEMLKEQTVK YNNDLLKYHG YSKDFVKALC VPFGYNLPRF KNLSIDALFD KNDKREKLKK | 900 |
| GFED. | 904 |

Exemplary CasRX/Cas13d proteins may comprise or consist of the sequence:

CasRX/Cas13d Metagenomic Hit (No Protein Accession): Contig emb|OIEN01002196.1:

| (SEQ ID NO: 106) | |
|---|---|
| MERQKRKMKS KSKMAGVKSV FVIGDELLMT SFGDGDDAVL EKDIDENGVV NDCRNPAAYD | 60 |
| AVYGTDSIRV KKTNNNIRAK VNNPLAKSNI RSEESALFRT RVNEYKREQK DKYETLFFGK | 120 |
| TFDDNIHIQL ISKILDIEKT FSVVIGNIVY AINNLSLEQS IDRPIDIFGD KNTQGISLRE | 180 |
| DNDYLKTMLP RCEYLFHNIL NSDSDNNSKM NYNKVNKGKE EKDNRNNENI EKLKKALEVI | 240 |
| KIIRVDSFHG VDGIKGDQKF PRSKYNLAVN YNEEIQKTIS EPFNRKVEEV QQDFYRNSCV | 300 |
| NIDFLKEIMY GSNYTDRGSD SLECSYFNFA ILKQNKNMGF SITSIRECLL DLYELNFESM | 360 |
| QNLRPRANSF CDFLIYDYYC KNESERANLV DCLRSAASEE EKKNIYFQTA ERVKEKFRNA | 420 |
| FNRISRFDAS YIKNSREKNL SGGSSLPKYS FIEGFTKRSK KINDNDEKNA DLFCNMLYYL | 480 |
| AQFLDGKEIN IFLTSIHNIF QNIDSFLKVM KEKGMECKFQ KDFKMFSHAG HVAKKIEIVI | 540 |
| SLAKMKKTLD FYNAQALKDA VTILGVSKKH QYLDMNSYLD FYMFDNRSGA TGKNAGKDHN | 600 |
| LRNFLVSNVI RSRKFNYLSR YSNLAEVKKL AQNPSLVQFV LSRIEPSLIC RYYESSQGIS | 660 |
| SEGITIDEQI KKLTGIIVDM NIDSFENINN GEIGMRYSKA TPQSIERRNQ MRVCVGLYLN | 720 |
| VLYQIEKNLM NVNARYVLAF AFAERDALML NFTLEECKKN KKRSSGGFSF IEMTQFFIDK | 780 |
| KLFKVATEAI KKNVLKYNGN PESLNHIPGE YICKNMEGYH ENTVRNFRNM VAHLTAVARV | 840 |
| PLYISEVTQI DSYYALYHYC MQMNILQGIE QSGKILDNIK LKNALENARV HRTYSKDAVK | 900 |
| YLCLPFAYNI SRYKALTIKD LFDWTEYSCK KDE. | 933 |

Exemplary CasRX/Cas13d proteins may comprise or consist of the sequence:

CasRX/Cas13d Metagenomic Hit (No Protein Accession): Contig e-k87_11092736:

| (SEQ ID NO: 107) | |
|---|---|
| MKRQKTFAKR IGIKSTVAYG QGKYAITTFG KGSKAEIAVR SADPPEETLP TESDATLSIH | 60 |
| AKFAKAGRDG REFKCGDVDE TRIHTSRSEY ESLISNPAES PREDYLGLKG TLERKFFGDE | 120 |
| YPKDNLRIQI IYSILDIQKI LGLYVEDILH FVDGLQDEPE DLVGLGLGDE KMQKLLSKAL | 180 |
| PYMGFFGSTD VFKVTKKREE RAAADEHNAK VFRALGAIRQ KLAHFKWKES LAIFGANANM | 240 |
| PIRFFQGATG GRQLWNDVIA PLWKKRIERV RKSFLSNSAK NLWVLYQVFK DDTDEKKKAR | 300 |
| ARQYYHFSVL KEGKNLGFNL TKTREYFLDK FFPIFHSSAP DVKRKVDTFR SKFYAILDFI | 360 |
| IYEASVSVAN SGQMGKVAPW KGAIDNALVK LREAPDEEAK EKIYNVLAAS IRNDSLFLRL | 420 |
| KSACDKFGAE QNRPVFPNEL RNNRDIRNVR SEWLEATQDV DAAAFVQLIA FLCNFLEGKE | 480 |
| INELVTALIK KFEGIQALID LLRNLEGVDS IRFENEFALF NDDKGNMAGR IARQLRLLAS | 540 |
| VGKMKPDMTD AKRVLYKSAL EILGAPPDEV SDEWLAENIL LDKSNNDYQK AKKTVNPFRN | 600 |
| YIAKNVITSR SFYYLVRYAK PTAVRKLMSN PKIVRYVLKR LPEKQVASYY SAIWTQSESN | 660 |
| SNEMVKLIEM IDRLTTEIAG FSFAVLKDKK DSIVSASRES RAVNLEVERL KKLTTLYMSI | 720 |
| AYIAVKSLVK VNARYFIAYS ALERDLYFFN EKYGEEFRLH FIPYELNGKT CQFEYLAILK | 780 |
| YYLARDEETL KRKCEICEEI KVGCEKHKKN ANPPYEYDQE WIDKKKALNS ERKACERRLH | 840 |
| FSTHWAQYAT KRDENMAKHP QKWYDILASH YDELLALQAT GWLATQARND AEHLNPVNEF | 900 |
| DVYIEDLRRY PEGTPKNKDY HIGSYFEIYH YIRQRAYLEE VLAKRKEYRD SGSFTDEQLD | 960 |
| KLQKILDDIR ARGSYDKNLL KLEYLPFAYN LPRYKNLTTE ALFDDDSVSG KKRVAEWRER | 1020 |
| EKTREAEREQ RRQR. | 1034 |

An exemplary direct repeat sequence of CasRX/Cas13d Metagenomic hit (no protein accession): contig e-k87_11092736 (SEQ ID NO: 107) comprises or consists of the nucleic acid sequence:

CasRX/Cas13d Direct repeat 1:
(SEQ ID NO: 108)
gtgagaagtc tccttatggg gagatgctac.

Exemplary CasRX/Cas13d proteins may comprise or consist of the sequence:

### CasRX/Cas13d Ga0129306_1000735:

| (SEQ ID NO: 109) | |
|---|---|
| MQKQREQQTV TDESERKKKP LKSGAKAAGL KSVFVLSEGK ELLTSFGRGN EAVPEKRVTG | 60 |
| GTIANARTDN KEAFSAALQN KRFEVFGRTA GSSDDPLAVS RAPGQDLIGA KTALEERYFG | 120 |
| RAFADNIHMQ VIYAIQDINK ILAVHANNIV YTLNNLDREA DPETDDFIGS GYLTLKNTFE | 180 |
| TYCDPAALNE REREKVTVSK QHFDAFMQNP RLAYYGNAFF RKLSKAERLA RGREIFDKES | 240 |
| PERRQEILGS RGKNKSVDDE IRALAPEWVK REERDVYSEL VLMSELRQSC FHGQQKNSAR | 300 |
| IFRLDNDLGP GVDGARELLD RLYAEKINDL RSFDKTSASS NFRLLFNAYH ADNEKKKELA | 360 |
| QEFYRFSVLK VSKNTGFSIR TLREKIIEDH AAQYRDKIYD SMRKKLFSTF DFFLWRFYEE | 420 |
| REDEAEELRA CLRAARSDEE KEQIYAEAAA SCWPSVKPFV ESVAATLCDV VKGRTKLNKL | 480 |
| KLSADESTLV RNAIDGVRIS PRASYFTKLI YLMTLFLDGK EINDLLTTLI HAFENIDSFL | 540 |
| SVLGSERLER TFDANYRIFA DSGVIAQELR AVNSFARMTT EPFNSKLVMF EDAAQLFGMS | 600 |
| GGLVEHAEEL REYLDNKMLD KTKLRLLPDG KVDTGFRNFI ISNVTESRRF RYLVRYCEPR | 660 |
| AVRDYMSCRP LIRLTLRDMP DTILRRYYEQ SVGAATVDRE RILDTLADKL LSLRFTDFEN | 720 |
| VNQRANAERN REKQKMMGII SLYLNVAYQI VKNLVYVNAR YTMAYHCAER DTELLLNAAG | 780 |
| EGNLLRRDRS WPARLHLPRR ALARRRDRVE VMERDVARGP EAYNRDEWLG LVRTLRREKR | 840 |
| VCDNLHNNYA YLCGADAEPG DASLSLLFVY RNKAAHLSVL NKGGRLSGDL KEAKSWFYVY | 900 |
| HFLMQRVLEE EFRNTQALPE RLRELLMMAE RYRGCSKDLI KVLNLTFAYN LPRYKNLSID | 960 |
| GRFDKNHPDP SDE. | 973 |

Exemplary CasRX/Cas13d proteins may comprise or consist of the sequence:

### CasRX/Cas13d Ga0129317_1008067:

| (SEQ ID NO: 110) | |
|---|---|
| MKKQKKSLVK AAGLKSAFVV GDSVYLTSFG KGNAARLDTK INPDNSTERY VSDSEKHTLK | 60 |
| INSITDTELR LSGPFPKQAE AKNPTHKKDN EQKNTRQDML GLKSTLEKFY FGSTFDDNIH | 120 |
| IQIIHNIQDI AKILAAHSNN AGYALDNMLA YQGVEFSDMI GYMGTSRTFD NYDPNHKNNK | 180 |
| DFFRFLKLPR LGYFGSAFYS QKGKDFEKRS DEEVYNICAL MGQIRQCCFH GKQEKYQLKW | 240 |
| LYNFHNFKSN KPFLDTLDKH FDEMIDRINK NFIKNNTPDL IILSGLYPDM AKKELVRLFY | 300 |
| DFTTVKEYKN MGFSVKKLRE KMLESEEASD FRDKDYDSVR RKLYKLMDFC IYYLYYSDSE | 360 |
| RNENLVSRLR ESLTDENKDI IYSKEAKIVW NELRKKFSTI LDNVKGSNIK KLENVKEKFI | 420 |
| SEDEFDDIKL DIDISYFSKL MYVMCYFLDG KEINDLLTTL VSKFDNIGSI IEAATQIGIN | 480 |
| IEFIDDFKFF DRSKDISVEL NIIRNFARMQ APVPNAKRAM QEDAIRILGG SEEDIFSILD | 540 |
| DMTGYDKSGK KLAQSKKGFR NFIINNVVES SRFKYIVRYS NPQKIRKLAN NSVVVGFVLG | 600 |
| KLPDAQIESY FNSCLPNRVY STPDKARESL RDMLHNISFN DFADVKQDDR RATPEEKVEK | 660 |
| ERYKAIIGLY LTVMYHLVKN LVYVNSRYVM AFHCLERDAM HYDVSLDNYR DLIRHLISEG | 720 |
| DSSCNHFISH NRRMRDCIEE NVKNSEQLIF GKEDAVIRFR NNVAHLSAIR NANEYIGDIR | 780 |
| EITSYFALYH YLMQRKLIDD CKVNDTAHKY FEQLTKYKTY VMDMVKALCS PFGYNLPRFK | 840 |
| NLSIEGKFDM HESK. | 854 |

Exemplary CasRX/Cas13d proteins may comprise or consist of the sequence:

### CasRX/Cas13d Ga0224415_10048792:

| (SEQ ID NO: 111) | |
|---|---|
| MSKKENRKSY VKGLGLKSTL VSDSKVYLTT FADGSNAKLE KCVENNKIIC ISNDKEAFAA | 60 |
| SIANKNVGYK IKNDEKFRHP KGYDIISNNP LLHNNSVQQD MLGLKNVLEK RYFGKSSGGD | 120 |
| NNLCIQIIHN IIDIEKILSE YIPNVVYAFN NIAGFKDEHN NIIDIIGTQT YNSSYTYADF | 180 |
| SKDKSDKKYI EFQKLLKNKR LGYWGKAFFT GQGNNAKVRQ ENQCFHIIAL LISLRNWATH | 240 |
| SNELDKHTKR TWLYKLDDTN ILNAEYVKTL NYLYDTIADE LTKSFSKNGA VNVNYLAKKY | 300 |
| NIKDDLPGFS EQYFRFSIMK EQKNLGFNIS KLRENMLDFK DMSVIRDDHN RYDKDRSKIY | 360 |
| TMMDFVIYRY YIDNNNDSID FINKLRSSID EKSKEKLYNE EANRLWNKLK EYMLYIKEFN | 420 |
| GKLASRTPDR DGNISEFVES LPKIHRLLPR GQKISNFSKL MYLLTMFLDG KEINDLLTTL | 480 |
| INKFENIQGF LDIMPEINVN AKFEPEYVFF NKSHEIAGEL KLIKGFAQMG EPAATLKLEM | 540 |
| TADAIKILGT EKEDAELIKL AESLFKDENG KLLGNKQHGM RNFIGNNVIK SKRFHYLIRY | 600 |
| GDPAHLHKIA TNKNVVRFVL GRIADMQKKQ GQKGKNQIDR YYEVCVGNKD IKKTIEEKID | 660 |
| ALTDIIVNMN YDQFEKKKAV IENQNRGKTF EEKNKYKRDN AEREKFKKII SLYLTVIYHI | 720 |
| LKNIVNVNSR YILGFHCLER DKQLYIEKYN KDKLDGFVAL TKFCLGDEER FEDLKAKAQA | 780 |
| SIQALETANP KLYAKYMNYS DEEKKEEFKK QLNRERVKNA RNAYLKNIKN YIMIRLQLRD | 840 |
| QTDSSGYLCG EFRDKVAHLE VARHAHEYIG NIKEVNSYFQ LYHYIMQCRL YDVLKNNTKA | 900 |
| EAMVKGKAKE YFEALEKEGT YNDKLLKIAC VPFGYCIPRY KNLSMEELFD MNEEKKFKKK | 960 |
| APENT. | 965 |

Exemplary CasRX/Cas13d proteins may comprise or consist of the sequence:

CasRX/Cas13d 160582958_gene49834:

| (SEQ ID NO: 112) | |
|---|---|
| MKNSVTFKLI QAQENKEAAR KKAKDIAEQA RIAKRNGVVK KEENRINRIQ IEIQTQKKSN | 60 |
| TQNAYHLKSL AKAAGVKSVF AIGNDLLMTG FGPGNDATIE KRVFQNRAIE TLSSPEQYSA | 120 |
| EFQNKQFKIK GNIKVLNHST QKMEEIQTEL QDNYNRPHFD LLGCKNVLEQ KYFGRTFSDN | 180 |
| IHVQIAYNIM DIEKLLTPYI NNIIYTLNEL MRDNSKDDFF GCDSHFSVAY LYDELKAGYS | 240 |
| DRLKTKPNLS KNIDRIWNNF CNYMNSDSGN TEARLAYFGE LFYKPKETGD AKSDYKTHLS | 300 |
| NNQKEEWELK SDKEVYNIFA ILCDLRHFCT HGESITPSGK PFPYNLEKNL FPEAKQVLNS | 360 |
| LFEEKAESLG AEAFGKTAGK TDVSILLKVF EKEQASQKEQ QALLKEYYDF KVQKTYKNMG | 420 |
| FSIKKLREAI MEIPDAAKFK DDLYSSLRHK LYGLFDFILV KHFLDTSDSE NLQNNDIFRQ | 480 |
| LRACRCEEEK DQVYRSIAVK VWEKVKKKEL NMFKQVVVIP SLSKDELKQM EMTKNTELLS | 540 |
| SIETISTQAS LFSEMIFMMT YLLDGKEINL LCTSLIEKFE NIASFNEVLK SPQIGYETKY | 600 |
| TEGYAFFKNA DKTAKELRQV NNMARMTKPL GGVNTKCVMY NEAAKILGAK PMSKAELESV | 660 |
| FNLDNHDYTY SPSGKKIPNK NFRNFIINNV ITSRRFLYLI RYGNPEKIRK IAINPSIISF | 720 |
| VLKQIPDEQI KRYYPPCIGK RTDDVTLMRD ELGKMLQSVN FEQFSRVNNK QNAKQNPNGE | 780 |
| KARLQACVRL YLTVPYLFIK NMVNINARYV LAFHCLERDH ALCFNSRKLN DDSYNEMANK | 840 |
| FQMVRKAKKE QYEKEYKCKK QETGTAHTKK IEKLNQQIAY IDKDIKNMHS YTCRNYRNLV | 900 |
| AHLNVVSKLQ NYVSELPNDY QITSYFSFYH YCMQLGLMEK VSSKNIPLVE SLKNEANDAQ | 960 |
| SYSAKKTLEY FDLIEKNRTY CKDFLKALNA PFSYNLPRFK NLSIEALFDK NIVYEQADLK | 1020 |
| KE. | 1022 |

An exemplary direct repeat sequence of CasRX/Cas13d proteins may comprise or consist of the sequence

CasRX/Cas13d 160582958_gene49834 (SEQ ID NO: 112) comprises or consists of the nucleic acid sequence:

### CasRX/Cas13d DR:

(SEQ ID NO: 113)
gaactacacc cctctgttct tgtaggggtc taacac.

Exemplary CasRX/Cas13d proteins may comprise or consist of the sequence:

CasRX/Cas13d 250twins_35838_GL0110300:

| (SEQ ID NO: 114) | |
|---|---|
| MGNKQRVSAQ KRRENAKLCN QQKARQAESQ RDKIKNMNVE KMKNINTNDI KHTKTTAKKL | 60 |
| GLKSTIIADK KIILTSFINE QSSKTANIEK VAGFKGDTID TISYTPRMFR SEINPGEIVI | 120 |
| SKGDDLSEFA NPANFPIGRD YVKIRSALEK QYFGKEFPED NLHVQIAYNV ADIKKILSVY | 180 |
| INNIIYMFYN LARSEEYDIF YNSQSENSGR DCDVIGSLYY QASYRNQDAN RFEKDGKKKA | 240 |
| IDSLLDDTRA YYTYFDGLFS VPKREDDGKI KESEKEKAKD QNFDVLRLLS VGRQLTFHSD | 300 |
| KSNNEAYLFD LSKLTRAAQD ENRRQDIQSL LNILNSTCRS NLEGVNGDFV KHAKNNLYVL | 360 |
| NQLYPSLKAN DLIGEYYNFI VKKENRNIGI RLITVRELII EHNYTNLKDS KYDTYRNKIY | 420 |
| TVLNFILFRE IQENSIAIKN FREKLRSTEK AEQPALYQAF ANKIYPMVQA KFAKAIDLFE | 480 |
| EQYKTKFKSE FKGGISIENM QQQNILLQTE NIDYFSKYVL FLTKFLDGKE INELLCALIN | 540 |
| KFDNIADLLD ISKQIGTPVV FCADYESLND AAKIAENIRL IKNIAHLRPA IQEAQSSKDN | 600 |
| ADAAGTPATL LIDAYNMLNT DIQLVYGEAA YEELRKDLFE RKNGTKYNKK GKKVDVYDHK | 660 |
| FRNFLINNVI KSKWFFYIAK YVKPADCAKM MSNKKMIEFA LRDLPETQIK RYYYTITGNE | 720 |
| ALGDAESLKG VIIEQLHAFS IKNTLLSIKN MGEGEYKIQQ IGSSKEKLKA IVNLYLTVAY | 780 |
| LLTKSLVKVN IRFSIAFGCL ERDLVLQKKS EKKFDAIINE ILLEDDKIRK ECDKERAQAK | 840 |
| TLPRELAQER FAQIKRRESG CYFKSYHVYD YLSKNSNEFK QNHIDFAVTS YRNNVEHLNV | 900 |
| VHCMTKYFSE VKDVKSYYGV YCYIMQRMLC DELIIKNQDK PDVRQTFEEY NRLLKDHGTY | 960 |
| SKNLMWLLNF PFAYNLARYK NLSNEDLFNA KNNDQKSK. | 998 |

Exemplary CasRX/Cas13d proteins may comprise or consist of the sequence:

CasRX/Cas13d 250twins_36050_GL0158985:

| (SEQ ID NO: 115) | |
|---|---|
| MKKKHQSAAE KRQVKKLKNQ EKAQKYASEP SPLQSDTAGV ECSQKKTVVS HIASSKTLAK | 60 |
| AMGLKSTLVM GDKLVITSFA ASKAVGGAGY KSANIEKITD LQGRVIEEHE RMFSADVGEK | 120 |
| NIELSKNDCH TNVNNPVVTN IGKDYIGLKS RLEQEFFGKT FENDNLHVQL AYNILDIKKI | 180 |
| LGTYVNNIIY IFYNLNRAGT GRDERMYDDL IGTLYAYKPM EAQQTYLLKG DKDMRRFEEV | 240 |
| KQLLQNTSAY YVYYGTLFEK VKAKSKKEQR AKEAEIDACT AHNYDVLRLL SLMRQLCMHS | 300 |
| VAGTAFKLAE SALFNIEDVL SADLKEILDE AFSGAVNKLN DGFVQHSGNN LYVLQQLYPN | 360 |
| ETIERIAEKY YRLTVRKEDL NMGVNIKKLR ELIVGQYFPE VLDKEYDLSK NGDSVVTYRS | 420 |
| KIYTVMNYIL LYYLEDHDSS RESMVEALRQ NREGDEGKEE IYRQFAKKVW NGVSGLFGVC | 480 |
| LNLFKTEKRN KFRSKVALPD VSGAAYMLSS ENIDYFVKML FFVCKFLDGK EINELLCALI | 540 |
| NKFDNIADIL DAAAQCGSSV WFVDSYRFFE RSRRISAQIR IVKNIASKDF KKSKKDSDES | 600 |
| YPEQLYLDAL ALLGDVISKY KQNRDGSVVI DDQGNAVLTE QYKRFRYEFF EEIKRDESGG | 660 |
| IKYKKSGKPE YNHQRRNFIL NNVLKSKWFF YVVKYNRPSS CRELMKNKEI LRFVLRDIPD | 720 |
| SQVRRYFKAV QGEEAYASAE AMRTRLVDAL SQFSVTACLD EVGGMTDKEF ASQRAVDSKE | 780 |
| KLRAIIRLYL TVAYLITKSM VKVNTRFSIA FSVLERDYYL LIDGKKKSSD YTGEDMLALT | 840 |
| RKFVGEDAGL YREWKEKNAE AKDKYFDKAE RKKVLRQNDK MIRKMHFTPH SLNYVQKNLE | 900 |
| SVQSNGLAAV IKEYRNAVAH LNIINRLDEY IGSARADSYY SLYCYCLQMY LSKNFSVGYL | 960 |
| INVQKQLEEH HTYMKDLMWL LNIPFAYNLA RYKNLSNEKL FYDEEAAAEK ADKAENERGE. | 1020 |

Yan et al. (2018) Mol Cell. 70(2):327-339 (doi: 10.1016/j.molcel.2018.02.2018) and Konermann et al. (2018) Cell 173(3):665-676 (doi: 10.1016/j.cell/2018.02.033) have described CasRX/Cas13d proteins and both of which are incorporated by reference herein in their entireties. Also see WO Publication Nos. WO2018/183703 (CasM) and WO2019/006471 (Cas13d), which are incorporated herein by reference in their entirety.

Exemplary wild type Cas13d proteins of the disclosure may comprise or consist of the amino acid sequence:

Cas13d (Ruminococcus flavefaciens XPD3002) Sequence:

(SEQ ID NO: 45)

| | |
|---|---|
| 1 | IEKKKSFAKG MGVKSTLVSG SKVYMTTFAE GSDARLEKIV |
| | EGDSIRSVNE GEAFSAEMAD |
| 61 | KNAGYKIGNA KFSHPKGYAV VANNPLYTGP VQQDMLGLKE |
| | TLEKRYFGES ADGNDNICIQ |
| 121 | VIHNILDIEK ILAEYITNAA YAVNNISGLD KDIIGFGKFS |
| | TVYTYDEFKD PEHHRAAFNN |
| 181 | NDKLINAIKA QYDEFDNFLD NPRLGYFGQA FFSKEGRNYI |
| | INYGNECYDI LALLSGLAHW |
| 241 | VVANNEEESR ISRTWLYNLD KNLDNEYIST LNYLYDRITN |
| | ELTNSFSKNS AANVNYIAET |
| 301 | LGINPAEFAE QYFRFSIMKE QKNLGFNITK LREVMLDRKD |
| | MSEIRKNHKV FDSIRTKVYT |
| 361 | MMDFVIYRYY IEEDAKVAAA NKSLPDNEKS LSEKDIFVIN |
| | LRGSFNDDQK DALYYDEANR |
| 421 | IWRKLENIMH NIKEFRGNKT REYKKKDAPR LPRILPAGRD |
| | VSAFSKLMYA LTMFLDGKEI |
| 481 | NDLLTTLINK FDNIQSFLKV MPLIGVNAKF VEEYAFFKDS |
| | AKIADELRLI KSFARMGEPI |
| 541 | ADARRAMYID AIRILGTNLS YDELKALADT FSLDENGNKL |
| | KKGKHGMRNF IINNVISNKR |
| 601 | FHYLIRYGDP AHLHEIAKNE AVVKFVLGRI ADIQKKQGQN |
| | GKNQIDRYYE TCIGKDKGKS |
| 661 | VSEKVDALTK IITGMNYDQF DKKRSVIEDT GRENAEREKF |
| | KKIISLYLTV IYHILKNIVN |
| 721 | INARYVIGFH CVERDAQLYK EKGYDINLKK LEEKGFSSVT |
| | KLCAGIDETA PDKRKDVEKE |
| 781 | MAERAKESID SLESANPKLY ANYIKYSDEK KAEEFTRQIN |
| | REKAKTALNA YLRNTKWNVI |
| 841 | IREDLLRIDN KTCTLFANKA VALEVARYVH AYINDIAEVN |
| | SYFQLYHYIM QRIIMNERYE |
| 901 | KSSGKVSEYF DAVNDEKKYN DRLLKLLCVP FGYCIPRFKN |
| | LSIEALFDRN EAAKFDKEKK |
| 961 | KVSGNS. |

Exemplary wild type Cas13d proteins of the disclosure may comprise or consist of the amino acid sequence:

Cas13d (contig e-k87_11092736):

(SEQ ID NO: 46)
MKRQKTFAKRIGIKSTVAYGQGKYAITTFGKGSKAEIAVRSADPPEETLP
TESDATLSIHAKFAKAGRDGREFKCGDVDETRIHTSRSEYESLISNPAES
PREDYLGLKGTLERKFFGDEYPKDNLRIQIIYSILDIQKILGLYVEDILH
FVDGLQDEPEDLVGLGLGDEKMQKLLSKALPYMGFFGSTDVFKVTKKREE
RAAADEHNAKVFRALGAIRQKLAHFKWKESLAIFGANANMPIRFFQGATG
GRQLWNDVIAPLWKKRIERVRKSFLSNSAKNLWVLYQVFKDDTDEKKKAR
ARQYYHFSVLKEGKNLGFNLTKTREYFLDKFFPIFHSSAPDVKRKVDTFR
SKFYAILDFIIYEASVSVANSGQMGKVAPWKGAIDNALVKLREAPDEEAK
EKIYNVLAASIRNDSLFLRLKSACDKFGAEQNRPVFPNELRNNRDIRNVR
SEWLEATQDVDAAAFVQLIAFLCNFLEGKEINELVTALIKKFEGIQALID
LLRNLEGVDSIRFENEFALFNDDKGNMAGRIARQLRLLASVGKMKPDMTD
AKRVLYKSALEILGAPPDEVSDEWLAENILLDKSNNDYQKAKKTVNPFRN
YIAKNVITSRSFYYLVRYAKPTAVRKLMSNPKIVRYVLKRLPEKQVASYY
SAIWTQSESNSNEMVKLIEMIDRLTTEIAGFSFAVLKDKKDSIVSASRES
RAVNLEVERLKKLTTLYMSIAYIAVKSLVKVNARYFIAYSALERDLYFFN
EKYGEEFRLHFIPYELNGKTCQFEYLAILKYYLARDEETLKRKCEICEEI
KVGCEKHKKNANPPYEYDQEWIDKKKALNSERKACERRLHFSTHWAQYAT
KRDENMAKHPQKWYDILASHYDELLALQATGWLATQARNDAEHLNPVNEF
DVYIEDLRRYPEGTPKNKDYHIGSYFEIYHYIRQRAYLEEVLAKRKEYRD
SGSFTDEQLDKLQKILDDIRARGSYDKNLLKLEYLPFAYNLPRYKNLTTE
ALFDDDSVSGKKRVAEWREREKTREAEREQRRQR.

An exemplary direct repeat sequence of Cas13d (contig e-k87_11092736) (SEQ ID NO: 46) comprises or consists of the nucleic acid sequence:

Cas13d (contig e-k87_11092736) Direct Repeat Sequence):

(SEQ ID NO: 47)
GTGAGAAGTCTCCTTATGGGGAGATGCTAC.

Exemplary wild type Cas13d proteins of the disclosure may comprise or consist of the amino acid sequence:

Cas13d (160582958_gene49834):

(SEQ ID NO: 48)
MKNSVTFKLIQAQENKEAARKKAKDIAEQARIAKRNGVVKKEENRINRIQ
IEIQTQKKSNTQNAYHLKSLAKAAGVKSVFAIGNDLLMTGFGPGNDATIE
KRVFQNRAIETLSSPEQYSAEFQNKQFKIKGNIKVLNHSTQKMEEIQTEL
QDNYNRPHFDLLGCKNVLEQKYFGRTFSDNIHVQIAYNIMDIEKLLTPYI
NNIIYTLNELMRDNSKDDFFGCDSHFSVAYLYDELKAGYSDRLKTKPNLS
KNIDRIWNNFCNYMNSDSGNTEARLAYFGELFYKPKETGDAKSDYKTHLS
NNQKEEWELKSDKEVYNIFAILCDLRHFCTHGESITPSGKPFPYNLEKNL
FPEAKQVLNSLFEEKAESLGAEAFGKTAGKTDVSILLKVFEKEQASQKEQ
QALLKEYYDFKVQKTYKNMGFSIKKLREAIMEIPDAAKFKDDLYSSLRHK
LYGLFDFILVKHFLDTSDSENLQNNDIFRQLRACRCEEEKDQVYRSIAVK
VWEKVKKKELNMFKQVVVIPSLSKDELKQMEMTKNTELLSSIETISTQAS
LFSEMIFMMTYLLDGKEINLLCTSLIEKFENIASFNEVLKSPQIGYETKY
TEGYAFFKNADKTAKELRQVNNMARMTKPLGGVNTKCVMYNEAAKILGAK
PMSKAELESVFNLDNHDYTYSPSGKKIPNKNFRNFIINNVITSRRFLYLI
RYGNPEKIRKIAINPSIISFVLKQIPDEQIKRYYPPCIGKRTDDVTLMRD
ELGKMLQSVNFEQFSRVNNKQNAKQNPNGEKARLQACVRLYLTVPYLFIK
NMVNINARYVLAFHCLERDHALCFNSRKLNDDSYNEMANKFQMVRKAKKE
QYEKEYKCKKQETGTAHTKKIEKLNQQIAYIDKDIKNMHSYTCRNYRNLV
AHLNVVSKLQNYVSELPNDYQITSYFSFYHYCMQLGLMEKVSSKNIPLVE
SLKNEANDAQSYSAKKTLEYFDLIEKNRTYCKDFLKALNAPFSYNLPRFK
NLSIEALFDKNIVYEQADLKKE.

An exemplary direct repeat sequence of Cas13d (160582958_gene49834) (SEQ ID NO: 48) comprises or consists of the nucleic acid sequence:

Cas13d (160582958_gene49834) Direct Repeat Sequence:

(SEQ ID NO: 49)
GAACTACACCCCTCTGTTCTTGTAGGGGTCTAACAC.

Exemplary wild type Cas13d proteins of the disclosure may comprise or consist of the amino acid sequence:

Cas13d (contig tpg|DJXD01000002.1|; uncultivated Ruminococcus assembly, UBA7013, from sheep gut metagenome):

(SEQ ID NO: 50)
MKKQKSKKTVSKTSGLKEALSVQGTVIMTSFGKGNMANLSYKIPSSQKPQ
NLNSSAGLKNVEVSGKKIKFQGRHPKIATTDNPLFKPQPGMDLLCLKDKL
EMHYFGKTFDDNIHIQLIYQILDIEKILAVHVNNIVFTLDNVLHPQKEEL
TEDFIGAGGWRINLDYQTLRGQTNKYDRFKNYIKRKELLYFGEAFYHENE
RRYEEDIFAILTLLSALRQFCFHSDLSSDESDHVNSFWLYQLEDQLSDEF
KETLSILWEEVTERIDSEFLKTNTVNLHILCHVFPKESKETIVRAYYEFL
IKKSFKNMGFSIKKLREIMLEQSDLKSFKEDKYNSVRAKLYKLFDFIITY
YYDHHAFEKEALVSSLRSSLTEENKEEIYIKTARTLASALGADFKKAAAD
VNAKNIRDYQKKANDYRISFEDIKIGNTGIGYFSELIYMLTLLLDGKEIN
DLLTTLINKFDNIISFIDILKKLNLEFKFKPEYADFFNMTNCRYTLEELR
VINSIARMQKPSADARKIMYRDALRILGMDNRPDEEIDRELERTMPVGAD
GKFIKGKQGFRNFIASNVIESSRFHYLVRYNNPHKTRTLVKNPNVVKFVL
EGIPETQIKRYFDVCKGQEIPPTSDKSAQIDVLARIISSVDYKIFEDVPQ
SAKINKDDPSRNFSDALKKQRYQAIVSLYLTVMYLITKNLVYVNSRYVIA
FHCLERDAFLHGVTLPKMNKKIVYSQLTTHLLTDKNYTTYGHLKNQKGHR
KWYVLVKNNLQNSDITAVSSFRNIVAHISVVRNSNEYISGIGELHSYFEL
YHYLVQSMIAKNNWYDTSHQPKTAEYLNNLKKHHTYCKDFVKAYCIPFGY
VVPRYKNLTINELFDRNNPNPEPKEEV.

An exemplary direct repeat sequence of Cas13d (contig tpg|DJXD01000002.1|; uncultivated Ruminococcus assembly, UBA7013, from sheep gut metagenome) (SEQ ID NO: 50) comprises or consists of the nucleic acid sequence:

Cas13d (contig tpg|DJXD01000002.1|; uncultivated Ruminococcus assembly, UBA7013, from sheep gut metagenome):

(SEQ ID NO: 51)

CAACTACAACCCCGTAAAAATACGGGGTTCTGAAAC.

In some embodiments of the disclosure, a CjeCas9-endonuclease fusions and gRNA molecule may comprise or consist of the nucleic acid sequence of:

E43-CjeCas9 and sgRNA plasmid
(U6: N's = sgRNA spacer, E43, CjeCas9)
(SEQ ID NO: 202)
gtttattacagggacagcagagatccagtttggttaattaaggtaccgag
ggcctatttcccatgattccttcatatttgcatatacgatacaaggctgt
tagagagataattagaattaatttgactgtaaacacaaagatattagtac
aaaatacgtgacgtagaaagtaataatttcttgggtagtttgcagtttta
aaattatgttttaaaatggactatcatatgcttaccgtaacttgaaagta
tttcgatttcttggctttatatatcttGTGGAAAGGACGAAACACCNNNN
NNNNNNNNNNNNNNNGTTTTAGTCCCTGAAGGGACTAAAATAAAGAGTTT
GCGGGACTCTGCGGGGTTACAATCCCCTAAAACCGCTTTTTTTCCTGCAG
CCCGGGGGATCCACTAGTTCTAGAGCGGCCGCCACCGCGGTGGAGCTCCA
GCTTTTGTTCCCTTTAGTGAGGGTTAATTGCGCGAATTCGCTAGCTAGGT
CTTGAAAGGAGTGGGAATTGGCTCCGGTGCCCGTCAGTGGGCAGAGCGCA
CATCGCCCACAGTCCCCGAGAAGTTGGGGGGAGGGGTCGGCAATTGATCC
GGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAG
GACCGGTTCTAGAGCGCTATTTAGAACCatgTGTTCTCCCCAAGAATCTG
GCATGACCGCTCTTTCAGCGAGGATGTTGACGCGAAGCAGATCCCTGGGA
CCTGGGGCCGGGCCACGAGGGTGTCGGGAAGAACCAGGACCGTTGCGACG
GAGGGAAGCAGCAGCGGAAGCTCGGAAATCCCATTCTCCGGTTAAACGAC
CCCGCAAGGCACAACGGCTCAGGGTTGCTTACGAGGGGAGCGATTCCGAA
AAGGGTGAAGGAGCAGAGCCCTTGAAGGTTCCAGTATGGGAACCCCAGGA
TTGGCAGCAGCAGCTTGTAAACATCCGAGCAATGAGGAACAAAAAAGATG
CACCTGTTGATCACCTCGGAACCGAACATTGTTATGATTCTAGTGCGCCG
CCAAAAGTCCGCCGGTATCAGGTTCTGTTGAGTTTGATGCTGAGTAGTCA
GACTAAGGACCAGGTTACGGCCGGAGCAATGCAACGGCTTCGGGCACGGG
GACTCACGGTCGATAGCATTTTGCAGACCGATGACGCAACATTGGGTAAA
CTCATATATCCAGTTGGCTTCTGGCGGAGCAAAGTGAAGTACATCAAGCA
GACCTCAGCCATTCTCCAACAACATTACGGAGGTGATATACCCGCAAGCG
TAGCTGAACTGGTAGCACTGCCGGGCGTCGGTCCCAAAATGGCACATCTG
GCTATGGCGGTTGCTTGGGGAACGGTGTCTGGTATCGCAGTTGATACGCA
TGTCCACCGCATCGCCAATCGGCTGAGGTGGACTAAAAAAGCCACTAAGT
CTCCTGAAGAAACACGGGCTGCTCTGGAAGAGTGGCTTCCACGAGAGCTG
TGGCATGAAATCAATGGATTGCTGGTTGGTTTCGGGCAGCAGACATGCTT
GCCCGTGCACCCCCGGTGTCATGCTTGCTTGAACCAGGCTTTGTGCCCAG
CTGCCCAGGGCCTGAGTGGAAGTGAGACACCGGGAACATCTGAGTCTGCG
ACCCCGGAGAGCacaaacGCGCGAATCCTGGCCTTCGcgATTGGCATTAG
CAGCATCGGCTGGGCATTCTCTGAAAACGACGAACTGAAGGATTGCGGCG
TGCGAATTTTCACTAAGGTCGAAAATCCCAAAACTGGTGAATCACTCGCT
CTCCCTAGACGACTGGCACGCTCCGCACGAAAGAGGCTTGCCCGCCGCAA
GGCACGCTTGAACCATCTTAAACACCTTATTGCAAATGAGTTTAAACTGA
ATTATGAGGACTACCAATCCTTTGACGAGTCTCTTGCTAAAGCCTACAAA
GGGAGCCTTATATCCCCGTATGAGCTCCGGTTCAGAGCACTCAACGAACT
GCTGTCCAAACAGGATTTTGCTCGCGTGATTCTCCACATAGCGAAGAGGC
GAGGATACGATGACATTAAAAACAGTGATGATAAGGAAAAAGGGGCCATA
CTCAAAGCGATTAAGCAAAATGAAGAGAAGCTCGCTAACTATCAATCAGT
AGGGGAGTATCTCTATAAAGAGTACTTCCAGAAGTTCAAAGAAAATAGCA
AGGAATTTACTAATGTCCGGAATAAAAAGGAGTCTTACGAAAGATGTATT
GCGCAATCTTTCCTCAAGGACGAGCTCAAATTGATTTTCAAGAAACAAAG
GGAATTTGGGTTCAGCTTCTCAAAAAAATTTGAGGAAGAGGTTCTGAGCG
TTGCCTTTTACAAACGCGCCCTTAAGGACTTCTCACATCTCGTAGGGAAT
TGTAGTTTCTTCACCGATGAAAAACGGGCGCCAAAAAATAGCCCTTTGGC
TTTTATGTTTGTCGCTCTGACTCGCATCATTAATCTGCTCAACAACCTTA
AAAACACGGAAGGGATTCTGTACACAAAGGATGATCTGAACGCTCTGCTT
AACGAAGTTTTGAAGAACGGGACTTTGACCTACAAACAAACCAAAAAGCT
TCTTGGTCTCAGTGATGACTACGAATTCAAGGGAGAAAAAGGGACATATT
TCATCGAATTCAAGAAGTATAAGGAGTTCATCAAAGCCTTGGGCGAGCAC
AACTTGTCTCAAGATGATCTCAACGAAATTGCTAAGGATATCACTCTGAT
TAAAGACGAGATCAAGCTCAAAAAGGCGTTGGCGAAGTATGACCTTAACC
AAAACCAAATAGATAGCCTCAGCAAGTTGGAATTTAAAGATCACTTGAAT
ATAAGTTTCAAGGCCCTTAAGTTGGTCACCCCCTTGATGCTTGAAGGAAA
GAAATATGATGAGGCATGTAATGAGCTGAATCTCAAGGTTGCTATTAACG
AAGACAAAAAAGATTTCCTCCCAGCTTTCAATGAGACTTACTATAAGGAC
GAGGTTACCAATCCTGTGGTGCTCCGAGCCATCAAAGAGTATCGAAAGGT
CCTGAATGCTTTGCTCAAAAAATACGGTAAGGTACACAAAATAAATATTG
AGCTCGCAAGGGAGGTCGGTAAGAACCACTCCCAGCGCGCCAAAATAGAA
AAGGAACAGAATGAAAATTACAAAGCGAAAAAGGACGCCGAGCTCGAGTG
CGAAAAGCTGGGCCTGAAAATAAACAGCAAGAACATTCTCAAACTCCGCC
TCTTCAAAGAACAAAAAGAATTTTGTGCTTATAGTGGTGAGAAAATAAAA
ATCTCCGATCTTCAAGACGAGAAGATGCTCGAAATAGACgcgATATATCC
ATATAGCAGGTCTTTTGACGATTCTTACATGAATAAAGTGCTTGTTTTCA
CTAAGCAGAATCAGGAAAAGTTGAATCAGACCCCCTTTGAGGCCTTTGGC
AACGACTCAGCAAAGTGGCAGAAGATCGAGGTCTTGGCTAAGAATCTTCC
TACTAAGAAACAGAAAAGGATATTGGATAAGAACTATAAAGACAAAGAAC
AAAAGAACTTTAAAGACCGCAACCTCAATGACACCAGATACATAGCAAGA
TTGGTTCTGAACTACACAAAAGATTATTTGGACTTCTTGCCGCTGTCTGA
TGATGAGAACACGAAACTCAACGACACGCAAAAGGGGTCTAAAGTCCACG
TCGAAGCTAAATCTGGGATGCTCACCTCAGCATTGAGGCATACGTGGGGA
TTCTCAGCAAAGGACCGAAACAATCACCTGCACCATGCCATTGACGCAGT
TATCATAGCGTATGCCAATAATTCAATAGTAAAAGCGTTTAGCGACTTCA
AGAAGGAACAAGAGTCCAACAGCGCCGAGCTCTACGCAAAAAAGATTAGT
GAACTCGACTACAAAAACAAAAGAAAATTCTTTGAGCCGTTCAGCGGATT
TCGACAGAAGGTATTGGATAAAATAGATGAAATTTTCGTGAGCAAACCCG
AAAGGAAAAAGCCCTCAGGCGCCTTGCACGAAGAGACTTTCAGGAAGGAA
GAGGAATTCTACCAAAGCTACGGCGGAAAAGAGGGAGTTTTGAAGGCTCT
CGAACTTGGAAAGATTAGGAAGGTGAACGGCAAGATAGTGAAAAACGGCG
ATATGTTCCGGGTTGATATCTTCAAACATAAAAAAACGAATAAATTTTAT
GCTGTGCCTATATACACTATGGACTTCGCACTTAAGGTCCTGCCGAATAA
GGCGGTAGCCCGATCTAAAAAAGGCGAAATTAAGGACTGGATTTTGATGG
ATGAAAATTACGAGTTCTGCTTTTCTCTCTACAAGGATTCCCTTATATTG
ATACAGACGAAAGATATGCAGGAACCGGAATTCGTGTATTACAACGCTTT
TACTTCCTCTACGGTATCTTTGATTGTCTCCAAACATGACAACAAATTCG
AAACACTCAGTAAAAACCAAAAGATTCTCTTTAAAAATGCGAACGAGAAA
GAAGTAATTGCAAAATCAATTGGCATCCAAAATTTGAAAGTTTTTGAAAA
ATATATAGTATCTGCCCTCGGAGAGGTTACTAAAGCGGAATTTAGACAGC
GAGAGGACTTCAAAAAATCAGGTCCACCCAAGAAAAAACGCAAGGTGGAA
GATCCGAAGAAAAAGCGAAAAGTGGATGTGtaaCGTTTTCCGGGACGCCG
GCTGGATGATCCTCCAGCGCGGGGATCTCATGCTGGAGTTCTTCGCCCAC
CCCAACTTGTTTATTGCAGCTTATAATGGTTACAAATAAAGCAATAGCAT
CACAAATTTCACAAATAAAGCATTTTTTTCACTGCATTCTAGTTGTGGTT
TGTCCAAACTCATCAATGTATCTTATCATGTCTGTATACCG.

In some embodiments of the disclosure, a CjeCas9-endonuclease fusions and gRNA molecule may comprise or consist of the nucleic acid sequence of:

E67-CjeCas9 and sgRNA plasmid
(U6: N's = sgRNA spacer, E67, CjeCas9)
(SEQ ID NO: 203)
gtttattacagggacagcagagatccagtttggttaattaaggtaccgag
ggcctatttcccatgattccttcatatttgcatatacgatacaaggctgt
tagagagataattagaattaatttgactgtaaacacaaagatattagtac
aaaatacgtgacgtagaaagtaataatttcttgggtagtttgcagtttta
aaattatgttttaaaatggactatcatatgcttaccgtaacttgaaagta
tttcgatttcttggctttatatatcttGTGGAAAGGACGAAACACCNNNN
NNNNNNNNNNNNNNNGTTTTAGTCCCTGAAGGGACTAAAATAAAGAGTTT
GCGGGACTCTGCGGGGTTACAATCCCCTAAAACCGCTTTTTTTCCTGCAG
CCCGGGGGATCCACTAGTTCTAGAGCGGCCGCCACCGCGGTGGAGCTCCA
GCTTTTGTTCCCTTTAGTGAGGGTTAATTGCGCGAATTCGCTAGCTAGGT
CTTGAAAGGAGTGGGAATTGGCTCCGGTGCCCGTCAGTGGGCAGAGCGCA
CATCGCCCACAGTCCCCGAGAAGTTGGGGGGAGGGGTCGGCAATTGATCC
GGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAG
GACCGGTTCTAGAGCGCTATTTAGAACCatgCAGGAGGTAATAGCGGGGC
TTGAGCGATTTACCTTTGCCTTCGAAAAAGACGTAGAGATGCAGAAGGGA
ACCGGCCTGCTCCCATTTCAAGGTATGGACAAATCAGCATCTGCCGTGTG
CAATTTTTTCACCAAGGGTCTGTGTGAAAAGGGGAAGCTCTGTCCATTTC
GCCATGATCGCGGAGAGAAGATGGTGGTGTGTAAGCACTGGCTGAGAGGG
CTTTGCAAAAAAGGCGACCACTGCAAATTTCTTCACCAATATGACCTGAC
TCGAATGCCTGAGTGTTATTTTTACAGTAAGTTCGGTGACTGTAGCAACA
AAGAATGCAGCTTCTTGCATGTCAAACCAGCATTCAAGTCACAGGATTGC
CCGTGGTACGATCAGGGTTTTTGCAAGGACGGTCCCCTCTGCAAATATCG
ACACGTACCCAGAATTATGTGCCTTAATTACCTGGTCGGCTTCTGTCCTG
AAGGGCCAAAATGTCAGTTTGCTCAAAAAATTCGCGAGTTCAAATTGCTC
CCTGGGTCTAAAATTTGGGAACCCCAGGATTGGCAGCAGCAGCTTGTAAA
CATCCGAGCAATGAGGAACAAAAAAGATGCACCTGTTGATCACCTCGGAA
CCGAACATTGTTATGATTCTAGTGCGCCGCCAAAAGTCCGCCGGTATCAG
GTTCTGTTGAGTTTGATGCTGAGTAGTCAGACTAAGGACCAGGTTACGGC
CGGAGCAATGCAACGGCTTCGGGCACGGGGACTCACGGTCGATAGCATTT
TGCAGACCGATGACGCAACATTGGGTAAACTCATATATCCAGTTGGCTTC
TGGCGGAGCAAAGTGAAGTACATCAAGCAGACCTCAGCCATTCTCCAACA
ACATTACGGAGGTGATATACCCGCAAGCGTAGCTGAACTGGTAGCACTGC
CGGGCGTCGGTCCCAAAATGGCACATCTGGCTATGGCGGTTGCTTGGGGA
ACGGTGTCTGGTATCGCAGTTGATACGCATGTCCACCGCATCGCCAATCG
GCTGAGGTGGACTAAAAAAGCCACTAAGTCTCCTGAAGAAACACGGGCTG
CTCTGGAAGAGTGGCTTCCACGAGAGCTGTGGCATGAAATCAATGGATTG
CTGGTTGGTTTCGGGCAGCAGACATGCTTGCCCGTGCACCCCCGGTGTCA
TGCTTGCTTGAACCAGGCTTTGTGCCCAGCTGCCCAGGGCCTGAGTGGAA
GTGAGACACCGGGAACATCTGAGTCTGCGACCCCGGAGAGCacaaacGCG
CGAATCCTGGCCTTCGcgATTGGCATTAGCAGCATCGGCTGGGCATTCTC
TGAAAACGACGAACTGAAGGATTGCGGCGTGCGAATTTTCACTAAGGTCG
AAAATCCCAAAACTGGTGAATCACTCGCTCTCCCTAGACGACTGGCACGC
TCCGCACGAAAGAGGCTTGCCCGCCGCAAGGCACGCTTGAACCATCTTAA
ACACCTTATTGCAAATGAGTTTAAACTGAATTATGAGGACTACCAATCCT
TTGACGAGTCTCTTGCTAAAGCCTACAAAGGGAGCCTTATATCCCCGTAT
GAGCTCCGGTTCAGAGCACTCAACGAACTGCTGTCCAAACAGGATTTTGC
TCGCGTGATTCTCCACATAGCGAAGAGGCGAGGATACGATGACATTAAAA
ACAGTGATGATAAGGAAAAAGGGGCCATACTCAAAGCGATTAAGCAAAAT
GAAGAGAAGCTCGCTAACTATCAATCAGTAGGGGAGTATCTCTATAAAGA
GTACTTCCAGAAGTTCAAAGAAAATAGCAAGGAATTTACTAATGTCCGGA
ATAAAAAGGAGTCTTACGAAAGATGTATTGCGCAATCTTTCCTCAAGGAC
GAGCTCAAATTGATTTTCAAGAAACAAAGGGAATTTGGGTTCAGCTTCTC
AAAAAAATTTGAGGAAGAGGTTCTGAGCGTTGCCTTTTACAAACGCGCCC
TTAAGGACTTCTCACATCTCGTAGGGAATTGTAGTTTCTTCACCGATGAA
AAACGGGCGCCAAAAAATAGCCCTTTGGCTTTTATGTTTGTCGCTCTGAC
TCGCATCATTAATCTGCTCAACAACCTTAAAAACACGGAAGGGATTCTGT
ACACAAAGGATGATCTGAACGCTCTGCTTAACGAAGTTTTGAAGAACGGG
ACTTTGACCTACAAACAAACCAAAAAGCTTCTTGGTCTCAGTGATGACTA
CGAATTCAAGGGAGAAAAAGGGACATATTTCATCGAATTCAAGAAGTATA
AGGAGTTCATCAAAGCCTTGGGCGAGCACAACTTGTCTCAAGATGATCTC
AACGAAATTGCTAAGGATATCACTCTGATTAAAGACGAGATCAAGCTCAA
AAAGGCGTTGGCGAAGTATGACCTTAACCAAAACCAAATAGATAGCCTCA
GCAAGTTGGAATTTAAAGATCACTTGAATATAAGTTTCAAGGCCCTTAAG
TTGGTCACCCCCTTGATGCTTGAAGGAAAGAAATATGATGAGGCATGTAA
TGAGCTGAATCTCAAGGTTGCTATTAACGAAGACAAAAAAGATTTCCTCC
CAGCTTTCAATGAGACTTACTATAAGGACGAGGTTACCAATCCTGTGGTG
CTCCGAGCCATCAAAGAGTATCGAAAGGTCCTGAATGCTTTGCTCAAAAA
ATACGGTAAGGTACACAAAATAAATATTGAGCTCGCAAGGGAGGTCGGTA
AGAACCACTCCCAGCGCGCCAAAATAGAAAAGGAACAGAATGAAAATTAC
AAAGCGAAAAAGGACGCCGAGCTCGAGTGCGAAAAGCTGGGCCTGAAAAT
AAACAGCAAGAACATTCTCAAACTCCGCCTCTTCAAAGAACAAAAAGAAT
TTTGTGCTTATAGTGGTGAGAAAATAAAAATCTCCGATCTTCAAGACGAG
AAGATGCTCGAAATAGACgcgATATATCCATATAGCAGGTCTTTTGACGA
TTCTTACATGAATAAAGTGCTTGTTTTCACTAAGCAGAATCAGGAAAAGT
TGAATCAGACCCCCTTTGAGGCCTTTGGCAACGACTCAGCAAAGTGGCAG
AAGATCGAGGTCTTGGCTAAGAATCTTCCTACTAAGAAACAGAAAAGGAT
ATTGGATAAGAACTATAAAGACAAAGAACAAAAGAACTTTAAAGACCGCA
ACCTCAATGACACCAGATACATAGCAAGATTGGTTCTGAACTACACAAAA
GATTATTTGGACTTCTTGCCGCTGTCTGATGATGAGAACACGAAACTCAA
CGACACGCAAAAGGGGTCTAAAGTCCACGTCGAAGCTAAATCTGGGATGC
TCACCTCAGCATTGAGGCATACGTGGGGATTCTCAGCAAAGGACCGAAAC
AATCACCTGCACCATGCCATTGACGCAGTTATCATAGCGTATGCCAATAA
TTCAATAGTAAAAGCGTTTAGCGACTTCAAGAAGGAACAAGAGTCCAACA
GCGCCGAGCTCTACGCAAAAAAGATTAGTGAACTCGACTACAAAAACAAA
AGAAAATTCTTTGAGCCGTTCAGCGGATTTCGACAGAAGGTATTGGATAA
AATAGATGAAATTTTCGTGAGCAAACCCGAAAGGAAAAAGCCCTCAGGCG
CCTTGCACGAAGAGACTTTCAGGAAGGAAGAGGAATTCTACCAAAGCTAC
GGCGGAAAAGAGGGAGTTTTGAAGGCTCTCGAACTTGGAAAGATTAGGAA
GGTGAACGGCAAGATAGTGAAAAACGGCGATATGTTCCGGGTTGATATCT
TCAAACATAAAAAAACGAATAAATTTTATGCTGTGCCTATATACACTATG
GACTTCGCACTTAAGGTCCTGCCGAATAAGGCGGTAGCCCGATCTAAAAA
AGGCGAAATTAAGGACTGGATTTTGATGGATGAAAATTACGAGTTCTGCT
TTTCTCTCTACAAGGATTCCCTTATATTGATACAGACGAAAGATATGCAG
GAACCGGAATTCGTGTATTACAACGCTTTTACTTCCTCTACGGTATCTTT
GATTGTCTCCAAACATGACAACAAATTCGAAACACTCAGTAAAAACCAAA
AGATTCTCTTTAAAAATGCGAACGAGAAAGAAGTAATTGCAAAATCAATT
GGCATCCAAAATTTGAAAGTTTTTGAAAAATATATAGTATCTGCCCTCGG
AGAGGTTACTAAAGCGGAATTTAGACAGCGAGAGGACTTCAAAAAATCAG
GTCCACCCAAGAAAAAACGCAAGGTGGAAGATCCGAAGAAAAAGCGAAAA
GTGGATGTGtaaCGTTTTCCGGGACGCCGGCTGGATGATCCTCCAGCGCG
GGGATCTCATGCTGGAGTTCTTCGCCCACCCCAACTTGTTTATTGCAGCT
TATAATGGTTACAAATAAAGCAATAGCATCACAAATTTCACAAATAAAGC
ATTTTTTTCACTGCATTCTAGTTGTGGTTTGTCCAAACTCATCAATGTAT
CTTATCATGTCTGTATACCG.

gRNA Target Sequences

In some embodiments of the compositions of the disclosure, a target sequence of an RNA molecule comprises a sequence motif corresponding to the first RNA binding protein and/or the second RNA binding protein.

In some embodiments of the compositions and methods of the disclosure, the sequence motif is a signature of a disease or disorder.

A sequence motif of the disclosure may be isolated or derived from a sequence of foreign or exogenous sequence found in a genomic sequence, and therefore translated into an mRNA molecule of the disclosure or a sequence of foreign or exogenous sequence found in an RNA sequence of the disclosure.

A sequence motif of the disclosure may comprise or consist of a mutation in an endogenous sequence that causes a disease or disorder. The mutation may comprise or consist of a sequence substitution, inversion, deletion, insertion, transposition, or any combination thereof.

A sequence motif of the disclosure may comprise or consist of a repeated sequence. In some embodiments, the repeated sequence may be associated with a microsatellite instability (MSI). MSI at one or more loci results from impaired DNA mismatch repair mechanisms of a cell of the disclosure. A hypervariable sequence of DNA may be transcribed into an mRNA of the disclosure comprising a target sequence comprising or consisting of the hypervariable sequence.

A sequence motif of the disclosure may comprise or consist of a biomarker. The biomarker may indicate a risk of developing a disease or disorder. The biomarker may indicate a healthy gene (low or no determinable risk of developing a disease or disorder. The biomarker may indicate an edited gene. Exemplary biomarkers include, but are not limited to, single nucleotide polymorphisms (SNPs), sequence variations or mutations, epigenetic marks, splice acceptor sites, exogenous sequences, heterologous sequences, and any combination thereof.

A sequence motif of the disclosure may comprise or consist of a secondary, tertiary or quaternary structure. The secondary, tertiary or quaternary structure may be endogenous or naturally occurring. The secondary, tertiary or quaternary structure may be induced or non-naturally occurring. The secondary, tertiary or quaternary structure may be encoded by an endogenous, exogenous, or heterologous sequence.

In some embodiments of the compositions and methods of the disclosure, a target sequence of an RNA molecule comprises or consists of between 2 and 100 nucleotides or nucleic acid bases, inclusive of the endpoints. In some embodiments, the target sequence of an RNA molecule comprises or consists of between 2 and 50 nucleotides or nucleic acid bases, inclusive of the endpoints. In some embodiments, the target sequence of an RNA molecule comprises or consists of between 2 and 20 nucleotides or nucleic acid bases, inclusive of the endpoints.

In some embodiments of the compositions and methods of the disclosure, a target sequence of an RNA molecule is continuous. In some embodiments, the target sequence of an RNA molecule is discontinuous. For example, the target sequence of an RNA molecule may comprise or consist of one or more nucleotides or nucleic acid bases that are not contiguous because one or more intermittent nucleotides are positioned in between the nucleotides of the target sequence.

In some embodiments of the compositions and methods of the disclosure, a target sequence of an RNA molecule is naturally occurring. In some embodiments, the target sequence of an RNA molecule is non-naturally occurring. Exemplary non-naturally occurring target sequences may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, a target sequence of an RNA molecule binds to a guide RNA of the disclosure.

In some embodiments of the compositions and methods of the disclosure, a target sequence of an RNA molecule binds to a first RNA binding protein of the disclosure.

In some embodiments of the compositions and methods of the disclosure, a target sequence of an RNA molecule binds to a second RNA binding protein of the disclosure.

### RNA Molecules

In some embodiments of the compositions and methods of the disclosure, an RNA molecule of the disclosure comprises a target sequence. In some embodiments, the RNA molecule of the disclosure comprises at least one target sequence. In some embodiments, the RNA molecule of the disclosure comprises one or more target sequence(s). In some embodiments, the RNA molecule of the disclosure comprises two or more target sequences.

In some embodiments of the compositions and methods of the disclosure, an RNA molecule of the disclosure is a naturally occurring RNA molecule. In some embodiments, the RNA molecule of the disclosure is a non-naturally occurring molecule. Exemplary non-naturally occurring RNA molecules may comprise or consist of sequence variations or mutations, chimeric sequences, exogenous sequences, heterologous sequences, chimeric sequences, recombinant sequences, sequences comprising a modified or synthetic nucleotide or any combination thereof.

In some embodiments of the compositions and methods of the disclosure, an RNA molecule of the disclosure comprises or consists of a sequence isolated or derived from a virus.

In some embodiments of the compositions and methods of the disclosure, an RNA molecule of the disclosure comprises or consists of a sequence isolated or derived from a prokaryotic organism. In some embodiments, an RNA molecule of the disclosure comprises or consists of a sequence isolated or derived from a species or strain of archaea or a species or strain of bacteria.

In some embodiments of the compositions and methods of the disclosure, the RNA molecule of the disclosure comprises or consists of a sequence isolated or derived from a eukaryotic organism. In some embodiments, an RNA molecule of the disclosure comprises or consists of a sequence isolated or derived from a species of protozoa, parasite, protist, algae, fungi, yeast, amoeba, worm, microorganism, invertebrate, vertebrate, insect, rodent, mouse, rat, mammal, or a primate. In some embodiments, an RNA molecule of the disclosure comprises or consists of a sequence isolated or derived from a human.

In some embodiments of the compositions and methods of the disclosure, the RNA molecule of the disclosure comprises or consists of a sequence derived from a coding sequence from a genome of an organism or a virus. In some embodiments, the RNA molecule of the disclosure comprises or consists of a primary RNA transcript, a precursor messenger RNA (pre-mRNA) or messenger RNA (mRNA). In some embodiments, the RNA molecule of the disclosure comprises or consists of a gene product that has not been processed (e.g. a transcript). In some embodiments, the RNA molecule of the disclosure comprises or consists of a gene product that has been subject to post-transcriptional processing (e.g. a transcript comprising a 5′cap and a 3′ polyadenylation signal). In some embodiments, the RNA molecule of the disclosure comprises or consists of a gene product that has been subject to alternative splicing (e.g. a splice variant). In some embodiments, the RNA molecule of the disclosure comprises or consists of a gene product that has been subject to removal of non-coding and/or intronic sequences (e.g. a messenger RNA (mRNA)).

In some embodiments of the compositions and methods of the disclosure, the RNA molecule of the disclosure comprises or consists of a sequence derived from a non-coding sequence (e.g. a non-coding RNA (ncRNA)). In some embodiments, the RNA molecule of the disclosure comprises or consists of a ribosomal RNA. In some embodiments, the RNA molecule of the disclosure comprises or consists of a small ncRNA molecule. Exemplary small RNA molecules of the disclosure include, but are not limited to, microRNAs (miRNAs), small interfering (siRNAs), piwi-interacting RNAs (piRNAs), small nucleolar RNAs (snoRNAs), small nuclear RNAs (snRNAs), extracellular or exosomal RNAs (exRNAs), and small Cajal body-specific RNAs (scaRNAs). In some embodiments, the RNA molecule of the disclosure comprises or consists of a long ncRNA molecule. Exemplary long RNA molecules of the disclosure include, but are not limited to, X-inactive specific transcript (Xist) and HOX transcript antisense RNA (HOTAIR).

In some embodiments of the compositions and methods of the disclosure, the RNA molecule of the disclosure contacted by a composition of the disclosure in an intracellular space. In some embodiments, the RNA molecule of the disclosure contacted by a composition of the disclosure in a cytosolic space. In some embodiments, the RNA molecule of the disclosure contacted by a composition of the disclosure in a nucleus. In some embodiments, the RNA molecule of the disclosure contacted by a composition of the disclosure in a vesicle, membrane-bound compartment of a cell, or an organelle.

In some embodiments of the compositions and methods of the disclosure, the RNA molecule of the disclosure contacted by a composition of the disclosure in an extracellular space. In some embodiments, the RNA molecule of the disclosure contacted by a composition of the disclosure in an exosome. In some embodiments, the RNA molecule of the disclosure contacted by a composition of the disclosure in a liposome, a polymersome, a micelle or a nanoparticle. In some embodiments, the RNA molecule of the disclosure contacted by a composition of the disclosure in an extracellular matrix. In some embodiments, the RNA molecule of the disclosure contacted by a composition of the disclosure in a droplet. In some embodiments, the RNA molecule of the disclosure contacted by a composition of the disclosure in a microfluidic droplet.

In some embodiments of the compositions and methods of the disclosure, a RNA molecule of the disclosure comprises or consists of a single-stranded sequence. In some embodiments, the RNA molecule of the disclosure comprises or consists of a double-stranded sequence. In some embodiments, the double-stranded sequence comprises two RNA molecules. In some embodiments, the double-stranded sequence comprises one RNA molecule and one DNA molecule. In some embodiments, including those wherein the double-stranded sequence comprises one RNA molecule and one DNA molecule, compositions of the disclosure selectively bind and, optionally, selectively cut the RNA molecule.

### Fusion Proteins

In some embodiments of the compositions and methods of the disclosure, the composition comprises a sequence encoding a target RNA-binding fusion protein comprising (a) a sequence encoding a first RNA-binding polypeptide or portion thereof; and (b) a sequence encoding a second RNA-binding polypeptide, wherein the first RNA-biding polypeptide binds a target RNA, and wherein the second RNA-binding polypeptide comprises RNA-nuclease activity.

In some embodiments, a target RNA-binding fusion protein is an RNA-guided target RNA-binding fusion protein. RNA-guided target RNA-binding fusion proteins comprise at least one RNA-binding polypeptide which corresponds to a gRNA which guides the RNA-binding polypeptide to target RNA. RNA-guided target RNA-binding fusion proteins include without limitation, RNA-binding polypeptides which are CRISPR/Cas-based RNA-binding polypeptides or portions thereof.

In some embodiments, a target RNA-binding fusion protein is not an RNA-guided target RNA-binding fusion protein and as such comprises at least one RNA-binding polypeptide which is capable of binding a target RNA without a corresponding gRNA sequence. Such non-guided RNA-binding polypeptides include, without limitation, at least one RNA-binding protein or RNA-binding portion thereof which is a PUF (Pumilio and FBF homology family). This type RNA-binding polypeptide can be used in place of a gRNA-guided RNA binding protein such as CRISPR/Cas. The unique RNA recognition mode of PUF proteins (named for Drosophila Pumilio and C. elegans fem-3 binding factor) that are involved in mediating mRNA stability and translation are well known in the art. The PUF domain of human Pumilio1, also known in the art, binds tightly to cognate RNA sequences and its specificity can be modified. It contains eight PUF repeats that recognize eight consecutive RNA bases with each repeat recognizing a single base. Since two amino acid side chains in each repeat recognize the Watson-Crick edge of the corresponding base and determine the specificity of that repeat, a PUF domain can be designed to specifically bind most 8-nt RNA. Wang et al., Nat Methods. 2009; 6(11): 825-830. See also WO2012/068627 which is incorporated by reference herein in its entirety.

In some embodiments of the non-guided RNA-binding fusion proteins of the disclosure, the fusion protein comprises at least one RNA-binding protein or RNA-binding portion thereof which is a PUMBY (Pumilio-based assembly) protein. RNA-binding protein PumHD (Pumilio homology domain, a member of the PUF family), which has been widely used in native and modified form for targeting RNA, has been engineered to yield a set of four canonical protein modules, each of which targets one RNA base. These modules (i.e., Pumby, for Pumilio-based assembly) can be concatenated in chains of varying composition and length, to bind desired target RNAs. The specificity of such Pumby-RNA interactions is high, with undetectable binding of a Pumby chain to RNA sequences that bear three or more mismatches from the target sequence. Katarzyna et al., PNAS, 2016; 113(19): E2579-E2588. See also US 2016/0238593 which is incorporated by reference herein in its entirety.

In some embodiments of the compositions of the disclosure, at least one of the RNA-binding proteins or RNA-binding portions thereof is a PPR protein. PPR proteins (proteins with pentatricopeptide repeat (PPR) motifs derived from plants) are nuclear-encoded and exclusively controlled at the RNA level organelles (chloroplasts and mitochondria), cutting, translation, splicing, RNA editing, genes specifically acting on RNA stability. PPR proteins are typically a motif of 35 amino acids and have a structure in which a PPR motif is about 10 contiguous amino acids. The combination of PPR motifs can be used for sequence-selective binding to RNA. PPR proteins are often comprised of PPR motifs of about 10 repeat domains. PPR domains or RNA-binding domains may be configured to be catalytically inactive. WO 2013/058404 incorporated herein by reference in its entirety.

In some embodiments, the fusion protein disclosed herein comprises a linker between the at least two RNA-binding polypeptides. In some embodiments, the linker is a peptide linker. In some embodiments, the peptide linker comprises one or more repeats of the tri-peptide GGS. In other embodiments, the linker is a non-peptide linker. In some embodiments, the non-peptide linker comprises polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the at least one RNA-binding protein does not require multimerization for RNA-binding activity. In some embodiments, the at least one RNA-binding protein is not a monomer of a multimer complex. In some embodiments, a multimer protein complex does not comprise the RNA binding protein. In some embodiments, the at least one of RNA-binding protein selectively binds to a target sequence within the RNA molecule. In some embodiments, the at least one RNA-binding protein does not comprise an affinity for a second sequence within the RNA molecule. In some embodiments, the at least one RNA-binding protein does not comprise a high affinity for or selectively bind a second sequence within the RNA molecule. In some embodiments, the at least one RNA-binding protein comprises between 2 and 1300 amino acids, inclusive of the endpoints.

In some embodiments, the sequence encoding the at least one RNA-binding protein of the fusion proteins disclosed herein further comprises a sequence encoding a nuclear localization signal (NLS). In some embodiments, the sequence encoding a nuclear localization signal (NLS) is positioned 3′ to the sequence encoding the RNA binding protein. In some embodiments, the at least one RNA-binding protein comprises an NLS at a C-terminus of the protein. In some embodiments, the sequence encoding the at least one RNA-binding protein further comprises a first sequence encoding a first NLS and a second sequence encoding a second NLS. In some embodiments, the sequence encoding the first NLS or the second NLS is positioned 3′ to the sequence encoding the RNA-binding protein. In some embodiments, the at least one RNA-binding protein comprises the first NLS or the second NLS at a C-terminus of the protein. In some embodiments, the at least one RNA-binding protein further comprises an NES (nuclear export signal) or other peptide tag or secretory signal.

In some embodiments, a fusion protein disclosed herein comprises the at least one RNA-binding protein as a first RNA-binding protein together with a second RNA-binding protein comprising or consisting of a nuclease domain. In some embodiments, the second RNA binding protein binds RNA in a manner in which it associates with RNA. In some embodiments, the second RNA binding protein associates with RNA in a manner in which it cleaves RNA.

In some embodiments, the second RNA-binding polypeptide is operably configured to the first RNA-binding polypeptide at the C-terminus of the first RNA-binding polypeptide. In some embodiments, the second RNA-binding polypeptide is operably configured to the first RNA-binding polypeptide at the N-terminus of the first RNA-binding polypeptide.

### Vectors

In some embodiments of the compositions and methods of the disclosure, a vector comprises a guide RNA of the disclosure. In some embodiments, the vector comprises at least one guide RNA of the disclosure. In some embodiments, the vector comprises one or more guide RNA(s) of the disclosure. In some embodiments, the vector comprises two or more guide RNAs of the disclosure. In some embodiments, the vector further comprises a fusion protein of the disclosure. In some embodiments, the fusion protein comprises a first RNA binding protein and a second RNA binding protein.

In some embodiments of the compositions and methods of the disclosure, a first vector comprises a guide RNA of the disclosure and a second vector comprises a fusion protein of the disclosure. In some embodiments, the first vector comprises at least one guide RNA of the disclosure. In some embodiments, the first vector comprises one or more guide RNA(s) of the disclosure. In some embodiments, the first vector comprises two or more guide RNA(s) of the disclosure. In some embodiments, the fusion protein comprises a first RNA binding protein and a second RNA binding protein. In some embodiments, the first vector and the second vector are identical. In some embodiments, the first vector and the second vector are not identical.

In some embodiments of the compositions and methods of the disclosure, the vector is or comprises a component of a “2-component RNA targeting system” comprising (a) nucleic acid sequence encoding a RNA-targeted fusion protein of the disclosure; and (b) a single guide RNA (sgRNA) sequence comprising: on its 5′ end, an RNA sequence (e.g., spacer sequence) that hybridizes to or specifically binds to a target RNA sequence; and on its 3′ end, an RNA sequence (e.g., scaffold sequence) capable of specifically binding to or associating with the CRISPR/Cas protein of the fusion protein; and wherein the 2-component RNA targeting system recognizes and alters the target RNA in a cell in the absence of a PAMmer. In some embodiments, the sequences of the 2-component system are comprised within a single (e.g., unitary) vector. In some embodiments, the spacer sequence of the 2-component system targets a repeat sequence selected from the group consisting of CUG, CCUG, CAG, and GGGGCC. In some embodiments, the spacer sequence of the 2-component system targets an RNA sequence involved in an adaptive immune response. In some embodiments, a spacer sequence of the 2-component system comprises a portion of a nucleic acid sequence encoding a protein component of an adaptive immune response, and wherein the protein component is selected from the group consisting of Beta-2-microglobulin (β2M), Human Leukocyte Antigen A (HLA-A), Human Leukocyte Antigen B (HLA-B), Human Leukocyte Antigen C (HLA-C), Cluster of Differentiation 28 (CD28), Cluster of Differentiation 80 (CD80), Cluster of Differentiation 86 (CD86), Inducible T-cell Costimulator (ICOS), ICOS Ligand (ICOSLG), OX40L, Interleukin 12 (IL12), and CC Chemokine Receptor 7 (CCR7). In some embodiments, the 2-component system comprises a spacer which is a portion of a nucleic acid sequence encoding a protein component of an adaptive immune response and which is about 20 or 21 nucleotides in length. In some embodiments, the 2-component system comprises a first and second spacer comprised within a singular gRNA. In some embodiments, the 2-component system comprises a first and second spacer sequence comprised within first and second gRNA sequences. In some embodiments, the first spacer targets a repeat sequence and the second spacer targets RNA involved in an adaptive immune response.

In some embodiments of the compositions and methods of the disclosure, a vector of the disclosure is a viral vector. In some embodiments, the viral vector comprises a sequence isolated or derived from a retrovirus. In some embodiments, the viral vector comprises a sequence isolated or derived from a lentivirus. In some embodiments, the viral vector comprises a sequence isolated or derived from an adenovirus. In some embodiments, the viral vector comprises a sequence isolated or derived from an adeno-associated virus (AAV). In some embodiments, the viral vector is replication incompetent. In some embodiments, the viral vector is isolated or recombinant. In some embodiments, the viral vector is self-complementary.

In some embodiments of the compositions and methods of the disclosure, the viral vector comprises a sequence isolated or derived from an adeno-associated virus (AAV). In some embodiments, the viral vector comprises an inverted terminal repeat sequence or a capsid sequence that is isolated or derived from an AAV of serotype AAV1, AAV2, AAV3, AAV4, AAV5, AAV6, AAV7, AAV8, AAV9, AAV10, AAV11 or AAV12. In some embodiments, the viral vector is replication incompetent. In some embodiments, the viral vector is isolated or recombinant (rAAV). In some embodiments, the viral vector is self-complementary (scAAV).

In some embodiments of the compositions and methods of the disclosure, a vector of the disclosure is a non-viral vector. In some embodiments, the vector comprises or consists of a nanoparticle, a micelle, a liposome or lipoplex, a polymersome, a polyplex or a dendrimer. In some embodiments, the vector is an expression vector or recombinant expression system. As used herein, the term “recombinant expression system” refers to a genetic construct for the expression of certain genetic material formed by recombination.

In some embodiments of the compositions and methods of the disclosure, an expression vector, viral vector or non-viral vector provided herein, includes without limitation, an expression control element. An “expression control element” as used herein refers to any sequence that regulates the expression of a coding sequence, such as a gene. Exemplary expression control elements include but are not limited to promoters, enhancers, microRNAs, post-transcriptional regulatory elements, polyadenylation signal sequences, and introns. Expression control elements may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. In some embodiments, expression control by a promoter is tissue-specific. Non-limiting exemplary promoters include CMV, CBA, CAG, Cbh, EF-1a, PGK, UBC, GUSB, UCOE, hAAT, TBG, Desmin, MCK, C5-12, NSE, Synapsin, PDGF, MecP2, CaMKII, mGluR2, NFL, NFH, nP2, PPE, ENK, EAAT2, GFAP, MBP, and U6 promoters. An “enhancer” is a region of DNA that can be bound by activating proteins to increase the likelihood or frequency of transcription. Non-limiting exemplary enhancers and posttranscriptional regulatory elements include the CMV enhancer and WPRE.

In some embodiments of the compositions and methods of the disclosure, an expression vector, viral vector or non-viral vector provided herein, includes without limitation, vector elements such as an IRES or 2A peptide sites for configuration of “multicistronic” or “polycistronic” or “bicistronic” or tricistronic” constructs, i.e., having double or triple or multiple coding areas or exons, and as such will have the capability to express from mRNA two or more proteins from a single construct. Multicistronic vectors simultaneously express two or more separate proteins from the same mRNA. The two strategies most widely used for constructing multicistronic configurations are through the use of an IRES or a 2A self-cleaving site. An “IRES” refers to an internal ribosome entry site or portion thereof of viral, prokaryotic, or eukaryotic origin which are used within polycistronic vector constructs. In some embodiments, an IRES is an RNA element that allows for translation initiation in a cap-independent manner. The term “self-cleaving peptides” or “sequences encoding self-cleaving peptides” or “2A self-cleaving site” refer to linking sequences which are used within vector constructs to incorporate sites to promote ribosomal skipping and thus to generate two polypeptides from a single promoter, such self-cleaving peptides include without limitation, T2A, and P2A peptides or sequences encoding the self-cleaving peptides.

In some embodiments, the vector is a viral vector. In some embodiments, the vector is an adenoviral vector, an adeno-associated viral (AAV) vector, or a lentiviral vector. In some embodiments, the vector is a retroviral vector, an adenoviral/retroviral chimera vector, a herpes simplex viral I or II vector, a parvoviral vector, a reticuloendotheliosis viral vector, a polioviral vector, a papillomaviral vector, a vaccinia viral vector, or any hybrid or chimeric vector incorporating favorable aspects of two or more viral vectors. In some embodiments, the vector further comprises one or more expression control elements operably linked to the polynucleotide. In some embodiments, the vector further comprises one or more selectable markers. In some embodiments, the AAV vector has low toxicity. In some embodiments, the AAV vector does not incorporate into the host genome, thereby having a low probability of causing insertional mutagenesis. In some embodiments, the AAV vector can encode a range total of polynucleotides from 4.5 kb to 4.75 kb. In some embodiments, exemplary AAV vectors that may be used in any of the herein described compositions, systems, methods, and kits can include an AAV1 vector, a modified AAV1 vector, an AAV2 vector, a modified AAV2 vector, an AAV3 vector, a modified AAV3 vector, an AAV4 vector, a modified AAV4 vector, an AAV5 vector, a modified AAV5 vector, an AAV6 vector, a modified AAV6 vector, an AAV7 vector, a modified AAV7 vector, an AAV8 vector, an AAV9 vector, an AAV.rh10 vector, a modified AAV.rh10 vector, an AAV.rh32/33 vector, a modified AAV.rh32/33 vector, an AAV.rh43 vector, a modified AAV.rh43 vector, an AAV.rh64R1 vector, and a modified AAV.rh64R1 vector and any combinations or equivalents thereof. In some embodiments, the lentiviral vector is an integrase-competent lentiviral vector (ICLV). In some embodiments, the lentiviral vector can refer to the transgene plasmid vector as well as the transgene plasmid vector in conjunction with related plasmids (e.g., a packaging plasmid, a rev expressing plasmid, an envelope plasmid) as well as a lentiviral-based particle capable of introducing exogenous nucleic acid into a cell through a viral or viral-like entry mechanism. Lentiviral vectors are well-known in the art (see, e.g., Trono D. (2002) Lentiviral vectors, New York: Spring-Verlag Berlin Heidelberg and Durand et al. (2011) Viruses 3(2):132-159 doi: 10.3390/v3020132). In some embodiments, exemplary lentiviral vectors that may be used in any of the herein described compositions, systems, methods, and kits can include a human immunodeficiency virus (HIV) 1 vector, a modified human immunodeficiency virus (HIV) 1 vector, a human immunodeficiency virus (HIV) 2 vector, a modified human immunodeficiency virus (HIV) 2 vector, a sooty mangabey simian immunodeficiency virus (SIV_{SM}) vector, a modified sooty mangabey simian immunodeficiency virus (SIV_{SM}) vector, a African green monkey simian immunodeficiency virus (SIV_{AGM}) vector, a modified African green monkey simian immunodeficiency virus (SIV_{AGM}) vector, an equine infectious anemia virus (EIAV) vector, a modified equine infectious anemia virus (EIAV) vector, a feline immunodeficiency virus (FIV) vector, a modified feline immunodeficiency virus (FIV) vector, a Visna/maedi virus (VNV/VMV) vector, a modified Visna/maedi virus (VNV/VMV) vector, a caprine arthritis-encephalitis virus (CAEV) vector, a modified caprine arthritis-encephalitis virus (CAEV) vector, a bovine immunodeficiency virus (BIV), or a modified bovine immunodeficiency virus (BIV).

### Nucleic Acids

Provided herein are the nucleic acid sequences encoding the fusion proteins disclosed herein for use in gene transfer and expression techniques described herein. It should be understood, although not always explicitly stated that the sequences provided herein can be used to provide the expression product as well as substantially identical sequences that produce a protein that has the same biological properties. These “biologically equivalent” or “biologically active” or “equivalent” polypeptides are encoded by equivalent polynucleotides as described herein. They may possess at least 60%, or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge. Additionally, an equivalent polynucleotide is one that hybridizes under stringent conditions to the reference polynucleotide or its complement or in reference to a polypeptide, a polypeptide encoded by a polynucleotide that hybridizes to the reference encoding polynucleotide under stringent conditions or its complementary strand. Alternatively, an equivalent polypeptide or protein is one that is expressed from an equivalent polynucleotide.

The nucleic acid sequences (e.g., polynucleotide sequences) disclosed herein may be codon-optimized which is a technique well known in the art. In some embodiments disclosed herein, exemplary Cas sequences, such as e.g., SEQ ID NO: 46 (Cas13d), are codon optimized for expression in human cells. Codon optimization refers to the fact that different cells differ in their usage of particular codons. This codon bias corresponds to a bias in the relative abundance of particular tRNAs in the cell type. By altering the codons in the sequence to match with the relative abundance of corresponding tRNAs, it is possible to increase expression. It is also possible to decrease expression by deliberately choosing codons for which the corresponding tRNAs are known to be rare in a particular cell type. Codon usage tables are known in the art for mammalian cells, as well as for a variety of other organisms. Based on the genetic code, nucleic acid sequences coding for, e.g., a Cas protein, can be generated. In some embodiments, such a sequence is optimized for expression in a host or target cell, such as a host cell used to express the Cas protein or a cell in which the disclosed methods are practiced (such as in a mammalian cell, e.g., a human cell). Codon preferences and codon usage tables for a particular species can be used to engineer isolated nucleic acid molecules encoding a Cas protein (such as one encoding a protein having at least 80%, at least 85%, at least 90%, at least 92%, at least 95%, at least 96%, at least 97%, at least 98%, at least 99%, or 100% sequence identity to its corresponding wild-type protein) that takes advantage of the codon usage preferences of that particular species. For example, the Cas proteins disclosed herein can be designed to have codons that are preferentially used by a particular organism of interest. In one example, a Cas nucleic acid sequence is optimized for expression in human cells, such as one having at least 70%, at least 80%, at least 85%, at least 90%, at least 92%, at least 95%, at least 98%, or at least 99% sequence identity to its corresponding wild-type or originating nucleic acid sequence. In some embodiments, an isolated nucleic acid molecule encoding at least one Cas protein (which can be part of a vector) includes at least one Cas protein coding sequence that is codon optimized for expression in a eukaryotic cell, or at least one Cas protein coding sequence codon optimized for expression in a human cell. In one embodiment, such a codon optimized Cas coding sequence has at least 80%, at least 85%, at least 90%, at least 92%, at least 95%, at least 96%, at least 97%, at least 98%, at least 99%, or 100% sequence identity to its corresponding wild-type or originating sequence. In another embodiment, a eukaryotic cell codon optimized nucleic acid sequence encodes a Cas protein having at least 85%, at least 90%, at least 92%, at least 95%, at least 96%, at least 97%, at least 98%, at least 99%, or 100% sequence identity to its corresponding wild-type or originating protein. In another embodiment, a variety of clones containing functionally equivalent nucleic acids may be routinely generated, such as nucleic acids which differ in sequence but which encode the same Cas protein sequence. Silent mutations in the coding sequence result from the degeneracy (i.e., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see, for example, Stryer, 1988, Biochemistry, 3.sup.rd Edition, W.H. 5 Freeman and Co., NY).

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. “Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

### Cells

In some embodiments of the compositions and methods of the disclosure, a cell of the disclosure is a prokaryotic cell.

In some embodiments of the compositions and methods of the disclosure, a cell of the disclosure is a eukaryotic cell. In some embodiments, the cell is a mammalian cell. In some embodiments, the cell is a bovine, murine, feline, equine, porcine, canine, simian, or human cell. In some embodiments, the cell is a non-human mammalian cell such as a non-human primate cell.

In some embodiments, a cell of the disclosure is a somatic cell. In some embodiments, a cell of the disclosure is a germline cell. In some embodiments, a germline cell of the disclosure is not a human cell.

In some embodiments of the compositions and methods of the disclosure, a cell of the disclosure is a stem cell. In some embodiments, a cell of the disclosure is an embryonic stem cell. In some embodiments, an embryonic stem cell of the disclosure is not a human cell. In some embodiments, a cell of the disclosure is a multipotent stem cell or a pluripotent stem cell. In some embodiments, a cell of the disclosure is an adult stem cell. In some embodiments, a cell of the disclosure is an induced pluripotent stem cell (iPSC). In some embodiments, a cell of the disclosure is a hematopoetic stem cell (HSC).

In some embodiments of the compositions and methods of the disclosure, a somatic cell of the disclosure is an immune cell. In some embodiments, an immune cell of the disclosure is a lymphocyte. In some embodiments, an immune cell of the disclosure is a T lymphocyte (also referred to herein as a T-cell). Exemplary T-cells of the disclosure include, but are not limited to, naïve T cells, effector T cells, helper T cells, memory T cells, regulatory T cells (Tregs) and Gamma delta T cells. In some embodiments, an immune cell of the disclosure is a B lymphocyte. In some embodiments, an immune cell of the disclosure is a natural killer cell. In some embodiments, an immune cell of the disclosure is an antigen-presenting cell.

In some embodiments of the compositions and methods of the disclosure, a somatic cell of the disclosure is a muscle cell. In some embodiments, a muscle cell of the disclosure is a myoblast or a myocyte. In some embodiments, a muscle cell of the disclosure is a cardiac muscle cell, skeletal muscle cell or smooth muscle cell. In some embodiments, a muscle cell of the disclosure is a striated cell.

In some embodiments of the compositions and methods of the disclosure, a somatic cell of the disclosure is an epithelial cell. In some embodiments, an epithelial cell of the disclosure forms a squamous cell epithelium, a cuboidal cell epithelium, a columnar cell epithelium, a stratified cell epithelium, a pseudostratified columnar cell epithelium or a transitional cell epithelium. In some embodiments, an epithelial cell of the disclosure forms a gland including, but not limited to, a pineal gland, a thymus gland, a pituitary gland, a thyroid gland, an adrenal gland, an apocrine gland, a holocrine gland, a merocrine gland, a serous gland, a mucous gland and a sebaceous gland. In some embodiments, an epithelial cell of the disclosure contacts an outer surface of an organ including, but not limited to, a lung, a spleen, a stomach, a pancreas, a bladder, an intestine, a kidney, a gallbladder, a liver, a larynx or a pharynx. In some embodiments, an epithelial cell of the disclosure contacts an outer surface of a blood vessel or a vein.

In some embodiments of the compositions and methods of the disclosure, a somatic cell of the disclosure is a neuronal cell. In some embodiments, a neuron cell of the disclosure is a neuron of the central nervous system. In some embodiments, a neuron cell of the disclosure is a neuron of the brain or the spinal cord. In some embodiments, a neuron cell of the disclosure is a neuron of the retina. In some embodiments, a neuron cell of the disclosure is a neuron of a cranial nerve or an optic nerve. In some embodiments, a neuron cell of the disclosure is a neuron of the peripheral nervous system. In some embodiments, a neuron cell of the disclosure is a neuroglial or a glial cell. In some embodiments, a glial of the disclosure is a glial cell of the central nervous system including, but not limited to, oligodendrocytes, astrocytes, ependymal cells, and microglia. In some embodiments, a glial of the disclosure is a glial cell of the peripheral nervous system including, but not limited to, Schwann cells and satellite cells.

In some embodiments of the compositions and methods of the disclosure, a somatic cell of the disclosure is a primary cell.

In some embodiments of the compositions and methods of the disclosure, a somatic cell of the disclosure is a cultured cell.

In some embodiments of the compositions and methods of the disclosure, a somatic cell of the disclosure is in vivo, in vitro, ex vivo or in situ.

In some embodiments of the compositions and methods of the disclosure, a somatic cell of the disclosure is autologous or allogeneic.

### Masking Modified Cells of the Disclosure

Compositions of the disclosure simultaneously deliver a gene therapy and prevent expression of antigens derived from the gene therapy construct or associated delivery vector from display on the surface of a modified cell of the disclosure.

By inhibiting or reducing expression of a component of an adaptive immune response in the modified cell, the modified cell is invisible to a host immune system. For example, compositions of the disclosure may simultaneously target an RNA molecule associated with a genetic disease or disorder and an RNA molecule that encodes the β2M subunit of the MHC I. By selectively targeting an RNA molecule that encodes the β2M subunit of the MHC I, the composition prevents the modified cell from displaying one or more antigen peptides derived from an RNA targeting construct, vector, or combination thereof on the surface of the modified cell. Consequently, a subject's immune system does not identify the modified cell as containing foreign sequences and does not attempt to mount an immune response directed at the modified cell. This method increases the therapeutic efficacy of the treatment of the genetic disease or disorder while avoiding a common side effect of gene therapy.

In some embodiments of the compositions and methods of the disclosure, the component of an adaptive immune response comprises or consists of a component of a type I major histocompatibility complex (MHC I), a type II major histocompatibility complex (MHC II), a T-cell receptor (TCR), a costimulatory molecule or a combination thereof. In some embodiments, the MHC I component comprises an α1 chain, an α2 chain, an α3 chain, or a β2M protein. In some embodiments, the component of an adaptive immune response comprises or consists of an MHC I β2M protein. In some embodiments, the MHC II component comprises an α1 chain, an α2 chain, a β1 chain, or a β2 chain. In some embodiments, the TCR component comprises an α-chain and a β-chain. In some embodiments, the costimulatory molecule comprises a Cluster of Differentiation 28 (CD28), a Cluster of Differentiation 80 (CD80), a Cluster of Differentiation 86 (CD86), an Inducible T-cell COStimulator (ICOS), or an ICOS Ligand (ICOSLG) protein.

An α-chain of an MHC I may be encoded by an HLA gene, including but not limited to, HLA-A, HLA-B and HLA-C.

Compositions of the disclosure may comprise a gRNA comprising a spacer sequence that specifically binds to a target sequence of an RNA molecule encoding an α-chain derived from an HLA-A gene comprising or consisting of 20 nucleotides of the sequence of

| (SEQ ID NO: 216) | |
|---|---|
| 1 | atggccgtca tggcgccccg aaccctcgtc ctgctactct cgggggctct ggccctgacc |
| 61 | cagacctggg cgggctctca ctccatgagg tatttcttca catccgtgtc ccggcccggc |
| 121 | cgcggggagc cccgcttcat cgcagtgggc tacgtggacg acacgcagtt cgtgcggttc |
| 181 | gacagcgacg ccgcgagcca gaggatggag ccgcgggcgc cgtggataga gcaggagggt |
| 241 | ccggagtatt gggacgggga gacacggaaa gtgaaggccc actcacagac tcaccgagtg |
| 301 | gacctgggga ccctgcgcgg ctactacaac cagagcgagg ccggttctca caccgtccag |
| 361 | aggatgtgtg gctgcgacgt ggggtcggac tggcgcttcc tccgcgggta ccaccagtac |
| 421 | gcctacgacg gcaaggatta catcgccctg aaagaggacc tgcgctcttg gaccgcggcg |
| 481 | gacatggcag ctcagaccac caagcacaag tgggaggcgg cccatgtggc ggagcagttg |
| 541 | agagcctacc tggagggcac gtgcgtggag tggctccgca gatacctgga gaacgggaag |
| 601 | gagacgctgc agcgcacgga cgcccccaaa acgcatatga ctcaccacgc tgtctctgac |
| 661 | catgaagcca ccctgaggtg ctgggccctg agcttctacc ctgcggagat cacactgacc |
| 721 | tggcagcggg atggggagga ccagacccag gacacggagc tcgtggagac caggcctgca |
| 781 | ggggatggaa ccttccagaa gtgggcggct gtggtggtgc cttctggaca ggagcagaga |
| 841 | taaacctgcc atgtgcagca tgagggtttg cccaagcccc tcaccctgag atgggagccg |
| 901 | tcttcccagc ccaccatccc catcgtgggc atcattgctg gcctggttct ctttggagct |
| 961 | gtgatcactg gagctgtggt cgctgctgtg atgtggagga ggaagagctc agatagaaaa |
| 1021 | ggagggagct actctcaggc tgcaagcagt gacagtgccc agggctctga tgtgtctctc |
| 1081 | acagcttgta aagtgtga. |

Compositions of the disclosure may comprise a gRNA comprising a spacer sequence that specifically binds to a target sequence of an RNA molecule encoding an α-chain derived from an HLA-B gene comprising or consisting of 20 nucleotides of the sequence of

| (SEQ ID NO: 217) | |
|---|---|
| 1 | tggtgtagga gaagagggat caggacgaag tcccaggccc cgggcggggc tctcagggtc |
| 61 | tcaggctccg agggccgcgt ctgcaatggg gaggcgcagc gttggggatt ccccactccc |
| 121 | acgagtttca cttcttctcc caacctatgt cgggtccttc ttccaggata ctcgtgacgc |
| 181 | gtccccattt cccactccca ttgggtgtcg ggtgtctaga gaagccaatc agcgtcgccg |
| 241 | tggtcccagt tctaaagtcc ccacgcaccc acccggactc agaatctcct cagacgccga |
| 301 | gatgcgggtc acggcacccc gaaccgtcct cctgctgctc tcggcggccc tggccctgac |
| 361 | cgagacctgg gccggtgagt gcgggtcggc agggaaatgg cctctgtggg gaggagcgag |
| 421 | gggaccgcag gcgggggcgc aggacccggg gagccgcgcc gggaggaggg tcgggcgggt |
| 481 | ctcagcccct cctcgccccc aggctcccac tccatgaggt atttccacac cgccatgtcc |
| 541 | cggcccggcc gcggggagcc ccgcttcatc accgtgggct acgtggacga cacgctgttc |
| 601 | gtgaggttcg acagcgacgc cacgagtccg aggaaggagc cgcgggcgcc atggatagag |
| 661 | caggaggggc cggagtattg ggaccgggag acacagatct ccaagaccaa cacacagact |
| 721 | taccgagaga gcctgcggaa cctgcgcggc tactacaacc agagcgaggc cggtgagtga |
| 781 | ccccggcccg gggcgcaggt cacgactccc catcccccac gtacggcccg ggtcgccccg |
| 841 | agtctccggg tccgagatcc gcccccctga ggccgcggga cccgcccaga ccctcgaccg |
| 901 | gcgagagccc caggcgcgtt tacccggttt cattttcagt tgaggccaaa atccccgcgg |
| 961 | gttggtcggg gcggggcggg gcggggctcg ggggacgggg ctgaccgcgg ggcctgggcc |
| 1021 | agggtctcac acttggcaga ggatgtatgg ctgcgacctg gggcccgacg ggcgcctcct |
| 1081 | ccgcgggtat aaccagttag cctacgacgg caaggattac atcgccctga acgaggacct |
| 1141 | gagctcctgg accgcggcgg acaccgcggc tcagatcacc cagcgcaagt gggaggcggc |
| 1201 | ccgtgtggcg gagcaggaca gagcctacct ggagggcctg tgcgtggagt cgctccgcag |
| 1261 | atacctggag aacgggaagg agacgctgca gcgcgcgggt accaggggca gtggggagcc |
| 1321 | ttccccatct cctataggtc gccggggatg gcctcccacg agaagaggag gaaaatggga |
| 1381 | tcagcgctag aatgtcgccc tcccttgaat ggagaatggc atgagttttc ctgagtttcc |
| 1441 | tctgagggcc ccctcttctc tctaggacaa taaggaatga cgtctctgag gaaatggagg |
| 1501 | ggaagacagt ccctagaata ctgatcaggg gtcccctttg acccctgcag cagccttggg |
| 1561 | aaccgtgact ttcctctcag gccttgttct ctgcctcaca ctcagtgtgt ttggggctct |
| 1621 | gattccagca cttctgagtc actttacctc cactcagatc gggagcagaa gtccctgttc |
| 1681 | cccgctcaga gactcgaact ttccaatgaa taggagatta tcccaggtgc ctgcgtccag |
| 1741 | gctggtgtct gggttctgtg ccccttcccc accccaggtg tcctgtccat tctcaggctg |
| 1801 | gtcacatggg tggtcctagg gtgtcccatg agagatgcaa agcgcctgaa ttttctgact |
| 1861 | cttcccatca gaccccccaa agacacatgt gacccaccac cccatctctg accatgaggc |
| 1921 | caccctgagg tgctgggccc tgggcttcta ccctgcggag atcacactga cctggcagcg |
| 1981 | ggatggcgag gaccaaactc aggacaccga gcttgtggag accagaccag caggagatag |
| 2041 | aaccttccag aagtgggcag ctgtggtggt gccttctgga gaagagcaga gatacacatg |
| 2101 | ccatgtacag catgaggggc tgccgaagcc cctcaccctg agatggggta aggaggggga |
| 2161 | tgaggggtca tatctgttct cagggaaagc aggagccctt ctggagccct tcagcagggt |
| 2221 | cagggcccct catcttcccc tcctttccca gagccatctt cccagtccac catccccatc |
| 2281 | gtgggcattg ttgctggcct ggctgtccta gcagttgtgg tcatcggagc tgtggtcgct |
| 2341 | actgtgatgt gtaggaggaa gagctcaggt agggaagggg tgaggggtgg ggtctgggtt |
| 2401 | ttcttgtccc actgggggtt tcaagcccca ggtagaagtg ttccctgcct cattactggg |
| 2461 | aagcagcatc cacacagggg ctaacgcagc ctgggaccct gtgtgccagc acttactctt |
| 2521 | ttgtgcagca catgtgacaa tgaaggacgg atgtatcgcc ttgatggttg tggtgttggg |
| 2581 | gtcctgattc cagcattcat gagtcagggg aaggtccctg ctaaggacag accttaggag |
| 2641 | ggcagttggt ccaggaccca cacttgcttt cctcgtgttt cctgatcctg ccttgggtct |
| 2701 | gtagtcatac ttctggaaat tccttttggt tccaagacga ggaggttcct ctaagatctc |
| 2761 | atggccctgc ttcctcccag tcccctcaca ggacattttc ttcccacagg tggaaaagga |
| 2821 | gggagctact ctcaggctgc gtgtaagtgg tgggggtggg agtgtggagg agctcaccca |
| 2881 | ccccataatt cctcctgtcc cacgtctcct gagggctctg accaggtcct gtttttgttc |
| 2941 | tactccagcc agcgacagtg cccagggctc tgatgtgtct ctcacagctt gaaaaggtga |
| 3001 | gattcttggg gtctagagtg ggtggggtgg cgggtctggg ggtgggtggg gcagtgggga |
| 3061 | aaggcctggg taatggagat tctttgattg ggatgtttcg cgtgtgtggt gggctgttca |
| 3121 | gagtgtcatc acttaccatg actaaccaga atttgttcat gactgttgtt ttctgtagcc |
| 3181 | tgagacagct gtcttgtgag ggactgagat gcaggatttc ttcacgcctc ccctttgtga |
| 3241 | cttcaagagc ctctggcatc tctttctgca aaggcacctg aatgtgtctg cgtccctgtt |
| 3301 | agcataatgt gaggaggtgg agagacagcc cacccttgtg tccactgtga cccctgttcg |
| 3361 | catgctgacc tgtgtttcct cccca. |

Compositions of the disclosure may comprise a gRNA comprising a spacer sequence that specifically binds to a target sequence of an RNA molecule encoding an α-chain derived from an HLA-C gene comprising or consisting of 20 nucleotides of the sequence of

| (SEQ ID NO: 218) | |
|---|---|
| 1 | tccgcagtcc cggttctaaa gtccccagtc acccacccgg actcacattc tccccagagg |
| 61 | ccgagatgcg ggtcatggcg ccccgagccc tcctcctgct gctctcggga ggcctggccc |
| 121 | tgaccgagac ctgggcctgc tcccactcca tgaggtattt cgacaccgcc gtgtcccggc |
| 181 | ccggccgcgg agagccccgc ttcatctcag tgggctacgt ggacgacacg cagttcgtgc |
| 241 | ggttcgacag cgacgccgcg agtccgagag gggagccgcg ggcgccgtgg gtggagcagg |
| 301 | aggggccgga gtattgggac cgggagacac agaagtacaa gcgccaggca caggctgacc |
| 361 | gagtgagcct gcggaacctg cgcggctact acaaccagag cgaggacggg tctcacaccc |
| 421 | tccagaggat gtctggctgc gacctggggc ccgacgggcg cctcctccgc gggtatgacc |
| 481 | agtccgccta cgacggcaag gattacatcg ccctgaacga ggacctgcgc tcctggaccg |
| 541 | ccgcggacac cgcggctcag atcacccagc gcaagttgga ggcggcccgt gcggcggagc |
| 601 | agctgagagc ctacctggag ggcacgtgcg tggagtggct ccgcagatac ctggagaacg |
| 661 | ggaaggagac gctgcagcgc gcagaacccc caaagacaca cgtgacccac caccccctct |
| 721 | ctgaccatga ggccaccctg aggtgctggg ccctgggctt ctaccctgcg gagatcacac |
| 781 | tgacctggca gcgggatggg gaggaccaga cccaggacac cgagcttgtg gagaccaggc |
| 841 | cagcaggaga tggaaccttc cagaagtggg cagctgtggt ggtgccttct ggacaagagc |
| 901 | agagatacac gtgccatatg cagcacgagg ggctgcaaga gcccctcacc ctgagctggg |
| 961 | agccatcttc ccagcccacc atccccatca tgggcatcgt tgctggcctg gctgtcctgg |
| 1021 | ttgtcctagc tgtccttgga gctgtggtca ccgctatgat gtgtaggagg aagagctcag |
| 1081 | gtggaaaagg agggagctgc tctcaggctg cgtgcagcaa cagtgcccag ggctctgatg |
| 1141 | agtctctcat cacttgtaaa gcctgagaca gctgcctgtg tgggactgag atgcaggatt |
| 1201 | tcttcacacc tctcctttgt gacttcaaga gcctctggca tctctttctg caaaggcacc |
| 1261 | tgaatgtgtc tgcgttcctg ttagcataat gtgaggaggt ggagagacag cccacccccg |
| 1321 | tgtccaccgt gacccctgtc cccacactga cctgtgttcc ctccccgatc atctttcctg |
| 1381 | ttccagagag gtggggctgg atgtctccat ctctgtctca aattcatggt gcactgagct |
| 1441 | gcaacttctt acttccctaa tgaagttaag aacctgaata taaatttgtg ttctcaaata |
| 1501 | tttgctatga agcgttgatg gattaattaa ataagtcaat tcctagaagt tgagagagca |
| 1561 | aataaagacc tgagaacctt ccagaa. |

Compositions of the disclosure may comprise a gRNA comprising a spacer sequence that specifically binds to a target sequence of an RNA molecule encoding an α-chain derived from an HLA-C gene comprising or consisting of 20 nucleotides of the sequence of

| (SEQ ID NO: 219) | |
|---|---|
| 1 | tccgcagtcc cggttctaaa gtccccagtc acccacccgg actcacattc tccccagagg |
| 61 | ccgagatgcg ggtcatggcg ccccgagccc tcctcctgct gctctcggga ggcctggccc |
| 121 | tgaccgagac ctgggcctgc tcccactcca tgaggtattt cgacaccgcc gtgtcccggc |
| 181 | ccggccgcgg agagccccgc ttcatctcag tgggctacgt ggacgacacg cagttcgtgc |
| 241 | ggttcgacag cgacgccgcg agtccgagag gggagccgcg ggcgccgtgg gtggagcagg |
| 301 | aggggccgga gtattgggac cgggagacac agaactacaa gcgccaggca caggctgacc |
| 361 | gagtgagcct gcggaacctg cgcggctact acaaccagag cgaggacggg tctcacaccc |
| 421 | tccagaggat gtatggctgc gacctggggc ccgacgggcg cctcctccgc gggtatgacc |
| 481 | agtccgccta cgacggcaag gattacatcg ccctgaacga ggacctgcgc tcctggaccg |
| 541 | ccgcggacac cgcggctcag atcacccagc gcaagttgga ggcggcccgt gcggcggagc |
| 601 | agctgagagc ctacctggag ggcacgtgcg tggagtggct ccgcagatac ctggagaacg |
| 661 | ggaaggagac gctgcagcgc gcagaacccc caaagacaca cgtgacccac caccccctct |
| 721 | ctgaccatga ggccaccctg aggtgctggg ccctgggctt ctaccctgcg gagatcacac |
| 781 | tgacctggca gcgggatggg gaggaccaga cccaggacac cgagcttgtg gagaccaggc |
| 841 | cagcaggaga tggaaccttc cagaagtggg cagctgtggt ggtgccttct ggacaagagc |
| 901 | agagatacac gtgccatatg cagcacgagg ggctgcaaga gcccctcacc ctgagctggg |
| 961 | agccatcttc ccagcccacc atccccatca tgggcatcgt tgctggcctg gctgtcctgg |
| 1021 | ttgtcctagc tgtccttgga gctgtggtca ccgctatgat gtgtaggagg aagagctcag |
| 1081 | gtggaaaagg agggagctgc tctcaggctg cgtgcagcaa cagtgcccag ggctctgatg |
| 1141 | agtctctcat cacttgtaaa gcctgagaca gctgcctgtg tgggactgag atgcaggatt |
| 1201 | tcttcacacc tctcctttgt gacttcaaga gcctctggca tctctttctg caaaggcgtc |
| 1261 | tgaatgtgtc tgcgttcctg ttagcataat gtgaggaggt ggagagacag cccacccccg |
| 1321 | tgtccaccgt gacccctgtc cccacactga cctgtgttcc ctccccgatc atctttcctg |
| 1381 | ttccagagag gtggggctgg atgtctccat ctctgtctca aattcatggt gcactgagct |
| 1441 | gcaacttctt acttccctaa tgaagttaag aacctgaata taaatttgtg ttctcaaata |
| 1501 | tttgctatga agcgttgatg gattaattaa ataagtcaat tcctagaagt tgagagagca |
| 1561 | aataaagacc tgagaacctt ccagaa. |

Compositions of the disclosure may comprise a gRNA comprising a spacer sequence that specifically binds to a target sequence of an RNA molecule encoding an β2M protein comprising or consisting of 20 nucleotides of the sequence of

| (SEQ ID NO: 220) | |
|---|---|
| 1 | attcctgaag ctgacagcat tcgggccgag atgtctcgct ccgtggcctt agctgtgctc |
| 61 | gcgctactct ctctttctgg cctggaggct atccagcgta ctccaaagat tcaggtttac |
| 121 | tcacgtcatc cagcagagaa tggaaagtca aatttcctga attgctatgt gtctgggttt |
| 181 | catccatccg acattgaagt tgacttactg aagaatggag agagaattga aaaagtggag |
| 241 | cattcagact tgtctttcag caaggactgg tctttctatc tcttgtacta cactgaattc |
| 301 | acccccactg aaaaagatga gtatgcctgc cgtgtgaacc atgtgacttt gtcacagccc |
| 361 | aagatagtta agtgggatcg agacatgtaa gcagcatcat ggaggtttga agatgccgca |
| 421 | tttggattgg atgaattcca aattctgctt gcttgctttt taatattgat atgcttatac |
| 481 | acttacactt tatgcacaaa atgtagggtt ataataatgt taacatggac atgatcttct |
| 541 | ttataattct actttgagtg ctgtctccat gtttgatgta tctgagcagg ttgctccaca |
| 601 | ggtagctcta ggagggctgg caacttagag gtggggagca gagaattctc ttatccaaca |
| 661 | tcaacatctt ggtcagattt gaactcttca atctcttgca ctcaaagctt gttaagatag |
| 721 | ttaagcgtgc ataagttaac ttccaattta catactctgc ttagaatttg ggggaaaatt |
| 781 | tagaaatata attgacagga ttattggaaa tttgttataa tgaatgaaac attttgtcat |
| 841 | ataagattca tatttacttc ttatacattt gataaagtaa ggcatggttg tggttaatct |
| 901 | ggtttatttt tgttccacaa gttaaataaa tcataaaact tgatgtgtta tctcttatat |
| 961 | ctcactccca ctattacccc tttattttca aacagggaaa cagtcttcaa gttccacttg |
| 1021 | gtaaaaaatg tgaacccctt gtatatagag tttggctcac agtgtaaagg gcctcagtga |
| 1081 | ttcacatttt ccagattagg aatctgatgc tcaaagaagt taaatggcat agttggggtg |
| 1141 | acacagctgt ctagtgggag gccagccttc tatattttag ccagcgttct ttcctgcggg |
| 1201 | ccaggtcatg aggagtatgc agactctaag agggagcaaa agtatctgaa ggatttaata |
| 1261 | ttttagcaag gaatagatat acaatcatcc cttggtctcc ctgggggatt ggtttcagga |
| 1321 | ccccttcttg gacaccaaat ctatggatat ttaagtccct tctataaaat ggtatagtat |
| 1381 | ttgcatataa cctatccaca tcctcctgta tactttaaat catttctaga ttacttgtaa |
| 1441 | tacctaatac aatgtaaatg ctatgcaaat agttgttatt gtttaaggaa taatgacaag |
| 1501 | aaaaaaaagt ctgtacatgc tcagtaaaga cacaaccatc cctttttttc cccagtgttt |
| 1561 | ttgatccatg gtttgctgaa tccacagatg tggagcccct ggatacggaa ggcccgctgt |
| 1621 | actttgaatg acaaataaca gatttaaaat tttcaaggca tagttttata cctga. |

Compositions of the disclosure may comprise a gRNA comprising a spacer sequence that specifically binds to a target sequence of an RNA molecule encoding CD28 protein comprising or consisting of 20 nucleotides of the sequence of

| (SEQ ID NO: 221) | |
|---|---|
| 1 | taaagtcatc aaaacaacgt tatatcctgt gtgaaatgct gcagtcagga tgccttgtgg |
| 61 | tttgagtgcc ttgatcatgt gccctaaggg gatggtggcg gtggtggtgg ccgtggatga |
| 121 | cggagactct caggccttgg caggtgcgtc tttcagttcc cctcacactt cgggttcctc |
| 181 | ggggaggagg ggctggaacc ctagcccatc gtcaggacaa agatgctcag gctgctcttg |
| 241 | gctctcaact tattcccttc aattcaagta acaggaaaca agattttggt gaagcagtcg |
| 301 | cccatgcttg tagcgtacga caatgcggtc aaccttagct gcaagtattc ctacaatctc |
| 361 | ttctcaaggg agttccgggc atcccttcac aaaggactgg atagtgctgt ggaagtctgt |
| 421 | gttgtatatg ggaattactc ccagcagctt caggtttact caaaaacggg gttcaactgt |
| 481 | gatgggaaat tgggcaatga atcagtgaca ttctacctcc agaatttgta tgttaaccaa |
| 541 | acagatattt acttctgcaa aattgaagtt atgtatcctc ctccttacct agacaatgag |
| 601 | aagagcaatg gaaccattat ccatgtgaaa gggaaacacc tttgtccaag tcccctattt |
| 661 | cccggacctt ctaagccctt ttgggtgctg gtggtggttg gtggagtcct ggcttgctat |
| 721 | agcttgctag taacagtggc ctttattatt ttctgggtga ggagtaagag gagcaggctc |
| 781 | ctgcacagtg actacatgaa catgactccc cgccgccccg ggcccacccg caagcattac |
| 841 | cagccctatg ccccaccacg cgacttcgca gcctatcgct cctgacacgg acgcctatcc |
| 901 | agaagccagc cggctggcag cccccatctg ctcaatatca ctgctctgga taggaaatga |
| 961 | ccgccatctc cagccggcca cctcaggccc ctgttgggcc accaatgcca atttttctcg |
| 1021 | agtgactaga ccaaatatca agatcatttt gagactctga aatgaagtaa aagagatttc |
| 1081 | ctgtgacagg ccaagtctta cagtgccatg gcccacattc caacttacca tgtacttagt |
| 1141 | gacttgactg agaagttagg gtagaaaaca aaaagggagt ggattctggg agcctcttcc |
| 1201 | ctttctcact cacctgcaca tctcagtcaa gcaaagtgtg gtatccacag acattttagt |
| 1261 | tgcagaagaa aggctaggaa atcattcctt ttggttaaat gggtgtttaa tcttttggtt |
| 1321 | agtgggttaa acggggtaag ttagagtagg gggagggata ggaagacata tttaaaaacc |
| 1381 | attaaaacac tgtctcccac tcatgaaatg agccacgtag ttcctattta atgctgtttt |
| 1441 | cctttagttt agaaatacat agacattgtc ttttatgaat tctgatcata tttagtcatt |
| 1501 | ttgaccaaat gagggatttg gtcaaatgag ggattccctc aaagcaatat caggtaaacc |
| 1561 | aagttgcttt cctcactccc tgtcatgaga cttcagtgtt aatgttcaca atatactttc |
| 1621 | gaaagaataa aatagttctc ctacatgaag aaagaatatg tcaggaaata aggtcacttt |
| 1681 | atgtcaaaat tatttgagta ctatgggacc tggcgcagtg gctcatgctt gtaatcccag |
| 1741 | cactttggga ggccgaggtg ggcagatcac ttgagatcag gaccagcctg gtcaagatgg |
| 1801 | tgaaactccg tctgtactaa aaatacaaaa tttagcttgg cctggtggca ggcacctgta |
| 1861 | atcccagctg cccaagaggc tgaggcatga gaatcgcttg aacctggcag gcggaggttg |
| 1921 | cagtgagccg agatagtgcc acagctctcc agcctgggcg acagagtgag actccatctc |
| 1981 | aaacaacaac aacaacaaca acaacaacaa caaaccacaa aattatttga gtactgtgaa |
| 2041 | ggattatttg tctaacagtt cattccaatc agaccaggta ggagctttcc tgtttcatat |
| 2101 | gtttcagggt tgcacagttg gtctctttaa tgtcggtgtg gagatccaaa gtgggttgtg |
| 2161 | gaaagagcgt ccataggaga agtgagaata ctgtgaaaaa gggatgttag cattcattag |
| 2221 | agtatgagga tgagtcccaa gaaggttctt tggaaggagg acgaatagaa tggagtaatg |
| 2281 | aaattcttgc catgtgctga ggagatagcc agcattaggt gacaatcttc cagaagtggt |
| 2341 | caggcagaag gtgccctggt gagagctcct ttacagggac tttatgtggt ttagggctca |
| 2401 | gagctccaaa actctgggct cagctgctcc tgtaccttgg aggtccattc acatgggaaa |
| 2461 | gtattttgga atgtgtcttt tgaagagagc atcagagttc ttaagggact gggtaaggcc |
| 2521 | tgaccctgaa atgaccatgg atatttttct acctacagtt tgagtcaact agaatatgcc |
| 2581 | tggggacctt gaagaatggc ccttcagtgg ccctcaccat ttgttcatgc ttcagttaat |
| 2641 | tcaggtgttg aaggagctta ggttttagag gcacgtagac ttggttcaag tctcgttagt |
| 2701 | agttgaatag cctcaggcaa gtcactgccc acctaagatg atggttcttc aactataaaa |
| 2761 | tggagataat ggttacaaat gtctcttcct atagtataat ctccataagg gcatggccca |
| 2821 | agtctgtctt tgactctgcc tatccctgac atttagtagc atgcccgaca tacaatgtta |
| 2881 | gctattggta ttattgccat atagataaat tatgtataaa aattaaactg ggcaatagcc |
| 2941 | taagaagggg ggaatattgt aacacaaatt taaacccact acgcagggat gaggtgctat |
| 3001 | aatatgagga ccttttaact tccatcattt tcctgtttct tgaaatagtt tatcttgtaa |
| 3061 | tgaaatataa ggcacctccc acttttatgt atagaaagag gtcttttaat ttttttttaa |
| 3121 | tgtgagaagg aagggaggag taggaatctt gagattccag atcgaaaata ctgtactttg |
| 3181 | gttgattttt aagtgggctt ccattccatg gatttaatca gtcccaagaa gatcaaactc |
| 3241 | agcagtactt gggtgctgaa gaactgttgg atttaccctg gcacgtgtgc cacttgccag |
| 3301 | cttcttgggc acacagagtt cttcaatcca agttatcaga ttgtatttga aaatgacaga |
| 3361 | gctggagagt tttttgaaat ggcagtggca aataaataaa tacttttttt taaatggaaa |
| 3421 | gacttgatct atggtaataa atgattttgt tttctgactg gaaaaatagg cctactaaag |
| 3481 | atgaatcaca cttgagatgt ttcttactca ctctgcacag aaacaaagaa gaaatgttat |
| 3541 | acagggaagt ccgttttcac tattagtatg aaccaagaaa tggttcaaaa acagtggtag |
| 3601 | gagcaatgct ttcatagttt cagatatggt agttatgaag aaaacaatgt catttgctgc |
| 3661 | tattattgta agagtcttat aattaatggt actcctataa tttttgattg tgagctcacc |
| 3721 | tatttgggtt aagcatgcca atttaaagag accaagtgta tgtacattat gttctacata |
| 3781 | ttcagtgata aaattactaa actactatat gtctgcttta aatttgtact ttaatattgt |
| 3841 | cttttggtat taagaaagat atgctttcag aatagatatg cttcgctttg gcaaggaatt |
| 3901 | tggatagaac ttgctattta aaagaggtgt ggggtaaatc cttgtataaa tctccagttt |
| 3961 | agcctttttt gaaaaagcta gactttcaaa tactaatttc acttcaagca gggtacgttt |
| 4021 | ctggtttgtt tgcttgactt cagtcacaat ttcttatcag accaatggct gacctctttg |
| 4081 | agatgtcagg ctaggcttac ctatgtgttc tgtgtcatgt gaatgctgag aagtttgaca |
| 4141 | gagatccaac ttcagccttg accccatcag tccctcgggt taactaactg agccaccggt |
| 4201 | cctcatggct attttaatga gggtattgat ggttaaatgc atgtctgatc ccttatccca |
| 4261 | gccatttgca ctgccagctg ggaactatac cagacctgga tactgatccc aaagtgttaa |
| 4321 | attcaactac atgctggaga ttagagatgg tgccaataaa ggacccagaa ccaggatctt |
| 4381 | gattgctata gacttattaa taatccaggt caaagagagt gacacacact ctctcaagac |
| 4441 | ctggggtgag ggagtctgtg ttatctgcaa ggccatttga ggctcagaaa gtctctcttt |
| 4501 | cctatagata tatgcatact ttctgacata taggaatgta tcaggaatac tcaaccatca |
| 4561 | caggcatgtt cctacctcag ggcctttaca tgtcctgttt actctgtcta gaatgtcctt |
| 4621 | ctgtagatga cctggcttgc ctcgtcaccc ttcaggtcct tgctcaagtg tcatcttctc |
| 4681 | ccctagttaa actaccccac accctgtctg ctttccttgc ttatttttct ccatagcatt |
| 4741 | ttaccatctc ttacattaga catttttctt atttatttgt agtttataag cttcatgagg |
| 4801 | caagtaactt tgctttgttt cttgctgtat ctccagtgcc cagagcagtg cctggtatat |
| 4861 | aataaatatt tattgactga gtgaaaaaaa aaaaaaaaaa. |

Compositions of the disclosure may comprise a gRNA comprising a spacer sequence that specifically binds to a target sequence of an RNA molecule encoding CD28 protein comprising or consisting of 20 nucleotides of the sequence of

| (SEQ ID NO: 222) | |
|---|---|
| 1 | taaagtcatc aaaacaacgt tatatcctgt gtgaaatgct gcagtcagga tgccttgtgg |
| 61 | tttgagtgcc ttgatcatgt gccctaaggg gatggtggcg gtggtggtgg ccgtggatga |
| 121 | cggagactct caggccttgg caggtgcgtc tttcagttcc cctcacactt cgggttcctc |
| 181 | ggggaggagg ggctggaacc ctagcccatc gtcaggacaa agatgctcag gctgctcttg |
| 241 | gctctcaact tattcccttc aattcaagta acaggaaaca agattttggt gaagcagtcg |
| 301 | cccatgcttg tagcgtacga caatgcggtc aaccttagct ggaaacacct ttgtccaagt |
| 361 | cccctatttc ccggaccttc taagcccttt tgggtgctgg tggtggttgg tggagtcctg |
| 421 | gcttgctata gcttgctagt aacagtggcc tttattattt tctgggtgag gagtaagagg |
| 481 | agcaggctcc tgcacagtga ctacatgaac atgactcccc gccgccccgg gcccacccgc |
| 541 | aagcattacc agccctatgc cccaccacgc gacttcgcag cctatcgctc ctgacacgga |
| 601 | cgcctatcca gaagccagcc ggctggcagc ccccatctgc tcaatatcac tgctctggat |
| 661 | aggaaatgac cgccatctcc agccggccac ctcaggcccc tgttgggcca ccaatgccaa |
| 721 | tttttctcga gtgactagac caaatatcaa gatcattttg agactctgaa atgaagtaaa |
| 781 | agagatttcc tgtgacaggc caagtcttac agtgccatgg cccacattcc aacttaccat |
| 841 | gtacttagtg acttgactga gaagttaggg tagaaaacaa aaagggagtg gattctggga |
| 901 | gcctcttccc tttctcactc acctgcacat ctcagtcaag caaagtgtgg tatccacaga |
| 961 | cattttagtt gcagaagaaa ggctaggaaa tcattccttt tggttaaatg ggtgtttaat |
| 1021 | cttttggtta gtgggttaaa cggggtaagt tagagtaggg ggagggatag gaagacatat |
| 1081 | ttaaaaacca ttaaaacact gtctcccact catgaaatga gccacgtagt tcctatttaa |
| 1141 | tgctgttttc ctttagttta gaaatacata gacattgtct tttatgaatt ctgatcatat |
| 1201 | ttagtcattt tgaccaaatg agggatttgg tcaaatgagg gattccctca aagcaatatc |
| 1261 | aggtaaacca agttgctttc ctcactccct gtcatgagac ttcagtgtta atgttcacaa |
| 1321 | tatactttcg aaagaataaa atagttctcc tacatgaaga aagaatatgt caggaaataa |
| 1381 | ggtcacttta tgtcaaaatt atttgagtac tatgggacct ggcgcagtgg ctcatgcttg |
| 1441 | taatcccagc actttgggag gccgaggtgg gcagatcact tgagatcagg accagcctgg |
| 1501 | tcaagatggt gaaactccgt ctgtactaaa aatacaaaat ttagcttggc ctggtggcag |
| 1561 | gcacctgtaa tcccagctgc ccaagaggct gaggcatgag aatcgcttga acctggcagg |
| 1621 | cggaggttgc agtgagccga gatagtgcca cagctctcca gcctgggcga cagagtgaga |
| 1681 | ctccatctca aacaacaaca acaacaacaa caacaacaac aaaccacaaa attatttgag |
| 1741 | tactgtgaag gattatttgt ctaacagttc attccaatca gaccaggtag gagctttcct |
| 1801 | gtttcatatg tttcagggtt gcacagttgg tctctttaat gtcggtgtgg agatccaaag |
| 1861 | tgggttgtgg aaagagcgtc cataggagaa gtgagaatac tgtgaaaaag ggatgttagc |
| 1921 | attcattaga gtatgaggat gagtcccaag aaggttcttt ggaaggagga cgaatagaat |
| 1981 | ggagtaatga aattcttgcc atgtgctgag gagatagcca gcattaggtg acaatcttcc |
| 2041 | agaagtggtc aggcagaagg tgccctggtg agagctcctt tacagggact ttatgtggtt |
| 2101 | tagggctcag agctccaaaa ctctgggctc agctgctcct gtaccttgga ggtccattca |
| 2161 | catgggaaag tattttggaa tgtgtctttt gaagagagca tcagagttct taagggactg |
| 2221 | ggtaaggcct gaccctgaaa tgaccatgga tatttttcta cctacagttt gagtcaacta |
| 2281 | gaatatgcct ggggaccttg aagaatggcc cttcagtggc cctcaccatt tgttcatgct |
| 2341 | tcagttaatt caggtgttga aggagcttag gttttagagg cacgtagact tggttcaagt |
| 2401 | ctcgttagta gttgaatagc ctcaggcaag tcactgccca cctaagatga tggttcttca |
| 2461 | actataaaat ggagataatg gttacaaatg tctcttccta tagtataatc tccataaggg |
| 2521 | catggcccaa gtctgtcttt gactctgcct atccctgaca tttagtagca tgcccgacat |
| 2581 | acaatgttag ctattggtat tattgccata tagataaatt atgtataaaa attaaactgg |
| 2641 | gcaatagcct aagaaggggg gaatattgta acacaaattt aaacccacta cgcagggatg |
| 2701 | aggtgctata atatgaggac cttttaactt ccatcatttt cctgtttctt gaaatagttt |
| 2761 | atcttgtaat gaaatataag gcacctccca cttttatgta tagaaagagg tcttttaatt |
| 2821 | tttttttaat gtgagaagga agggaggagt aggaatcttg agattccaga tcgaaaatac |
| 2881 | tgtactttgg ttgattttta agtgggcttc cattccatgg atttaatcag tcccaagaag |
| 2941 | atcaaactca gcagtacttg ggtgctgaag aactgttgga tttaccctgg cacgtgtgcc |
| 3001 | acttgccagc ttcttgggca cacagagttc ttcaatccaa gttatcagat tgtatttgaa |
| 3061 | aatgacagag ctggagagtt ttttgaaatg gcagtggcaa ataaataaat actttttttt |
| 3121 | aaatggaaag acttgatcta tggtaataaa tgattttgtt ttctgactgg aaaaataggc |
| 3181 | ctactaaaga tgaatcacac ttgagatgtt tcttactcac tctgcacaga aacaaagaag |
| 3241 | aaatgttata cagggaagtc cgttttcact attagtatga accaagaaat ggttcaaaaa |
| 3301 | cagtggtagg agcaatgctt tcatagtttc agatatggta gttatgaaga aaacaatgtc |
| 3361 | atttgctgct attattgtaa gagtcttata attaatggta ctcctataat ttttgattgt |
| 3421 | gagctcacct atttgggtta agcatgccaa tttaaagaga ccaagtgtat gtacattatg |
| 3481 | ttctacatat tcagtgataa aattactaaa ctactatatg tctgctttaa atttgtactt |
| 3541 | taatattgtc ttttggtatt aagaaagata tgctttcaga atagatatgc ttcgctttgg |
| 3601 | caaggaattt ggatagaact tgctatttaa aagaggtgtg gggtaaatcc ttgtataaat |
| 3661 | ctccagttta gccttttttg aaaaagctag actttcaaat actaatttca cttcaagcag |
| 3721 | ggtacgtttc tggtttgttt gcttgacttc agtcacaatt tcttatcaga ccaatggctg |
| 3781 | acctctttga gatgtcaggc taggcttacc tatgtgttct gtgtcatgtg aatgctgaga |
| 3841 | agtttgacag agatccaact tcagccttga ccccatcagt ccctcgggtt aactaactga |
| 3901 | gccaccggtc ctcatggcta ttttaatgag ggtattgatg gttaaatgca tgtctgatcc |
| 3961 | cttatcccag ccatttgcac tgccagctgg gaactatacc agacctggat actgatccca |
| 4021 | aagtgttaaa ttcaactaca tgctggagat tagagatggt gccaataaag gacccagaac |
| 4081 | caggatcttg attgctatag acttattaat aatccaggtc aaagagagtg acacacactc |
| 4141 | tctcaagacc tggggtgagg gagtctgtgt tatctgcaag gccatttgag gctcagaaag |
| 4201 | tctctctttc ctatagatat atgcatactt tctgacatat aggaatgtat caggaatact |
| 4261 | caaccatcac aggcatgttc ctacctcagg gcctttacat gtcctgttta ctctgtctag |
| 4321 | aatgtccttc tgtagatgac ctggcttgcc tcgtcaccct tcaggtcctt gctcaagtgt |
| 4381 | catcttctcc cctagttaaa ctaccccaca ccctgtctgc tttccttgct tatttttctc |
| 4441 | catagcattt taccatctct tacattagac atttttctta tttatttgta gtttataagc |
| 4501 | ttcatgaggc aagtaacttt gctttgtttc ttgctgtatc tccagtgccc agagcagtgc |
| 4561 | ctggtatata ataaatattt attgactgag tgaaaaaaaa aaaaaaaaa. |

Compositions of the disclosure may comprise a gRNA comprising a spacer sequence that specifically binds to a target sequence of an RNA molecule encoding CD28 protein comprising or consisting of 20 nucleotides of the sequence of

| (SEQ ID NO: 223) | |
|---|---|
| 1 | taaagtcatc aaaacaacgt tatatcctgt gtgaaatgct gcagtcagga tgccttgtgg |
| 61 | tttgagtgcc ttgatcatgt gccctaaggg gatggtggcg gtggtggtgg ccgtggatga |
| 121 | cggagactct caggccttgg caggtgcgtc tttcagttcc cctcacactt cgggttcctc |
| 181 | ggggaggagg ggctggaacc ctagcccatc gtcaggacaa agatgctcag gctgctcttg |
| 241 | gctctcaact tattcccttc aattcaagta acagggaaac acctttgtcc aagtccccta |
| 301 | tttcccggac cttctaagcc cttttgggtg ctggtggtgg ttggtggagt cctggcttgc |
| 361 | tatagcttgc tagtaacagt ggcctttatt attttctggg tgaggagtaa gaggagcagg |
| 421 | ctcctgcaca gtgactacat gaacatgact ccccgccgcc ccgggcccac ccgcaagcat |
| 481 | taccagccct atgccccacc acgcgacttc gcagcctatc gctcctgaca cggacgccta |
| 541 | tccagaagcc agccggctgg cagcccccat ctgctcaata tcactgctct ggataggaaa |
| 601 | tgaccgccat ctccagccgg ccacctcagg cccctgttgg gccaccaatg ccaatttttc |
| 661 | tcgagtgact agaccaaata tcaagatcat tttgagactc tgaaatgaag taaaagagat |
| 721 | ttcctgtgac aggccaagtc ttacagtgcc atggcccaca ttccaactta ccatgtactt |
| 781 | agtgacttga ctgagaagtt agggtagaaa acaaaaaggg agtggattct gggagcctct |
| 841 | tccctttctc actcacctgc acatctcagt caagcaaagt gtggtatcca cagacatttt |
| 901 | agttgcagaa gaaaggctag gaaatcattc cttttggtta aatgggtgtt taatcttttg |
| 961 | gttagtgggt taaacggggt aagttagagt agggggaggg ataggaagac atatttaaaa |
| 1021 | accattaaaa cactgtctcc cactcatgaa atgagccacg tagttcctat ttaatgctgt |
| 1081 | tttcctttag tttagaaata catagacatt gtcttttatg aattctgatc atatttagtc |
| 1141 | attttgacca aatgagggat ttggtcaaat gagggattcc ctcaaagcaa tatcaggtaa |
| 1201 | accaagttgc tttcctcact ccctgtcatg agacttcagt gttaatgttc acaatatact |
| 1261 | ttcgaaagaa taaaatagtt ctcctacatg aagaaagaat atgtcaggaa ataaggtcac |
| 1321 | tttatgtcaa aattatttga gtactatggg acctggcgca gtggctcatg cttgtaatcc |
| 1381 | cagcactttg ggaggccgag gtgggcagat cacttgagat caggaccagc ctggtcaaga |
| 1441 | tggtgaaact ccgtctgtac taaaaataca aaatttagct tggcctggtg gcaggcacct |
| 1501 | gtaatcccag ctgcccaaga ggctgaggca tgagaatcgc ttgaacctgg caggcggagg |
| 1561 | ttgcagtgag ccgagatagt gccacagctc tccagcctgg gcgacagagt gagactccat |
| 1621 | ctcaaacaac aacaacaaca acaacaacaa caacaaacca caaaattatt tgagtactgt |
| 1681 | gaaggattat ttgtctaaca gttcattcca atcagaccag gtaggagctt tcctgtttca |
| 1741 | tatgtttcag ggttgcacag ttggtctctt taatgtcggt gtggagatcc aaagtgggtt |
| 1801 | gtggaaagag cgtccatagg agaagtgaga atactgtgaa aaagggatgt tagcattcat |
| 1861 | tagagtatga ggatgagtcc caagaaggtt ctttggaagg aggacgaata gaatggagta |
| 1921 | atgaaattct tgccatgtgc tgaggagata gccagcatta ggtgacaatc ttccagaagt |
| 1981 | ggtcaggcag aaggtgccct ggtgagagct cctttacagg gactttatgt ggtttagggc |
| 2041 | tcagagctcc aaaactctgg gctcagctgc tcctgtacct tggaggtcca ttcacatggg |
| 2101 | aaagtatttt ggaatgtgtc ttttgaagag agcatcagag ttcttaaggg actgggtaag |
| 2161 | gcctgaccct gaaatgacca tggatatttt tctacctaca gtttgagtca actagaatat |
| 2221 | gcctggggac cttgaagaat ggcccttcag tggccctcac catttgttca tgcttcagtt |
| 2281 | aattcaggtg ttgaaggagc ttaggtttta gaggcacgta gacttggttc aagtctcgtt |
| 2341 | agtagttgaa tagcctcagg caagtcactg cccacctaag atgatggttc ttcaactata |
| 2401 | aaatggagat aatggttaca aatgtctctt cctatagtat aatctccata agggcatggc |
| 2461 | ccaagtctgt ctttgactct gcctatccct gacatttagt agcatgcccg acatacaatg |
| 2521 | ttagctattg gtattattgc catatagata aattatgtat aaaaattaaa ctgggcaata |
| 2581 | gcctaagaag gggggaatat tgtaacacaa atttaaaccc actacgcagg gatgaggtgc |
| 2641 | tataatatga ggacctttta acttccatca ttttcctgtt tcttgaaata gtttatcttg |
| 2701 | taatgaaata taaggcacct cccactttta tgtatagaaa gaggtctttt aatttttttt |
| 2761 | taatgtgaga aggaagggag gagtaggaat cttgagattc cagatcgaaa atactgtact |
| 2821 | ttggttgatt tttaagtggg cttccattcc atggatttaa tcagtcccaa gaagatcaaa |
| 2881 | ctcagcagta cttgggtgct gaagaactgt tggatttacc ctggcacgtg tgccacttgc |
| 2941 | cagcttcttg ggcacacaga gttcttcaat ccaagttatc agattgtatt tgaaaatgac |
| 3001 | agagctggag agttttttga aatggcagtg gcaaataaat aaatactttt ttttaaatgg |
| 3061 | aaagacttga tctatggtaa taaatgattt tgttttctga ctggaaaaat aggcctacta |
| 3121 | aagatgaatc acacttgaga tgtttcttac tcactctgca cagaaacaaa gaagaaatgt |
| 3181 | tatacaggga agtccgtttt cactattagt atgaaccaag aaatggttca aaaacagtgg |
| 3241 | taggagcaat gctttcatag tttcagatat ggtagttatg aagaaaacaa tgtcatttgc |
| 3301 | tgctattatt gtaagagtct tataattaat ggtactccta taatttttga ttgtgagctc |
| 3361 | acctatttgg gttaagcatg ccaatttaaa gagaccaagt gtatgtacat tatgttctac |
| 3421 | atattcagtg ataaaattac taaactacta tatgtctgct ttaaatttgt actttaatat |
| 3481 | tgtcttttgg tattaagaaa gatatgcttt cagaatagat atgcttcgct ttggcaagga |
| 3541 | atttggatag aacttgctat ttaaaagagg tgtggggtaa atccttgtat aaatctccag |
| 3601 | tttagccttt tttgaaaaag ctagactttc aaatactaat ttcacttcaa gcagggtacg |
| 3661 | tttctggttt gtttgcttga cttcagtcac aatttcttat cagaccaatg gctgacctct |
| 3721 | ttgagatgtc aggctaggct tacctatgtg ttctgtgtca tgtgaatgct gagaagtttg |
| 3781 | acagagatcc aacttcagcc ttgaccccat cagtccctcg ggttaactaa ctgagccacc |
| 3841 | ggtcctcatg gctattttaa tgagggtatt gatggttaaa tgcatgtctg atcccttatc |
| 3901 | ccagccattt gcactgccag ctgggaacta taccagacct ggatactgat cccaaagtgt |
| 3961 | taaattcaac tacatgctgg agattagaga tggtgccaat aaaggaccca gaaccaggat |
| 4021 | cttgattgct atagacttat taataatcca ggtcaaagag agtgacacac actctctcaa |
| 4081 | gacctggggt gagggagtct gtgttatctg caaggccatt tgaggctcag aaagtctctc |
| 4141 | tttcctatag atatatgcat actttctgac atataggaat gtatcaggaa tactcaacca |
| 4201 | tcacaggcat gttcctacct cagggccttt acatgtcctg tttactctgt ctagaatgtc |
| 4261 | cttctgtaga tgacctggct tgcctcgtca cccttcaggt ccttgctcaa gtgtcatctt |
| 4321 | ctcccctagt taaactaccc cacaccctgt ctgctttcct tgcttatttt tctccatagc |
| 4381 | attttaccat ctcttacatt agacattttt cttatttatt tgtagtttat aagcttcatg |
| 4441 | aggcaagtaa ctttgctttg tttcttgctg tatctccagt gcccagagca gtgcctggta |
| 4501 | tataataaat atttattgac tgagtgaaaa aaaaaaaaaa aaa. |

Compositions of the disclosure may comprise a gRNA comprising a spacer sequence that specifically binds to a target sequence of an RNA molecule encoding CD80 protein comprising or consisting of 20 nucleotides of the sequence of

| (SEQ ID NO: 224) | |
|---|---|
| 1 | gacaagtact gagtgaactc aaaccctctg taaagtaaca gaagttagaa ggggaaatgt |
| 61 | cgcctctctg aagattaccc aaagaaaaag tgatttgtca ttgctttata gactgtaaga |
| 121 | agagaacatc tcagaagtgg agtcttaccc tgaaatcaaa ggatttaaag aaaaagtgga |
| 181 | atttttcttc agcaagctgt gaaactaaat ccacaacctt tggagaccca ggaacaccct |
| 241 | ccaatctctg tgtgttttgt aaacatcact ggagggtctt ctacgtgagc aattggattg |
| 301 | tcatcagccc tgcctgtttt gcacctggga agtgccctgg tcttacttgg gtccaaattg |
| 361 | ttggctttca cttttgaccc taagcatctg aagccatggg ccacacacgg aggcagggaa |
| 421 | catcaccatc caagtgtcca tacctcaatt tctttcagct cttggtgctg gctggtcttt |
| 481 | ctcacttctg ttcaggtgtt atccacgtga ccaaggaagt gaaagaagtg gcaacgctgt |
| 541 | cctgtggtca caatgtttct gttgaagagc tggcacaaac tcgcatctac tggcaaaagg |
| 601 | agaagaaaat ggtgctgact atgatgtctg gggacatgaa tatatggccc gagtacaaga |
| 661 | accggaccat ctttgatatc actaataacc tctccattgt gatcctggct ctgcgcccat |
| 721 | ctgacgaggg cacatacgag tgtgttgttc tgaagtatga aaaagacgct ttcaagcggg |
| 781 | aacacctggc tgaagtgacg ttatcagtca aagctgactt ccctacacct agtatatctg |
| 841 | actttgaaat tccaacttct aatattagaa ggataatttg ctcaacctct ggaggttttc |
| 901 | cagagcctca cctctcctgg ttggaaaatg gagaagaatt aaatgccatc aacacaacag |
| 961 | tttcccaaga tcctgaaact gagctctatg ctgttagcag caaactggat ttcaatatga |
| 1021 | caaccaacca cagcttcatg tgtctcatca agtatggaca tttaagagtg aatcagacct |
| 1081 | tcaactggaa tacaaccaag caagagcatt ttcctgataa cctgctccca tcctgggcca |
| 1141 | ttaccttaat ctcagtaaat ggaatttttg tgatatgctg cctgacctac tgctttgccc |
| 1201 | caagatgcag agagagaagg aggaatgaga gattgagaag ggaaagtgta cgccctgtat |
| 1261 | aacagtgtcc gcagaagcaa ggggctgaaa agatctgaag gtcccacctc catttgcaat |
| 1321 | tgacctcttc tgggaacttc ctcagatgga caagattacc ccaccttgcc ctttacgtat |
| 1381 | ctgctcttag gtgcttcttc acttcagttg ctttgcagga agtgtctaga ggaatatggt |
| 1441 | gggcacagaa gtagctctgg tgaccttgat caaggtgttt tgaaatgcag aattcttgag |
| 1501 | ttctggaagg gactttagag aataccagtg ttattaatga caaaggcact gaggcccagg |
| 1561 | gaggtgaccc gaattataaa ggccagcgcc agaacccaga tttcctaact ctggtgctct |
| 1621 | ttccctttat cagtttgact gtggcctgtt aactggtata tacatatata tgtcaggcaa |
| 1681 | agtgctgctg gaagtagaat ttgtccaata acaggtcaac ttcagagact atctgatttc |
| 1741 | ctaatgtcag agtagaagat tttatgctgc tgtttacaaa agcccaatgt aatgcatagg |
| 1801 | aagtatggca tgaacatctt taggagacta atggaaatat tattggtgtt tacccagtat |
| 1861 | tccatttttt tcattgtgtt ctctattgct gctctctcac tcccccatga ggtacagcag |
| 1921 | aaaggagaac tatccaaaac taatttcctc tgacatgtaa gacgaatgat ttaggtacgt |
| 1981 | caaagcagta gtcaaggagg aaagggatag tccaaagact taactggttc atattggact |
| 2041 | gataatctct ttaaatggct ttatgctagt ttgacctcat ttgtaaaata tttatgagaa |
| 2101 | agttctcatt taaaatgaga tcgttgttta cagtgtatgt actaagcagt aagctatctt |
| 2161 | caaatgtcta aggtagtaac tttccatagg gcctccttag atccctaaga tggctttttc |
| 2221 | tccttggtat ttctgggtct ttctgacatc agcagagaac tggaaagaca tagccaactg |
| 2281 | ctgttcatgt tactcatgac tcctttctct aaaactgcct tccacaattc actagaccag |
| 2341 | aagtggacgc aacttaagct gggataatca cattatcatc tgaaaatctg gagttgaaca |
| 2401 | gcaaaagaag acaacatttc tcaaatgcac atctcatggc agctaagcca catggctggg |
| 2461 | atttaaagcc tttagagcca gcccatggct ttagctacct cactatgctg cttcacaaac |
| 2521 | cttgctcctg tgtaaaacta tattctcagt gtagggcaga gaggtctaac accaacataa |
| 2581 | ggtactagca gtgtttcccg tattgacagg aatacttaac tcaataattc ttttcttttc |
| 2641 | catttagtaa cagttgtgat gactatgttt ctattctaag taattcctgt attctacagc |
| 2701 | agatactttg tcagcaatac taagggaaga aacaaagttg aaccgtttct ttaataa |

Exemplary gRNA spacer sequences of the disclosure that specifically bind to a target sequence of an RNA molecule encoding a CD80 protein of the disclosure may comprise or consist of a nucleic acid having a sequence selected from any one of comprising SEQ ID NO: 330 to SEQ ID NO: 3067.

Compositions of the disclosure may comprise a gRNA comprising a spacer sequence that specifically binds to a target sequence of an RNA molecule encoding CD86 protein comprising or consisting of 20 nucleotides of the sequence of:

| (SEQ ID NO: 226) | |
|---|---|
| 1 | agtcattgcc gaggaaggct tgcacagggt gaaagctttg cttctctgct gctgtaacag |
| 61 | ggactagcac agacacacgg atgagtgggg tcatttccag atattaggtc acagcagaag |
| 121 | cagccaaaat ggatccccag tgcactatgg gactgagtaa cattctcttt gtgatggcct |
| 181 | tcctgctctc tggtgctgct cctctgaaga ttcaagctta tttcaatgag actgcagacc |
| 241 | tgccatgcca atttgcaaac tctcaaaacc aaagcctgag tgagctagta gtattttggc |
| 301 | aggaccagga aaacttggtt ctgaatgagg tatacttagg caaagagaaa tttgacagtg |
| 361 | ttcattccaa gtatatgggc cgcacaagtt ttgattcgga cagttggacc ctgagacttc |
| 421 | acaatcttca gatcaaggac aagggcttgt atcaatgtat catccatcac aaaaagccca |
| 481 | caggaatgat tcgcatccac cagatgaatt ctgaactgtc agtgcttgct aacttcagtc |
| 541 | aacctgaaat agtaccaatt tctaatataa cagaaaatgt gtacataaat ttgacctgct |
| 601 | catctataca cggttaccca gaacctaaga agatgagtgt tttgctaaga accaagaatt |
| 661 | caactatcga gtatgatggt attatgcaga aatctcaaga taatgtcaca gaactgtacg |
| 721 | acgtttccat cagcttgtct gtttcattcc ctgatgttac gagcaatatg accatcttct |
| 781 | gtattctgga aactgacaag acgcggcttt tatcttcacc tttctctata gagcttgagg |
| 841 | accctcagcc tcccccagac cacattcctt ggattacagc tgtacttcca acagttatta |
| 901 | tatgtgtgat ggttttctgt ctaattctat ggaaatggaa gaagaagaag cggcctcgca |
| 961 | actcttataa atgtggaacc aacacaatgg agagggaaga gagtgaacag accaagaaaa |
| 1021 | gagaaaaaat ccatatacct gaaagatctg atgaagccca gcgtgttttt aaaagttcga |
| 1081 | agacatcttc atgcgacaaa agtgatacat gtttttaatt aaagagtaaa gcccatacaa |
| 1141 | gtattcattt tttctaccct ttcctttgta agttcctggg caaccttttt gatttcttcc |
| 1201 | agaaggcaaa aagacattac catgagtaat aagggggctc caggactccc tctaagtgga |
| 1261 | atagcctccc tgtaactcca gctctgctcc gtatgccaag aggagacttt aattctctta |
| 1321 | ctgcttcttt tcacttcaga gcacacttat gggccaagcc cagcttaatg gctcatgacc |
| 1381 | tggaaataaa atttaggacc aatacctcct ccagatcaga ttcttctctt aatttcatag |
| 1441 | attgtgtttt ttttttaaat agacctctca atttctggaa aactgccttt tatctgccca |
| 1501 | gaattctaag ctggtgcccc actgaatttt gtgtacctgt gactaaacaa ctacctcctc |
| 1561 | agtctgggtg ggacttatgt atttatgacc ttatagtgtt aatatcttga aacatagaga |
| 1621 | tctatgtact gtaatagtgt gattactatg ctctagagaa aagtctaccc ctgctaagga |
| 1681 | gttctcatcc ctctgtcagg gtcagtaagg aaaacggtgg cctagggtac aggcaacaat |
| 1741 | gagcagacca acctaaattt ggggaaatta ggagaggcag agatagaacc tggagccact |
| 1801 | tctatctggg ctgttgctaa tattgaggag gcttgcccca cccaacaagc catagtggag |
| 1861 | agaactgaat aaacaggaaa atgccagagc ttgtgaaccc tgtttctctt gaagaactga |
| 1921 | ctagtgagat ggcctgggga agctgtgaaa gaaccaaaag agatcacaat actcaaaaga |
| 1981 | gagagagaga gaaaaaagag agatcttgat ccacagaaat acatgaaatg tctggtctgt |
| 2041 | ccaccccatc aacaagtctt gaaacaagca acagatggat agtctgtcca aatggacata |
| 2101 | agacagacag cagtttccct ggtggtcagg gaggggtttt ggtgataccc aagttattgg |
| 2161 | gatgtcatct tcctggaagc agagctgggg agggagagcc atcaccttga taatgggatg |
| 2221 | aatggaagga ggcttaggac tttccactcc tggctgagag aggaagagct gcaacggaat |
| 2281 | taggaagacc aagacacaga tcacccgggg cttacttagc ctacagatgt cctacgggaa |
| 2341 | cgtgggctgg cccagcatag ggctagcaaa tttgagttgg atgattgttt ttgctcaagg |
| 2401 | caaccagagg aaacttgcat acagagacag atatactggg agaaatgact ttgaaaacct |
| 2461 | ggctctaagg tgggatcact aagggatggg gcagtctctg cccaaacata aagagaactc |
| 2521 | tggggagcct gagccacaaa aatgttcctt tattttatgt aaaccctcaa gggttataga |
| 2581 | ctgccatgct agacaagctt gtccatgtaa tattcccatg tttttaccct gcccctgcct |
| 2641 | tgattagact cctagcacct ggctagtttc taacatgttt tgtgcagcac agtttttaat |
| 2701 | aaatgcttgt tacattcatt taaaaaaaaa aaaaa. |

Exemplary gRNA spacer sequences of the disclosure that specifically bind to a target sequence of an RNA molecule encoding a CD86 protein of the disclosure may comprise or consist of a nucleic acid having a sequence selected from any one of SEQ ID NO: 3068 to SEQ ID NO: 5783.

Compositions of the disclosure may comprise a gRNA comprising a spacer sequence that specifically binds to a target sequence of an RNA molecule encoding CD86 protein comprising or consisting of 20 nucleotides of the sequence of

| (SEQ ID NO: 227) | |
|---|---|
| 1 | ccctttctgt atttgagttc taccgtcagt cctggcatta tttctctctc tacaaggagc |
| 61 | cttaggaggt acggggagct cgcaaatact ccttttggtt tattcttacc accttgcttc |
| 121 | tgtgttcctt gggaatgctg ctgtgcttat gcatctggtc tctttttgga gctacagtgg |
| 181 | acaggcattt gtgacagcac tatgggactg agtaacattc tctttgtgat ggccttcctg |
| 241 | ctctctggtg ctgctcctct gaagattcaa gcttatttca atgagactgc agacctgcca |
| 301 | tgccaatttg caaactctca aaaccaaagc ctgagtgagc tagtagtatt ttggcaggac |
| 361 | caggaaaact tggttctgaa tgaggtatac ttaggcaaag agaaatttga cagtgttcat |
| 421 | tccaagtata tgggccgcac aagttttgat tcggacagtt ggaccctgag acttcacaat |
| 481 | cttcagatca aggacaaggg cttgtatcaa tgtatcatcc atcacaaaaa gcccacagga |
| 541 | atgattcgca tccaccagat gaattctgaa ctgtcagtgc ttgctaactt cagtcaacct |
| 601 | gaaatagtac caatttctaa tataacagaa aatgtgtaca taaatttgac ctgctcatct |
| 661 | atacacggtt acccagaacc taagaagatg agtgttttgc taagaaccaa gaattcaact |
| 721 | atcgagtatg atggtattat gcagaaatct caagataatg tcacagaact gtacgacgtt |
| 781 | tccatcagct tgtctgtttc attccctgat gttacgagca atatgaccat cttctgtatt |
| 841 | ctggaaactg acaagacgcg gcttttatct tcacctttct ctatagagct tgaggaccct |
| 901 | cagcctcccc cagaccacat tccttggatt acagctgtac ttccaacagt tattatatgt |
| 961 | gtgatggttt tctgtctaat tctatggaaa tggaagaaga agaagcggcc tcgcaactct |
| 1021 | tataaatgtg gaaccaacac aatggagagg gaagagagtg aacagaccaa gaaaagagaa |
| 1081 | aaaatccata tacctgaaag atctgatgaa gcccagcgtg tttttaaaag ttcgaagaca |
| 1141 | tcttcatgcg acaaaagtga tacatgtttt taattaaaga gtaaagccca tacaagtatt |
| 1201 | cattttttct accctttcct ttgtaagttc ctgggcaacc tttttgattt cttccagaag |
| 1261 | gcaaaaagac attaccatga gtaataaggg ggctccagga ctccctctaa gtggaatagc |
| 1321 | ctccctgtaa ctccagctct gctccgtatg ccaagaggag actttaattc tcttactgct |
| 1381 | tcttttcact tcagagcaca cttatgggcc aagcccagct taatggctca tgacctggaa |
| 1441 | ataaaattta ggaccaatac ctcctccaga tcagattctt ctcttaattt catagattgt |
| 1501 | gttttttttt taaatagacc tctcaatttc tggaaaactg ccttttatct gcccagaatt |
| 1561 | ctaagctggt gccccactga attttgtgta cctgtgacta aacaactacc tcctcagtct |
| 1621 | gggtgggact tatgtattta tgaccttata gtgttaatat cttgaaacat agagatctat |
| 1681 | gtactgtaat agtgtgatta ctatgctcta gagaaaagtc tacccctgct aaggagttct |
| 1741 | catccctctg tcagggtcag taaggaaaac ggtggcctag ggtacaggca acaatgagca |
| 1801 | gaccaaccta aatttgggga aattaggaga ggcagagata gaacctggag ccacttctat |
| 1861 | ctgggctgtt gctaatattg aggaggcttg ccccacccaa caagccatag tggagagaac |
| 1921 | tgaataaaca ggaaaatgcc agagcttgtg aaccctgttt ctcttgaaga actgactagt |
| 1981 | gagatggcct ggggaagctg tgaaagaacc aaaagagatc acaatactca aaagagagag |
| 2041 | agagagaaaa aagagagatc ttgatccaca gaaatacatg aaatgtctgg tctgtccacc |
| 2101 | ccatcaacaa gtcttgaaac aagcaacaga tggatagtct gtccaaatgg acataagaca |
| 2161 | gacagcagtt tccctggtgg tcagggaggg gttttggtga tacccaagtt attgggatgt |
| 2221 | catcttcctg gaagcagagc tggggaggga gagccatcac cttgataatg ggatgaatgg |
| 2281 | aaggaggctt aggactttcc actcctggct gagagaggaa gagctgcaac ggaattagga |
| 2341 | agaccaagac acagatcacc cggggcttac ttagcctaca gatgtcctac gggaacgtgg |
| 2401 | gctggcccag catagggcta gcaaatttga gttggatgat tgtttttgct caaggcaacc |
| 2461 | agaggaaact tgcatacaga gacagatata ctgggagaaa tgactttgaa aacctggctc |
| 2521 | taaggtggga tcactaaggg atggggcagt ctctgcccaa acataaagag aactctgggg |
| 2581 | agcctgagcc acaaaaatgt tcctttattt tatgtaaacc ctcaagggtt atagactgcc |
| 2641 | atgctagaca agcttgtcca tgtaatattc ccatgttttt accctgcccc tgccttgatt |
| 2701 | agactcctag cacctggcta gtttctaaca tgttttgtgc agcacagttt ttaataaatg |
| 2761 | cttgttacat tcatttaaaa aaaaaaaaaa. |

Compositions of the disclosure may comprise a gRNA comprising a spacer sequence that specifically binds to a target sequence of an RNA molecule encoding CD86 protein comprising or consisting of 20 nucleotides of the sequence of

| (SEQ ID NO: 228) | |
|---|---|
| 1 | ccctttctgt atttgagttc taccgtcagt cctggcatta tttctctctc tacaaggagc |
| 61 | cttaggaggt acggggagct cgcaaatact ccttttggtt tattcttacc accttgcttc |
| 121 | tgtgttcctt gggaatgctg ctgtgcttat gcatctggtc tctttttgga gctacagtgg |
| 181 | acaggcattt gtgacagcac tatgggactg agtaacattc tctttgtgat ggccttcctg |
| 241 | ctctctggtg ctgctcctct gaagattcaa gcttatttca atgagactgc agacctgcca |
| 301 | tgccaatttg caaactctca aaaccaaagc ctgagtgagc tagtagtatt ttggcaggac |
| 361 | caggaaaact tggttctgaa tgaggtatac ttaggcaaag agaaatttga cagtgttcat |
| 421 | tccaagtata tgggccgcac aagttttgat tcggacagtt ggaccctgag acttcacaat |
| 481 | cttcagatca aggacaaggg cttgtatcaa tgtatcatcc atcacaaaaa gcccacagga |
| 541 | atgattcgca tccaccagat gaattctgaa ctgtcagtgc ttgctaactt cagtcaacct |
| 601 | gaaatagtac caatttctaa tataacagaa aatgtgtaca taaatttgac ctgctcatct |
| 661 | atacacggtt acccagaacc taagaagatg agtgttttgc taagaaccaa gaattcaact |
| 721 | atcgagtatg atggtattat gcagaaatct caagataatg tcacagaact gtacgacgtt |
| 781 | tccatcagct tgtctgtttc attccctgat gttacgagca atatgaccat cttctgtatt |
| 841 | ctggaaactg acaagacgcg gcttttatct tcacctttct ctataggaac caacacaatg |
| 901 | gagagggaag agagtgaaca gaccaagaaa agagaaaaaa tccatatacc tgaaagatct |
| 961 | gatgaagccc agcgtgtttt taaaagttcg aagacatctt catgcgacaa aagtgataca |
| 1021 | tgtttttaat taaagagtaa agcccataca agtattcatt ttttctaccc tttcctttgt |
| 1081 | aagttcctgg gcaacctttt tgatttcttc cagaaggcaa aaagacatta ccatgagtaa |
| 1141 | taagggggct ccaggactcc ctctaagtgg aatagcctcc ctgtaactcc agctctgctc |
| 1201 | cgtatgccaa gaggagactt taattctctt actgcttctt ttcacttcag agcacactta |
| 1261 | tgggccaagc ccagcttaat ggctcatgac ctggaaataa aatttaggac caatacctcc |
| 1321 | tccagatcag attcttctct taatttcata gattgtgttt tttttttaaa tagacctctc |
| 1381 | aatttctgga aaactgcctt ttatctgccc agaattctaa gctggtgccc cactgaattt |
| 1441 | tgtgtacctg tgactaaaca actacctcct cagtctgggt gggacttatg tatttatgac |
| 1501 | cttatagtgt taatatcttg aaacatagag atctatgtac tgtaatagtg tgattactat |
| 1561 | gctctagaga aaagtctacc cctgctaagg agttctcatc cctctgtcag ggtcagtaag |
| 1621 | gaaaacggtg gcctagggta caggcaacaa tgagcagacc aacctaaatt tggggaaatt |
| 1681 | aggagaggca gagatagaac ctggagccac ttctatctgg gctgttgcta atattgagga |
| 1741 | ggcttgcccc acccaacaag ccatagtgga gagaactgaa taaacaggaa aatgccagag |
| 1801 | cttgtgaacc ctgtttctct tgaagaactg actagtgaga tggcctgggg aagctgtgaa |
| 1861 | agaaccaaaa gagatcacaa tactcaaaag agagagagag agaaaaaaga gagatcttga |
| 1921 | tccacagaaa tacatgaaat gtctggtctg tccaccccat caacaagtct tgaaacaagc |
| 1981 | aacagatgga tagtctgtcc aaatggacat aagacagaca gcagtttccc tggtggtcag |
| 2041 | ggaggggttt tggtgatacc caagttattg ggatgtcatc ttcctggaag cagagctggg |
| 2101 | gagggagagc catcaccttg ataatgggat gaatggaagg aggcttagga ctttccactc |
| 2161 | ctggctgaga gaggaagagc tgcaacggaa ttaggaagac caagacacag atcacccggg |
| 2221 | gcttacttag cctacagatg tcctacggga acgtgggctg gcccagcata gggctagcaa |
| 2281 | atttgagttg gatgattgtt tttgctcaag gcaaccagag gaaacttgca tacagagaca |
| 2341 | gatatactgg gagaaatgac tttgaaaacc tggctctaag gtgggatcac taagggatgg |
| 2401 | ggcagtctct gcccaaacat aaagagaact ctggggagcc tgagccacaa aaatgttcct |
| 2461 | ttattttatg taaaccctca agggttatag actgccatgc tagacaagct tgtccatgta |
| 2521 | atattcccat gtttttaccc tgcccctgcc ttgattagac tcctagcacc tggctagttt |
| 2581 | ctaacatgtt ttgtgcagca cagtttttaa taaatgcttg ttacattcat ttaaaaaaaa |
| 2641 | aaaaaa. |

Compositions of the disclosure may comprise a gRNA comprising a spacer sequence that specifically binds to a target sequence of an RNA molecule encoding CD86 protein comprising or consisting of 20 nucleotides of the sequence of

| (SEQ ID NO: 229) | |
|---|---|
| 1 | agtcattgcc gaggaaggct tgcacagggt gaaagctttg cttctctgct gctgtaacag |
| 61 | ggactagcac agacacacgg atgagtgggg tcatttccag atattaggtc acagcagaag |
| 121 | cagccaaaat ggatccccag tgcactatgg gactgagtaa cattctcttt gtgatggcct |
| 181 | tcctgctctc tgctaacttc agtcaacctg aaatagtacc aatttctaat ataacagaaa |
| 241 | atgtgtacat aaatttgacc tgctcatcta tacacggtta cccagaacct aagaagatga |
| 301 | gtgttttgct aagaaccaag aattcaacta tcgagtatga tggtattatg cagaaatctc |
| 361 | aagataatgt cacagaactg tacgacgttt ccatcagctt gtctgtttca ttccctgatg |
| 421 | ttacgagcaa tatgaccatc ttctgtattc tggaaactga caagacgcgg cttttatctt |
| 481 | cacctttctc tatagagctt gaggaccctc agcctccccc agaccacatt ccttggatta |
| 541 | cagctgtact tccaacagtt attatatgtg tgatggtttt ctgtctaatt ctatggaaat |
| 601 | ggaagaagaa gaagcggcct cgcaactctt ataaatgtgg aaccaacaca atggagaggg |
| 661 | aagagagtga acagaccaag aaaagagaaa aaatccatat acctgaaaga tctgatgaag |
| 721 | cccagcgtgt ttttaaaagt tcgaagacat cttcatgcga caaaagtgat acatgttttt |
| 781 | aattaaagag taaagcccat acaagtattc attttttcta ccctttcctt tgtaagttcc |
| 841 | tgggcaacct ttttgatttc ttccagaagg caaaaagaca ttaccatgag taataagggg |
| 901 | gctccaggac tccctctaag tggaatagcc tccctgtaac tccagctctg ctccgtatgc |
| 961 | caagaggaga ctttaattct cttactgctt cttttcactt cagagcacac ttatgggcca |
| 1021 | agcccagctt aatggctcat gacctggaaa taaaatttag gaccaatacc tcctccagat |
| 1081 | cagattcttc tcttaatttc atagattgtg tttttttttt aaatagacct ctcaatttct |
| 1141 | ggaaaactgc cttttatctg cccagaattc taagctggtg ccccactgaa ttttgtgtac |
| 1201 | ctgtgactaa acaactacct cctcagtctg ggtgggactt atgtatttat gaccttatag |
| 1261 | tgttaatatc ttgaaacata gagatctatg tactgtaata gtgtgattac tatgctctag |
| 1321 | agaaaagtct acccctgcta aggagttctc atccctctgt cagggtcagt aaggaaaacg |
| 1381 | gtggcctagg gtacaggcaa caatgagcag accaacctaa atttggggaa attaggagag |
| 1441 | gcagagatag aacctggagc cacttctatc tgggctgttg ctaatattga ggaggcttgc |
| 1501 | cccacccaac aagccatagt ggagagaact gaataaacag gaaaatgcca gagcttgtga |
| 1561 | accctgtttc tcttgaagaa ctgactagtg agatggcctg gggaagctgt gaaagaacca |
| 1621 | aaagagatca caatactcaa aagagagaga gagagaaaaa agagagatct tgatccacag |
| 1681 | aaatacatga aatgtctggt ctgtccaccc catcaacaag tcttgaaaca agcaacagat |
| 1741 | ggatagtctg tccaaatgga cataagacag acagcagttt ccctggtggt cagggagggg |
| 1801 | ttttggtgat acccaagtta ttgggatgtc atcttcctgg aagcagagct ggggagggag |
| 1861 | agccatcacc ttgataatgg gatgaatgga aggaggctta ggactttcca ctcctggctg |
| 1921 | agagaggaag agctgcaacg gaattaggaa gaccaagaca cagatcaccc ggggcttact |
| 1981 | tagcctacag atgtcctacg ggaacgtggg ctggcccagc atagggctag caaatttgag |
| 2041 | ttggatgatt gtttttgctc aaggcaacca gaggaaactt gcatacagag acagatatac |
| 2101 | tgggagaaat gactttgaaa acctggctct aaggtgggat cactaaggga tggggcagtc |
| 2161 | tctgcccaaa cataaagaga actctgggga gcctgagcca caaaaatgtt cctttatttt |
| 2221 | atgtaaaccc tcaagggtta tagactgcca tgctagacaa gcttgtccat gtaatattcc |
| 2281 | catgttttta ccctgcccct gccttgatta gactcctagc acctggctag tttctaacat |
| 2341 | gttttgtgca gcacagtttt taataaatgc ttgttacatt catttaaaaa aaaaaaaaa. |

Compositions of the disclosure may comprise a gRNA comprising a spacer sequence that specifically binds to a target sequence of an RNA molecule encoding CD86 protein comprising or consisting of 20 nucleotides of the sequence of

| (SEQ ID NO: 230) | |
|---|---|
| 1 | agtcattgcc gaggaaggct tgcacagggt gaaagctttg cttctctgct gctgtaacag |
| 61 | ggactagcac agacacacgg atgagtgggg tcatttccag atattaggtc acagcagaag |
| 121 | cagccaaaat ggatccccag tggtgctgct cctctgaaga ttcaagctta tttcaatgag |
| 181 | actgcagacc tgccatgcca atttgcaaac tctcaaaacc aaagcctgag tgagctagta |
| 241 | gtattttggc aggaccagga aaacttggtt ctgaatgagg tatacttagg caaagagaaa |
| 301 | tttgacagtg ttcattccaa gtatatgggc cgcacaagtt ttgattcgga cagttggacc |
| 361 | ctgagacttc acaatcttca gatcaaggac aagggcttgt atcaatgtat catccatcac |
| 421 | aaaaagccca caggaatgat tcgcatccac cagatgaatt ctgaactgtc agtgcttgct |
| 481 | aacttcagtc aacctgaaat agtaccaatt tctaatataa cagaaaatgt gtacataaat |
| 541 | ttgacctgct catctataca cggttaccca gaacctaaga agatgagtgt tttgctaaga |
| 601 | accaagaatt caactatcga gtatgatggt attatgcaga aatctcaaga taatgtcaca |
| 661 | gaactgtacg acgtttccat cagcttgtct gtttcattcc ctgatgttac gagcaatatg |
| 721 | accatcttct gtattctgga aactgacaag acgcggcttt tatcttcacc tttctctata |
| 781 | gagcttgagg accctcagcc tcccccagac cacattcctt ggattacagc tgtacttcca |
| 841 | acagttatta tatgtgtgat ggttttctgt ctaattctat ggaaatggaa gaagaagaag |
| 901 | cggcctcgca actcttataa atgtggaacc aacacaatgg agagggaaga gagtgaacag |
| 961 | accaagaaaa gagaaaaaat ccatatacct gaaagatctg atgaagccca gcgtgttttt |
| 1021 | aaaagttcga agacatcttc atgcgacaaa agtgatacat gtttttaatt aaagagtaaa |
| 1081 | gcccatacaa gtattcattt tttctaccct ttcctttgta agttcctggg caaccttttt |
| 1141 | gatttcttcc agaaggcaaa aagacattac catgagtaat aagggggctc caggactccc |
| 1201 | tctaagtgga atagcctccc tgtaactcca gctctgctcc gtatgccaag aggagacttt |
| 1261 | aattctctta ctgcttcttt tcacttcaga gcacacttat gggccaagcc cagcttaatg |
| 1321 | gctcatgacc tggaaataaa atttaggacc aatacctcct ccagatcaga ttcttctctt |
| 1381 | aatttcatag attgtgtttt ttttttaaat agacctctca atttctggaa aactgccttt |
| 1441 | tatctgccca gaattctaag ctggtgcccc actgaatttt gtgtacctgt gactaaacaa |
| 1501 | ctacctcctc agtctgggtg ggacttatgt atttatgacc ttatagtgtt aatatcttga |
| 1561 | aacatagaga tctatgtact gtaatagtgt gattactatg ctctagagaa aagtctaccc |
| 1621 | ctgctaagga gttctcatcc ctctgtcagg gtcagtaagg aaaacggtgg cctagggtac |
| 1681 | aggcaacaat gagcagacca acctaaattt ggggaaatta ggagaggcag agatagaacc |
| 1741 | tggagccact tctatctggg ctgttgctaa tattgaggag gcttgcccca cccaacaagc |
| 1801 | catagtggag agaactgaat aaacaggaaa atgccagagc ttgtgaaccc tgtttctctt |
| 1861 | gaagaactga ctagtgagat ggcctgggga agctgtgaaa gaaccaaaag agatcacaat |
| 1921 | actcaaaaga gagagagaga gaaaaaagag agatcttgat ccacagaaat acatgaaatg |
| 1981 | tctggtctgt ccaccccatc aacaagtctt gaaacaagca acagatggat agtctgtcca |
| 2041 | aatggacata agacagacag cagtttccct ggtggtcagg gaggggtttt ggtgataccc |
| 2101 | aagttattgg gatgtcatct tcctggaagc agagctgggg agggagagcc atcaccttga |
| 2161 | taatgggatg aatggaagga ggcttaggac tttccactcc tggctgagag aggaagagct |
| 2221 | gcaacggaat taggaagacc aagacacaga tcacccgggg cttacttagc ctacagatgt |
| 2281 | cctacgggaa cgtgggctgg cccagcatag ggctagcaaa tttgagttgg atgattgttt |
| 2341 | ttgctcaagg caaccagagg aaacttgcat acagagacag atatactggg agaaatgact |
| 2401 | ttgaaaacct ggctctaagg tgggatcact aagggatggg gcagtctctg cccaaacata |
| 2461 | aagagaactc tggggagcct gagccacaaa aatgttcctt tattttatgt aaaccctcaa |
| 2521 | gggttataga ctgccatgct agacaagctt gtccatgtaa tattcccatg tttttaccct |
| 2581 | gcccctgcct tgattagact cctagcacct ggctagtttc taacatgttt tgtgcagcac |
| 2641 | agtttttaat aaatgcttgt tacattcatt taaaaaaaaa aaaaa. |

Compositions of the disclosure may comprise a gRNA comprising a spacer sequence that specifically binds to a target sequence of an RNA molecule encoding ICOSLG protein comprising or consisting of 20 nucleotides of the sequence of

| (SEQ ID NO: 231) | |
|---|---|
| 1 | AGTTAGAGCC GATCTCCCGC GCCCCGAGGT TGCTCCTCTC CGAGGTCTCC CGCGGCCCAA |
| 61 | GTTCTCCGCG CCCCGAGGTC TCCGCGCCCC GAGGTCTCCG CGGCCCGAGG TCTCCGCCCG |
| 121 | CACCATGCGG CTGGGCAGTC CTGGACTGCT CTTCCTGCTC TTCAGCAGCC TTCGAGCTGA |
| 181 | TACTCAGGAG AAGGAAGTCA GAGCGATGGT AGGCAGCGAC GTGGAGCTCA GCTGCGCTTG |
| 241 | CCCTGAAGGA AGCCGTTTTG ATTTAAATGA TGTTTACGTA TATTGGCAAA CCAGTGAGTC |
| 301 | GAAAACCGTG GTGACCTACC ACATCCCACA GAACAGCTCC TTGGAAAACG TGGACAGCCG |
| 361 | CTACCGGAAC CGAGCCCTGA TGTCACCGGC CGGCATGCTG CGGGGCGACT TCTCCCTGCG |
| 421 | CTTGTTCAAC GTCACCCCCC AGGACGAGCA GAAGTTTCAC TGCCTGGTGT TGAGCCAATC |
| 481 | CCTGGGATTC CAGGAGGTTT TGAGCGTTGA GGTTACACTG CATGTGGCAG CAAACTTCAG |
| 541 | CGTGCCCGTC GTCAGCGCCC CCCACAGCCC CTCCCAGGAT GAGCTCACCT TCACGTGTAC |
| 601 | ATCCATAAAC GGCTACCCCA GGCCCAACGT GTACTGGATC AATAAGACGG ACAACAGCCT |
| 661 | GCTGGACCAG GCTCTGCAGA ATGACACCGT CTTCTTGAAC ATGCGGGGCT TGTATGACGT |
| 721 | GGTCAGCGTG CTGAGGATCG CACGGACCCC CAGCGTGAAC ATTGGCTGCT GCATAGAGAA |
| 781 | CGTGCTTCTG CAGCAGAACC TGACTGTCGG CAGCCAGACA GGAAATGACA TCGGAGAGAG |
| 841 | AGACAAGATC ACAGAGAATC CAGTCAGTAC CGGCGAGAAA AACGCGGCCA CGTGGAGCAT |
| 901 | CCTGGCTGTC CTGTGCCTGC TTGTGGTCGT GGCGGTGGCC ATAGGCTGGG TGTGCAGGGA |
| 961 | CCGATGCCTC CAACACAGCT ATGCAGGTGC CTGGGCTGTG AGTCCGGAGA CAGAGCTCAC |
| 1021 | TGGTGAGTTT GCCGTGGGAA GCAGCAGGTT CTGGGGGGCC CAGGGGAGGC TTGGCTGCCA |
| 1081 | GCTGTCTTTC AGAGTTTCAA AAAACTTTCA AAAGGCAAAA GTCCCTTGCC TTGAACAACT |
| 1141 | GTTGTTCCTG GAGACGCAGC GAAGCCCTCG ATGGTGCGCA TGGCATTTCC TGCAGCCTCC |
| 1201 | CCTTGGCATG GGATGGCATC CTGGTGTGCA CTTTGTCACA CTGCGATGGG ATTTTCCCAA |
| 1261 | CATGCACAGA AGCAGAGAGA CGAGTGCTAG ACCCCCGCGC TCCCCAGTGC CCAGCCCCGA |
| 1321 | CCAGGGTGTC CAGGGCGGGT CCAGGCACCG GCGCCCAGCC CCCATGGGGT GTCCGGAGTG |
| 1381 | GGTCCAGGCA CCGGCGCCCA GCCCCCGTGG GGTGTCCAGG GCGGGTCCAG GCACCGGCGC |
| 1441 | CCAGCCCCTG TGGGGTGTCC GGAGTGGGTC CGGGCACCGC CAGCTTCTCT CTGTGGCAGC |
| 1501 | CACTCCTGCA GCTCTCGTTT GCCCCTCAGT TCCAGGAGCA ACATAGATGT GGATTCCTGT |
| 1561 | CCAATTTGGG AAAAATGTCC ACACACGGTC ACCCACCTGG CAGGTGCCTC TGGCTGCAAG |
| 1621 | GGGCGCTGGG CTTCGCAGGC AGGCCAGCCG GGCTCCCCGC CATGGGCCAG GATCCCCTCC |
| 1681 | GAGCCCTGTT TGCCGCCCAG GAGAAGGGGT TCCCCGGGGA CAGTGGGCTC AGGGTGTGCG |
| 1741 | CAGCCACCAT GCTGTGGTGT CACCTGTGGA CCCAGGCGAG CTGATGGCCG ACCGCAGAAA |
| 1801 | CGCACTTCCA AGGCCAGGTC GGCCCATCCA GATGATGCAG GAACACAGCT TGCTAAAAAC |
| 1861 | ACGGCCGGCC TGTTCCCGTC GGAGCCAGTC GAAGTTCCCT GAACAGGCCG CTGTTTCCGA |
| 1921 | AGCTTTAAAC CCTGTGTTTC CACCAAGCTG AGTCCTGAGA AAACCGACGT CTGCCTGCAG |
| 1981 | AAGGGAAAGG GGTGCTTCAT GTTCCTCTCT CTCCTTCATC TCCCT. |

Exemplary gRNA spacer sequences of the disclosure that specifically bind to a target sequence of an RNA molecule encoding a IOSLG protein of the disclosure may comprise or consist of a nucleic acid having a sequence selected from any one of any one of SEQ ID NO: 5784 to SEQ ID NO: 7789.

Compositions of the disclosure may comprise a gRNA comprising a spacer sequence that specifically binds to a target sequence of an RNA molecule encoding OX40L protein comprising or consisting of 20 nucleotides of the sequence of

| (SEQ ID NO: 232) | |
|---|---|
| 1 | GGCCCTGGGA CCTTTGCCTA TTTTCTGATT GATAGGCTTT GTTTTGTCTT TACCTCCTTC |
| 61 | TTTCTGGGGA AAACTTCAGT TTTATCGCAC GTTCCCCTTT TCCATATCTT CATCTTCCCT |
| 121 | CTACCCAGAT TGTGAAGATG GAAAGGGTCC AACCCCTGGA AGAGAATGTG GGAAATGCAG |
| 181 | CCAGGCCAAG ATTCGAGAGG AACAAGCTAT TGCTGGTGGC CTCTGTAATT CAGGGACTGG |
| 241 | GGCTGCTCCT GTGCTTCACC TACATCTGCC TGCACTTCTC TGCTCTTCAG GTATCACATC |
| 301 | GGTATCCTCG AATTCAAAGT ATCAAAGTAC AATTTACCGA ATATAAGAAG GAGAAAGGTT |
| 361 | TCATCCTCAC TTCCCAAAAG GAGGATGAAA TCATGAAGGT GCAGAACAAC TCAGTCATCA |
| 421 | TCAACTGTGA TGGGTTTTAT CTCATCTCCC TGAAGGGCTA CTTCTCCCAG GAAGTCAACA |
| 481 | TTAGCCTTCA TTACCAGAAG GATGAGGAGC CCCTCTTCCA ACTGAAGAAG GTCAGGTCTG |
| 541 | TCAACTCCTT GATGGTGGCC TCTCTGACTT ACAAAGACAA AGTCTACTTG AATGTGACCA |
| 601 | CTGACAATAC CTCCCTGGAT GACTTCCATG TGAATGGCGG AGAACTGATT CTTATCCATC |
| 661 | AAAATCCTGG TGAATTCTGT GTCCTTTGAG GGGCTGATGG CAATATCTAA AACCAGGCAC |
| 721 | CAGCATGAAC ACCAAGCTGG GGGTGGACAG GGCATGGATT CTTCATTGCA AGTGAAGGAG |
| 781 | CCTCCCAGCT CAGCCACGTG GGATGTGACA AGAAGCAGAT CCTGGCCCTC CCGCCCCCAC |
| 841 | CCCTCAGGGA TATTTAAAAC TTATTTTATA TACCAGTTAA TCTTATTTAT CCTTATATTT |
| 901 | TCTAAATTGC CTAGCCGTCA CACCCCAAGA TTGCCTTGAG CCTACTAGGC ACCTTTGTGA |
| 961 | GAAAGAAAAA ATAGATGCCT CTTCTTCAAG ATGCATTGTT TCTATTGGTC AGGCAATTGT |
| 1021 | CATAATAAAC TTATGTCATT GAAAACGGTA CCTGACTACC ATTTGCTGGA AATTTGACAT |
| 1081 | GTGTGTGGCA TTATCAAAAT GAAGAGGAGC AAGGAGTGAA GGAGTGGGGT TATGAATCTG |
| 1141 | CCAAAGGTGG TATGAACCAA CCCCTGGAAG CCAAAGCGGC CTCTCCAAGG TTAAATTGAT |
| 1201 | TGCAGTTTGC ATATTGCCTA AATTTAAACT TTCTCATTTG GTGGGGGTTC AAAAGAAGAA |
| 1261 | TCAGCTTGTG AAAAATCAGG ACTTGAAGAG AGCCGTCTAA GAAATACCAC GTGCTTTTTT |
| 1321 | TCTTTACCAT TTTGCTTTCC CAGCCTCCAA ACATAGTTAA TAGAAATTTC CCTTCAAAGA |
| 1381 | ACTGTCTGGG GATGTGATGC TTTGAAAAAT CTAATCAGTG ACTTAAGAGA GATTTTCTTG |
| 1441 | TATACAGGGA GAGTGAGATA ACTTATTGTG AAGGGTTAGC TTTACTGTAC AGGATAGCAG |
| 1501 | GGAACTGGAC ATCTCAGGGT AAAAGTCAGT ACGGATTTTA ATAGCCTGGG GAGGAAAACA |
| 1561 | CATTCTTTGC CACAGACAGG CAAAGCAACA CATGCTCATC CTCCTGCCTA TGCTGAGATA |
| 1621 | CGCACTCAGC TCCATGTCTT GTACACACAG AAACATTGCT GGTTTCAAGA AATGAGGTGA |
| 1681 | TCCTATTATC AAATTCAATC TGATGTCAAA TAGCACTAAG AAGTTATTGT GCCTTATGAA |
| 1741 | AAATAATGAT CTCTGTCTAG AAATACCATA GACCATATAT AGTCTCACAT TGATAATTGA |
| 1801 | AACTAGAAGG GTCTATAATC AGCCTATGCC AGGGCTTCAA TGGAATAGTA TCCCCTTATG |
| 1861 | TTTAGTTGAA ATGTCCCCTT AACTTGATAT AATGTGTTAT GCTTATGGCG CTGTGGACAA |
| 1921 | TCTGATTTTT CATGTCAACT TTCCAGATGA TTTGTAACTT CTCTGTGCCA AACCTTTTAT |
| 1981 | AAACATAAAT TTTTGAGATA TGTATTTTAA AATTGTAGCA CATGTTTCCC TGACATTTTC |
| 2041 | AATAGAGGAT ACAACATCAC AGAATCTTTC TGGATGATTC TGTGTTATCA AGGAATTGTA |
| 2101 | CTGTGCTACA ATTATCTCTA GAATCTCCAG AAAGGTGGAG GGCTGTTCGC CCTTACACTA |
| 2161 | AATGGTCTCA GTTGGATTTT TTTTTCCTGT TTTCTATTTC CTCTTAAGTA CACCTTCAAC |
| 2221 | TATATTCCCA TCCCTCTATT TTAATCTGTT ATGAAGGAAG GTAAATAAAA ATGCTAAATA |
| 2281 | GAAGAAATTG TAGGTAAGGT AAGAGGAATC AAGTTCTGAG TGGCTGCCAA GGCACTCACA |
| 2341 | GAATCATAAT CATGGCTAAA TATTTATGGA GGGCCTACTG TGGACCAGGC ACTGGGCTAA |
| 2401 | ATACTTACAT TTACAAGAAT CATTCTGAGA CAGATATTCA ATGATATCTG GCTTCACTAC |
| 2461 | TCAGAAGATT GTGTGTGTGT TTGTGTGTGT GTGTGTGTGT GTATTTCACT TTTTGTTATT |
| 2521 | GACCATGTTC TGCAAAATTG CAGTTACTCA GTGAGTGATA TCCGAAAAAG TAAACGTTTA |
| 2581 | TGACTATAGG TAATATTTAA GAAAATGCAT GGTTCATTTT TAAGTTTGGA ATTTTTATCT |
| 2641 | ATATTTCTCA CAGATGTGCA GTGCACATGC AGGCCTAAGT ATATGTTGTG TGTGTTGTTT |
| 2701 | GTCTTTGATG TCATGGTCCC CTCTCTTAGG TGCTCACTCG CTTTGGGTGC ACCTGGCCTG |
| 2761 | CTCTTCCCAT GTTGGCCTCT GCAACCACAC AGGGATATTT CTGCTATGCA CCAGCCTCAC |
| 2821 | TCCACCTTCC TTCCATCAAA AATATGTGTG TGTGTCTCAG TCCCTGTAAG TCATGTCCTT |
| 2881 | CACAGGGAGA ATTAACCCTT CGATATACAT GGCAGAGTTT TGTGGGAAAA GAATTGAATG |
| 2941 | AAAAGTCAGG AGATCAGAAT TTTAAATTTG ACTTAGCCAC TAACTAGCCA TGTAACCTTG |
| 3001 | GGAAAGTCAT TTCCCATTTC TGGGTCTTGC TTTTCTTTCT GTTAAATGAG AGGAATGTTA |
| 3061 | AATATCTAAC AGTTTAGAAT CTTATGCTTA CAGTGTTATC TGTGAATGCA CATATTAAAT |
| 3121 | GTCTATGTTC TTGTTGCTAT GAGTCAAGGA GTGTAACCTT CTCCTTTACT ATGTTGAATG |
| 3181 | TATTTTTTTC TGGACAAGCT TACATCTTCC TCAGCCATCT TTGTGAGTCC TTCAAGAGCA |
| 3241 | GTTATCAATT GTTAGTTAGA TATTTTCTAT TTAGAGAATG CTTAAGGGAT TCCAATCCCG |
| 3301 | ATCCAAATCA TAATTTGTTC TTAAGTATAC TGGGCAGGTC CCCTATTTTA AGTCATAATT |
| 3361 | TTGTATTTAG TGCTTTCCTG GCTCTCAGAG AGTATTAATA TTGATATTAA TAATATAGTT |
| 3421 | AATAGTAATA TTGCTATTTA CATGGAAACA AATAAAAGAT CTCAGAATTC ACTAAAAAAA |
| 3481 | AAAA. |

Exemplary gRNA spacer sequences of the disclosure that specifically bind to a target sequence of an RNA molecule encoding a OX40L protein of the disclosure may comprise or consist of a nucleic acid having a sequence selected from any one of any one of SEQ ID NO: 7790 to SEQ ID NO: 11254.

Compositions of the disclosure may comprise a gRNA comprising a spacer sequence that specifically binds to a target sequence of an RNA molecule encoding IL12 protein comprising or consisting of 20 nucleotides of the sequence of

| (SEQ ID NO: 233) | |
|---|---|
| 1 | TTTCGCTTTC ATTTTGGGCC GAGCTGGAGG CGGCGGGGCC GTCCCGGAAC GGCTGCGGCC |
| 61 | GGGCACCCCG GGAGTTAATC CGAAAGCGCC GCAAGCCCCG CGGGCCGGCC GCACCGCACG |
| 121 | TGTCACCGAG AAGCTGATGT AGAGAGAGAC ACAGAAGGAG ACAGAAAGCA AGAGACCAGA |
| 181 | GTCCCGGGAA AGTCCTGCCG CGCCTCGGGA CAATTATAAA AATGTGGCCC CCTGGGTCAG |
| 241 | CCTCCCAGCC ACCGCCCTCA CCTGCCGCGG CCACAGGTCT GCATCCAGCG GCTCGCCCTG |
| 301 | TGTCCCTGCA GTGCCGGCTC AGCATGTGTC CAGCGCGCAG CCTCCTCCTT GTGGCTACCC |
| 361 | TGGTCCTCCT GGACCACCTC AGTTTGGCCA GAAACCTCCC CGTGGCCACT CCAGACCCAG |
| 421 | GAATGTTCCC ATGCCTTCAC CACTCCCAAA ACCTGCTGAG GGCCGTCAGC AACATGCTCC |
| 481 | AGAAGGCCAG ACAAACTCTA GAATTTTACC CTTGCACTTC TGAAGAGATT GATCATGAAG |
| 541 | ATATCACAAA AGATAAAACC AGCACAGTGG AGGCCTGTTT ACCATTGGAA TTAACCAAGA |
| 601 | ATGAGAGTTG CCTAAATTCC AGAGAGACCT CTTTCATAAC TAATGGGAGT TGCCTGGCCT |
| 661 | CCAGAAAGAC CTCTTTTATG ATGGCCCTGT GCCTTAGTAG TATTTATGAA GACTTGAAGA |
| 721 | TGTACCAGGT GGAGTTCAAG ACCATGAATG CAAAGCTTCT GATGGATCCT AAGAGGCAGA |
| 781 | TCTTTCTAGA TCAAAACATG CTGGCAGTTA TTGATGAGCT GATGCAGGCC CTGAATTTCA |
| 841 | ACAGTGAGAC TGTGCCACAA AAATCCTCCC TTGAAGAACC GGATTTTTAT AAAACTAAAA |
| 901 | TCAAGCTCTG CATACTTCTT CATGCTTTCA GAATTCGGGC AGTGACTATT GATAGAGTGA |
| 961 | TGAGCTATCT GAATGCTTCC TAAAAAGCGA GGTCCCTCCA AACCGTTGTC ATTTTTATAA |
| 1021 | AACTTTGAAA TGAGGAAACT TTGATAGGAT GTGGATTAAG AACTAGGGAG GGGGAAAGAA |
| 1081 | GGATGGGACT ATTACATCCA CATGATACCT CTGATCAAGT ATTTTTGACA TTTACTGTGG |
| 1141 | ATAAATTGTT TTTAAGTTTT CATGAATGAA TTGCTAAGAA GGGAAAATAT CCATCCTGAA |
| 1201 | GGTGTTTTTC ATTCACTTTA ATAGAAGGGC AAATATTTAT AAGCTATTTC TGTACCAAAG |
| 1261 | TGTTTGTGGA AACAAACATG TAAGCATAAC TTATTTTAAA ATATTTATTT ATATAACTTG |
| 1321 | GTAATCATGA AAGCATCTGA GCTAACTTAT ATTTATTTAT GTTATATTTA TTAAATTATT |
| 1381 | TATCAAGTGT ATTTGAAAAA TATTTTTAAG TGTTCTAAAA ATAAAAGTAT TGAATTAAAG |
| 1441 | TGAAAAAAAA. |

Exemplary gRNA spacer sequences of the disclosure that specifically bind to a target sequence of an RNA molecule encoding an IL12 protein of the disclosure may comprise or consist of a nucleic acid having a sequence selected from any one of any one of SEQ ID NO: 11255 to SEQ ID NO: 12685.

Compositions of the disclosure may comprise a gRNA comprising a spacer sequence that specifically binds to a target sequence of an RNA molecule encoding CCR7 protein comprising or consisting of 20 nucleotides of the sequence of

| (SEQ ID NO: 234) | |
|---|---|
| 1 | CACTTCCTCC CCAGACAGGG GTAGTGCGAG GCCGGGCACA GCCTTCCTGT GTGGTTTTAC |
| 61 | CGCCCAGAGA GCGTCATGGA CCTGGGGAAA CCAATGAAAA GCGTGCTGGT GGTGGCTCTC |
| 121 | CTTGTCATTT TCCAGGTATG CCTGTGTCAA GATGAGGTCA CGGACGATTA CATCGGAGAC |
| 181 | AACACCACAG TGGACTACAC TTTGTTCGAG TCTTTGTGCT CCAAGAAGGA CGTGCGGAAC |
| 241 | TTTAAAGCCT GGTTCCTCCC TATCATGTAC TCCATCATTT GTTTCGTGGG CCTACTGGGC |
| 301 | AATGGGCTGG TCGTGTTGAC CTATATCTAT TTCAAGAGGC TCAAGACCAT GACCGATACC |
| 361 | TACCTGCTCA ACCTGGCGGT GGCAGACATC CTCTTCCTCC TGACCCTTCC CTTCTGGGCC |
| 421 | TACAGCGCGG CCAAGTCCTG GGTCTTCGGT GTCCACTTTT GCAAGCTCAT CTTTGCCATC |
| 481 | TACAAGATGA GCTTCTTCAG TGGCATGCTC CTACTTCTTT GCATCAGCAT TGACCGCTAC |
| 541 | GTGGCCATCG TCCAGGCTGT CTCAGCTCAC CGCCACCGTG CCCGCGTCCT TCTCATCAGC |
| 601 | AAGCTGTCCT GTGTGGGCAT CTGGATACTA GCCACAGTGC TCTCCATCCC AGAGCTCCTG |
| 661 | TACAGTGACC TCCAGAGGAG CAGCAGTGAG CAAGCGATGC GATGCTCTCT CATCACAGAG |
| 721 | CATGTGGAGG CCTTTATCAC CATCCAGGTG GCCCAGATGG TGATCGGCTT TCTGGTCCCC |
| 781 | CTGCTGGCCA TGAGCTTCTG TTACCTTGTC ATCATCCGCA CCCTGCTCCA GGCACGCAAC |
| 841 | TTTGAGCGCA ACAAGGCCAT CAAGGTGATC ATCGCTGTGG TCGTGGTCTT CATAGTCTTC |
| 901 | CAGCTGCCCT ACAATGGGGT GGTCCTGGCC CAGACGGTGG CCAACTTCAA CATCACCAGT |
| 961 | AGCACCTGTG AGCTCAGTAA GCAACTCAAC ATCGCCTACG ACGTCACCTA CAGCCTGGCC |
| 1021 | TGCGTCCGCT GCTGCGTCAA CCCTTTCTTG TACGCCTTCA TCGGCGTCAA GTTCCGCAAC |
| 1081 | GATCTCTTCA AGCTCTTCAA GGACCTGGGC TGCCTCAGCC AGGAGCAGCT CCGGCAGTGG |
| 1141 | TCTTCCTGTC GGCACATCCG GCGCTCCTCC ATGAGTGTGG AGGCCGAGAC CACCACCACC |
| 1201 | TTCTCCCCAT AGGCGACTCT TCTGCCTGGA CTAGAGGGAC CTCTCCCAGG GTCCCTGGGG |
| 1261 | TGGGGATAGG GAGCAGATGC AATGACTCAG GACATCCCCC CGCCAAAAGC TGCTCAGGGA |
| 1321 | AAAGCAGCTC TCCCCTCAGA GTGCAAGCCC CTGCTCCAGA AGATAGCTTC ACCCCAATCC |
| 1381 | CAGCTACCTC AACCAATGCC AAAAAAAGAC AGGGCTGATA AGCTAACACC AGACAGACAA |
| 1441 | CACTGGGAAA CAGAGGCTAT TGTCCCCTAA ACCAAAAACT GAAAGTGAAA GTCCAGAAAC |
| 1501 | TGTTCCCACC TGCTGGAGTG AAGGGGCCAA GGAGGGTGAG TGCAAGGGGC GTGGGAGTGG |
| 1561 | CCTGAAGAGT CCTCTGAATG AACCTTCTGG CCTCCCACAG ACTCAAATGC TCAGACCAGC |
| 1621 | TCTTCCGAAA ACCAGGCCTT ATCTCCAAGA CCAGAGATAG TGGGGAGACT TCTTGGCTTG |
| 1681 | GTGAGGAAAA GCGGACATCA GCTGGTCAAA CAAACTCTCT GAACCCCTCC CTCCATCGTT |
| 1741 | TTCTTCACTG TCCTCCAAGC CAGCGGGAAT GGCAGCTGCC ACGCCGCCCT AAAAGCACAC |
| 1801 | TCATCCCCTC ACTTGCCGCG TCGCCCTCCC AGGCTCTCAA CAGGGGAGAG TGTGGTGTTT |
| 1861 | CCTGCAGGCC AGGCCAGCTG CCTCCGCGTG ATCAAAGCCA CACTCTGGGC TCCAGAGTGG |
| 1921 | GGATGACATG CACTCAGCTC TTGGCTCCAC TGGGATGGGA GGAGAGGACA AGGGAAATGT |
| 1981 | CAGGGGCGGG GAGGGTGACA GTGGCCGCCC AAGGCCCACG AGCTTGTTCT TTGTTCTTTG |
| 2041 | TCACAGGGAC TGAAAACCTC TCCTCATGTT CTGCTTTCGA TTCGTTAAGA GAGCAACATT |
| 2101 | TTACCCACAC ACAGATAAAG TTTTCCCTTG AGGAAACAAC AGCTTTAAAA GAAAAAGAAA |
| 2161 | AAAAAAGTCT TTGGTAAATG GCAAAAAAAA AAAAAAAAAA AAAAAAA. |

Exemplary gRNA spacer sequences of the disclosure that specifically bind to a target sequence of an RNA molecule encoding a CCR7 protein of the disclosure may comprise or consist of a nucleic acid having a sequence selected from any one of any one of SEQ ID NO: 12686 to SEQ ID NO: 14872.

Compositions of the disclosure may comprise a gRNA comprising a spacer sequence that specifically binds to a target sequence of an RNA molecule, wherein the spacer sequence and the target sequence are reverse complements of one another. In some embodiments, compositions of the disclosure may comprise a single (i.e., singular) gRNA comprising a) a first spacer sequence that specifically binds to a first target RNA sequence and b) a second spacer sequence that specifically binds to a second target RNA sequence, wherein the first and second spacer sequences each bind different target RNA sequences. In some embodiments, first and second spacer sequences which bind different target RNA sequences are not comprised within a single (i.e., singular) gRNA but rather a first spacer sequence is comprised within a first gRNA and a second spacer sequence is comprised within a second gRNA sequence. In some embodiments, a spacer sequence disclosed herein comprises a portion of a nucleic acid sequence encoding a protein component of the adaptive immune response, wherein the protein component is selected from the group consisting of Beta-2-microglobulin (β2M), Human Leukocyte Antigen A (HLA-A), Human Leukocyte Antigen B (HLA-B), Human Leukocyte Antigen C (HLA-C), Cluster of Differentiation 28 (CD28), Cluster of Differentiation 80 (CD80), Cluster of Differentiation 86 (CD86), Inducible T-cell Costimulator (ICOS), ICOS Ligand (ICOSLG), OX40L, Interleukin 12 (IL12), and CC Chemokine Receptor 7 (CCR7). In some embodiments, a spacer which is a portion of a nucleic acid sequence encoding a protein component of an adaptive immune response is about 20 or 21 nucleotides in length.

All nucleotide sequences of the disclosure may include a uracil (U) or a thymine (T) interchangeably.

Exemplary, non-limiting Zika NS5 targeting spacer sequences of sgRNAs include, but are not limited to: gcaatgatcttcatgttgggagc (SEQ ID NO: 196), gaaccttgttgatgaactcttc (SEQ ID NO: 197), gttggtgattagagcttcattc (SEQ ID NO: 198), and gagtgatcctcgttcaagaatcc (SEQ ID NO: 199).

Exemplary, non-limiting lambda NS5 targeting spacer sequences of sgRNAs include, but are not limited to: GTGATAAGTGGAATGCCATG (SEQ ID NO: 200) and GNNNNNNNNNNNNNNNNNNNNGUUUAAGAGCUAUGCUGGAAACAGCAUAGCAAG UUUAAAUAAGGCUAGUCCGUUAUCAACUUGAAAAAGUGGCACCGAGUCGGUGCU UUUUUU (SEQ ID NO: 201).

### Methods of Simultaneous Treatment of Disease and Prevention of Immune Response

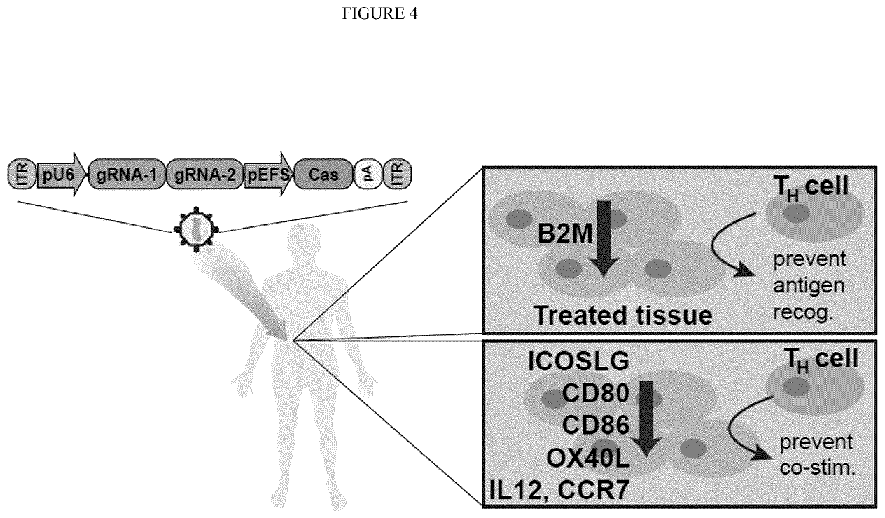

The disclosure provides compostions and methods for the simultaneous treatment of a disease or disorder in a subject by delivering a gene therapy to a cell and prevention of an immune response to the cell receiving the gene therapy. For example, the composition shown in FIG. 4 may be administered to a subject wherein gRNA 1 binds to a target sequence within an RNA molecule that encodes a component of an adaptive immune response and gRNA2 binds to a target sequence within an RNA molecule associated with a disease or disorder. By targeting an RNA molecule that encodes a component of an adaptive immune response gRNA1 prevents the display of an antigen associated with the composition or a vector comprising the composition on the surface of the cell, thereby masking the cell from the subject's immune system. gRNA2 simultaneously targets a second RNA molecule to treat a disease or disorder of the disclosure.

In alternative embodiments, gRNA1 and gRNA2 of the composition shown in FIG. 4, for example, can each target a distinct RNA molecule encoding a component of the adaptive immune response. For example, while gRNA1 targets an RNA molecule encoding a β2M polypeptide, gRNA2 targets a costimulatory molecule (ICOSLG, CD80, CD86, OX40L, IL12 or CCR7).

In some embodiments, compositions of the disclosure may comprise or consist of at least 1, 2, 3, 4, 5, 6, 7, 8, 9, or 10 gRNAs.

In some embodiments, compositions of the disclosure may comprise or consist of at least 1, 2, 3, 4, 5, 6, 7, 8, 9, or 10 gRNAs, the expression of which is under the control of a constitutive promoter (e.g. U6) and a fusion protein comprising a first RNA binding protein and a second RNA binding protein, the expression of which fusion is under the control of a viral promoter, which may be optionally constitutive (e.g. EFS).

In some embodiments, compositions of the disclosure may comprise or consist of at least 1, 2, 3, 4, 5, 6, 7, 8, 9, or 10 gRNAs, the expression of which is under the control of a first promoter and a fusion protein comprising a first RNA binding protein and a second RNA binding protein, the expression of which fusion is under the control of a second promoter, wherein the first promoter drives stronger expression of at least 1, 2, 3, 4, 5, 6, 7, 8, 9, or 10 gRNAs that the second promoter drives expression of the fusion protein. In some embodiments, compositions of the disclosure may comprise or consist of at least 1, 2, 3, 4, 5, 6, 7, 8, 9, or 10 gRNAs, the expression of which is under the control of a first promoter and a fusion protein comprising a first RNA binding protein and a second RNA binding protein, the expression of which fusion is under the control of a second promoter, wherein the first promoter drives weaker expression of at least 1, 2, 3, 4, 5, 6, 7, 8, 9, or 10 gRNAs that the second promoter drives expression of the fusion protein. By varying the relative strength of the promoters driving expression of the gRNA versus fusion protein components of the compositions of the disclosure, the compositions may be provided in ratiometric doses while expressing the gRNA and fusion protein form the same vector. Thus, the compositions of the disclosure may comprise gRNAs that bind RNA molecules associated with two or more diseases as well as two or more components of an adaptive immune response. In some embodiments, the compositions of the disclosure may comprise fusion proteins disclosed herein, wherein at least one of the fusion partner proteins is an endonuclease such as, without limitation, RNAse1, RNAse4, RNAse6, RNAse7, RNAse8, RNAse2, RNAse6PL, RNAseL, RNAseT2, RNAse11, RNAseT2-like, NOB1, ENDOV, ENDOG, ENDOD1, hFEN1, hSLFN14, hLACTB2, APEX2, ANG, HRSP12, ZC3H12A, RIDA, PDL6, NTHL, KIAA0391, APEX1, AGO2, EXOG, ZC3H12D, ERN2, PELO, YBEY, CPSF4L, hCG_2002731, ERCC1, RAC1, RAA1, RAB1, DNA2, FLJ35220, FLJ13173, ERCC4, RNAse1(K41R), RNAse1(K41R, D121E), RNAse1(K41R, D121E, H119N), RNAse1(H119N), RNAse1(R39D, N67D, N88A, G89D, R91D, H119N), RNAse1(R39D, N67D, N88A, G89D, R91D, H119N, K41R, D121E), RNAse1(R39D, N67D, N88A, G89D, R91D), TENM1, TENM2, RNAseK, TALEN, ZNF638, or PIN of hSMG6.

### Methods of Use

The disclosure provides a method of modifying level of expression of an RNA molecule of the disclosure or a protein encoded by the RNA molecule comprising contacting the composition and the RNA molecule under conditions suitable for binding of one or more of the guide RNA or the fusion protein (or a portion thereof) to the RNA molecule.

The disclosure provides a method of modifying an activity of a protein encoded by an RNA molecule comprising contacting the composition and the RNA molecule under conditions suitable for binding of one or more of the guide RNA or the fusion protein (or a portion thereof) to the RNA molecule.

The disclosure provides a method of modifying level of expression of an RNA molecule of the disclosure or a protein encoded by the RNA molecule comprising contacting the composition and a cell comprising the RNA molecule under conditions suitable for binding of one or more of the guide RNA or the fusion protein (or a portion thereof) to the RNA molecule. In some embodiments, the cell is in vivo, in vitro, ex vivo or in situ. In some embodiments, the composition comprises a vector comprising composition comprising a guide RNA of the disclosure and a fusion protein of the disclosure. In some embodiments, the vector is an AAV.

The disclosure provides a method of modifying an activity of a protein encoded by an RNA molecule comprising contacting the composition and a cell comprising the RNA molecule under conditions suitable for binding of one or more of the guide RNA or the fusion protein (or a portion thereof) to the RNA molecule. In some embodiments, the cell is in vivo, in vitro, ex vivo or in situ. In some embodiments, the composition comprises a vector comprising composition comprising a guide RNA of the disclosure and a fusion protein of the disclosure. In some embodiments, the vector is an AAV.

The disclosure provides a method of modifying level of expression of an RNA molecule of the disclosure or a protein encoded by the RNA molecule comprising contacting the composition and the RNA molecule under conditions suitable for RNA nuclease activity wherein the fusion protein induces a break in the RNA molecule.

The disclosure provides a method of modifying an activity of a protein encoded by an RNA molecule comprising contacting the composition and the RNA molecule under conditions suitable for RNA nuclease activity wherein the fusion protein induces a break in the RNA molecule.

The disclosure provides a method of modifying a level of expression of an RNA molecule of the disclosure or a protein encoded by the RNA molecule comprising contacting the composition and a cell comprising the RNA molecule under conditions suitable for RNA nuclease activity wherein the fusion protein induces a break in the RNA molecule. In some embodiments, the cell is in vivo, in vitro, ex vivo or in situ. In some embodiments, the composition comprises a vector comprising composition comprising a guide RNA of the disclosure and a fusion protein of the disclosure. In some embodiments, the vector is an AAV.

The disclosure provides a method of modifying an activity of a protein encoded by an RNA molecule comprising contacting the composition and a cell comprising the RNA molecule under conditions suitable for RNA nuclease activity wherein the fusion protein induces a break in the RNA molecule. In some embodiments, the cell is in vivo, in vitro, ex vivo or in situ. In some embodiments, the composition comprises a vector comprising composition comprising a guide RNA of the disclosure and a fusion protein of the disclosure. In some embodiments, the vector is an AAV.

The disclosure provides a method of treating a disease or disorder comprising administering to a subject a therapeutically effective amount of a composition of the disclosure.

The disclosure provides a method of treating a disease or disorder comprising administering to a subject a therapeutically effective amount of a composition of the disclosure, wherein the composition comprises a vector comprising composition comprising a guide RNA of the disclosure and a fusion protein of the disclosure and wherein the composition modifies a level of expression of an RNA molecule of the disclosure or a protein encoded by the RNA molecule.

The disclosure provides a method of treating a disease or disorder comprising administering to a subject a therapeutically effective amount of a composition of the disclosure, wherein the composition comprises a vector comprising composition comprising a guide RNA of the disclosure and a fusion protein of the disclosure and wherein the composition modifies an activity of a protein encoded by an RNA molecule.

In some embodiments of the compositions and methods of the disclosure, a disease or disorder of the disclosure includes, but is not limited to, a genetic disease or disorder. In some embodiments, the genetic disease or disorder is a single-gene disease or disorder. In some embodiments, the single-gene disease or disorder is an autosomal dominant disease or disorder, an autosomal recessive disease or disorder, an X-chromosome linked (X-linked) disease or disorder, an X-linked dominant disease or disorder, an X-linked recessive disease or disorder, a Y-linked disease or disorder or a mitochondrial disease or disorder. In some embodiments, the genetic disease or disorder is a multiple-gene disease or disorder. In some embodiments, the genetic disease or disorder is a multiple-gene disease or disorder. In some embodiments, the single-gene disease or disorder is an autosomal dominant disease or disorder including, but not limited to, Huntington's disease, neurofibromatosis type 1, neurofibromatosis type 2, Marfan syndrome, hereditary nonpolyposis colorectal cancer, hereditary multiple exostoses, Von Willebrand disease, and acute intermittent porphyria. In some embodiments, the single-gene disease or disorder is an autosomal recessive disease or disorder including, but not limited to, Albinism, Medium-chain acyl-CoA dehydrogenase deficiency, cystic fibrosis, sickle-cell disease, Tay-Sachs disease, Niemann-Pick disease, spinal muscular atrophy, and Roberts syndrome. In some embodiments, the single-gene disease or disorder is X-linked disease or disorder including, but not limited to, muscular dystrophy, Duchenne muscular dystrophy, Hemophilia, Adrenoleukodystrophy (ALD), Rett syndrome, and Hemophilia A. In some embodiments, the single-gene disease or disorder is a mitochondrial disorder including, but not limited to, Leber's hereditary optic neuropathy.

In some embodiments of the compositions and methods of the disclosure, a disease or disorder of the disclosure includes, but is not limited to, an immune disease or disorder. In some embodiments, the immune disease or disorder is an immunodeficiency disease or disorder including, but not limited to, B-cell deficiency, T-cell deficiency, neutropenia, asplenia, complement deficiency, acquired immunodeficiency syndrome (AIDS) and immunodeficiency due to medical intervention (immunosuppression as an intended or adverse effect of a medical therapy). In some embodiments, the immune disease or disorder is an autoimmune disease or disorder including, but not limited to, Achalasia, Addison's disease, Adult Still's disease, Agammaglobulinemia, Alopecia areata, Amyloidosis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Bald disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjagren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, or Wegener's granulomatosis.

In some embodiments of the compositions and methods of the disclosure, a disease or disorder of the disclosure includes, but is not limited to, an inflammatory disease or disorder. In some embodiments, the inflammatory disease or disorder includes, but is not limited to, Alzheimer's disease, ankylosing spondylitis, arthritis, osteoarthritis, rheumatoid arthritis, psoriatic arthritis, asthma, atherosclerosis, Crohn's disease, colitis, dermatitis, diverticulitis, fibromyalgia, hepatitis, irritable bowel syndrome (IBS), systemic lupus erythematous (SLE), nephritis, Parkinson's disease, ulcerative colitis, acute bronchitis, acute appendicitis, tonsillitis, infective meningitis, sinusitis, asthma, chronic peptic ulcer, tuberculosis, rheumatoid arthritis, periodontitis, gout, Scleroderma, vasculitis, and myositis.

In some embodiments of the compositions and methods of the disclosure, a disease or disorder of the disclosure includes, but is not limited to, a metabolic disease or disorder. In some embodiments of the compositions and methods of the disclosure, a disease or disorder of the disclosure includes, but is not limited to, a degenerative or a progressive disease or disorder. In some embodiments, the degenerative or a progressive disease or disorder includes, but is not limited to, amyotrophic lateral sclerosis (ALS), Huntington's disease, Alzheimer's disease, and aging.

In some embodiments of the compositions and methods of the disclosure, a disease or disorder of the disclosure includes, but is not limited to, an infectious disease or disorder.

In some embodiments of the compositions and methods of the disclosure, a disease or disorder of the disclosure includes, but is not limited to, a pediatric or a developmental disease or disorder.

In some embodiments of the compositions and methods of the disclosure, a disease or disorder of the disclosure includes, but is not limited to, a cardiovascular disease or disorder.

In some embodiments of the compositions and methods of the disclosure, a disease or disorder of the disclosure includes, but is not limited to, a proliferative disease or disorder. In some embodiments, the proliferative disease or disorder is a cancer. In some embodiments, the cancer includes, but is not limited to, Acute Lymphoblastic Leukemia (ALL), Acute Myeloid Leukemia (AML), Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Appendix Cancer, Gastrointestinal Carcinoid Tumors, Astrocytomas, Atypical Teratoid/Rhabdoid Tumor, Central Nervous System (Brain Cancer), Basal Cell Carcinoma, Bile Duct Cancer, Bladder Cancer, Bone Cancer, Ewing Sarcoma, Osteosarcoma, Malignant Fibrous Histiocytoma, Brain Tumors, Breast Cancer, Burkitt Lymphoma, Carcinoid Tumor, Carcinoma, Cardiac (Heart) Tumors, Embryonal Tumors, Germ Cell Tumor, Primary CNS Lymphoma, Cervical Cancer, Cholangiocarcinoma, Chordoma, Chronic Lymphocytic Leukemia (CLL), Chronic Myelogenous Leukemia (CML), Chronic Myeloproliferative Neoplasms, Colorectal Cancer, Craniopharyngioma, Cutaneous T-Cell Lymphoma, Ductal Carcinoma In Situ, Embryonal Tumors, Endometrial Cancer (Uterine Cancer), Ependymoma, Esophageal Cancer, Esthesioneuroblastoma (Head and Neck Cancer), Ewing Sarcoma (Bone Cancer), Extracranial Germ Cell Tumor, Extragonadal Germ Cell Tumor, Eye Cancer, Childhood Intraocular Melanoma, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Childhood Gastrointestinal Stromal Tumors, Germ Cell Tumors, Childhood Extracranial Germ Cell Tumors, Extragonadal Germ Cell Tumors, Ovarian Germ Cell Tumors, Testicular Cancer, Gestational Trophoblastic Disease, Hairy Cell Leukemia, Head and Neck Cancer, Heart Tumors, Hepatocellular (Liver) Cancer, Histiocytosis, Hodgkin Lymphoma, Hypopharyngeal Cancer (Head and Neck Cancer), Intraocular Melanoma, Islet Cell Tumors, Pancreatic Neuroendocrine Tumors, Kaposi Sarcoma (Soft Tissue Sarcoma), Kidney (Renal Cell) Cancer, Langerhans Cell Histiocytosis, Laryngeal Cancer (Head and Neck Cancer), Leukemia, Lip and Oral Cavity Cancer (Head and Neck Cancer), Liver Cancer, Lung Cancer (Non-Small Cell and Small Cell), Childhood Lung Cancer, Lymphoma, Male Breast Cancer, Malignant Fibrous Histiocytoma of Bone and Osteosarcoma, Melanoma, Merkel Cell Carcinoma (Skin Cancer), Mesothelioma, Metastatic Squamous Neck Cancer with Occult Primary (Head and Neck Cancer), Midline Tract Carcinoma With NUT Gene Changes, Mouth Cancer (Head and Neck Cancer), Multiple Endocrine Neoplasia Syndromes, Multiple Myeloma/Plasma Cell Neoplasms, Mycosis Fungoides (Lymphoma), Myelodysplastic Syndromes, Myelodysplastic/Myeloproliferative Neoplasms, Nasal Cavity and Paranasal Sinus Cancer (Head and Neck Cancer), Nasopharyngeal Cancer (Head and Neck Cancer), Neuroblastoma, Non-Hodgkin Lymphoma, Non-Small Cell Lung Cancer, Oral Cancer, Lip and Oral Cavity Cancer and Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis, Paraganglioma, Parathyroid Cancer, Penile Cancer, Pharyngeal Cancer (Head and Neck Cancer), Pheochromocytoma, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Rhabdomyosarcoma, Childhood (Soft Tissue Sarcoma), Salivary Gland Cancer (Head and Neck Cancer), Sarcoma, Childhood Rhabdomyosarcoma (Soft Tissue Sarcoma), Childhood Vascular Tumors (Soft Tissue Sarcoma), Ewing Sarcoma (Bone Cancer), Kaposi Sarcoma (Soft Tissue Sarcoma), Osteosarcoma (Bone Cancer), Uterine Sarcoma, Sézary Syndrome, Lymphoma, Skin Cancer, Small Cell Lung Cancer, Small Intestine Cancer, Soft Tissue Sarcoma, Squamous Cell Carcinoma of the Skin, Squamous Neck Cancer, Stomach (Gastric) Cancer, T-Cell Lymphoma, Testicular Cancer, Throat Cancer (Head and Neck Cancer), Nasopharyngeal Cancer, Oropharyngeal Cancer, Hypopharyngeal Cancer, Thymoma and Thymic Carcinoma, Thyroid Cancer, Transitional Cell Cancer of the Renal Pelvis and Ureter, Renal Cell Cancer, Urethral Cancer, Uterine Sarcoma, Vaginal Cancer, Vascular Tumors (Soft Tissue Sarcoma), Vulvar Cancer, Wilms Tumor and Other Childhood Kidney Tumors.

In some embodiments of the methods of the disclosure, a subject of the disclosure has been diagnosed with the disease or disorder. In some embodiments, the subject of the disclosure presents at least one sign or symptom of the disease or disorder. In some embodiments, the subject has a biomarker predictive of a risk of developing the disease or disorder. In some embodiments, the biomarker is a genetic mutation.

In some embodiments of the methods of the disclosure, a subject of the disclosure is female. In some embodiments of the methods of the disclosure, a subject of the disclosure is male. In some embodiments, a subject of the disclosure has two XX or XY chromosomes. In some embodiments, a subject of the disclosure has two XX or XY chromosomes and a third chromosome, either an X or a Y.

In some embodiments of the methods of the disclosure, a subject of the disclosure is a neonate, an infant, a child, an adult, a senior adult, or an elderly adult. In some embodiments of the methods of the disclosure, a subject of the disclosure is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 days old. In some embodiments of the methods of the disclosure, a subject of the disclosure is at least 1, 2, 3, 4, 5, 6, 7, 8, 9, 10, 11 or 12 months old. In some embodiments of the methods of the disclosure, a subject of the disclosure is at least 1, 2, 3, 4, 5, 6, 7, 8, 9, 10, 15, 20, 25, 30, 35, 40, 45, 50, 55, 60, 65, 70, 75, 80, 85, 90, 95, 100 or any number of years or partial years in between of age.

In some embodiments of the methods of the disclosure, a subject of the disclosure is a mammal. In some embodiments, a subject of the disclosure is a non-human mammal.

In some embodiments of the methods of the disclosure, a subject of the disclosure is a human.

In some embodiments of the methods of the disclosure, a therapeutically effective amount comprises a single dose of a composition of the disclosure. In some embodiments, a therapeutically effective amount comprises a therapeutically effective amount comprises at least one dose of a composition of the disclosure. In some embodiments, a therapeutically effective amount comprises a therapeutically effective amount comprises one or more dose(s) of a composition of the disclosure.

In some embodiments of the methods of the disclosure, a therapeutically effective amount eliminates a sign or symptom of the disease or disorder. In some embodiments, a therapeutically effective amount reduces a severity of a sign or symptom of the disease or disorder.

In some embodiments of the methods of the disclosure, a therapeutically effective amount eliminates the disease or disorder.

In some embodiments of the methods of the disclosure, a therapeutically effective amount prevents an onset of a disease or disorder. In some embodiments, a therapeutically effective amount delays the onset of a disease or disorder. In some embodiments, a therapeutically effective amount reduces the severity of a sign or symptom of the disease or disorder. In some embodiments, a therapeutically effective amount improves a prognosis for the subject.

In some embodiments of the methods of the disclosure, a composition of the disclosure is administered to the subject systemically. In some embodiments, the composition of the disclosure is administered to the subject by an intravenous route. In some embodiments, the composition of the disclosure is administered to the subject by an injection or an infusion.

In some embodiments of the methods of the disclosure, a composition of the disclosure is administered to the subject locally. In some embodiments, the composition of the disclosure is administered to the subject by an intraosseous, intraocular, intracerebrospinal or intraspinal route. In some embodiments, the composition of the disclosure is administered directly to the cerebral spinal fluid of the central nervous system. In some embodiments, the composition of the disclosure is administered directly to a tissue or fluid of the eye and does not have bioavailability outside of ocular structures. In some embodiments, the composition of the disclosure is administered to the subject by an injection or an infusion.

In some embodiments, the compositions comprising the RNA-binding fusion proteins disclosed herein are formulated as pharmaceutical compositions. Briefly, pharmaceutical compositions for use as disclosed herein may comprise a fusion protein(s) or a polynucleotide encoding the fusion protein(s), optionally comprised in an AAV, which is optionally also immune orthogonal, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the disclosure may be formulated for oral, intravenous, topical, enteral, intraocular, and/or parenteral administration. In certain embodiments, the compositions of the present disclosure are formulated for intravenous administration.

### EXAMPLES

### Example 1: RNA-Guided Cleavage of Viral RNA Molecules

A549 cells were cultured in DMEM with 10% FBS and 1% penicillin/streptomycin (GIBCO) and passaged at 90%-100% confluency. Cells were seeded at 1×10\hat{ }5 cells per well of a 24-well plate for RNA isolation or 0.5×10\hat{ }5 cells per well. Cells were transfected with plasmids encoding Campylobacter jejuni Cas9 (CjeCas9) fused to the gene NTHL1 (residues 31-312, E43) or CPSF4L (full length, E67) with plasmids encoding one of four sites in Zika NS5 RNA. CjeCas9 was driven by an EFS promoter while the guide RNAs were driven by U6 promoter. The sequences of the sgRNAs are presented in Table 8. The sequences of the constructs used in this stud are presented below (SEQ ID NO: 13656 and SEQ ID NO: 13657).

RNA isolations were carried out with RNAeasy columns (Qiagen) according to the manufacturer's protocol. RNA quality and concentrations were estimated using the Nanodrop spectrophotometer. cDNA preparation was done using Superscript III (Thermo) with random primers according to the manufacturer's protocol. qPCR was carried out with the following primers as listed in Table 7.

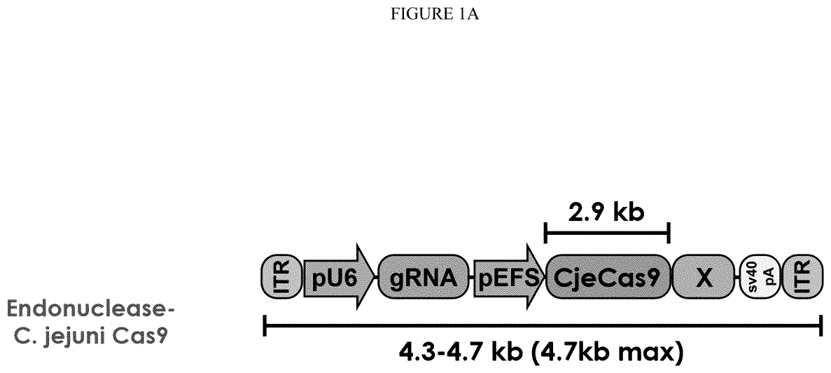

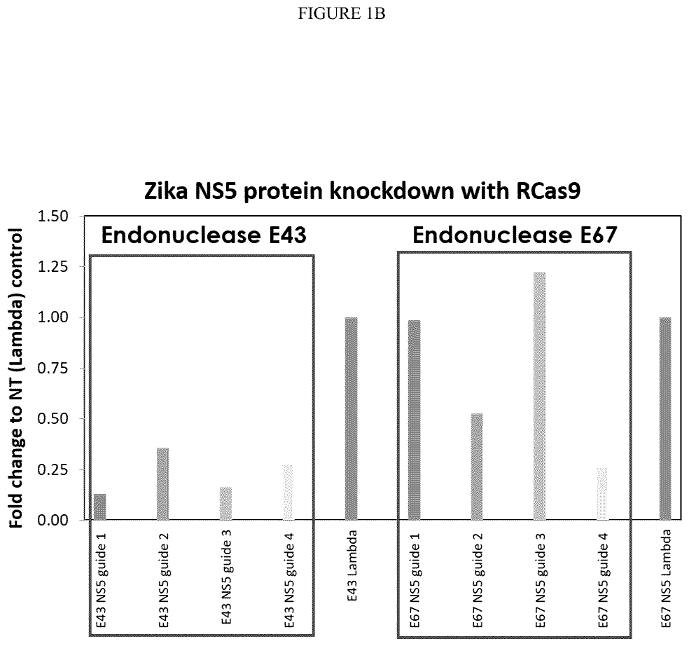

FIG. 1 shows expression levels of Zika NS5 assessed in the presence of both E43 and E67 endonucleases with sgRNAs containing the various NS5-targeting spacer sequences as indicated in Table 8. Zika NS5 expression is displayed as fold change relative to the endonuclease loaded with an sgRNA containing a control (Lambda) spacer sequence.

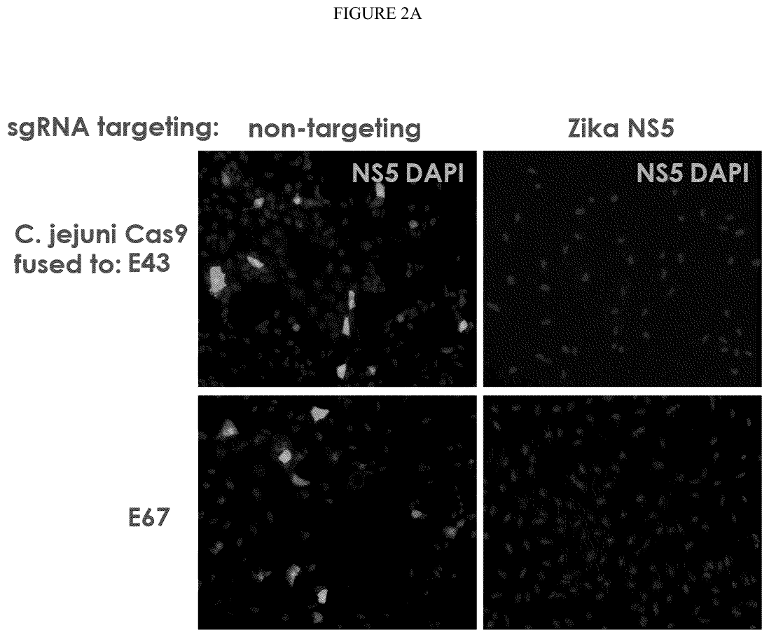

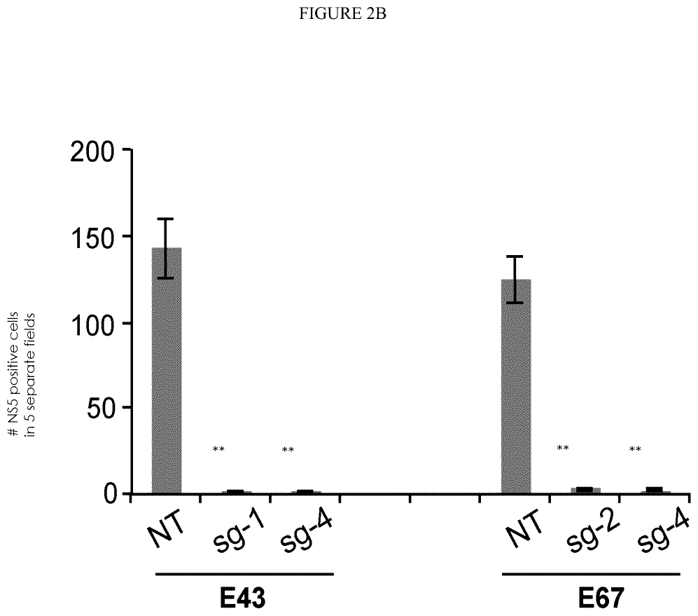

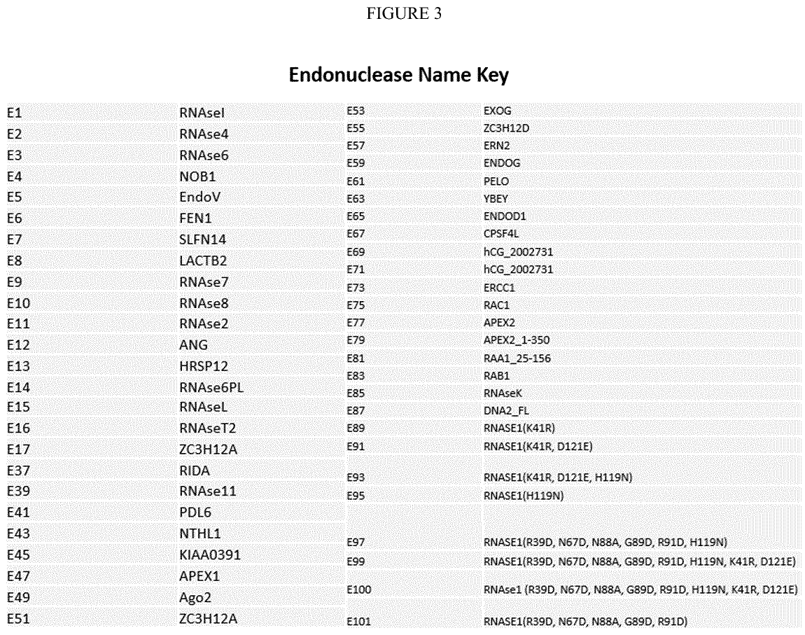

Immunofluorescence microscopy was used to visualize Zika NS5 expression in the presence of E43 or E67 endonucleases fused to CjeCas9. FIG. 2A shows a fluorescence microscopy image of cells transfected with CjeCas9-endonuclease fusions loaded with an sgRNA containing a Zika NS5-targeting spacer sequence. Expression of Zika NS5 is markedly decreased in the presence of CjeCas9-endonuclease fusions loaded with the appropriate Zika NS5-targeting sgRNA as compared to a CjeCas9-endonuclease fusion loaded with a non-Zika NS5 targeting sgRNA (FIGS. 2A and 2B). FIG. 3 is a list of exemplary endonucleases for use in the compositions of the disclosure.

TABLE 7
qPCR primers

| | |
|---|---|
| GAPDH_F | CAGCCTCAAGATCATCAGCAA (SEQ ID NO: 192) |
| GAPDH_R | TGTGGTCATGAGTCCTTCCA (SEQ ID NO: 193) |
| NS5_F | GAGGAGAGTGCCAGAGTTGT (SEQ ID NO: 194) |
| NS5_R | TCTCTCTCCCCATCCAGTGA (SEQ ID NO: 195) |

TABLE 8
sgRNA sequences

| | |
|---|---|
| NS5-targeting spacer 1 | gcaatgatcttcatgttgggagc (SEQ ID NO: 196) |
| NS5-targeting spacer 2 | gaaccttgttgatgaactcttc (SEQ ID NO: 197) |
| NS5-targeting spacer 3 | gttggtgattagagcttcattc (SEQ ID NO: 198) |
| NS5-targeting spacer 4 | gagtgatcctcgttcaagaatcc (SEQ ID NO: 199) |
| Non-targeting control | GTGATAAGTGGAATGCCATG (SEQ ID NO: 200) |
| spacer (λ2) | |
| sgRNA scaffold (N's | GNNNNNNNNNNNNNNNNNNNNGUUUAAGAGCUAUG |
| indicate spacer) | CUGGAAACAGCAUAGCAAGUUUAAAUAAGGCUAGU |
| | CCGUUAUCAACUUGAAAAAGUGGCACCGAGUCGGU |
| | GCUUUUUUU (SEQ ID NO: 201) |

A E43-CjeCas9 and sgRNA plasmid may comprise or consist of the sequence (U6: N's=sgRNA spacer, E43, CjeCas9):

(SEQ ID NO: 202)
gtttattacagggacagcagagatccagtttggttaattaaggtaccgag
ggcctatttcccatgattccttcatatttgcatatacgatacaaggctgt
tagagagataattagaattaatttgactgtaaacacaaagatattagtac
aaaatacgtgacgtagaaagtaataatttcttgggtagtttgcagtttta
aaattatgttttaaaatggactatcatatgcttaccgtaacttgaaagta
tttcgatttcttggctttatatatcttGTGGAAAGGACGAAACACCNNNN
NNNNNNNNNNNNNNNGTTTTAGTCCCTGAAGGGACTAAAATAAAGAGTTT
GCGGGACTCTGCGGGGTTACAATCCCCTAAAACCGCTTTTTTTCCTGCAG
CCCGGGGGATCCACTAGTTCTAGAGCGGCCGCCACCGCGGTGGAGCTCCA
GCTTTTGTTCCCTTTAGTGAGGGTTAATTGCGCGAATTCGCTAGCTAGGT
CTTGAAAGGAGTGGGAATTGGCTCCGGTGCCCGTCAGTGGGCAGAGCGCA
CATCGCCCACAGTCCCCGAGAAGTTGGGGGGAGGGGTCGGCAATTGATCC
GGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAG
GACCGGTTCTAGAGCGCTATTTAGAACCatgTGTTCTCCCCAAGAATCTG
GCATGACCGCTCTTTCAGCGAGGATGTTGACGCGAAGCAGATCCCTGGGA
CCTGGGGCCGGGCCACGAGGGTGTCGGGAAGAACCAGGACCGTTGCGACG
GAGGGAAGCAGCAGCGGAAGCTCGGAAATCCCATTCTCCGGTTAAACGAC
CCCGCAAGGCACAACGGCTCAGGGTTGCTTACGAGGGGAGCGATTCCGAA
AAGGGTGAAGGAGCAGAGCCCTTGAAGGTTCCAGTATGGGAACCCCAGGA
TTGGCAGCAGCAGCTTGTAAACATCCGAGCAATGAGGAACAAAAAAGATG
CACCTGTTGATCACCTCGGAACCGAACATTGTTATGATTCTAGTGCGCCG
CCAAAAGTCCGCCGGTATCAGGTTCTGTTGAGTTTGATGCTGAGTAGTCA
GACTAAGGACCAGGTTACGGCCGGAGCAATGCAACGGCTTCGGGCACGGG
GACTCACGGTCGATAGCATTTTGCAGACCGATGACGCAACATTGGGTAAA
CTCATATATCCAGTTGGCTTCTGGCGGAGCAAAGTGAAGTACATCAAGCA
GACCTCAGCCATTCTCCAACAACATTACGGAGGTGATATACCCGCAAGCG
TAGCTGAACTGGTAGCACTGCCGGGCGTCGGTCCCAAAATGGCACATCTG
GCTATGGCGGTTGCTTGGGGAACGGTGTCTGGTATCGCAGTTGATACGCA
TGTCCACCGCATCGCCAATCGGCTGAGGTGGACTAAAAAAGCCACTAAGT
CTCCTGAAGAAACACGGGCTGCTCTGGAAGAGTGGCTTCCACGAGAGCTG
TGGCATGAAATCAATGGATTGCTGGTTGGTTTCGGGCAGCAGACATGCTT
GCCCGTGCACCCCCGGTGTCATGCTTGCTTGAACCAGGCTTTGTGCCCAG
CTGCCCAGGGCCTGAGTGGAAGTGAGACACCGGGAACATCTGAGTCTGCG
ACCCCGGAGAGCacaaacGCGCGAATCCTGGCCTTCGcgATTGGCATTAG
CAGCATCGGCTGGGCATTCTCTGAAAACGACGAACTGAAGGATTGCGGCG
TGCGAATTTTCACTAAGGTCGAAAATCCCAAAACTGGTGAATCACTCGCT
CTCCCTAGACGACTGGCACGCTCCGCACGAAAGAGGCTTGCCCGCCGCAA
GGCACGCTTGAACCATCTTAAACACCTTATTGCAAATGAGTTTAAACTGA
ATTATGAGGACTACCAATCCTTTGACGAGTCTCTTGCTAAAGCCTACAAA
GGGAGCCTTATATCCCCGTATGAGCTCCGGTTCAGAGCACTCAACGAACT
GCTGTCCAAACAGGATTTTGCTCGCGTGATTCTCCACATAGCGAAGAGGC
GAGGATACGATGACATTAAAAACAGTGATGATAAGGAAAAAGGGGCCATA
CTCAAAGCGATTAAGCAAAATGAAGAGAAGCTCGCTAACTATCAATCAGT
AGGGGAGTATCTCTATAAAGAGTACTTCCAGAAGTTCAAAGAAAATAGCA
AGGAATTTACTAATGTCCGGAATAAAAAGGAGTCTTACGAAAGATGTATT
GCGCAATCTTTCCTCAAGGACGAGCTCAAATTGATTTTCAAGAAACAAAG
GGAATTTGGGTTCAGCTTCTCAAAAAAATTTGAGGAAGAGGTTCTGAGCG
TTGCCTTTTACAAACGCGCCCTTAAGGACTTCTCACATCTCGTAGGGAAT
TGTAGTTTCTTCACCGATGAAAAACGGGCGCCAAAAAATAGCCCTTTGGC
TTTTATGTTTGTCGCTCTGACTCGCATCATTAATCTGCTCAACAACCTTA
AAAACACGGAAGGGATTCTGTACACAAAGGATGATCTGAACGCTCTGCTT
AACGAAGTTTTGAAGAACGGGACTTTGACCTACAAACAAACCAAAAAGCT
TCTTGGTCTCAGTGATGACTACGAATTCAAGGGAGAAAAAGGGACATATT
TCATCGAATTCAAGAAGTATAAGGAGTTCATCAAAGCCTTGGGCGAGCAC
AACTTGTCTCAAGATGATCTCAACGAAATTGCTAAGGATATCACTCTGAT
TAAAGACGAGATCAAGCTCAAAAAGGCGTTGGCGAAGTATGACCTTAACC
AAAACCAAATAGATAGCCTCAGCAAGTTGGAATTTAAAGATCACTTGAAT
ATAAGTTTCAAGGCCCTTAAGTTGGTCACCCCCTTGATGCTTGAAGGAAA
GAAATATGATGAGGCATGTAATGAGCTGAATCTCAAGGTTGCTATTAACG
AAGACAAAAAAGATTTCCTCCCAGCTTTCAATGAGACTTACTATAAGGAC
GAGGTTACCAATCCTGTGGTGCTCCGAGCCATCAAAGAGTATCGAAAGGT
CCTGAATGCTTTGCTCAAAAAATACGGTAAGGTACACAAAATAAATATTG
AGCTCGCAAGGGAGGTCGGTAAGAACCACTCCCAGCGCGCCAAAATAGAA
AAGGAACAGAATGAAAATTACAAAGCGAAAAAGGACGCCGAGCTCGAGTG
CGAAAAGCTGGGCCTGAAAATAAACAGCAAGAACATTCTCAAACTCCGCC
TCTTCAAAGAACAAAAAGAATTTTGTGCTTATAGTGGTGAGAAAATAAAA
ATCTCCGATCTTCAAGACGAGAAGATGCTCGAAATAGACgcgATATATCC
ATATAGCAGGTCTTTTGACGATTCTTACATGAATAAAGTGCTTGTTTTCA
CTAAGCAGAATCAGGAAAAGTTGAATCAGACCCCCTTTGAGGCCTTTGGC
AACGACTCAGCAAAGTGGCAGAAGATCGAGGTCTTGGCTAAGAATCTTCC
TACTAAGAAACAGAAAAGGATATTGGATAAGAACTATAAAGACAAAGAAC
AAAAGAACTTTAAAGACCGCAACCTCAATGACACCAGATACATAGCAAGA
TTGGTTCTGAACTACACAAAAGATTATTTGGACTTCTTGCCGCTGTCTGA
TGATGAGAACACGAAACTCAACGACACGCAAAAGGGGTCTAAAGTCCACG
TCGAAGCTAAATCTGGGATGCTCACCTCAGCATTGAGGCATACGTGGGGA
TTCTCAGCAAAGGACCGAAACAATCACCTGCACCATGCCATTGACGCAGT
TATCATAGCGTATGCCAATAATTCAATAGTAAAAGCGTTTAGCGACTTCA
AGAAGGAACAAGAGTCCAACAGCGCCGAGCTCTACGCAAAAAAGATTAGT
GAACTCGACTACAAAAACAAAAGAAAATTCTTTGAGCCGTTCAGCGGATT
TCGACAGAAGGTATTGGATAAAATAGATGAAATTTTCGTGAGCAAACCCG
AAAGGAAAAAGCCCTCAGGCGCCTTGCACGAAGAGACTTTCAGGAAGGAA
GAGGAATTCTACCAAAGCTACGGCGGAAAAGAGGGAGTTTTGAAGGCTCT
CGAACTTGGAAAGATTAGGAAGGTGAACGGCAAGATAGTGAAAAACGGCG
ATATGTTCCGGGTTGATATCTTCAAACATAAAAAAACGAATAAATTTTAT
GCTGTGCCTATATACACTATGGACTTCGCACTTAAGGTCCTGCCGAATAA
GGCGGTAGCCCGATCTAAAAAAGGCGAAATTAAGGACTGGATTTTGATGG
ATGAAAATTACGAGTTCTGCTTTTCTCTCTACAAGGATTCCCTTATATTG
ATACAGACGAAAGATATGCAGGAACCGGAATTCGTGTATTACAACGCTTT
TACTTCCTCTACGGTATCTTTGATTGTCTCCAAACATGACAACAAATTCG
AAACACTCAGTAAAAACCAAAAGATTCTCTTTAAAAATGCGAACGAGAAA
GAAGTAATTGCAAAATCAATTGGCATCCAAAATTTGAAAGTTTTTGAAAA
ATATATAGTATCTGCCCTCGGAGAGGTTACTAAAGCGGAATTTAGACAGC
GAGAGGACTTCAAAAAATCAGGTCCACCCAAGAAAAAACGCAAGGTGGAA
GATCCGAAGAAAAAGCGAAAAGTGGATGTGtaaCGTTTTCCGGGACGCCG
GCTGGATGATCCTCCAGCGCGGGGATCTCATGCTGGAGTTCTTCGCCCAC
CCCAACTTGTTTATTGCAGCTTATAATGGTTACAAATAAAGCAATAGCAT
CACAAATTTCACAAATAAAGCATTTTTTTCACTGCATTCTAGTTGTGGTT
TGTCCAAACTCATCAATGTATCTTATCATGTCTGTATACCG.

A E67-CjeCas9 and sgRNA plasmid may comprise or consist of the sequence (U6: N's=sgRNA spacer, E67, CjeCas9):

(SEQ ID NO: 203)
gtttattacagggacagcagagatccagtttggttaattaaggtaccgag
ggcctatttcccatgattccttcatatttgcatatacgatacaaggctgt
tagagagataattagaattaatttgactgtaaacacaaagatattagtac
aaaatacgtgacgtagaaagtaataatttcttgggtagtttgcagtttta
aaattatgttttaaaatggactatcatatgcttaccgtaacttgaaagta
tttcgatttcttggctttatatatcttGTGGAAAGGACGAAACACCNNNN
NNNNNNNNNNNNNNNGTTTTAGTCCCTGAAGGGACTAAAATAAAGAGTTT
GCGGGACTCTGCGGGGTTACAATCCCCTAAAACCGCTTTTTTTCCTGCAG
CCCGGGGGATCCACTAGTTCTAGAGCGGCCGCCACCGCGGTGGAGCTCCA
GCTTTTGTTCCCTTTAGTGAGGGTTAATTGCGCGAATTCGCTAGCTAGGT
CTTGAAAGGAGTGGGAATTGGCTCCGGTGCCCGTCAGTGGGCAGAGCGCA
CATCGCCCACAGTCCCCGAGAAGTTGGGGGGAGGGGTCGGCAATTGATCC
GGTGCCTAGAGAAGGTGGCGCGGGGTAAACTGGGAAAGTGATGTCGTGTA
CTGGCTCCGCCTTTTTCCCGAGGGTGGGGGAGAACCGTATATAAGTGCAG
TAGTCGCCGTGAACGTTCTTTTTCGCAACGGGTTTGCCGCCAGAACACAG
GACCGGTTCTAGAGCGCTATTTAGAACCatgCAGGAGGTAATAGCGGGGC
TTGAGCGATTTACCTTTGCCTTCGAAAAAGACGTAGAGATGCAGAAGGGA
ACCGGCCTGCTCCCATTTCAAGGTATGGACAAATCAGCATCTGCCGTGTG
CAATTTTTTCACCAAGGGTCTGTGTGAAAAGGGGAAGCTCTGTCCATTTC
GCCATGATCGCGGAGAGAAGATGGTGGTGTGTAAGCACTGGCTGAGAGGG
CTTTGCAAAAAAGGCGACCACTGCAAATTTCTTCACCAATATGACCTGAC
TCGAATGCCTGAGTGTTATTTTTACAGTAAGTTCGGTGACTGTAGCAACA
AAGAATGCAGCTTCTTGCATGTCAAACCAGCATTCAAGTCACAGGATTGC
CCGTGGTACGATCAGGGTTTTTGCAAGGACGGTCCCCTCTGCAAATATCG
ACACGTACCCAGAATTATGTGCCTTAATTACCTGGTCGGCTTCTGTCCTG
AAGGGCCAAAATGTCAGTTTGCTCAAAAAATTCGCGAGTTCAAATTGCTC
CCTGGGTCTAAAATTTGGGAACCCCAGGATTGGCAGCAGCAGCTTGTAAA
CATCCGAGCAATGAGGAACAAAAAAGATGCACCTGTTGATCACCTCGGAA
CCGAACATTGTTATGATTCTAGTGCGCCGCCAAAAGTCCGCCGGTATCAG
GTTCTGTTGAGTTTGATGCTGAGTAGTCAGACTAAGGACCAGGTTACGGC
CGGAGCAATGCAACGGCTTCGGGCACGGGGACTCACGGTCGATAGCATTT
TGCAGACCGATGACGCAACATTGGGTAAACTCATATATCCAGTTGGCTTC
TGGCGGAGCAAAGTGAAGTACATCAAGCAGACCTCAGCCATTCTCCAACA
ACATTACGGAGGTGATATACCCGCAAGCGTAGCTGAACTGGTAGCACTGC
CGGGCGTCGGTCCCAAAATGGCACATCTGGCTATGGCGGTTGCTTGGGGA
ACGGTGTCTGGTATCGCAGTTGATACGCATGTCCACCGCATCGCCAATCG
GCTGAGGTGGACTAAAAAAGCCACTAAGTCTCCTGAAGAAACACGGGCTG
CTCTGGAAGAGTGGCTTCCACGAGAGCTGTGGCATGAAATCAATGGATTG
CTGGTTGGTTTCGGGCAGCAGACATGCTTGCCCGTGCACCCCCGGTGTCA
TGCTTGCTTGAACCAGGCTTTGTGCCCAGCTGCCCAGGGCCTGAGTGGAA
GTGAGACACCGGGAACATCTGAGTCTGCGACCCCGGAGAGCacaaacGCG
CGAATCCTGGCCTTCGcgATTGGCATTAGCAGCATCGGCTGGGCATTCTC
TGAAAACGACGAACTGAAGGATTGCGGCGTGCGAATTTTCACTAAGGTCG
AAAATCCCAAAACTGGTGAATCACTCGCTCTCCCTAGACGACTGGCACGC
TCCGCACGAAAGAGGCTTGCCCGCCGCAAGGCACGCTTGAACCATCTTAA
ACACCTTATTGCAAATGAGTTTAAACTGAATTATGAGGACTACCAATCCT
TTGACGAGTCTCTTGCTAAAGCCTACAAAGGGAGCCTTATATCCCCGTAT
GAGCTCCGGTTCAGAGCACTCAACGAACTGCTGTCCAAACAGGATTTTGC
TCGCGTGATTCTCCACATAGCGAAGAGGCGAGGATACGATGACATTAAAA
ACAGTGATGATAAGGAAAAAGGGGCCATACTCAAAGCGATTAAGCAAAAT
GAAGAGAAGCTCGCTAACTATCAATCAGTAGGGGAGTATCTCTATAAAGA
GTACTTCCAGAAGTTCAAAGAAAATAGCAAGGAATTTACTAATGTCCGGA
ATAAAAAGGAGTCTTACGAAAGATGTATTGCGCAATCTTTCCTCAAGGAC
GAGCTCAAATTGATTTTCAAGAAACAAAGGGAATTTGGGTTCAGCTTCTC
AAAAAAATTTGAGGAAGAGGTTCTGAGCGTTGCCTTTTACAAACGCGCCC
TTAAGGACTTCTCACATCTCGTAGGGAATTGTAGTTTCTTCACCGATGAA
AAACGGGCGCCAAAAAATAGCCCTTTGGCTTTTATGTTTGTCGCTCTGAC
TCGCATCATTAATCTGCTCAACAACCTTAAAAACACGGAAGGGATTCTGT
ACACAAAGGATGATCTGAACGCTCTGCTTAACGAAGTTTTGAAGAACGGG
ACTTTGACCTACAAACAAACCAAAAAGCTTCTTGGTCTCAGTGATGACTA
CGAATTCAAGGGAGAAAAAGGGACATATTTCATCGAATTCAAGAAGTATA
AGGAGTTCATCAAAGCCTTGGGCGAGCACAACTTGTCTCAAGATGATCTC
AACGAAATTGCTAAGGATATCACTCTGATTAAAGACGAGATCAAGCTCAA
AAAGGCGTTGGCGAAGTATGACCTTAACCAAAACCAAATAGATAGCCTCA
GCAAGTTGGAATTTAAAGATCACTTGAATATAAGTTTCAAGGCCCTTAAG
TTGGTCACCCCCTTGATGCTTGAAGGAAAGAAATATGATGAGGCATGTAA
TGAGCTGAATCTCAAGGTTGCTATTAACGAAGACAAAAAAGATTTCCTCC
CAGCTTTCAATGAGACTTACTATAAGGACGAGGTTACCAATCCTGTGGTG
CTCCGAGCCATCAAAGAGTATCGAAAGGTCCTGAATGCTTTGCTCAAAAA
ATACGGTAAGGTACACAAAATAAATATTGAGCTCGCAAGGGAGGTCGGTA
AGAACCACTCCCAGCGCGCCAAAATAGAAAAGGAACAGAATGAAAATTAC
AAAGCGAAAAAGGACGCCGAGCTCGAGTGCGAAAAGCTGGGCCTGAAAAT
AAACAGCAAGAACATTCTCAAACTCCGCCTCTTCAAAGAACAAAAAGAAT
TTTGTGCTTATAGTGGTGAGAAAATAAAAATCTCCGATCTTCAAGACGAG
AAGATGCTCGAAATAGACgcgATATATCCATATAGCAGGTCTTTTGACGA
TTCTTACATGAATAAAGTGCTTGTTTTCACTAAGCAGAATCAGGAAAAGT
TGAATCAGACCCCCTTTGAGGCCTTTGGCAACGACTCAGCAAAGTGGCAG
AAGATCGAGGTCTTGGCTAAGAATCTTCCTACTAAGAAACAGAAAAGGAT
ATTGGATAAGAACTATAAAGACAAAGAACAAAAGAACTTTAAAGACCGCA
ACCTCAATGACACCAGATACATAGCAAGATTGGTTCTGAACTACACAAAA
GATTATTTGGACTTCTTGCCGCTGTCTGATGATGAGAACACGAAACTCAA
CGACACGCAAAAGGGGTCTAAAGTCCACGTCGAAGCTAAATCTGGGATGC
TCACCTCAGCATTGAGGCATACGTGGGGATTCTCAGCAAAGGACCGAAAC
AATCACCTGCACCATGCCATTGACGCAGTTATCATAGCGTATGCCAATAA
TTCAATAGTAAAAGCGTTTAGCGACTTCAAGAAGGAACAAGAGTCCAACA
GCGCCGAGCTCTACGCAAAAAAGATTAGTGAACTCGACTACAAAAACAAA
AGAAAATTCTTTGAGCCGTTCAGCGGATTTCGACAGAAGGTATTGGATAA
AATAGATGAAATTTTCGTGAGCAAACCCGAAAGGAAAAAGCCCTCAGGCG
CCTTGCACGAAGAGACTTTCAGGAAGGAAGAGGAATTCTACCAAAGCTAC
GGCGGAAAAGAGGGAGTTTTGAAGGCTCTCGAACTTGGAAAGATTAGGAA
GGTGAACGGCAAGATAGTGAAAAACGGCGATATGTTCCGGGTTGATATCT
TCAAACATAAAAAAACGAATAAATTTTATGCTGTGCCTATATACACTATG
GACTTCGCACTTAAGGTCCTGCCGAATAAGGCGGTAGCCCGATCTAAAAA
AGGCGAAATTAAGGACTGGATTTTGATGGATGAAAATTACGAGTTCTGCT
TTTCTCTCTACAAGGATTCCCTTATATTGATACAGACGAAAGATATGCAG
GAACCGGAATTCGTGTATTACAACGCTTTTACTTCCTCTACGGTATCTTT
GATTGTCTCCAAACATGACAACAAATTCGAAACACTCAGTAAAAACCAAA
AGATTCTCTTTAAAAATGCGAACGAGAAAGAAGTAATTGCAAAATCAATT
GGCATCCAAAATTTGAAAGTTTTTGAAAAATATATAGTATCTGCCCTCGG
AGAGGTTACTAAAGCGGAATTTAGACAGCGAGAGGACTTCAAAAAATCAG
GTCCACCCAAGAAAAAACGCAAGGTGGAAGATCCGAAGAAAAAGCGAAAA
GTGGATGTGtaaCGTTTTCCGGGACGCCGGCTGGATGATCCTCCAGCGCG
GGGATCTCATGCTGGAGTTCTTCGCCCACCCCAACTTGTTTATTGCAGCT
TATAATGGTTACAAATAAAGCAATAGCATCACAAATTTCACAAATAAAGC
ATTTTTTTCACTGCATTCTAGTTGTGGTTTGTCCAAACTCATCAATGTAT
CTTATCATGTCTGTATACCG.

### EXAMPLE EMBODIMENTS

Embodiment 1. A composition comprising:
   (a) a first sequence comprising a first guide RNA (gRNA) that specifically binds a target sequence within an RNA molecule, wherein the target sequence comprises a sequence encoding a component of an adaptive immune response and
   (b) a sequence encoding a fusion protein, the sequence comprising a sequence encoding a first RNA-binding polypeptide and a sequence encoding a second RNA-binding polypeptide,
   wherein neither the first RNA-binding polypeptide nor the second RNA-binding polypeptide comprises a significant DNA-nuclease activity,
   wherein the first RNA-binding polypeptide and the second RNA-binding polypeptide are not identical, and
   wherein the second RNA-binding polypeptide comprises an RNA-nuclease activity.
Embodiment 2. A composition comprising: (a) a first sequence comprising a first guide RNA (gRNA) that specifically binds a first target sequence within a first RNA molecule, wherein the first target sequence comprises a sequence encoding a component of an adaptive immune response and
   (b) a second sequence comprising a second guide RNA (gRNA) that specifically binds a second target sequence within a second RNA molecule and
   (c) a sequence encoding a fusion protein, the sequence comprising a sequence encoding a first RNA-binding polypeptide and a sequence encoding a second RNA-binding polypeptide,
   wherein neither the first RNA-binding polypeptide nor the second RNA-binding polypeptide comprises a significant DNA-nuclease activity,
   wherein the first RNA-binding polypeptide and the second RNA-binding polypeptide are not identical, and
   wherein the second RNA-binding polypeptide comprises an RNA-nuclease activity.
Embodiment 3. The composition of embodiment 2, wherein the first target sequence or the second target sequence comprises at least one repeated sequence.
Embodiment 4. The composition of embodiment 2, wherein the first sequence comprising the first gRNA further comprises a first promoter capable of expressing the gRNA in a eukaryotic cell and/or the second sequence comprising the second gRNA further comprises a second promoter capable of expressing the gRNA in a eukaryotic cell.
Embodiment 5. The composition of embodiment 2, wherein a sequence comprising the first sequence comprising the first gRNA and the second sequence comprising the second gRNA comprises a promoter capable of expressing the first gRNA and the second gRNA in a eukaryotic cell.
Embodiment 6. The composition of embodiment 4, wherein the first promoter and the second promoter are identical.
Embodiment 7. The composition of embodiment 4, wherein the first promoter and the second promoter are not identical.
Embodiment 8. The composition of any one of embodiments 4-7, wherein the eukaryotic cell is an animal cell.
Embodiment 9. The composition of embodiment 8, wherein the animal cell is a mammalian cell.
Embodiment 10. The composition of embodiment 9, wherein the animal cell is a human cell.
Embodiment 11. The composition of any one of embodiments 5-10, wherein the promoter is a constitutively active promoter.
Embodiment 12. The composition of any one of embodiments 5-11, wherein the promoter comprises a sequence isolated or derived from a promoter capable of driving expression of an RNA polymerase.
Embodiment 13. The composition of embodiment 12, wherein the promoter comprises a sequence isolated or derived from a U6 promoter.
Embodiment 14. The composition of any one of embodiments 5-12, wherein the promoter comprises a sequence isolated or derived from a promoter capable of driving expression of a transfer RNA (tRNA).
Embodiment 15. The composition of embodiment 14, wherein the promoter comprises a sequence isolated or derived from an alanine tRNA promoter, an arginine tRNA promoter, an asparagine tRNA promoter, an aspartic acid tRNA promoter, a cysteine tRNA promoter, a glutamine tRNA promoter, a glutamic acid tRNA promoter, a glycine tRNA promoter, a histidine tRNA promoter, an isoleucine tRNA promoter, a leucine tRNA promoter, a lysine tRNA promoter, a methionine tRNA promoter, a phenylalanine tRNA promoter, a proline tRNA promoter, a serine tRNA promoter, a threonine tRNA promoter, a tryptophan tRNA promoter, a tyrosine tRNA promoter, or a valine tRNA promoter.
Embodiment 16. The composition of embodiment 14, wherein the promoter comprises a sequence isolated or derived from a valine tRNA promoter.
Embodiment 17. The composition of any one of embodiments 2-16, wherein the sequence comprising the first gRNA further comprises a first spacer sequence that specifically binds to the first target RNA sequence.
Embodiment 18. The composition of embodiment 17, wherein the first spacer sequence has at least 50%, 55%, 60%, 65%, 70%, 75%, 80%, 87%, 90%, 95%, 97%, 99% or any percentage in between of complementarity to the first target RNA sequence.
Embodiment 19. The composition of embodiment 17, wherein the first spacer sequence has 100% complementarity to the target RNA sequence.
Embodiment 20. The composition of any one of embodiments 17-19, wherein the first spacer sequence comprises or consists of 20 nucleotides.
Embodiment 21. The composition of any one of embodiments 17-19, wherein the first spacer sequence comprises or consists of 21 nucleotides.
Embodiment 22. The composition of embodiment 21, wherein the first spacer sequence comprises or consists of 20 nucleotides of an amino acid sequence encoding a Beta-2-microglobulin (β2M) protein.
Embodiment 23. The composition of embodiment 22, wherein the first spacer sequence comprises or consists of 20 nucleotides of an amino acid sequence of

(SEQ ID NO: 88)
MSRSVALAVL ALLSLSGLEA IQRTPKIQVY SRHPADIEVD
LLKNGERIEK VEHSDLSFSK DWSFYLLYYT EFTPTEKDEY
ACRVNHVTLS QPKIVKWDRD M.

Embodiment 24. The composition of any one of embodiments 2-23, wherein the sequence comprising the first gRNA further comprises a first scaffold sequence that specifically binds to the first RNA binding protein.
Embodiment 25. The composition of embodiment 24, wherein the first scaffold sequence comprises a stem-loop structure.
Embodiment 26. The composition of embodiment 24 or 25, wherein the scaffold sequence comprises or consists of 90 nucleotides.
Embodiment 27. The composition of embodiment 24 or 25, wherein the scaffold sequence comprises or consists of 93 nucleotides.
Embodiment 28. The composition of embodiment 27, wherein the scaffold sequence comprises the sequence

(SEQ ID NO: 12)
GUUUAAGAGCUAUGCUGGAAACAGCAUAGCAAGUUUAAAUAAGGCUAGU
CCGUUAUCAACUUGAAAAAGUGGCACCGAGUCGGUGCUUUUUUU
or
(SEQ ID NO: 13)
GUUUUAGAGCUAGAAAUAGCAAGUUAAAAUAAGGCUAGUCCGUUAUCAA
CUUGAAAAAGUGGCACCGAGUCGGUGCUUUUUUU.

Embodiment 29. The composition of any one of embodiments 1-28, wherein the sequence comprising the second gRNA further comprises a second spacer sequence that specifically binds to the second target RNA sequence.
Embodiment 30. The composition of embodiment 29, wherein the second spacer sequence has at least 50%, 55%, 60%, 65%, 70%, 75%, 80%, 87%, 90%, 95%, 97%, 99% or any percentage in between of complementarity to the first target RNA sequence.
Embodiment 31. The composition of embodiment 29, wherein the second spacer sequence has 100% complementarity to the target RNA sequence.
Embodiment 32. The composition of any one of embodiments 29-31, wherein the second spacer sequence comprises or consists of 20 nucleotides.
Embodiment 33. The composition of any one of embodiments 29-31, wherein the second spacer sequence comprises or consists of 21 nucleotides.
Embodiment 34. The composition of any one of embodiments 2-34, wherein the second spacer sequence comprises or further comprises a sequence comprising at least 1, 2, 3, 4, 5, 6, or 7 repeats of the sequence CUG (SEQ ID NO: 18), CCUG (SEQ ID NO: 19), CAG (SEQ ID NO: 80), GGGGCC (SEQ ID NO: 81) or any combination thereof.
Embodiment 35. The composition of any one of embodiments 2-34, wherein the sequence comprising the second gRNA further comprises a second scaffold sequence that specifically binds to the first RNA binding protein.
Embodiment 36. The composition of embodiment 35, wherein the second scaffold sequence comprises a stem-loop structure.
Embodiment 37. The composition of embodiment 35 or 36, wherein the second scaffold sequence comprises or consists of 85 nucleotides.
Embodiment 38. The composition of embodiment 37, wherein the second scaffold sequence comprises the sequence

(SEQ ID NO: 12)
GUUUAAGAGCUAUGCUGGAAACAGCAUAGCAAGUUUAAAUAAGGCUAGU
CCGUUAUCAACUUGAAAAAGUGGCACCGAGUCGGUGCUUUUUUU
or
(SEQ ID NO: 13)
GUUUUAGAGCUAGAAAUAGCAAGUUAAAAUAAGGCUAGUCCGUUAUCAA
CUUGAAAAAGUGGCACCGAGUCGGUGCUUUUUUU.

Embodiment 39. The composition of embodiment 1, wherein the gRNA does not bind or does not selectively bind to a second sequence within the RNA molecule.
Embodiment 40. The composition of any one of embodiments 2-38, wherein the first gRNA does not bind or does not selectively bind to a second sequence within the first RNA molecule.
Embodiment 41. The composition of any one of embodiments 2-38, wherein the second gRNA does not bind or does not selectively bind to a second sequence within the second RNA molecule.
Embodiment 42. The composition of embodiment 39, wherein an RNA genome or an RNA transcriptome comprises the RNA molecule.
Embodiment 43. The composition of embodiment 40 or 41, wherein an RNA genome or an RNA transcriptome comprises the first RNA molecule or the second RNA molecule.
Embodiment 44. The composition of any one of embodiments 1-43, wherein the first RNA binding protein comprises a CRISPR-Cas protein.
Embodiment 45. The composition of embodiment 44, wherein the CRISPR-Cas protein is a Type II CRISPR-Cas protein.
Embodiment 46. The composition of embodiment 45, wherein the first RNA binding protein comprises a Cas9 polypeptide or an RNA-binding portion thereof.
Embodiment 47. The composition of embodiment 44, wherein the CRISPR-Cas protein is a Type V CRISPR-Cas protein.
Embodiment 48. The composition of embodiment 47, wherein the first RNA binding protein comprises a Cpf1 polypeptide or an RNA-binding portion thereof.
Embodiment 49. The composition of embodiment 44, wherein the CRISPR-Cas protein is a Type VI CRISPR-Cas protein.
Embodiment 50. The composition of embodiment 49, wherein the first RNA binding protein comprises a Cas13 polypeptide or an RNA-binding portion thereof.
Embodiment 51. The composition of any one of embodiments 44-50, wherein the CRISPR-Cas protein comprises a native RNA nuclease activity.
Embodiment 52. The composition of embodiment 51, wherein the native RNA nuclease activity is reduced or inhibited.
Embodiment 53. The composition of embodiment 52, wherein the native RNA nuclease activity is increased or induced.
Embodiment 54. The composition of any one of embodiments 44-53, wherein the CRISPR-Cas protein comprises a native DNA nuclease activity and wherein the native DNA nuclease activity is inhibited.
Embodiment 55. The composition of embodiment 54, wherein the CRISPR-Cas protein comprises a mutation.
Embodiment 56. The composition of embodiment 54 or 55, wherein a nuclease domain of the CRISPR-Cas protein comprises the mutation.
Embodiment 57. The composition of any one of embodiments 54-56, wherein the mutation occurs in a nucleic acid encoding the CRISPR-Cas protein.
Embodiment 58. The composition of any one of embodiments 54-56, wherein the mutation occurs in an amino acid encoding the CRISPR-Cas protein.
Embodiment 59. The composition of any one of embodiments 54-58, wherein the mutation comprises a substitution, an insertion, a deletion, a frameshift, an inversion, or a transposition.
Embodiment 60. The composition of embodiment 59, wherein the mutation comprises a deletion of a nuclease domain, a binding site within the nuclease domain, an active site within the nuclease domain, or at least one essential amino acid residue within the nuclease domain.
Embodiment 61. The composition of any one of embodiments 1-43, wherein the first RNA binding protein comprises a Pumilio and FBF (PUF) protein.
Embodiment 62. The composition of embodiment 61, wherein the first RNA binding protein comprises a Pumilio-based assembly (PUMBY) protein.
Embodiment 63. The composition of any one of embodiments 1-56, wherein the first RNA binding protein does not require multimerization for RNA-binding activity.
Embodiment 64. The composition of embodiment 63, wherein the first RNA binding protein is not a monomer of a multimer complex
Embodiment 65. The composition of embodiment 63, wherein a multimer protein complex does not comprise the first RNA binding protein.
Embodiment 66. The composition of any one of embodiments 1-65, wherein the first RNA binding protein selectively binds to a target sequence within the RNA molecule.
Embodiment 67. The composition of embodiment 66, wherein the first RNA binding protein does not comprise an affinity for a second sequence within the RNA molecule.
Embodiment 68. The composition of embodiment 66 or 67, wherein the first RNA binding protein does not comprise a high affinity for or selectively bind a second sequence within the RNA molecule.
Embodiment 69. The composition of embodiment 68, wherein an RNA genome or an RNA transcriptome comprises the RNA molecule.
Embodiment 70. The composition of any one of embodiments 1-69, wherein the first RNA binding protein comprises between 2 and 1300 amino acids, inclusive of the endpoints.
Embodiment 71. The composition of any one of embodiments 1-70, wherein the sequence encoding the first RNA binding protein further comprises a nuclear localization signal (NLS).
Embodiment 72. The composition of embodiment 71, wherein the sequence encoding a nuclear localization signal (NLS) is positioned 3′ to the sequence encoding the first RNA binding protein.
Embodiment 73. The composition of embodiment 72, wherein the first RNA binding protein comprises an NLS at a C-terminus of the protein.
Embodiment 74. The composition of any one of embodiments 1-70, wherein the sequence encoding the first RNA binding protein further comprises a first sequence encoding a first NLS and a second sequence encoding a second NLS.
Embodiment 75. The composition of embodiment 74, wherein the sequence encoding the first NLS or the second NLS is positioned 3′ to the sequence encoding the first RNA binding protein.
Embodiment 76. The composition of embodiment 75, wherein the first RNA binding protein comprises the first NLS or the second NLS at a C-terminus of the protein.
Embodiment 77. The composition of any one of embodiments 1-76, wherein the second RNA binding protein comprises or consists of a nuclease domain.
Embodiment 78. The composition of embodiment 77, wherein the second RNA binding protein comprises or consists of an RNAse.
Embodiment 79. The composition of embodiment 78, wherein the second RNA binding protein comprises or consists of an RNAse1.
Embodiment 80. The composition of embodiment 79, wherein the RNAse1 protein comprises or consists of SEQ ID NO: 20.
Embodiment 81. The composition of embodiment 78, wherein the second RNA binding protein comprises or consists of an RNAse4.
Embodiment 82. The composition of embodiment 81, wherein the RNAse4 protein comprises or consists of SEQ ID NO: 21.
Embodiment 83. The composition of embodiment 78, wherein the second RNA binding protein comprises or consists of an RNAse6.
Embodiment 84. The composition of embodiment 83, wherein the RNAse6 protein comprises or consists of SEQ ID NO: 22.
Embodiment 85. The composition of embodiment 78, wherein the second RNA binding protein comprises or consists of an RNAse7.
Embodiment 86. The composition of embodiment 85, wherein the RNAse7 protein comprises or consists of SEQ ID NO: 23.
Embodiment 87. The composition of embodiment 78, wherein the second RNA binding protein comprises or consists of an RNAse8.
Embodiment 88. The composition of embodiment 87, wherein the RNAse8 protein comprises or consists of SEQ ID NO: 24.
Embodiment 89. The composition of embodiment 88, wherein the second RNA binding protein comprises or consists of an RNAse2.
Embodiment 90. The composition of embodiment 89, wherein the RNAse2 protein comprises or consists of SEQ ID NO: 25.
Embodiment 91. The composition of embodiment 78, wherein the second RNA binding protein comprises or consists of an RNAse6PL.
Embodiment 92. The composition of embodiment 91, wherein the RNAse6PL protein comprises or consists of SEQ ID NO: 26.
Embodiment 93. The composition of embodiment 78, wherein the second RNA binding protein comprises or consists of an RNAseL.
Embodiment 94. The composition of embodiment 93, wherein the RNAseL protein comprises or consists of SEQ ID NO: 27.
Embodiment 95. The composition of embodiment 78, wherein the second RNA binding protein comprises or consists of an RNAseT2.
Embodiment 96. The composition of embodiment 95, wherein the RNAseT2 protein comprises or consists of SEQ ID NO: 28.
Embodiment 97. The composition of embodiment 78, wherein the second RNA binding protein comprises or consists of an RNAse11.
Embodiment 98. The composition of embodiment 97, wherein the RNAse11 protein comprises or consists of SEQ ID NO: 29.
Embodiment 99. The composition of embodiment 78, wherein the second RNA binding protein comprises or consists of an RNAseT2-like.
Embodiment 100. The composition of embodiment 99, wherein the RNAseT2-like protein comprises or consists of SEQ ID NO: 30.
Embodiment 101. The composition of embodiment 77, wherein the second RNA binding protein comprises or consists of a NOB1 polypeptide.
Embodiment 102. The composition of embodiment 101, wherein the NOB1 polypeptide comprises or consists of SEQ ID NO: 31.
Embodiment 103. The composition of embodiment 77, wherein the second RNA binding protein comprises or consists of an endonuclease.
Embodiment 104. The composition of embodiment 103, wherein the second RNA binding protein comprises or consists of an endonuclease V (ENDOV) polypeptide.
Embodiment 105. The composition of embodiment 104, wherein the ENDOV protein comprises or consists of SEQ ID NO: 32.
Embodiment 106. The composition of embodiment 103, wherein the second RNA binding protein comprises or consists of an endonuclease G (ENDOG).
Embodiment 107. The composition of embodiment 106, wherein the ENDOG protein comprises or consists of SEQ ID NO: 33.
Embodiment 108. The composition of embodiment 103, wherein the second RNA binding protein comprises or consists of an endonuclease D1 (ENDOD1) polypeptide.
Embodiment 109. The composition of embodiment 108, wherein the ENDOD1 comprises or consists of SEQ ID NO: 34.
Embodiment 110. The composition of embodiment 77, wherein the second RNA binding protein comprises or consists of a Human flap endonuclease-1 (hFEN1) polypeptide.
Embodiment 111. The composition of embodiment 110, wherein the hFEN1 protein comprises or consists of SEQ ID NO: 35.
Embodiment 112. The composition of embodiment 77, wherein the second RNA binding protein comprises or consists of a human Schlafen 14 (hSLFN14) polypeptide.
Embodiment 113. The composition of embodiment 112, wherein the hSLFN14 polypeptide comprises or consists of SEQ ID NO: 36.
Embodiment 114. The composition of embodiment 77, wherein the second RNA binding protein comprises or consists of a human beta-lactamase-like protein 2 (hLACTB2) polypeptide.
Embodiment 115. The composition of embodiment 114, wherein the hLACTB2 polypeptide comprises or consists of SEQ ID NO: 37.
Embodiment 116. The composition of embodiment 77, wherein the second RNA binding protein comprises or consists of an apurinic/apyrimidinic (AP) endodeoxyribonuclease (APEX2) polypeptide.
Embodiment 117. The composition of embodiment 116, wherein the APEX2 polypeptide comprises or consists of SEQ ID NO: 38.
Embodiment 118. The composition of embodiment 116, wherein the APEX2 polypeptide comprises or consists of SEQ ID NO: 39.
Embodiment 119. The composition of embodiment 77, wherein the second RNA binding protein comprises or consists of an angiogenin (ANG) polypeptide.
Embodiment 120. The composition of embodiment 119, wherein the ANG polypeptide comprises or consists of SEQ ID NO: 40.
Embodiment 121. The composition of embodiment 77, wherein the second RNA binding protein comprises or consists of a heat responsive protein 12 (HRSP12) polypeptide.
Embodiment 122. The composition of embodiment 121, wherein the HRSP12 polypeptide comprises or consists of SEQ ID NO: 41.
Embodiment 123. The composition of embodiment 77, wherein the second RNA binding protein comprises or consists of a Zinc Finger CCCH-Type Containing 12A (ZC3H12A) polypeptide.
Embodiment 124. The composition of embodiment 123, wherein the ZC3H12A polypeptide comprises or consists of SEQ ID NO: 42.
Embodiment 125. The composition of embodiment 124, wherein the ZC3H12A polypeptide comprises or consists of SEQ ID NO: 43.
Embodiment 126. The composition of embodiment 77, wherein the second RNA binding protein comprises or consists of a Reactive Intermediate Imine Deaminase A (RIDA) polypeptide.
Embodiment 127. The composition of embodiment 126, wherein the RIDA polypeptide comprises or consists of SEQ ID NO: 44.
Embodiment 128. The composition of embodiment 77, wherein the second RNA binding protein comprises or consists of a Phospholipase D Family Member 6 (PDL6) polypeptide.
Embodiment 129. The composition of embodiment 128, wherein the PDL6 polypeptide comprises or consists of SEQ ID NO: 126.
Embodiment 130. The composition of embodiment 77, wherein the second RNA binding protein comprises or consists of a Endonuclease III-like protein 1 (NTHL) polypeptide.
Embodiment 131. The composition of embodiment 130, wherein the NTHL polypeptide comprises or consists of SEQ ID NO: 123.
Embodiment 132. The composition of embodiment 77, wherein the second RNA binding protein comprises or consists of a Mitochondrial ribonuclease P catalytic subunit (KIAA0391) polypeptide.
Embodiment 133. The composition of embodiment 132, wherein the KIAA0391 polypeptide comprises or consists of SEQ ID NO: 127.
Embodiment 134. The composition of embodiment 77, wherein the second RNA binding protein comprises or consists of an apurinic or apyrimidinic site lyase (APEX1) polypeptide.
Embodiment 135. The composition of embodiment 134, wherein the APEX1 polypeptide comprises or consists of SEQ ID NO: 125.
Embodiment 136. The composition of embodiment 77, wherein the second RNA binding protein comprises or consists of an argonaute 2 (AGO2) polypeptide.
Embodiment 137. The composition of embodiment 136, wherein the AGO2 polypeptide comprises or consists of SEQ ID NO: 128.
Embodiment 138. The composition of embodiment 67, wherein the second RNA binding protein comprises or consists of a mitochondrial nuclease EXOG (EXOG) polypeptide.
Embodiment 139. The composition of embodiment 138, wherein the EXOG polypeptide comprises or consists of SEQ ID NO: 129.
Embodiment 140. The composition of embodiment 77, wherein the second RNA binding protein comprises or consists of a Zinc Finger CCCH-Type Containing 12D (ZC3H12D) polypeptide.
Embodiment 141. The composition of embodiment 140, wherein the ZC3H12D polypeptide comprises or consists of SEQ ID NO: 130.
Embodiment 142. The composition of embodiment 77, wherein the second RNA binding protein comprises or consists of an endoplasmic reticulum to nucleus signaling 2 (ERN2) polypeptide.
Embodiment 143. The composition of embodiment 142, wherein the ERN2 polypeptide comprises or consists of SEQ ID NO: 131.
Embodiment 144. The composition of embodiment 77, wherein the second RNA binding protein comprises or consists of a pelota mRNA surveillance and ribosome rescue factor (PELO) polypeptide.
Embodiment 145. The composition of embodiment 144, wherein the PELO polypeptide comprises or consists of SEQ ID NO: 132.
Embodiment 146. The composition of embodiment 77, wherein the second RNA binding protein comprises or consists of a YBEY metallopeptidase (YBEY) polypeptide.
Embodiment 147. The composition of embodiment 146, wherein the YBEY polypeptide comprises or consists of SEQ ID NO: 133.
Embodiment 148. The composition of embodiment 77, wherein the second RNA binding protein comprises or consists of a cleavage and polyadenylation specific factor 4 like (CPSF4L) polypeptide.
Embodiment 149. The composition of embodiment 148, wherein the CPSF4L polypeptide comprises or consists of SEQ ID NO: 134.
Embodiment 150. The composition of embodiment 77, wherein the second RNA binding protein comprises or consists of an hCG_2002731 polypeptide.
Embodiment 151. The composition of embodiment 150, wherein the hCG_2002731 polypeptide comprises or consists of SEQ ID NO: 135.
Embodiment 152. The composition of embodiment 150, wherein the hCG_2002731 polypeptide comprises or consists of SEQ ID NO: 136.
Embodiment 153. The composition of embodiment 77, wherein the second RNA binding protein comprises or consists of an Excision Repair Cross-Complementation Group 1 (ERCC1) polypeptide.
Embodiment 154. The composition of embodiment 153, wherein the ERCC1 polypeptide comprises or consists of SEQ ID NO: 137.
Embodiment 155. The composition of embodiment 77, wherein the second RNA binding protein comprises or consists of a ras-related C3 botulinum toxin substrate 1 isoform (RAC1) polypeptide.
Embodiment 156. The composition of embodiment 155, wherein the RAC1 polypeptide comprises or consists of SEQ ID NO: 138.
Embodiment 157. The composition of embodiment 77, wherein the second RNA binding protein comprises or consists of a Ribonuclease A A1 (RAA1) polypeptide.
Embodiment 158. The composition of embodiment 157, wherein the RAA1 polypeptide comprises or consists of SEQ ID NO: 139.
Embodiment 159. The composition of embodiment 77, wherein the second RNA binding protein comprises or consists of a Ras Related Protein (RAB1) polypeptide.
Embodiment 160. The composition of embodiment 159, wherein the RAB1 polypeptide comprises or consists of SEQ ID NO: 140.
Embodiment 161. The composition of embodiment 77, wherein the second RNA binding protein comprises or consists of a DNA Replication Helicase/Nuclease 2 (DNA2) polypeptide.
Embodiment 162. The composition of embodiment 161, wherein the DNA2 polypeptide comprises or consists of SEQ ID NO: 141.
Embodiment 163. The composition of embodiment 77, wherein the second RNA binding protein comprises or consists of a FLJ35220 polypeptide.
Embodiment 164. The composition of embodiment 163, wherein the FLJ35220 polypeptide comprises or consists of SEQ ID NO: 142.
Embodiment 165. The composition of embodiment 77, wherein the second RNA binding protein comprises or consists of a FLJ13173 polypeptide.
Embodiment 166. The composition of embodiment 165, wherein the FLJ13173 polypeptide comprises or consists of SEQ ID NO: 143.
Embodiment 167. The composition of embodiment 77, wherein the second RNA binding protein comprises or consists of a DNA repair endonuclease XPF (ERCC4) polypeptide.
Embodiment 168. The composition of embodiment 167, wherein the ERCC4 polypeptide comprises or consists of SEQ ID NO: 124.
Embodiment 169. The composition of embodiment 77, wherein the second RNA binding protein comprises or consists of a mutated Rnase1 (Rnase1(K41R)) polypeptide.
Embodiment 170. The composition of embodiment 169, wherein the Rnase1(K41R) polypeptide comprises or consists of SEQ ID NO: 116.
Embodiment 171. The composition of embodiment 77, wherein the second RNA binding protein comprises or consists of a mutated Rnase1 (Rnase1(K41R, D121E)) polypeptide.
Embodiment 172. The composition of embodiment 171, wherein the Rnase1 (Rnase1(K41R, D121E)) polypeptide comprises or consists of SEQ ID NO: 117.
Embodiment 173. The composition of embodiment 77, wherein the second RNA binding protein comprises or consists of a mutated Rnase1 (Rnase1(K41R, D121E, H119N)) polypeptide.
Embodiment 174. The composition of embodiment 173, wherein the Rnase1 (Rnase1(K41R, D121E, Hi19N)) polypeptide comprises or consists of SEQ ID NO: 118.
Embodiment 175. The composition of embodiment 77, wherein the second RNA binding protein comprises or consists of a mutated Rnase1 (Rnase1(H119N)) polypeptide.
Embodiment 166. The composition of embodiment 175, wherein the Rnase1 (Rnase1(H119N)) polypeptide comprises or consists of SEQ ID NO: 119.
Embodiment 177. The composition of embodiment 77, wherein the second RNA binding protein comprises or consists of a mutated Rnase1 (Rnase1(R39D, N67D, N88A, G89D, R91D, Hi19N)) polypeptide.
Embodiment 178. The composition of embodiment 177, wherein the Rnase1 (Rnase1(R39D, N67D, N88A, G89D, R91D, Hi19N)) polypeptide comprises or consists of SEQ ID NO: 120.
Embodiment 179. The composition of embodiment 77, wherein the second RNA binding protein comprises or consists of a mutated Rnase1 (Rnase1(R39D, N67D, N88A, G89D, R91D, Hi19N)) polypeptide.
Embodiment 180. The composition of embodiment 179, wherein the Rnase1 (Rnase1(R39D, N67D, N88A, G89D, R91D, H119N, K41R, D121E)) polypeptide comprises or consists of SEQ ID NO: 121.
Embodiment 181. The composition of embodiment 77, wherein the second RNA binding protein comprises or consists of a mutated Rnase1 (Rnase1(R39D, N67D, N88A, G89D, R91D, H119N)) polypeptide.
Embodiment 182. The composition of embodiment 181, wherein the Rnase1 (Rnase1(R39D, N67D, N88A, G89D, R91D)) polypeptide comprises or consists of SEQ ID NO: 122.
Embodiment 183. The composition of embodiment 77, wherein the second RNA binding protein comprises or consists of Teneurin Transmembrane Protein 1 (TENM1) polypeptide.
Embodiment 184. The composition of embodiment 173, wherein the TENM1 polypeptide comprises or consists of SEQ ID NO: 144.
Embodiment 185. The composition of embodiment 77, wherein the second RNA binding protein comprises or consists of Teneurin Transmembrane Protein 2 (TENM2) polypeptide.
Embodiment 186. The composition of embodiment 185, wherein the TENM2 polypeptide comprises or consists of SEQ ID NO: 145.
Embodiment 187. The composition of any one of embodiments 1-77, wherein the second RNA binding protein comprises or consists of a transcription activator-like effector nuclease (TALEN) polypeptide or a nuclease domain thereof.
Embodiment 188. The composition of embodiment 187, wherein the TALEN polypeptide comprises or consists of:

| (SEQ ID NO: 205) | |
|---|---|
| 1 | MRIGKSSGWL NESVSLEYEH VSPPTRPRDT RRRPRAAGDG GLAHLHRRLA VGYAEDTPRT |
| 61 | EARSPAPRRP LPVAPASAPP APSLVPEPPM PVSLPAVSSP RFSAGSSAAI TDPFPSLPPT |
| 121 | PVLYAMAREL EALSDATWQP AVPLPAEPPT DARRGNTVFD EASASSPVIA SACPQAFASP |
| 181 | PRAPRSARAR RARTGGDAWP APTFLSRPSS SRIGRDVFGK LVALGYSREQ IRKLKQESLS |
| 241 | EIAKYHTTLT GQGFTHADIC RISRRRQSLR VVARNYPELA AALPELTRAH IVDIARQRSG |
| 301 | DLALQALLPV ATALTAAPLR LSASQIATVA QYGERPAIQA LYRLRRKLTR APLHLTPQQV |
| 361 | VAIASNTGGK RALEAVCVQL PVLRAAPYRL STEQVVAIAS NKGGKQALEA VKAHLLDLLG |
| 421 | APYVLDTEQV VAIASHNGGK QALEAVKADL LDLRGAPYAL STEQVVAIAS HNGGKQALEA |
| 481 | VKADLLELRG APYALSTEQV VAIASHNGGK QALEAVKAHL LDLRGVPYAL STEQVVAIAS |
| 541 | HNGGKQALEA VKAQLLDLRG APYALSTAQV VAIASNGGGK QALEGIGEQL LKLRTAPYGL |
| 601 | STEQVVAIAS HDGGKQALEA VGAQLVALRA APYALSTEQV VAIASNKGGK QALEAVKAQL |
| 661 | LELRGAPYAL STAQVVAIAS HDGGNQALEA VGTQLVALRA APYALSTEQV VAIASHDGGK |
| 721 | QALEAVGAQL VALRAAPYAL NTEQVVAIAS SHGGKQALEA VRALFPDLRA APYALSTAQL |
| 781 | VAIASNPGGK QALEAVRALF RELRAAPYAL STEQVVAIAS NHGGKQALEA VRALFRGLRA |
| 841 | APYGLSTAQV VAIASSNGGK QALEAVWALL PVLRATPYDL NTAQIVAIAS HDGGKPALEA |
| 901 | VWAKLPVLRG APYALSTAQV VAIACISGQQ ALEAIEAHMP TLRQASHSLS PERVAAIACI |
| 961 | GGRSAVEAVR QGLPVKAIRR IRREKAPVAG PPPASLGPTP QELVAVLHFF RAHQQPRQAF |
| 1021 | VDALAAFQAT RPALLRLLSS VGVTEIEALG GTIPDATERW QRLLGRLGFR PATGAAAPSP |
| 1081 | DSLQGFAQSL ERTLGSPGMA GQSACSPHRK RPAETAIAPR SIRRSPNNAG QPSEPWPDQL |
| 1141 | AWLQRRKRTA RSHIRADSAA SVPANLHLGT RAQFTPDRLR AEPGPIMQAH TSPASVSFGS |
| 1201 | HVAFEPGLPD PGTPTSADLA SFEAEPFGVG PLDFHLDWLL QILET. |

Embodiment 189. The composition of embodiment 187, wherein the TALEN polypeptide comprises or consists of:

| (SEQ ID NO: 206) | |
|---|---|
| 1 | mdpirsrtps parellpgpq pdrvqptadr ggappaggpl dglparrtms rtrlpsppap |
| 61 | spafsagsfs dllrqfdpsl ldtslldsmp avgtphtaaa paecdevqsg lraaddpppt |
| 121 | vrvavtaarp prakpaprrr aaqpsdaspa aqvdlrtlgy sqqqqekikp kvgstvaqhh |
| 181 | ealvghgfth ahivalsrhp aalgtvavky qdmiaalpea thedivgvgk qwsgaralea |
| 241 | lltvagelrg pplqldtgql vkiakrggvt aveavhasrn altgaplnlt paqvvaiasn |
| 301 | nggkqaletv qrllpvlcqa hgltpaqvva iashdggkqa letmqrllpv lcqahglppd |
| 361 | qvvaiasnig gkqaletvqr llpvlcqahg ltpdqvvaia shgggkqale tvqrllpvlc |
| 421 | qahgltpdqv vaiashdggk qaletvqrll pvlcqahglt pdqvvaiasn gggkqaletv |
| 481 | qrllpvlcqa hgltpdqvva iasnggkqal etvqrllpvl cqahgltpdq vvaiashdgg |
| 541 | kqaletvqrl lpvlcqthgl tpaqvvaias hdggkqalet vqqllpvlcq ahgltpdqvv |
| 601 | aiasniggkq alatvqrllp vlcqahgltp dqvvaiasng ggkqaletvq rllpvlcqah |
| 661 | gltpdqvvai asngggkqal etvqrllpvl cqahgltqvq vvaiasnigg kqaletvqrl |
| 721 | lpvlcqahgl tpaqvvaias hdggkqalet vqrllpvlcq ahgltpdqvv aiasngggkq |
| 781 | aletvqrllp vlcqahgltq eqvvaiasnn ggkqaletvq rllpvlcqah gltpdqvvai |
| 841 | asngggkqal etvqrllpvl cqahgltpaq vvaiasnigg kqaletvqrl lpvlcqdhgl |
| 901 | tlaqvvaias niggkqalet vqrllpvlcq ahgltqdqvv aiasniggkq aletvqrllp |
| 961 | vlcqdhgltp dqvvaiasni ggkqaletvq rllpvlcqdh gltldqvvai asnggkqale |
| 1021 | tvqrllpvlc qdhgltpdqv vaiasnsggk qaletvqrll pvlcqdhglt pnqvvaiasn |
| 1081 | ggkqalesiv aqlsrpdpal aaltndhlva laclggrpam davkkglpha pelirrvnrr |
| 1141 | igertshrva dyaqvvrvle ffqchshpay afdeamtqfg msrnglvqlf rrvgvtelea |
| 1201 | rggtlppasq rwdrilqasg mkrakpspts aqtpdqaslh afadslerdl dapspmhegd |
| 1261 | qtgassrkrs rsdravtgps aqhsfevrvp eqrdalhlpl swrvkrprtr iggglpdpgt |
| 1321 | piaadlaass tvmweqdaap fagaaddfpa fneeelawlm ellpqsgsvg gti. |

Embodiment 190. The composition of any one of embodiments 1-77, wherein the second RNA binding protein comprises or consists of a zinc finger nuclease polypeptide or a nuclease domain thereof.
Embodiment 191. The composition of embodiment 190, wherein the zinc finger nuclease polypeptide comprises or consists of:

| (SEQ ID NO: 207) | |
|---|---|
| 1 | MSRPRFNPRG DFPLQRPRAP NPSGMRPPGP FMRPGSMGLP RFYPAGRARG IPHRFAGHES |
| 61 | YQNMGPQRMN VQVTQHRTDP RLTKEKLDFH EAQQKKGKPH GSRWDDEPHI SASVAVKQSS |
| 121 | VTQVTEQSPK VQSRYTKESA SSILASFGLS NEDLEELSRY PDEQLTPENM PLILRDIRMR |
| 181 | KMGRRLPNLP SQSRNKETLG SEAVSSNVID YGHASKYGYT EDPLEVRIYD PEIPTDEVEN |
| 241 | EFQSQQNISA SVPNPNVICN SMFPVEDVFR QMDFPGESSN NRSFFSVESG TKMSGLHISG |
| 301 | GQSVLEPIKS VNQSINQTVS QTMSQSLIPP SMNQQPFSSE LISSVSQQER IPHEPVINSS |
| 361 | NVHVGSRGSK KNYQSQADIP IRSPFGIVKA SWLPKFSHAD AQKMKRLPTP SMMNDYYAAS |
| 421 | PRIFPHLCSL CNVECSHLKD WIQHQNTSTH IESCRQLRQQ YPDWNPEILP SRRNEGNRKE |
| 481 | NETPRRRSHS PSPRRSRRSS SSHRFRRSRS PMHYMYRPRS RSPRICHRFI SRYRSRSRSR |
| 541 | SPYRIRNPFR GSPKCFRSVS PERMSRRSVR SSDRKKALED VVQRSGHGTE FNKQKHLEAA |
| 601 | DKGHSPAQKP KTSSGTKPSV KPTSATKSDS NLGGHSIRCK SKNLEDDTLS ECKQVSDKAV |
| 661 | SLQRKLRKEQ SLHYGSVLLI TELPEDGCTE EDVRKLFQPF GKVNDVLIVP YRKEAYLEME |
| 721 | FKEAITAIMK YIETTPLTIK GKSVKICVPG KKKAQNKEVK KKTLESKKVS ASTLKRDADA |
| 781 | SKAVEIVTST SAAKTGQAKA SVAKVNKSTG KSASSVKSVV TVAVKGNKAS IKTAKSGGKK |
| 841 | SLEAKKTGNV KNKDSNKPVT IPENSEIKTS IEVKATENCA KEAISDAALE ATENEPLNKE |
| 901 | TEEMCVMLVS NLPNKGYSVE EVYDLAKPFG GLKDILILSS HKKAYIEINR KAAESMVKFY |
| 961 | TCFPVLMDGN QLSISMAPEN MNIKDEEAIF ITLVKENDPE ANIDTIYDRF VHLDNLPEDG |
| 1021 | LQCVLCVGLQ FGKVDHHVFI SNRNKAILQL DSPESAQSMY SFLKQNPQNI GDHMLTCSLS |
| 1081 | PKIDLPEVQI EHDPELEKES PGLKNSPIDE SEVQTATDSP SVKPNELEEE STPSIQTETL |
| 1141 | VQQEEPCEEE AEKATCDSDF AVETLELETQ GEEVKEEIPL VASASVSIEQ FTENAEECAL |
| 1201 | NQQMFNSDLE KKGAEIINPK TALLPSDSVF AEERNLKGIL EESPSEAEDF ISGITQTMVE |
| 1261 | AVAEVEKNET VSEILPSTCI VTLVPGIPTG DEKTVDKKNI SEKKGNMDEK EEKEFNTKET |
| 1321 | RMDLQIGTEK AEKNEGRMDA EKVEKMAAMK EKPAENTLFK AYPNKGVGQA NKPDETSKTS |
| 1381 | ILAVSDVSSS KPSIKAVIVS SPKAKATVSK TENQKSFPKS VPRDQINAEK KLSAKEFGLL |
| 1441 | KPTSARSGLA ESSSKFKPTQ SSLTRGGSGR ISALQGKLSK LDYRDITKQS QETEARPSIM |
| 1501 | KRDDSNNKTL AEQNTKNPKS TTGRSSKSKE EPLFPFNLDE FVTVDEVIEE VNPSQAKQNP |
| 1561 | LKGKRKETLK NVPFSELNLK KKKGKTSTPR GVEGELSFVT LDEIGEEEDA AAHLAQALVT |
| 1621 | VDEVIDEEEL NMEEMVKNSN SLFTLDELID QDDCISHSEP KDVTVLSVAE EQDLLKQERL |
| 1681 | VTVDEIGEVE ELPLNESADI TFATLNTKGN EGDTVRDSIG FISSQVPEDP STLVTVDEIQ |
| 1741 | DDSSDLHLVT LDEVTEEDED SLADFNNLKE ELNFVTVDEV GEEEDGDNDL KVELAQSKND |
| 1801 | HPTDKKGNRK KRAVDTKKTK LESLSQVGPV NENVMEEDLK TMIERHLTAK TPTKRVRIGK |
| 1861 | TLPSEKAVVT EPAKGEEAFQ MSEVDEESGL KDSEPERKRK KTEDSSSGKS VASDVPEELD |
| 1921 | FLVPKAGFFC PICSLFYSGE KAMTNHCKST RHKQNTEKFM AKQRKEKEQN EAEERSSR. |

Embodiment 192. The composition of any one of embodiments 1-191, wherein the composition further comprises (a) a sequence comprising a gRNA that specifically binds within an RNA molecule and (b) a sequence encoding a nuclease.
Embodiment 193. The composition of embodiment 192, wherein the nuclease comprises a sequence isolated or derived from a CRISPR/Cas protein.
Embodiment 194. The composition of embodiment 193, wherein the CRISPR/Cas protein is isolated or derived from any one of a type I, a type IA, a type IB, a type IC, a type ID, a type IE, a type IF, a type IU, a type III, a type IIIA, a type IIIB, a type IIIC, a type HID, a type IV, a type IVA, a type IVB, a type II, a type IIA, a type IIB, a type IIC, a type V, or a type VI CRISPR/Cas protein.
Embodiment 195. The composition of embodiment 192, wherein the nuclease comprises a sequence isolated or derived from a TALEN or a nuclease domain thereof.
Embodiment 196. The composition of embodiment 192, wherein the nuclease comprises a sequence isolated or derived from a zinc finger nuclease or a nuclease domain thereof.
Embodiment 197. The composition of any one of embodiments 191-196, wherein the target sequence comprises a sequence encoding a component of an adaptive immune response.
Embodiment 198. A vector comprising the composition of any one of embodiments 1-197.
Embodiment 199. The vector of embodiment 198, wherein the vector is a viral vector.
Embodiment 200. The vector of embodiment 199, wherein the vector comprises a sequence isolated or derived from a lentivirus, an adenovirus, an adeno-associated virus (AAV) vector, or a retrovirus.
Embodiment 201. The vector of embodiment 199 or 200, wherein the vector is replication incompetent.
Embodiment 202. The vector of embodiment any one of embodiments 100-201, wherein the vector comprises a sequence isolated or derived from an adeno-associated vector (AAV).
Embodiment 203. The vector of embodiment 202, wherein the adeno-associated virus (AAV) is an isolated AAV.
Embodiment 204. The vector of embodiment 202 or 203, wherein the adeno-associated virus (AAV) is a self-complementary adeno-associated virus (scAAV).
Embodiment 205. The vector of any one of embodiments 202-204, wherein the adeno-associated virus (AAV) is a recombinant adeno-associated virus (rAAV).
Embodiment 206. The vector of any one of embodiments 202-205, wherein the adeno-associated virus (AAV) comprises a sequence isolated or derived from an AAV of serotype AAV1, AAV2, AAV3, AAV4, AAV5, AAV6, AAV7, AAV8, AAV9, AAV10, AAV11, or AAV12.
Embodiment 207. The vector of any one of embodiments 202-206, wherein the adeno-associated virus (AAV) comprises a sequence isolated or derived from an AAV of serotype AAV9.
Embodiment 208. The vector of any one of embodiments 202-206, wherein the adeno-associated virus (AAV) comprise a sequence isolated or derived from Anc80
Embodiment 209. The vector of any one of embodiments 100-201, wherein the vector is a retrovirus.
Embodiment 210. The vector of embodiment any one of claims 100-201, wherein the retrovirus is a lentivirus.
Embodiment 211. The vector of embodiment 198, wherein the vector is a non-viral vector.
Embodiment 212. The vector of embodiment 211, wherein the non-viral vector comprises a nanoparticle, a micelle, a liposome or lipoplex, a polymersome, a polyplex or a dendrimer.
Embodiment 213. A composition comprising the vector of any one of embodiments 198-212.
Embodiment 214. A cell comprising the vector of any one of embodiments 198-212.
Embodiment 215. A cell comprising the composition of embodiment 214.
Embodiment 216. The cell of embodiment 214 or 215, wherein the cell is a mammalian cell.
Embodiment 217. The cell of embodiment 216, wherein the cell is a human cell.
Embodiment 218. The cell of any one of embodiments 215-217, wherein the cell is an immune cell.
Embodiment 219. The cell of embodiment 218, wherein the immune cell is a T lymphocyte (T-cell).
Embodiment 220. The cell of embodiment 219, wherein the T-cell is an effector T-cell, a helper T-cell, a memory T-cell, a regulatory T-cell, a natural Killer T-cell, a mucosal-associated invariant T-cell, or a gamma delta T cell.
Embodiment 221. The cell of any one of embodiments 215-217, wherein the immune cell is an antigen presenting cell.
Embodiment 222. The cell of embodiment 221, wherein the antigen presenting cell is a dendritic cell, a macrophage, or a B cell.
Embodiment 223. The cell of embodiment 221, wherein the antigen presenting cell is a somatic cell.
Embodiment 224. The cell of any one of embodiments 215-223, wherein the cell is a healthy cell.
Embodiment 225. The cell of any one of embodiments 215-223, wherein the cell is not a healthy cell.
Embodiment 226. The cell of embodiment 225, where the cell is isolated or derived from a subject having a disease or disorder.
Embodiment 227. A composition comprising the cell of any one of embodiments 215-226.
Embodiment 228. A method of masking a cell from an adaptive immune response comprising contacting a composition of any one of embodiments 1-197, 213 or 227 to the cell to produce a modified cell, wherein the composition modifies a level of expression of an RNA molecule of the modified cell and wherein the RNA molecule encodes a component of an adaptive immune response.
Embodiment 229. The method of embodiment 228, wherein the cell is in vivo, in vitro, ex vivo or in situ.
Embodiment 230. The method of embodiment 228, wherein the cell is in vitro or ex vivo.
Embodiment 231. The method of any one of embodiments 228-230, wherein a plurality of cells comprises the cell.
Embodiment 232. The method of embodiment 231, wherein each cell of the plurality of cells contacts the composition, thereby producing a plurality of modified cells.
Embodiment 233. The method of any one of embodiments 228-230, wherein the method further comprises administering the modified cell to a subject.
Embodiment 234. The method of any one of embodiments 231-232, wherein the method further comprises administering the plurality of modified cells to a subject.
Embodiment 235. The method of embodiment 233, wherein the cell is autologous.
Embodiment 236. The method of embodiment 233, wherein the cell is allogeneic.
Embodiment 237. The method of embodiment 233, wherein the plurality of modified cells is autologous.
Embodiment 238. The method of embodiment 233, wherein the plurality of modified cells is allogeneic.
Embodiment 239. The method of any one of embodiments 228-238, wherein the component of an adaptive immune response comprises or consists of a component of a type I major histocompatibility complex (MHC I), a type II major histocompatibility complex (MHC II), a T-cell receptor (TCR), a costimulatory molecule or a combination thereof.
Embodiment 240. The method of embodiment 239, wherein the MHC I component comprises an α1 chain, an α2 chain, an α3 chain, or a β2M protein.
Embodiment 241. The method of any one of embodiments 228-238, wherein the component of an adaptive immune response comprises or consists of an MHC I β2M protein.
Embodiment 242. The method of embodiment 239, wherein the MHC II component comprises an α1 chain, an α2 chain, a β1 chain, or a β2 chain.
Embodiment 243. The method of embodiment 239, wherein the TCR component comprises an α-chain and a β-chain.
Embodiment 244. The method of embodiment 239, wherein the costimulatory molecule comprises a Cluster of Differentiation 28 (CD28), a Cluster of Differentiation 80 (CD80), a Cluster of Differentiation 86 (CD86), an Inducible T-cell COStimulator (ICOS), or an ICOS Ligand (ICOSLG) protein.
Embodiment 245, A method of preventing or reducing an adaptive immune response in a subject comprising administering a therapeutically effective amount of a composition of any one of embodiments 1-197, 213 or 227 to the subject, wherein the composition contacts at least one cell in the subject producing a modified cell, wherein the composition modifies a level of expression of an RNA molecule of the modified cell and wherein the RNA molecule encodes a component of an adaptive immune response.
Embodiment 246. A method of treating a disease or disorder in a subject comprising administering a therapeutically effective amount of a composition of any one of embodiments 1-197, 213 or 227 to the subject, wherein the composition contacts at least one cell in the subject producing a modified cell, wherein the composition modifies a level of expression of an RNA molecule of the modified cell and wherein the composition prevents or reduces an adaptive immune response to the modified cell.
Embodiment 247. The method of embodiment 246, wherein the component of an adaptive immune response comprises or consists of a component of a type I major histocompatibility complex (MHC I), a type II major histocompatibility complex (MHC II), a T-cell receptor (TCR), a costimulatory molecule or a combination thereof.
Embodiment 248. The method of embodiment 247, wherein the MHC I component comprises an α1 chain, an α2 chain, an α3 chain, or a β2M protein.
Embodiment 249. The method of embodiment 247 or 248, wherein the component of an adaptive immune response comprises or consists of an MHC I β2M protein.
Embodiment 250. The method of embodiment 249, wherein the MHC II component comprises an α1 chain, an α2 chain, a β1 chain, or a β2 chain.
Embodiment 251. The method of embodiment 247, wherein the TCR component comprises an α-chain and a β-chain.
Embodiment 252. The method of embodiment 247, wherein the costimulatory molecule comprises a Cluster of Differentiation 28 (CD28), a Cluster of Differentiation 80 (CD80), a Cluster of Differentiation 86 (CD86), an Inducible T-cell COStimulator (ICOS), or an ICOS Ligand (ICOSLG) protein.
Embodiment 253. The method of any one of embodiments 246-252, wherein the disease or disorder is a genetic disease or disorder.
Embodiment 254. The method of embodiment 253, wherein the disease or disorder is a single gene genetic disease or disorder.
Embodiment 255. The method of embodiment 254, wherein the disease or disorder results from microsatellite instability.
Embodiment 256. The method of embodiment 255, wherein the microsatellite instability occurs in a DNA sequence at least 1, 2, 3, 4, 5 or 6 repeated motifs.
Embodiment 257. The method of embodiment 256, wherein an RNA molecule comprises a transcript of the DNA sequence and wherein the composition binds to a target sequence of the RNA molecule comprising at least 1, 2, 3, 4, 5, or 6 repeated motifs.
Embodiment 258. The method of any one of embodiments 246-257, wherein the composition is administered systemically.
Embodiment 259. The method of embodiment 259, wherein the composition is administered intravenously.
Embodiment 260. The method of embodiment 258 or 259, wherein the composition is administered by an injection or an infusion.
Embodiment 261. The method of any one of embodiments 246-257, wherein the composition is administered locally.
Embodiment 262. The method of embodiment 261, wherein the composition is administered by an intraosseous, intraocular, intracerebral, or intraspinal route.
Embodiment 263. The method of embodiment 261 or 262, wherein the composition is administered by an injection or an infusion.
Embodiment 264. The method of any one of embodiments 265-263, wherein the therapeutically effective amount is a single dose.
Embodiment 265. The method of any one of embodiments 265-264, wherein the composition is non-genome integrating.

### INCORPORATION BY REFERENCE

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### OTHER EMBODIMENTS

While particular embodiments of the disclosure have been illustrated and described, various other changes and modifications can be made without departing from the spirit and scope of the disclosure. The scope of the appended claims includes all such changes and modifications that are within the scope of this disclosure.

1. A composition comprising a nucleic acid sequence comprising a guide RNA (gRNA) sequence that specifically binds a target RNA sequence, wherein the target RNA sequence encodes a protein component of an adaptive immune response, and wherein the gRNA sequence comprises a spacer sequence comprising a portion of a nucleic acid sequence encoding the protein component, and wherein the protein component is selected from the group consisting of Beta-2-microglobulin (β2M), Human Leukocyte Antigen A (HLA-A), Human Leukocyte Antigen B (HLA-B), Human Leukocyte Antigen C (HLA-C), Cluster of Differentiation 28 (CD28), Cluster of Differentiation 80 (CD80), Cluster of Differentiation 86 (CD86), Inducible T-cell Costimulator (ICOS), ICOS Ligand (ICOSLG), OX40L, Interleukin 12 (IL12), and CC Chemokine Receptor 7 (CCR7).

2. The composition of claim 1, wherein the adaptive immune response is selected from the group consisting of type I major histocompatibility complex (MHC I), type II major histocompatibility complex (MHC II), T-cell receptor (TCR), costimulatory molecule and a combination thereof.

3. (canceled)

4. The composition of claim 1, wherein the spacer sequence and the target RNA sequence are reverse complements of one another.

5. The composition of claim 1, wherein the gRNA sequence comprises a scaffold sequence that specifically binds to a CRISPR/Cas polypeptide or portion thereof.

6. The composition of claim 5, wherein the CRISPR/Cas polypeptide or portion thereof is selected from the group consisting of Cas9, Cpf1, Cas13a, Cas13b, Cas13c and CasRX/Cas13d, wherein the CRISPR/Cas polypeptide has native, reduced or null activity.

7. The composition of claim 1, wherein the nucleic acid sequence comprises a promoter which drives expression of the gRNA sequence.

8. The composition of claim 7, wherein the promoter is selected from the group consisting of a polymerase III promoter and a tRNA promoter.

9. (canceled)

10. The composition of claim 1, wherein the spacer sequence is a first spacer sequence that specifically binds a first target RNA sequence, and wherein the composition further comprises a second spacer sequence which specifically binds a second target RNA sequence, wherein the first spacer sequence and the second spacer sequence bind different target RNA sequences.

11. The composition of claim 10, wherein the gRNA sequence is a first gRNA sequence, and wherein the second spacer sequence is comprised within a second gRNA sequence.

12. The composition of claim 10, wherein the second target RNA sequence encodes a protein component of an adaptive immune response.

13. The composition of claim 10, wherein the second spacer sequence comprises a portion of a nucleic acid sequence encoding a protein component is selected from the group consisting of Beta-2-microglobulin (β2M), Human Leukocyte Antigen A (HLA-A), Human Leukocyte Antigen B (HLA-B), Human Leukocyte Antigen C (HLA-C), Cluster of Differentiation 28 (CD28), Cluster of Differentiation 80 (CD80), Cluster of Differentiation 86 (CD86), Inducible T-cell Costimulator (ICOS), ICOS Ligand (ICOSLG), OX40L, Interleukin 12 (IL12), and CC Chemokine Receptor 7 (CCR7).

14. The composition of claim 10, wherein the second spacer sequence comprises at least 1, 2, 3, 4, 5, 6, or 7 repeats of a nucleic acid sequence selected from the group consisting of: CUG (SEQ ID NO: 18), CCUG (SEQ ID NO: 19), CAG (SEQ ID NO: 80), GGGGCC (SEQ ID NO: 81), and a combination thereof.

15. A composition comprising a nucleic acid sequence comprising: (a) a first guide RNA (gRNA) sequence that specifically binds a first target RNA sequence, and (b) a second gRNA that specifically binds a second target RNA sequence, wherein the first target RNA sequence encodes a protein component of an adaptive immune response, and wherein the first gRNA sequence comprises a spacer sequence comprising a portion of a nucleic acid sequence encoding the protein component, and wherein the protein component is selected from the group consisting of Beta-2-microglobulin (β2M), Human Leukocyte Antigen A (HLA-A), Human Leukocyte Antigen B (HLA-B), Human Leukocyte Antigen C (HLA-C), Cluster of Differentiation 28 (CD28), Cluster of Differentiation 80 (CD80), Cluster of Differentiation 86 (CD86), Inducible T-cell Costimulator (ICOS), ICOS Ligand (ICOSLG), OX40L, Interleukin 12 (IL12), and CC Chemokine Receptor 7 (CCR7).

16. (canceled)

17. (canceled)

18. A composition comprising a nucleic acid sequence comprising: (a) a first guide RNA (gRNA) that specifically binds a first target RNA sequence within a first RNA molecule, wherein the first target RNA sequence encodes a protein component of an adaptive immune response (b) a second guide RNA (gRNA) that specifically binds a second target RNA sequence within a second RNA molecule and (c) a nucleic acid sequence encoding a fusion protein, wherein the fusion protein comprises a first RNA-binding polypeptide a second RNA-binding polypeptide, wherein neither the first RNA-binding polypeptide nor the second RNA-binding polypeptide comprises a significant DNA-nuclease activity, wherein the first RNA-binding polypeptide and the second RNA-binding polypeptide are not identical, and wherein the second RNA-binding polypeptide comprises an RNA-nuclease activity.

19.-26. (canceled)

27. A vector comprising the composition of claim 18.

28. The vector of claim 27, wherein the vector is selected from the group consisting of: adeno-associated virus, retrovirus, lentivirus, adenovirus, nanoparticle, micelle, liposome, lipoplex, polymersome, polyplex, and dendrimer.

29. A cell comprising the vector of claim 28.

30. The composition of claim 18, wherein the second RNA-binding polypeptide is selected from the group consisting of: RNAse1, RNAse4, RNAse6, RNAse7, RNAse8, RNAse2, RNAse6PL, RNAseL, RNAseT2, RNAse11, RNAseT2-like, NOB1, ENDOV, ENDOG, ENDOD1, hFEN1, hSLFN14, hLACTB2, APEX2, ANG, HRSP12, ZC3H12A, RIDA, PDL6, NTHL, KIAA0391, APEX1, AGO2, EXOG, ZC3H12D, ERN2, PELO, YBEY, CPSF4L, hCG_2002731, ERCC1, RAC1, RAA1, RAB1, DNA2, FLJ35220, FLJ13173, ERCC4, RNAse1(K41R), RNAse1(K41R, D121E), RNAse1(K41R, D121E, H119N), RNAse1(H119N), RNAse1(R39D, N67D, N88A, G89D, R91D, H119N), RNAse1(R39D, N67D, N88A, G89D, R91D, H119N, K41R, D121E), RNAse1(R39D, N67D, N88A, G89D, R91D), TENM1, TENM2, RNAseK, TALEN, ZNF638, and hSMG6 PIN.
